(19) United States
(12) Patent Application Publication
(10) Pub. No.: US 2019/0308031 A1
(43) Pub. Date: Oct. 10, 2019

(54) SKIN DISORDER THERAPEUTICS AND METHODS OF USE

(71) Applicant: NAKED BIOME, INC., San Francisco, CA (US)

(72) Inventors: Emma TAYLOR, San Francisco, CA (US); David HANZEL, San Francisco, CA (US)

(21) Appl. No.: 16/095,275

(22) PCT Filed: Apr. 21, 2017
(86) PCT No.: PCT/US2017/028912

§ 371 Date: Oct. 19, 2018

Related U.S. Application Data
(60) Provisional application No. 62/325,829, filed on Apr. 21, 2016.
(60) Provisional application No. 62/385,837, filed on Sep. 9, 2016.
(60) Provisional application No. 62/441,923, filed on Jan. 3, 2017.

(51) Int. Cl. A61N 5/06; A61K 41/00; A61N 1/18

(52) U.S. Cl. CPC A61N 5/0616; A61N 2005/067; A61N 1/18; A61K 41/0019

(57) ABSTRACT

Disclosed and methods for light-based treatment of skin disorders such as acne. Also disclosed are devices for providing said light-based treatments. These treatments can be used in conjunction with and augmented by topical formulations of health-associated bacteria.

### CROSS-REFERENCE TO RELATED APPLICATIONS

This application is a U.S. National Phase of International Application No. PCT/US2017/028912 filed Apr. 21, 2017, which claims the benefit of U.S. Provisional Ser. Nos. 62/325,829 filed on Apr. 21, 2016; 62/385,837 filed on Sep. 9, 2016; and 62/441,923 filed on Jan. 3, 2017 all of which are incorporated herein in their entirety.

### SEQUENCE LISTING

The instant application contains a Sequence Listing which has been filed electronically in ASCII format and is hereby incorporated by reference in its entirety. Said ASCII copy, created on Apr. 19, 2017, is named 48236-704_601_SL.txt and is 98,280 bytes in size.

### BACKGROUND OF THE INVENTION

Acne vulgaris, or simply acne, is a relatively common chronic inflammatory skin disorder that occurs when follicles on the surface of the skin become blocked, forming a plug or comedone. Acne affects an estimated 80-90% of adolescents, as well as adults of all ages. Despite the clinical importance of the disease, the etiology of acne is not yet clear. Nevertheless, a variety of conditions have been identified as associated with acne, including: a higher than normal amount of sebum production, excessive deposition of keratin, a local release of pro-inflammatory chemicals in the skin, and colonization of follicles by certain strains of Propionibacterium acnes (P. acnes).

### SUMMARY

Propionibacterium acnes is an important skin commensal, but it is also considered a pathogenic factor in several diseases including acne vulgaris. Type IA-2 (primarily ribotype 4 [RT4] and RT5) strains have been associated with acne, while type II strains, in particular RT6 and some RT2 strains, have rarely been found in acneic skin or are considered low porphyrin producers and thus are defined as health-associated strains in the context of acne. Implicated in the difference between health- and disease-associated strains of P. acnes are porphyrins—a group of pro-inflammatory metabolites important in acne development. Disease-associated P. acnes have been found to inherently produce significantly higher levels of these porphyrins than healthy-associated P. acnes. Studies have shown correlations between acne severity and porphyrin producing bacteria in follicles. Further, laser therapy that targets porphyrin chromophores within bacteria has been shown to reduce acne severity.

P. acnes porphyrins, such as coproporphyrin III and protoporphyrin IX, absorb light at wavelengths between 400 and 700 nm. Light absorption leads to photo-excitation of the porphyrins and subsequent release of singlet oxygen and reactive free radicals that exert bactericidal effects on P. acnes. Specifically, coproporphyrin III has an absorption spectrum around 399-415 nm, while protoporphyrin has an absorption spectrum around 633 nm. Accordingly, exposure of P. acnes porphyrins to specific wavelengths of light is a viable method for reducing P. acnes and treating acne. The light therapy methods disclosed herein may be used for a wide range of skin disorders including acne, eczema, psoriasis, rosacea and seborrheic dermatitis. The methods described herein comprise light treatments that are advantageous because the light therapy selectively targets high porphyrin producers and leaves healthy strains remaining, and this light therapy is coupled with adding back healthy strains (i.e. that are selected to be low porphyrin producers).

In one aspect of the disclosure is a method for treating an individual affected with disease-associated bacteria, the method comprising exposing an affected site of the individual to electromagnetic radiation, wherein the exposure of the affected site specifically reduces the amount of the disease-associated bacteria; followed by administering a topical formulation of health-associated bacteria. The disease associated bacteria in this aspect are defined as disease-associated by virtue of high production/secretion of disease associated metabolites, such as for example, porphyrin. Porphyrin can be excited using the light therapy methods described herein specifically killing bacteria that produce/secrete high levels. Treatment using the health-associated or probiotic bacteria described herein can prevent the regrowth of these disease causing bacteria, therefore treating and reducing the appearance of a skin disorder, such as, for example, acne, eczema, psoriasis, rosacea and seborrheic dermatitis. In certain embodiments, the health associated comprise bacteria that are low porphyrin secretors or producers and produce less than about 4, 3, 2, 1, 0.5, or 0.25 micromolar of porphyrin. In certain embodiments, the bacteria produce porphyrin at a level less than or about equal to that of P. acnes strains HL042PA3, HL103PA1, or HL001PA1.

In one aspect of the disclosure, provided herein is method for treating an individual affected with acne-associated bacteria, the method comprising exposing an affected site of the individual to electromagnetic radiation at a wavelength from about 300 nm to about 1400 nm; provided that the wavelength corresponds to a wavelength between about 90 nm below and about 90 nm above an absorption maxima of a metabolite from the acne-associated bacteria; and provided that the electromagnetic radiation is emitted from a source having a power output from about 0.5 mW to about 20 mW. In some embodiments, the affected site comprises skin, pilosebaceous glands, sebaceous oil glands, sebaceous ducts, sebocytes, or a combination thereof. In some embodiments, the acne-associated bacteria have been demonstrated to be present with individuals having acne, acne skin, acne lesions, or a combination thereof. In some embodiments, the acne-associated bacteria is a P. acnes Type IA clade comprising P. acnes ribotype 4, P. acnes ribotype 5 P. acnes ribotype 1, P. acnes ribotype 3, P. acnes ribotype 7, P. acnes ribotype 8, P. acnes ribotype 9, P. acnes ribotype 10, or a combination thereof. In some embodiments, that the metabolite from the acne-associated bacteria has a concentration within the affected site greater than a threshold concentration of the metabolite. In some embodiments, the threshold concentration is about 4 micromolar. In some embodiments, the metabolite from the acne-associated bacteria has a concentration within the affected site greater than a concentration of the metabolite from a health-associated bacteria within the affected site.

In some embodiments, the affected site is exposed to narrowband electromagnetic radiation. In some embodiments, the affected site is exposed to a range of wavelengths spanning less than about 50 nm. In some embodiments, the affected site is exposed to a range of wavelengths spanning less than about 20 nm. In some embodiments, the affected site is exposed to a range of wavelengths spanning less than about 10 nm. In some embodiments, the electromagnetic radiation source has a power output from about 1 mW to about 10 mW. In some embodiments, the electromagnetic radiation source has a power output of about 4 mW.

In some embodiments, the metabolite has an absorption maxima between about 390 nm and about 1300 nm. In some embodiments, the metabolite has an absorption maxima between about 390 nm and about 700 nm. In some embodiments, the metabolite is a porphyrin. In some embodiments, the porphyrin is coproporphyrin III, protoporphyrin IX, or a combination thereof. In some embodiments, the metabolite has an absorption maxima between about 390 nm and about 420 nm.

In some embodiments, the affected site is exposed to electromagnetic radiation at a wavelength of 400 nm, 420 nm, 430 nm, 445 nm, 475 nm, 590 nm, 635 nm, 655 nm, 660 nm, 670 nm, 780 nm, 785 nm, 810 nm, 830 nm, 840 nm, 860 nm, 904 nm, 915 nm, 980 nm, 1015 nm, 1060 nm, 1260 nm, 1400 nm, or a combination thereof. In some embodiments, the electromagnetic radiation source has an energy output of about 4 to about 90 mW/cm². In some embodiments, the electromagnetic radiation source comprises a light emitting diode. In some embodiments, the electromagnetic radiation source comprises a light emitting diode that emits blue light, red light, or both blue light and red light. In some embodiments, the electromagnetic radiation source comprises a light emitting diode having a dominant wavelength selected from the group of 400 nm, 420 nm, 430 nm, 445 nm, 475 nm, 590 nm, 635 nm, 655 nm, 660 nm, 670 nm, 780 nm, 785 nm, 810 nm, 830 nm, 840 nm, 860 nm, 904 nm, 915 nm, 980 nm, 1015 nm, 1060 nm, 1260 nm, and 1400 nm.

In some embodiments, the affected site is exposed to one or more doses of the electromagnetic radiation during a treatment period. In some embodiments, the treatment period is from about 1 day to about 3 months. In some embodiments, the treatment period is from about 1 week to about 12 weeks. In some embodiments, each dose is applied from about 1 to about 24 times during the treatment period. In some embodiments, each dose is applied about once every 1-4 weeks during the treatment period. In some embodiments, each dose is applied about once every 2-4 weeks for a treatment period of about 8-12 weeks. In some embodiments, each dose exposes the affected site to about 2 J/cm² to about 325 J/cm² of energy. In some embodiments, the electromagnetic radiation source is positioned within 5 cm of a surface of the affected site.

In some embodiments, the affected site is exposed to a dose of the electromagnetic radiation in one or more intermittent pulses. In some embodiments, the dose comprises from about 2 to about 2000 pulses. In some embodiments, the dose comprises from about 2 to about 200 pulses. In some embodiments, the dose comprises about 100 pulses. In some embodiments, each pulse is applied to the affected site for a period of between about 1 millisecond and about 1000 milliseconds. In some embodiments, each pulse is applied to the affected site for a period of between about 100 milliseconds and about 500 milliseconds. In some embodiments, each pulse is applied to the affected site for a period of about 250 milliseconds. In some embodiments, each pulse is separated from a next pulse by a time period of from about 1 millisecond to about 1000 milliseconds. In some embodiments, each pulse is separated from a next pulse by a time period of from about 50 milliseconds to about 500 milliseconds. In some embodiments, each pulse is separated from a next pulse by about 100 milliseconds.

In some embodiments, the affected site is exposed to a continuous dose of the electromagnetic radiation for a duration of about 0.5 minutes to about 60 minutes. In some embodiments, the affected site is exposed to a continuous dose of the electromagnetic radiation for a duration of about 0.5 minutes to about 30 minutes. In some embodiments, the affected site is exposed to a continuous dose of the electromagnetic radiation for a duration of about 15 minutes.

In some embodiments, the affected site of the individual comprises an acne lesion or has a history of comprising an acne lesion; and provided that the average number of acne lesions in the affected site, after exposure of the affected site to the electromagnetic radiation, is less than the average number of acne lesions in the affected site prior to the electromagnetic radiation exposure. In some embodiments, the average number of acne lesions in the affected site of the individual is decreased by at least about 30% after the electromagnetic radiation exposure. In some embodiments, the acne lesions comprise inflammatory acne lesions, non-inflammatory acne lesions, or a combination thereof. In some embodiments, the colony count of viable acne-associated bacteria within the affected site is decreased after the affected site is exposed to the electromagnetic radiation. In some embodiments, the colony count of viable acne-associated bacteria within the affected site after exposure to the electromagnetic radiation decreases by at least about 50%. In some embodiments, the affected site of the individual comprises health-associated bacteria; and provided that the colony count of health-associated bacteria is not decreased or is decreased by less than about 50% after exposure of the affected site to the electromagnetic radiation. In some embodiments, the number of acne flares in the affected site in one month following exposure to the electromagnetic radiation is less than the number of acne flares in the affected site in the month prior to the electromagnetic radiation exposure. In some embodiments, the number of acne flares following exposure to the electromagnetic radiation is less than or equal to about 2. In some embodiments, the electromagnetic radiation is not cytotoxic to host cells in the affected site.

In some embodiments, the method further comprises administering a probiotic to the individual. In some embodiments, the probiotic is applied to the affected site. In some cases, the individual is one who has undergone a treatment using radiation therapy as described elsewhere herein. In some embodiments, the probiotic is applied topically or by microinjection. In some embodiments, the probiotic is orally administered to the individual. In some embodiments, the probiotic comprises a health-associated bacteria. In some embodiments, 10⁴ to 10¹⁰ of the health-associated bacteria is administered to the individual. In some embodiments, the health-associated bacteria belong to P. acnes Type II clade. In some embodiments, the health-associated bacteria comprises P. acnes ribotype 1, P. acnes ribotype 2, P. acnes ribotype 6, or a combination thereof. In some embodiments, the probiotic comprises an engineered, transformed or selected bacteria. In some embodiments, the probiotic is administered within about 1 min to about 24 hours after exposure of the affected site to the electromagnetic radiation; and provided that the probiotic is optionally administered in one or more additional doses after the exposure. In some embodiments, provided that the affected site of the individual comprises health-associated bacteria; the colony count of the health-associated bacteria in the affected site increases after administration of the probiotic. In some embodiments, provided that the affected site of the individual comprises health-associated bacteria; the colony count of health-associated bacteria is not decreased or is decreased by less than about 50% after exposure of the affected site to the electromagnetic radiation and administration of the probiotic.

In some embodiments, the method further comprises administering to the individual a photomodulation enhancing agent prior to the electromagnetic radiation exposure. In some embodiments, the photomodulation enhancing agent is applied topically to the affected site, an area surrounding the affected site, or a combination thereof. In some embodiments, the area surrounding the affected site comprises an area within about 2 cm of the affected site in any direction from the affected site. In some embodiments, the photomodulation enhancing agent is administered within the 1 hour prior to the electromagnetic radiation exposure. In some embodiments, the photomodulation enhancing agent is applied topically or by microinjection. In some embodiments, the photomodulation enhancing agent is orally administered to the individual. In some embodiments, the photomodulation enhancing agent comprises chlorophyll, carotenoid, bacteriochlorophyll, phycobilin, porphyrin, or a derivative or combination thereof. In some embodiments, the photomodulation enhancing agent comprises levulinic acid, alpha aminolevulinic acid, alpha levulinic acid, vitamin C, vitamin E, vitamin A, vitamin K, vitamin F, retin A (Tretinoin), adapalene, retinol, hydroquinone, kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, an antioxidant, a phytoanthocyanin, epigallocatechin3-ganate, a phytonutrient, a botanical product, a herbaceous product, a hormone, an enzyme, a mineral, a genetically engineered substance, a cofactor, a catalyst, an antiaging substance, insulin, trace elements, minerals, Rogaine, a hair growth stimulating substance, a hair growth inhibiting substance, a dye, a natural or synthetic melanin, a metalloproteinase inhibitor, proline, hydroxyproline, an anesthetic substance, chlorophyll, copper chlorophyllin, chloroplasts, carotenoids, bacteriochlorophyll, phycobilins, carotene, xanthophyll, anthocyanin, and derivatives and combinations thereof. In some embodiments, the photomodulation enhancing agent comprises levulinic acid, alpha aminolevulinic acid, alpha levulinic acid, and derivatives and combinations thereof. In some embodiments, the acne-associated bacteria produce an increased concentration of metabolite within the affected site after administration of the photomodulation enhancing agent. In some embodiments, the photomodulation enhancing agent is vitamin B12.

In some embodiments, the method further comprises subjecting the affected site to a penetration enhancing procedure after administration of the photomodulation enhancing agent and prior to the electromagnetic radiation exposure. In some embodiments, the penetration enhancing procedure is performed within 1 to 30 minutes after administration of the photomodulation enhancing agent, and within 10 to 30 minutes before the electromagnetic radiation exposure. In some embodiments, the penetration enhancing procedure comprises an enzyme peel, microderm abrasion, solvent stripping, tape stripping, scrubbing, laser ablation, laser vaporization, chemical peeling, electrical stimulation, laser treatments using high peak power and short pulse durations, ultrasound, or a combination thereof. In some embodiments, the penetration enhancing procedure comprises microdermabrasion. In some embodiments, the penetration enhancing procedure comprises exposing the photomodulation enhancing agent to ultrasound.

In another aspect of the disclosure, provided herein is a therapeutic device comprising a radiation source configured to emit electromagnetic radiation at a wavelength from about 300 nm to about 1400 nm, the electromagnetic radiation emitted in a narrowband of about 50 nm or less; provided that the radiation source has a power output from about 0.5 mW to about 20 mW. In some embodiments, the therapeutic device is configured for use by a practitioner, an individual as an in-home device, or a combination thereof. In some embodiments, the therapeutic device further comprises a mechanism that controls a total amount of energy emitted from the radiation source per dose. In some embodiments, the mechanism prohibits emission of energy from the radiation source beyond a threshold level designated by a prescribing practitioner. In some embodiments, the therapeutic device further comprises a control interface for a user to control operation of the radiation source. In some embodiments, the control interface provides a feature for emitting a continuous dose of radiation, a plurality of radiation pulses over the span of a dose, or a combination thereof. In some embodiments, the narrowband wavelength range is about 20 nm or less. In some embodiments, the narrowband wavelength range is about 10 nm or less. In some embodiments, the radiation source has a power output from about 1 mW to about 10 mW. In some embodiments, the radiation source has a power output of about 4 mW. In some embodiments, the radiation source is configured to emit electromagnetic radiation from about a 1 cm² to about 5 cm² region of the device. In some embodiments, the radiation source is configured to emit electromagnetic radiation from a customizable area of the device. In some embodiments, the customizable area of the device comprises a tip. In some embodiments, the customizable area has a length or diameter from about 1 mm to about 20 mm. In some embodiments, the customizable area has a length or diameter from about 1 mm, 3 mm, 5 mm, 7 mm, or 10 mm. In some embodiments, the radiation source is configured to emit electromagnetic radiation at a wavelength from about 390 nm to about 1400 nm. In some embodiments, the radiation source is configured to emit electromagnetic radiation at a wavelength of about 400 nm, 420 nm, 430 nm, 445 nm, 475 nm, 590 nm, 635 nm, 655 nm, 660 nm, 670 nm, 780 nm, 785 nm, 810 nm, 830 nm, 840 nm, 860 nm, 904 nm, 915 nm, 980 nm, 1015 nm, 1060 nm, 1260 nm, 1400 nm, or a combination thereof. In some embodiments, the radiation source is configured to emit electromagnetic radiation at a wavelength from about 390 nm to about 700 nm. In some embodiments, the radiation source is configured to emit electromagnetic radiation at a wavelength from about 390 nm to about 420 nm. In some embodiments, the radiation source comprises one or more light emitting diodes. In some embodiments, the radiation source comprises a blue light emitting diode, a red light emitting diode, or a combination thereof.

In another aspect of the disclosure, provided herein is a method of affecting the microbiome of an individual, the method comprising administering a probiotic to the individual, provided that the individual has undergone a treatment targeting a disease-associated bacteria on an affected site of the individual. In some embodiments, the probiotic is administered topically or by microinjection to: the affected site, an area within 2 cm surrounding the affected site, or a combination thereof. In some embodiments, the probiotic is administered orally. In some embodiments, the probiotic comprises an engineered bacteria. In some embodiments, 10⁴-10¹⁰ of the engineered bacteria is administered to the individual. In some embodiments, the probiotic comprises a first health-associated bacteria. In some embodiments, 10⁴-10¹⁰ of the first health-associated bacteria is administered to the individual. In some embodiments, the first health-associated bacteria belong to P. acnes Type II or Type I_{A} clade. In some embodiments, the first health-associated bacteria comprises P. acnes ribotype 1, P. acnes ribotype 2, P. acnes ribotype 6, or a combination thereof. In some embodiments, the probiotic is administered within about 1 min to about 24 hours after the treatment. In some embodiments, the probiotic is administered a plurality of times over a course of time within 1 month of the treatment. In some embodiments, the probiotic is administered once every 1, 2, 3, 4, 5, 6, or 7 days after the treatment. In some embodiments, the affected site comprises skin, pilosebaceous glands, sebaceous oil glands, sebaceous ducts, sebocytes, or a combination thereof. In some embodiments, the disease-associated bacteria belong to P. acnes Type IA clade. In some embodiments, the P. acnes Type IA clade comprises P. acnes ribotype 4, P. acnes ribotype 5 P. acnes ribotype 1, P. acnes ribotype 3, P. acnes ribotype 7, P. acnes ribotype 8, P. acnes ribotype 9, P. acnes ribotype 10, or a combination thereof. In certain embodiments, the individual is administered a topical anti-acne medication before undergoing treatment. In certain embodiments, the individual is administered a topical anti-acne medication after undergoing treatment. In certain embodiments, the topical anti-acne medication comprise benzoyl peroxide, salicylic acid, a topical antibiotic, or any combination thereof.

Provided herein are microbiome-based approaches to skin therapy using beneficial bacteria on the skin to eliminate or reduce harmful bacteria and restore skin to a healthy state. The methods and compositions disclosed herein may be used for a wide range of skin disorders including acne, eczema, psoriasis, rosacea and seborrheic dermatitis. Problems with current treatments for these disorders include antibiotic resistance, side effects, complicated regimens, and lack of long-term effectiveness. Treatments disclosed herein may provide alternatives to antibiotics, use healthy bacteria, present few side effects, with simple treatment regimens and long-term effectiveness.

Many of the compositions and methods disclosed herein comprise bacteria known as Propionibacterium acnes, abbreviated P. acnes, and uses thereof, respectively. However, other bacteria, including genetically modified strains, and uses thereof are also contemplated herein. Some strains of P. acnes used in compositions and methods disclosed herein, are referred to herein as healthy strains of P. acnes or, for simplicity, “healthy P. acnes.” Healthy strains of P. acnes generally promote skin health by preventing a skin disorder or reducing symptoms of a skin disorder. Healthy strains of P. acnes may even promote skin health by eliminating the cause of a skin disorder. Other strains of P. acnes are referred to herein as pathogenic strains or “pathogenic P. acnes.” Pathogenic P. acnes generally promotes or causes a skin disorder or symptoms thereof. Generally, pathogenic P. acnes are not used in compositions and methods described herein. Instead, methods and compositions disclosed herein may be useful in reducing or preventing growth of pathogenic P. acnes on the skin of a subject. In some cases, an amount of P. acnes can be considered healthy or pathogenic, too much or too little being desirable or undesirable. In some cases, a combination of multiple P. acnes strains confers health. Conversely, in some cases, a different combination of multiple P. acnes strains can be pathogenic. Strains of P. acnes, combinations thereof, and amounts thereof that are healthy and pathogenic are described herein, including health strains of P. avidum and P. granulosom.

In some embodiments, a colony count of the first health-associated bacteria in the affected site increases after administration of the probiotic to a level greater than before probiotic administration and after treatment. In some embodiments, provided that prior to the treatment, the affected site of the individual comprises the first health-associated bacteria; the colony count of the first health-associated bacteria is not decreased or is decreased by less than about 50% after treatment of the affected site and administration of the probiotic. In some embodiments, provided that the affected site of the individual comprised an acne lesion prior to the treatment; the average number of acne lesions in the affected site after probiotic administration is less than the average number of acne lesions in the affected site prior to the treatment. In some embodiments, the average number of acne lesions in the affected site of the individual decreases by at least about 50% after probiotic administration as compared to the average number of acne lesions before the treatment. In some embodiments, the acne lesions comprise inflammatory acne lesions, non-inflammatory acne lesions, or a combination thereof. In some embodiments, the number of acne flares in the affected site in one month following probiotic administration is less than the number of acne flares in the affected site in the month prior to the treatment. In some embodiments, the number of acne flares following probiotic administration is less than or equal to about 2. In some embodiments, the average colony count of the disease-associated bacteria in the affected site after probiotic administration is less than the average colony count of the disease-associated bacteria in the affected site prior to the treatment. In some embodiments, the average colony count of the disease-associated bacteria in the affected site of the individual decreases by at least about 50% after probiotic administration as compared to the average colony count of the disease-associated bacteria before the treatment.

In some embodiments, the treatment comprises exposing the affected site of the individual to electromagnetic radiation at a wavelength from about 300 nm to about 1400 nm. In some embodiments, the affected site is exposed to electromagnetic radiation at a wavelength from about 390 nm to about 700 nm. In some embodiments, the affected site is exposed to electromagnetic radiation at a wavelength from about 390 nm to about 420 nm. In some embodiments, the affected site is exposed to electromagnetic radiation at a wavelength of 400 nm, 420 nm, 430 nm, 445 nm, 475 nm, 590 nm, 635 nm, 655 nm, 660 nm, 670 nm, 780 nm, 785 nm, 810 nm, 830 nm, 840 nm, 860 nm, 904 nm, 915 nm, 980 nm, 1015 nm, 1060 nm, 1260 nm, 1400 nm, or a combination thereof. In some embodiments, the wavelength corresponds to a wavelength between about 90 nm below and about 90 nm above an absorption maxima of a metabolite from the disease-associated bacteria. In some embodiments, the metabolite has an absorption maxima between about 390 nm and about 1300 nm. In some embodiments, the metabolite has an absorption maxima between about 390 nm and about 700 nm. In some embodiments, the metabolite is a porphyrin. In some embodiments, the porphyrin is coproporphyrin III, protoporphyrin IX, or a combination thereof. In some embodiments, the metabolite has an absorption maxima between about 390 nm and about 420 nm. In some embodiments, the metabolite from the disease-associated bacteria has a concentration within the affected site greater than a threshold concentration of the metabolite. In some embodiments, the threshold concentration is about 4 micromolar. In some embodiments, the treatment is configured to reduce the amount of porphyrin containing or secreting bacteria by at least 10%, 25%, 50%, 75%, or 100%. In some embodiments, the treatment is configured to reduce the amount of porphyrin containing or secreting bacteria by at least 2-fold, 3-fold, 4-fold, 5-fold, 6-fold, 7-fold, 8-fold, 9-fold, 10-fold or more. In some embodiments, the treatment is configured to reduce the amount of porphyrin associated with a skin surface by at least 10%, 25%, 50%, 75%, or 100%. In some embodiments, the treatment is configured to reduce the amount of porphyrin associated with a skin surface at least 2-fold, 3-fold, 4-fold, 5-fold, 6-fold, 7-fold, 8-fold, 9-fold, 10-fold or more. Methods of measuring porphyrin associated with the skin or with bacteria on the skin are known in the art, and include, for example, facial imaging using UV light.

In some embodiments, the electromagnetic radiation is provided by a light emitting diode. In some embodiments, the electromagnetic radiation is provided by a light emitting diode that emits blue light, red light, or both blue light and red light. In some embodiments, the affected site is exposed to narrowband electromagnetic radiation. In some embodiments, the affected site is exposed to a range of wavelengths spanning less than about 50 nm. In some embodiments, the affected site is exposed to a range of wavelengths spanning less than about 20 nm. In some embodiments, the affected site is exposed to a range of wavelengths spanning less than about 10 nm. In some embodiments, the electromagnetic radiation is provided by an energy source having a power output from about 1 mW to about 20 mW. In some embodiments, the electromagnetic radiation is provided by an energy source having a power output from about 1 mW to about 10 mW. In some embodiments, the electromagnetic radiation is provided by an energy source having a power output of about 4 mW.

In some embodiments, the affected site is exposed to one or more doses of the electromagnetic radiation during a treatment period. In some embodiments, the treatment period is from about 4 weeks to about 16 weeks. In some embodiments, each dose is applied about once every 2-4 weeks during the treatment period. In some embodiments, each dose exposes the affected site to about 2 J/cm² to about 325 J/cm² of energy. In some embodiments, the electromagnetic radiation is applied from a radiation source positioned within about 5 mm of a surface of the affected site. In some embodiments, the affected site is exposed to a dose of the electromagnetic radiation in one or more intermittent pulses. In some embodiments, the dose comprises from about 2 to about 2000 pulses. In some embodiments, the dose comprises from about 2 to about 200 pulses. In some embodiments, each pulse is applied to the affected site for a period of between about 1 millisecond and about 1000 milliseconds. In some embodiments, each pulse is applied to the affected site for a period of between about 100 milliseconds and about 500 milliseconds. In some embodiments, each pulse is separated from a next pulse by a time period of from about 1 millisecond to about 1000 milliseconds. In some embodiments, each pulse is separated from a next pulse by a time period of from about 50 milliseconds to about 500 milliseconds. In some embodiments, the affected site is exposed to a continuous dose of the electromagnetic radiation for a duration of about 0.5 minutes to about 60 minutes. In some embodiments, the affected site is exposed to a continuous dose of the electromagnetic radiation for a duration of about 0.5 minutes to about 30 minutes. In some embodiments, the affected site is exposed to a continuous dose of the electromagnetic radiation for a duration of about 15 minutes. In some embodiments, the electromagnetic radiation is not cytotoxic to host cells in the affected site.

In some embodiments, the method further comprises administering to the individual a photomodulation enhancing agent prior to the electromagnetic radiation treatment. In some embodiments, the photomodulation enhancing agent is applied topically to the affected site, an area surrounding the affected site, or a combination thereof. In some embodiments, the photomodulation enhancing agent is administered within the 1 hour prior to the electromagnetic radiation treatment. In some embodiments, the photomodulation enhancing agent is applied topically or by microinjection. In some embodiments, the photomodulation enhancing agent is orally administered to the individual. In some embodiments, the photomodulation enhancing agent comprises chlorophyll, carotenoid, bacteriochlorophyll, phycobilin, porphyrin, or a derivative or combination thereof. In some embodiments, photomodulation enhancing agent comprises levulinic acid, alpha aminolevulinic acid, alpha levulinic acid, vitamin C, vitamin E, vitamin A, vitamin K, vitamin F, retin A (Tretinoin), adapalene, retinol, hydroquinone, kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, an antioxidant, a phytoanthocyanin, epigallocatechin3-ganate, a phytonutrient, a botanical product, a herbaceous product, a hormone, an enzyme, a mineral, a genetically engineered substance, a cofactor, a catalyst, an antiaging substance, insulin, trace elements, minerals, Rogaine, a hair growth stimulating substance, a hair growth inhibiting substance, a dye, a natural or synthetic melanin, a metalloproteinase inhibitor, proline, hydroxyproline, an anesthetic substance, chlorophyll, copper chlorophyllin, chloroplasts, carotenoids, bacteriochlorophyll, phycobilins, carotene, xanthophyll, anthocyanin, and derivatives and combinations thereof. In some embodiments, the photomodulation enhancing agent comprises levulinic acid, alpha aminolevulinic acid, alpha levulinic acid, and derivatives and combinations thereof.

In some embodiments, the method further comprises subjecting the affected site to a penetration enhancing procedure after administration of the photomodulation enhancing agent and prior to the electromagnetic radiation treatment. In some embodiments, the penetration enhancing procedure is performed within 1 to 30 minutes after administration of the photomodulation enhancing agent, and within 10 to 30 minutes before the electromagnetic radiation exposure. In some embodiments, the penetration enhancing procedure comprises an enzyme peel, microderm abrasion, solvent stripping, tape stripping, scrubbing, laser ablation, laser vaporization, chemical peeling, electrical stimulation, laser treatments using high peak power and short pulse durations, ultrasound, or a combination thereof. In some embodiments, the penetration enhancing procedure comprises microdermabrasion. In some embodiments, the penetration enhancing procedure comprises exposing the photomodulation enhancing agent to ultrasound. In another aspect of the disclosure, provided herein is probiotic composition to affect the microbiome of an individual, the composition comprising a therapeutically effective amount of P. acnes phylotype clade Type I_{A} or Type II bacteria; provided that the therapeutically effective amount is sufficient to provide P. acnes phylotype clade Type I_{A} or Type II bacteria to a target site of the individual at a colony count of at least about 10⁶, after the composition is administered to the individual for a therapeutically effective period of time. In some embodiments, the therapeutically effective amount is between about 10⁶ and 10⁹ P. acnes phylotype clade Type I_{A} or Type II bacteria. In some embodiments, that the target site comprises skin affected with, or previously affected with, acne. In some embodiments, the therapeutically effective period of time is from about 1 day to about 30 days. In some embodiments, the composition is a liquid composition for topical application to the target site. In some embodiments, the therapeutically effective amount is administered in about 1-3 doses daily. In some embodiments, the composition is an oral formulation. In some embodiments, the oral formulation is a gel, capsule, liquid, or capsule. In some embodiments, the therapeutically effective amount is about 1-2 daily doses, each dose having between about 10⁶ and 10⁹ P. acnes phylotype clade Type I_{A} or Type II bacteria. In some embodiments, the probiotic composition further comprises an engineered bacteria. In some embodiments, the engineered bacteria comprises an increase or decrease in expression of hyaluronidase or an increase or decrease of activity of hyaluronidase. In some embodiments, the engineered bacteria comprises the deoR gene. In some embodiments, the engineered bacteria comprises a type II lipase gene. In some embodiments, the type II lipase gene comprises gehA or gehB. In some embodiments, the engineered bacteria lacks a pIMPLE plasmid. In some embodiments, the engineered bacteria comprises a CRISPR locus or portion thereof.

Compositions and methods disclosed herein may comprise bacteria with a given genetic signature and uses thereof, respectively. While P. acnes is the bacteria primarily exemplified herein, it is contemplated that other bacteria having a particular genetic signature that is similar to healthy P. acnes could likewise be useful for compositions and methods disclosed herein. For example, many P. acnes strains that are identified as healthy herein express combination of a deoxyribose operon repressor (deoR), a type II lipase, and a CRISPR associated Cas endonuclease. Additionally, healthy P. acnes strains are generally associated with an absence or only small amounts of an extrachromosomal plasmid known in the art as pIMPLE plasmids. It has previously been reported that some strains of P. acnes harbor an extrachromosomal plasmid, given the term, “pIMPLE plasmid,” by those in the field. Such plasmids are readily found in the art. pIMPLE plasmids may have multiple open reading frames (ORFs). The presence of these aforementioned genes (e.g., deoR, lipase, Cas) and/or a low presence of pIMPLE plasmid may provide a healthy skin promoting genetic signature that can be used to identify bacteria other than P. acnes that are useful in compositions and methods of treating acnes described herein.

A method for treating an individual affected with disease-associated bacteria, the method comprising exposing an affected site of the individual to electromagnetic radiation at a wavelength from about 300 nm to about 1400 nm; provided that the wavelength corresponds to a wavelength between about 90 nm below and about 90 nm above an absorption maxima of a metabolite from the disease-associated bacteria; and provided that the electromagnetic radiation is emitted from a source having a power output from about 0.5 mW to about 20 mW. In certain embodiments, the disease-associated bacteria comprises a P. acnes ribotype 3, P. acnes ribotype 4, P. acnes ribotype 5, P. acnes ribotype 7, P. acnes ribotype 8, P. acnes ribotype 9, P. acnes ribotype 10, or a combination thereof. In certain embodiments, the disease-associated bacteria has a concentration within the affected site greater than a threshold concentration of the metabolite. In certain embodiments, the metabolite has an absorption maxima between about 390 nm and about 700 nm. In certain embodiments, the metabolite is a porphyrin. In certain embodiments, the electromagnetic radiation source comprises a light emitting diode that emits blue light, red light, or both blue light and red light. In certain embodiments, the electromagnetic radiation source comprises a light emitting diode having a dominant wavelength selected from the group of 390 nm, 400 nm, 420 nm, 430 nm, 445 nm, 475 nm, 590 nm, 635 nm, 655 nm, 660 nm, 670 nm, 780 nm, 785 nm, 810 nm, 830 nm, 840 nm, 860 nm, 904 nm, 915 nm, 980 nm, 1015 nm, 1060 nm, 1260 nm, and 1400 nm. In certain embodiments, the affected site is exposed to from about 1 to about 24 doses of the electromagnetic radiation during a treatment period spanning from about 1 day to about 24 weeks. In certain embodiments, each dose exposes the affected site to about 2 J/cm² to about 325 J/cm² of energy. In certain embodiments, the affected site is exposed to the electromagnetic radiation in one or more intermittent pulses or continuously for a set period of time. In certain embodiments, the exposure of the affected site specifically reduces the amount of the disease-associated bacteria by 2-fold. In certain embodiments, the method further comprises administering a composition comprising a health-associated bacteria to the individual. In certain embodiments, the health-associated bacteria comprises a P. acnes ribotype 1, a P. acnes ribotype 2, or a combination thereof. In certain embodiments, the health-associated bacteria comprises at least one gene encoding at least one of a deoxyribose operon repressor and a type II lipase, and less than about 10% pIMPLE plasmid. In certain embodiments, the health-associated bacteria comprises less than about 1% pIMPLE plasmid. In certain embodiments, the health-associated bacteria does not comprises a P. acnes ribotype 6. In certain embodiments, the health-associated bacteria does not comprise a Cas5 protein. In certain embodiments, the health-associated bacteria comprises a mixture of health-associated bacteria wherein the mixture comprises two separate strains of health-associated bacteria. In certain embodiments, the health-associated bacteria are administered after exposing the affected site of the individual to electromagnetic radiation. In certain embodiments, 10⁴-10¹⁰ colony forming units of the health-associated bacteria is administered to the individual. In certain embodiments, a photomodulation enhancing agent is administered prior to the electromagnetic radiation exposure. In certain embodiments, a penetration enhancing procedure is conducted or administered prior to the electromagnetic radiation exposure. In certain embodiments, the health-associated bacteria is a low porphyrin producer. In certain embodiments, the health-associated bacteria produces porphyrin at a level about equal to or less than P. acnes strain HL103PA1. In certain embodiments, the health-associated bacteria is formulated as a topical composition. In certain embodiments, the health-associated bacteria is formulated with an excipient or biological stabilizer. In certain embodiments, the bacteria is genetically engineered to be a low porphyrin producer.

In one aspect, described herein, is a method for treating an individual affected with disease-associated bacteria, the method comprising: exposing an affected site of the individual to electromagnetic radiation at a wavelength from about 300 nm to about 1400 nm; wherein the wavelength corresponds to a wavelength between about 90 nm below and about 90 nm above an absorption maxima of a metabolite from the disease-associated bacteria; wherein the electromagnetic radiation is emitted from a source having a power output from about 0.5 mW to about 20 mW, wherein the exposure of the affected site specifically reduces the amount of the disease-associated bacteria; and administering a topical formulation of health-associated bacteria. In certain embodiments, the disease-associated bacteria comprises a P. acnes ribotype 3, P. acnes ribotype 4, P. acnes ribotype 5, P. acnes ribotype 7, P. acnes ribotype 8, P. acnes ribotype 9, P. acnes ribotype 10, or a combination thereof. In certain embodiments, the disease-associated bacteria has a concentration within the affected site greater than a threshold concentration of the metabolite. In certain embodiments, the metabolite has an absorption maxima between about 390 nm and about 700 nm. In certain embodiments, the metabolite is a porphyrin. In certain embodiments, the electromagnetic radiation source comprises a light emitting diode that emits blue light, red light, or both blue light and red light. In certain embodiments, the electromagnetic radiation source comprises a light emitting diode having a dominant wavelength selected from the group of 390 nm, 400 nm, 420 nm, 430 nm, 445 nm, 475 nm, 590 nm, 635 nm, 655 nm, 660 nm, 670 nm, 780 nm, 785 nm, 810 nm, 830 nm, 840 nm, 860 nm, 904 nm, 915 nm, 980 nm, 1015 nm, 1060 nm, 1260 nm, and 1400 nm. In certain embodiments, the affected site is exposed to from about 1 to about 24 doses of the electromagnetic radiation during a treatment period spanning from about 1 day to about 24 weeks. In certain embodiments, each dose exposes the affected site to about 2 J/cm² to about 325 J/cm² of energy. In certain embodiments, the affected site is exposed to the electromagnetic radiation in one or more intermittent pulses or continuously for a set period of time. In certain embodiments, the exposure of the affected site specifically reduces the amount of the disease-associated bacteria by 2-fold. In certain embodiments, the health-associated bacteria comprises a P. acnes ribotype 1, a P. acnes ribotype 2, or a combination thereof. In certain embodiments, the health-associated bacteria comprises at least one gene encoding at least one of a deoxyribose operon repressor and a type II lipase, and less than about 10% pIMPLE plasmid. In certain embodiments, the health-associated bacteria comprises less than about 1% pIMPLE plasmid. In certain embodiments, the health-associated bacteria does not comprises a P. acnes ribotype 6. In certain embodiments, the health-associated bacteria does not comprise a Cas5 protein. In certain embodiments, the health-associated bacteria comprises a mixture of health-associated bacteria wherein the mixture comprises two separate strains of health-associated bacteria. In certain embodiments, the health-associated bacteria are administered after exposing the affected site of the individual to electromagnetic radiation. In certain embodiments, 10⁴-10¹⁰ colony forming units of the health-associated bacteria is administered to the individual. In certain embodiments, a photomodulation enhancing agent is administered prior to the electromagnetic radiation exposure. In certain embodiments, a penetration enhancing procedure is conducted or administered prior to the electromagnetic radiation exposure. In certain embodiments, the health-associated bacteria is a low porphyrin producer. In certain embodiments, the health-associated bacteria produces porphyrin at a level about equal to or less than P. acnes strain HL103PA1. In certain embodiments, the health-associated bacteria is formulated as a topical composition. In certain embodiments, the health-associated bacteria is formulated with an excipient or biological stabilizer. In certain embodiments, the bacteria is genetically engineered to be a low porphyrin producer.

In one aspect, described herein, is a method of affecting the microbiome of an individual, the method comprising administering a health-associated bacteria to the individual, provided that the individual has undergone a treatment targeting a disease-associated bacteria on an affected site of the individual. In certain embodiments, 10⁴-10¹0 colony forming units of the health-associated bacteria is administered to the individual. In certain embodiments, the treatment comprises exposing the affected site of the individual to electromagnetic radiation at a wavelength from about 300 nm to about 1400 nm. In certain embodiments, the wavelength corresponds to a wavelength between about 90 nm below and about 90 nm above an absorption maxima of a metabolite from the disease-associated bacteria. In certain embodiments, the method comprises administering to the individual a photomodulation enhancing agent and/or performing a penetration enhancing procedure prior to the electromagnetic radiation treatment. In certain embodiments, the health-associated bacteria comprises a P. acnes ribotype 1, a P. acnes ribotype 2, or a combination thereof. In certain embodiments, the health-associated bacteria comprises at least one gene encoding at least one of a deoxyribose operon repressor and a type II lipase, and less than about 10% pIMPLE plasmid. In certain embodiments, the health-associated bacteria comprises less than about 1% pIMPLE plasmid. In certain embodiments, the health-associated bacteria does not comprises a P. acnes ribotype 6. In certain embodiments, the health-associated bacteria does not comprise a Cas5 protein. In certain embodiments, the health-associated bacteria comprises a mixture of health-associated bacteria wherein the mixture comprises two separate strains of health-associated bacteria. In certain embodiments, the health-associated bacteria is a low porphyrin producer. In certain embodiments, the health-associated bacteria produces porphyrin at a level about equal to or less than P. acnes strain HL103PA1. In certain embodiments, the health-associated bacteria is formulated as a topical composition. In certain embodiments, the health-associated bacteria is formulated with an excipient or biological stabilizer. In certain embodiments, the bacteria is genetically engineered to be a low porphyrin producer.

### BRIEF DESCRIPTION OF THE DRAWINGS

FIG. 1 shows the percentage of reads from a sample of a healthy volunteer (free of acne) that map to P. acnes to ribotype RT1, which are both deoR+ and type II lipase positive, versus the percentage of reads from the samples that map to P. acnes RT2.

FIG. 2 shows results of an assay for P. acnes viability under different preservation conditions.

FIG. 3 shows a portion of a 23S, ribosomal RNA sequence from bacteria commonly found on the human face that enables characterization of a subject's skin microbiome. Numbers listed to the left of sequences correspond to bacterial strains as follows: (1) P. acnes_KPA171202RT1_2; (2) P. acnes_KPA171202_RT1_3; (3) P. acnes ATCC 11828_RT2_1; (4) P. acnes ATCC 11828_RT2_2; (5) P. avidum 44067; (6) P. acidipropionici ATCC 4875; (7) S. aureus 04-02981; (8) S. aureus Bmb9393; (9) S. aureus FDA209P; (10) S. epidermidis ATCC 12228; and (11) S. epidermidis PM221. Sequences 1-5 correspond to SEQ ID NOs: 20 to 24. Sequence 6 corresponds to SEQ ID NO: 25. Sequences 7-11 correspond to SEQ ID NOs: 26-30.

FIG. 4 shows a standard curve generated with serial dilutions of a combination of health-associated P. acnes and S. epidermidis that can be used to quantitate a percentage of health-associated P. acnes in a collected sample.

FIG. 5 shows qPCR of successful CRISPR editing in P. acnes.

FIG. 6 shows mutations in a gene encoding a P. acnes type I lipase that result in a gene encoding a P. acnes type II lipase. Type I lipase Intergenic Region corresponds to SEQ ID NO: 15. Type I lipase Second Lipase (region) (HMPREF0675_4856) corresponds to SEQ ID NO: 16. Type II lipase Intergenic Region corresponds to SEQ ID NO: 17. Type II lipase Second Lipase (region) (HMPREF0675_4856) corresponds to SEQ ID NO: 18.

### DETAILED DESCRIPTION OF THE INVENTION

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by about 10%, 5%, or 1%.

As used herein “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating skin disorders like acen, eczema, psoriasis, and rosacea.

As used herein “health-associated” means a microbe that is more prevalent in healthy or disease-free individuals than in individuals diagnosed with a given disorder. In certain embodiments, the disease may be acne, eczema, psoriasis, rosacea, or any combination thereof. A health-associated microbe can be determined statistically. For example, by comparing the microbiome or the prevalence of certain microbial species or strains on the skin, in the oral cavity, or in the digestive system of a healthy non-disease afflicted individual as compared to an individual with a given disease. Genera, species, or strains that are more prevalent in/on healthy individuals are health-associated. A health-associated strain can also be a strain that has been genetically modified or selected to express low levels of virulence factors that are associated with a given skin disease. A health-associated strain can also be a strain that has been genetically modified or selected to express high levels of beneficial genes, RNAs, or proteins that are associated with protection from a given skin disease. A health-associated strain can also be a strain that has been genetically modified or selected to express, not express, or express desirable levels of markers, as described herein.

As used herein a “probiotic′ is a microbe that provides health benefits when consumed or applied. In some instances, probiotics disclosed herein are microbes that inhibit the activity or growth of a disease causing bacteria associated with acne, eczema, psoriasis, or rosacea.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

Strains of Propionibacterium acnes, HP3A11, HP4G1 and HP5G4, described herein, were deposited in the American Tissue Culture Collection (10801 University Boulevard Manassas, Va. 20110-2209 USA) on Apr. 6, 2017 in accordance with and under the provisions of the Budapest Treaty for the International Recognition of the Deposit of Microorganisms for the Purpose of Patent Procedure.

Provided throughout this application are compositions and methods for the treatment of skin disorders. It should be understood that compositions disclosed herein may be used according to methods described herein. Conversely, methods disclosed herein may appropriately employ compositions disclosed herein.

In one aspect of the disclosure, described herein are methods of treating disease or disorder related to bacteria on an individual in need thereof, by applying selective wavelengths of electromagnetic radiation to the site of the individual affected with the disease or disorder to target the bacteria. Specifically, bacteria associated with a skin disorder or disease are candidate targets for the therapeutic methods described herein. An exemplary skin disorder is acne, where certain bacteria are demonstrated to be associated with individuals having acne, for example, disease-associated strains of P. acnes. Disease-associated strains of P. acnes include, without limitation, P. acnes Type IA clade, such as, P. acnes ribotype 4, P. acnes ribotype 5, P. acnes ribotype 1, P. acnes ribotype 3, P. acnes ribotype 7, P. acnes ribotype 8, P. acnes ribotype 9, and P. acnes ribotype 10.

The wavelength of electromagnetic radiation applied to a site of an individual affected with disease-associated bacteria is often specific for the disease-associated bacteria or a biomolecule produced from said bacteria. For example, wherein the disease-associated bacteria produces a target biomolecule that elicits a bactericidal effect when exposed to radiation, electromagnetic radiation applied to the affected skin targets the biomolecule. The biomolecule is then activated to affect the disease-associated bacteria, for example, by attenuating or otherwise inhibiting the disease-causing effect of the bacteria on the individual. In some cases, the disease-associated bacteria and/or biomolecule thereof are targeted if the concentration of the biomolecule meets or exceeds a threshold level.

In certain aspects of the disclosure, electromagnetic radiation is applied to an affected site of an individual at a wavelength that corresponds to a wavelength maxima of a biomolecule of a disease-associated bacteria, or within about 90 nm below and 90 nm above the absorption maxima. In some embodiments, the electromagnetic radiation is applied at a wavelength of from about 300 nm to about 1400 nm. For cases wherein the disease-associated bacteria is that of a P. acnes strain, the wavelength maxima corresponds to the biomolecule metabolite porphyrin. As non-limiting examples, porphyrins include coproporphyrin III and protoporphyrin IX. Because health-associated P. acnes strains also comprise porphyrins, but at a lower concentration than disease-associated P. acnes, in some instances the electromagnetic radiation targets P. acnes with a porphyrin concentration at or above a given threshold. For example, a suitable threshold is about 4 micromolar porphyrin.

In order to modulate effects of electromagnetic radiation therapy on an individual as described herein, in some instances the individual is administered a photomodulation enhancing agent prior to and/or during a light therapy session. In many cases, the photomodulation enhancing agent is administered the same day as the treatment, for example, within about 10 minutes to about 4 hours prior to light therapy, about 30 minutes prior to light therapy, at least 4 hours before light therapy, at least 8 hours prior to light therapy, or at least 12 hours prior to light therapy. Wherein the photomodulation enhancing agent is a topical composition, the composition may be applied to the affected area and optionally an area within about 2 cm of the affected site. Non-limiting examples of photomodulation enhancing agents include levulinic acid, alpha aminolevulinic acid, and alpha levulinic acid. In some embodiments, a photomodulation enhancing agent enhances production of a biomolecule of disease-associated bacteria such that the light therapy readily targets the disease-associated bacteria. For example, the disease-associated bacteria is a P. acnes strain and the biomolecule is porphyrin. In addition, a photomodulation enhancing agent includes an agent that decreases intracellular Vitamin B12 production within the bacteria, shunting metabolism towards an increase in porphyrin production. In certain embodiments the a photomodulation enhancing agent includes a B12 supplement, this supplement can be a oral or topical. This may facilitate targeting of the disease-associated P. acnes strains relative to the health-associated P. acnes strains.

Electromagnetic radiation therapy methods described herein are useful for targeting disease-associated bacteria. However, in some instances, the electromagnetic radiation affects health-associated bacteria as well, albeit to a lesser degree. As such, further provided are probiotic compositions and methods of using said probiotics with individuals exposed to electromagnetic radiation. In some embodiments, health-associated bacteria are administered to the individual prior to light therapy, during the course of light therapy, and/or after light therapy. For example, about 10⁶ to about 10⁹ health-associated bacteria are topically applied to the affected site of the individual. In some cases, the disease-associated bacteria comprise one or more strains of P. acnes and the health-associated bacteria comprise one or more strains of P. acnes. In certain embodiments, the health-associated bacteria is isolated and purified. In certain embodiments, the isolated and purified bacteria are mixed with an excipient or stabilizer in order to promote storage, activity, or topical application. Non-limiting examples of health-associated P. acnes useful for probiotic compositions include P. acnes ribotype 1, 2, 3, and P. acnes ribotype 6. In certain embodiments, the health-associated bacteria is a P. acnes strain associated with skin health, oral health, digestive health, or any combination thereof. In certain embodiments, the P. acnes strain comprises a health-associated phylotype. In certain embodiments, the phylotype comprises type I, type II, or Type III, or any combination thereof. In certain embodiments, the Type I phylotype comprises IA, IB, or IC. In certain embodiments, the Type IA phylotype comprises IA₁ or IA₂. In certain embodiments, the health-associated bacteria comprise a mixture of 1, 2, 3, 4, 5, 6, 7, 8, 9, 10, or more health-associated species, strains, phylotypes, or ribotypes. In certain embodiments, the mixture may comprise an excipient or stabilizer that promotes topical application of the mixture, or activity of any of the health-associated strains in a topical formulation.

### Treatment of Acne Using Light Therapy

Therapeutic devices for emitting electromagnetic radiation are further provided herein. In general, a therapeutic device comprises a radiation source configured to emit electromagnetic radiation at a wavelength from about 300 nm to about 1400 nm, or at a wavelength suitable for uses in the therapy methods described herein. The radiation source emits a continuous dose of radiation or one or more pulses of radiation. Some devices may be configured to emit both continuous and pulsed doses of radiation, and thus more than one operation mode of the device is envisioned. Devices include both in-office devices configured for use by a practitioner such as a clinician, as well as at-home devices for use by an individual. Devices optionally comprise a fail-safe mechanism such that a user is preventing from administering more electromagnetic radiation than prescribed. In many instances, the radiation source of a device has a power output from about 0.5 mW to about 20 mW.

Methods of Treatment with Radiation Therapy

In one aspect of the disclosure, provided herein are methods for treating an individual affected with a disease-associated bacteria, the methods comprising exposing an affected site of the individual to electromagnetic radiation at a wavelength from about 300 nm to about 1400 nm. In general, the wavelength of the electromagnetic radiation emitted targets an absorption maxima of a biomolecule of the disease-associated bacteria. The electromagnetic radiation emitted further includes wavelengths between about 100 nm below and about 100 nm above the absorption maxima of the biomolecule from the disease-associated bacteria.

Methods comprising exposing an individual to electromagnetic radiation are useful for treating diseases and conditions in an individual having a disease-associated bacteria, including, without limitation, acne, sarcoidosis, SAPHO syndrome, endodontic lesions, eye infections, prosthetic joint infections, and prostate cancer. In some embodiments, the individual is affected with acne and the disease-associated bacteria includes bacteria demonstrated to be associated with individuals having acne, acneic skin and/or acne lesions. In exemplary embodiments, the acne-associated bacteria belong to P. acnes Type IA clade, which includes, without limitation, P. acnes ribotype 4, P. acnes ribotype 5, P. acnes ribotype 1, P. acnes ribotype 3, P. acnes ribotype 7, P. acnes ribotype 8, P. acnes ribotype 9, and P. acnes ribotype 10. In some embodiments, a biomolecule from the disease-associated bacteria has a concentration within the affected site greater than a threshold concentration of the biomolecule. This threshold concentration may correlate with a concentration of a biomolecule that distinguishes disease-associated bacteria from health-associated bacteria. For cases wherein the disease-associated bacteria is a strain of P. acnes, the biomolecule includes porphyrins and the threshold concentration is about 1, 2, 3, 4, 5, 6 micromolar or greater. In some cases, the threshold concentration is about 4 micromolar or greater. Non-limiting examples of porphyrins include coproporphyrin III and protoporphyrin IX. In some embodiments, the threshold concentration is measured in vitro. In some embodiments, the threshold concentration is measured in situ, due to fluorescent properties of porphyrins.

In some embodiments, after disease-associated high porphyrin producing bacteria are reduced or eliminated by electromagnetic radiation a composition comprising health-associated bacteria is administered to the individual. In some embodiments, the strain produces undetectable levels of the at least one porphyrin. Non-limiting examples of porphyrins include coproporphyrin III and protoporphyrin IX. In some embodiments, a low level of the at least one porphyrin is less than about 1 micromolar. In some embodiments, a low level of the at least one porphyrin is less than about 750 nanomolar. In some embodiments, a low level of the at least one porphyrin is less than about 500 nanomolar. In some embodiments, a low level of the at least one porphyrin is less than about 250 nanomolar. In some embodiments, a low level of the at least one porphyrin is less than about 100 nanomolar. In some embodiments, a low level of the at least one porphyrin is less than about 50 nanomolar. In some embodiments, a low level of the at least one porphyrin is less than about 10 nanomolar. In some embodiments, a low level of the at least one porphyrin is less than about 1 nanomolar. In some embodiments, the low level is a level measured in vitro. In some embodiments, the low level is a level measured in situ, due to fluorescent properties of porphyrins. In some embodiments, a strain is genetically modified to delete or mutate a nucleic acid encoding an enzyme in a porphyrin production pathway to effectively create or select for strains with low porphyrin production. In some embodiments, the strain has low lipase activity as compared to a pathogenic strain of bacteria. In some embodiments, the strain has low immunogenicity as compared to a pathogenic strain of bacteria.

In some embodiments, an individual treated with electromagnetic radiation has acne and an acne-associated bacteria, and the affected site of the individual comprises an acne lesion or has a history of having an acne lesion. Acne lesions include inflammatory acne lesions and/or non-inflammatory acne lesions. In some cases, the average number of acne lesions in the affected site, after exposure of the affected site to the electromagnetic radiation, is less than the average number of acne lesions in the affected site prior to the electromagnetic radiation exposure. As non-limiting examples, the average number of acne lesions in the affected site of the individual is decreased by at least about 10%, 20%, 30%, 40%, 50%, 60%, 70%, 80%, 90% or more after the electromagnetic radiation exposure. The time period after electromagnetic radiation exposure includes any period of time after one or more doses and/or after a treatment period. In some embodiments, the colony count of viable acne-associated bacteria within the affected site is decreased after the affected site is exposed to the electromagnetic radiation. As non-limiting examples, the colony count of viable acne-associated bacteria within the affected site after exposure to the electromagnetic radiation decreases by at least about 10%, 20%, 30%, 40%, 50%, 60%, 70%, 80%, 90% or more after the electromagnetic radiation exposure. In some embodiments, the number of acne flares in the affected site in one month following exposure to the electromagnetic radiation is less than the number of acne flares in the affected site in the month prior to the electromagnetic radiation exposure. In some embodiments, the number of acne flares in a month following the conclusion of a treatment period of exposure to electromagnetic radiation as described herein, is less than or equal to about 4, 3, 2 or 1.

In many instances, it is desirable to target disease-associated bacteria while minimizing detrimental effects to health-associated bacteria within the affected site of the individual. In some embodiments, the affected site of the individual comprises health-associated bacteria and the colony count of health-associated bacteria is not decreased or is decreased by less than about 10%, 20%, 30%, 40%, or 50% after exposure of the affected site to the electromagnetic radiation. In some embodiments, the electromagnetic radiation is not cytotoxic to host cells in and/or around the affected site and/or skin adjacent the affected site exposed to the electromagnetic radiation.

Affected sites on an individual suitable for treatment include skin, pilosebaceous glands, sebaceous oil glands, sebaceous ducts, and sebocytes. In some cases, a therapy provided herein targets skin and one or more of pilosebaceous glands, sebaceous oil glands, sebaceous ducts, and sebocytes. Skin may be located on regions of an individual suitable for exposure to electromagnetic radiation and include, without limitation, face, including forehead, checks, nose, chin; head, including hairlines and neck; back; shoulders; arms; chest; torso; legs; feet; and the like.

In some embodiments, a radiation therapy method described herein that targets disease-associated bacteria in an affected site of an individual in need thereof, further comprises administering a probiotic comprising a health-associated bacteria to the individual. In some embodiments, health-associated bacteria are administered to the individual prior to radiation therapy, during the course of radiation therapy, and/or after radiation therapy. In some cases, a probiotic is orally administered to the individual. In some cases, a probiotic is topically administered by a cream, lotion, emulsion, or microinjection. As a non-limiting example, about 10⁶ to about 10⁹ health-associated bacteria are topically applied to the affected site of the individual. In some cases, the disease-associated bacteria comprise one or more strains of health-associated bacteria. In some embodiments the health-associated bacteria comprises a Propionibacterium genus. In some embodiments, the Propionibacterium genus comprise one or more strains of P. acnes. Non-limiting examples of health-associated P. acnes useful in a probiotic composition include P. acnes in the ribotype 1, ribotype 2 or ribotype 6. Additionally, specific markers of probiotic strains are described throughout this disclosure are described specifically in Table 1. Strains of Propionibacterium acnes, HP3A11, HP4G1 and HP5G4 are also suitable to be used as probiotics in combination with the light therapy methods described herein.

Probiotics are administered, for instance, within about 1 minute to about 24 hours after exposure to a dose of electromagnetic radiation during radiation therapy, including, within about 1 min, 10 min, 20 min, 30 min, 45 min, 60 min, 90 min, 2 hrs, 4 hrs, 6 hrs, 12 hrs, and 24 hrs. In some cases, a probiotic is administered over a course of time, in one or more doses. As a non-limiting example, a probiotic is administered once or twice daily, or every other day, for the course of time. In some embodiments, after electromagnetic radiation, a colony count of health-associated bacteria in the affected site decreases. This colony count can then be recovered and/or improved by administration of the probiotic after the radiation therapy. In some embodiments, the colony count of health-associated bacteria is not decreased or is decreased by less than about 10%, 20%, 30%, 40%, or 50% after exposure of the affected site to the electromagnetic radiation and administration of the probiotic. As a comparison, for example, without probiotic administration, the colony count of the health-associated bacteria would be reduced by a greater extent, such as more than about 10%, 20%, 30%, 40%, or 50%, respectively.

Electromagnetic radiation is emitted from a radiation source configured to emit electromagnetic radiation at desired wavelengths and a given power output. In some cases, the radiation source used in methods described herein has a power output from about 0.5 mW to about 20 mW, from about 1 mW to about 10 mW, or about 1 mW, 2 mW, 3 mW, 4 mW, 5 mW, 6 mW, 7 mW, 8 mW, 9 mW or 10 mW. In some cases, a radiation source has an energy output of about 4 to about 90 mW/cm².

In order to specifically target disease-associated bacteria and/or target biomolecules thereof, in many instances electromagnetic radiation is emitted as narrowband electromagnetic radiation. This narrowband radiation includes ranges of wavelengths spanning less than about 50 nm, 40 nm, 30 nm, 20 nm, 10 nm, and 5 nm. In some cases, a radiation source is configured to emit light at a dominant wavelength of about 400 nm, 420 nm, 430 nm, 445 nm, 475 nm, 590 nm, 635 nm, 655 nm, 660 nm, 670 nm, 780 nm, 785 nm, 810 nm, 830 nm, 840 nm, 860 nm, 904 nm, 915 nm, 980 nm, 1015 nm, 1060 nm, 1260 nm, 1400 nm, or any combination thereof. A combination includes a radiation source comprised of one or more radiation sources. In some embodiments, a light emitting diode is a radiation source. For example, the light emitting diode emits blue light, red light, or both blue and red light. In some embodiments, electromagnetic radiation is applied to an affected area of an individual such that the radiation source is close in distance to the individual. This may mitigate exposure of some areas of unaffected skin to the radiation. As a non-limiting example, the radiation source is positioned less than about 5 cm, 4 cm, 3 cm, 2 cm, 1 cm, 9 mm, 8 mm, 7 mm, 6 mm, 5 mm, 4 mm, 3 mm, 2 mm, 1 mm adjacent the affected skin, or directly or in near-direct contact with the skin such that the skin is touching the radiation source.

In some embodiments, the disease-associated bacteria has a target biomolecule with an absorption maxima between about 390 nm and about 1300 nm, or between about 390 nm and about 700 nm. In some cases, the target biomolecule has an absorption maxima of about 400 nm, 420 nm, 430 nm, 445 nm, 475 nm, 590 nm, 635 nm, 655 nm, 660 nm, 670 nm, 780 nm, 785 nm, 810 nm, 830 nm, 840 nm, 860 nm, 904 nm, 915 nm, 980 nm, 1015 nm, 1060 nm, 1260 nm, 1400 nm, or a combination thereof. For instances wherein the biomolecule is the metabolite porphyrin, the biomolecule has an absorption maxima between about 390 nm and about 420 nm.

For certain methods described herein, a photomodulation enhancing agent is administered to an individual undergoing electromagnetic radiation exposure. Methods include administering a photomodulation enhancing agent to an affected site of an individual prior to exposing the site to electromagnetic radiation. A photomodulation enhancing agent is administered, for example, topically or orally, about 1 min to about 24 hours prior to radiation exposure. For topical administration, the agent is applied topically to the affected area, and optionally a perimeter of the affected area, for example, within about 2 cm of the affected area. In some cases, the photomodulation enhancing agent is administered within about 5, 15, 30, 45, 60, 90, 120, or 240 minutes prior to radiation exposure. In some cases the photomodulation enhancing agent is administered at least 2, 4, 6, 8, 12, 16, 20, or 24 hours before treatment. Non-limiting examples of photomodulation enhancing agents include levulinic acid, alpha aminolevulinic acid, alpha levulinic acid, vitamin C vitamin E, vitamin A, vitamin K, vitamin F, retin a (tretinoin), adapalene, retinol, hydroquinone, kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, an antioxidant, a phytoanthocyanin, epigallocatechin3-ganate, a phytonutrient, a botanical product, a herbaceous product, a hormone, an enzyme, a mineral, a genetically engineered substance, a cofactor, a catalyst, an antiaging substance, insulin, trace elements, minerals, Rogaine, a hair growth stimulating substance, a hair growth inhibiting substance, a dye, a natural or synthetic melanin, a metalloproteinase inhibitor, proline, hydroxyproline, an anesthetic substance, chlorophyll, copper chlorophyllin, chloroplasts, carotenoids, bacteriochlorophyll, phycobilins, carotene, xanthophyll, anthocyanin, and subcomponents, derivatives and combinations thereof. In some cases, a photomodulation enhancing agent is levulinic acid. In some cases, a photomodulation enhancing agent is alpha aminolevulinic acid. In some cases, a photomodulation enhancing agent is alpha levulinic acid. In some embodiments, a photomodulation enhancing agent comprises chlorophyll, carotenoid, bacteriochlorophyll, phycobilin, porphyrin, or a derivative or combination thereof. In some embodiments, a photomodulation enhancing agent affects a biomolecule targeted in the disease-associated bacteria. In some such cases, the biomolecule is a B12 supplement either oral or topical and, increases porphyrin levels, thereby increasing the targetability of the laser.

In some embodiments, a method for electromagnetic radiation comprises subjecting an affected site of an individual to a penetration enhancing procedure after administration of the photomodulation enhancing agent. For instance, the penetration enhancing procedure is performed within about 1-30 minutes after administration of the photomodulation enhancing agent and within about 10-30 minutes prior to radiation exposure. Non-limiting examples of penetration enhancing procedures include an enzyme peel, microderm abrasion, solvent stripping, tape stripping, scrubbing, laser ablation, laser vaporization, chemical peeling, electrical stimulation, laser treatments using high peak power and short pulse durations, ultrasound, and any combination thereof. In some embodiments, the penetration enhancing procedure comprises microdermabrasion. In some embodiments, the penetration enhancing procedure comprises exposing the photomodulation enhancing agent to ultrasound.

Methods for providing electromagnetic radiation therapies described herein are performed over one or more treatment periods. A treatment period includes a course of about 2 weeks to about 6 months, and may be repeated if necessary. In some embodiments, a treatment period spans about 2-24 weeks, about 2-18 weeks, about 2-12 weeks, about 4-24 weeks, about 4-18 weeks, about 4-12 weeks, about 6-24 weeks, about 6-12 weeks, about 8-24 weeks, or about 8-12 weeks. In some cases, the treatment is for a site of an individual affected with acne, and the treatment period spans about 8-12 weeks, or about 8, 9, 10, 11 or 12 weeks. During a treatment period a dose of electromagnetic radiation, continuous and/or pulsed, is applied to an individual one or more times. In some embodiments, a dose is applied from about 1 to about 24 times, from about 1 to about 12 times, or from about 1 to about 6 times during a treatment period. In some embodiments, a dose is applied once about every day, 2 days, 3 days, 4 days, 5 days, 6 days, 7 days, 1 week, 2 weeks, 3 weeks, 4 weeks, 5 weeks, 6 weeks, 7 weeks, or 8 weeks. In some cases, the dose frequency is modulated according to the individual and/or the status/progression/regression of the disease in the individual during treatment. Energy supplied to an affected area in each total dose includes, for example, from about 2 J/cm² to about 325 J/cm² of energy. This dose may be applied continuously and/or as multiple pulses.

Certain methods provided herein comprise treating an individual affected with a disease-associated bacteria by exposing an affected site of the individual to electromagnetic radiation at a wavelength from about 300 nm to about 1400 nm as generally described, the affected site being exposed to a dose of the electromagnetic radiation in or more intermittent pulses. In some embodiments, the affected site is exposed to about 2-2000, 2-1000, 2-500, 2-400, 2-300, 2-200, 2-100, 2-50, 10-2000, 10-1000, 10-500, 10-400, 10-300, 10-200, 10-100, 10-50, 50-500, 50-400, 50-300, 50-200, or 50-100 pulses. In some cases, the dose comprises about 10, 20, 30, 40, 50, 60, 70, 80, 90, 100, 120, or 150 pulses. The pulses are spaced apart by, for example, between about 1 ms and about 10 s. In some embodiments, two pulses in a dose are spaced apart by about 1 ms and 1 s, 1 ms and 500 ms, 10 ms and 1 s, 10 ms and 500 ms, 100 ms and 1 s, or 100 ms and 500 ms. In some cases, two pulses are spaced apart by about 100, 150, 200, 250, 300, 350, 400, 450, 500, 550, or 600 ms. In some embodiments, one or more pulses in a dose is emitted for a period of about 1 ms to 1 s, 1 ms to 500 ms, 10 ms to 1 s, 10 ms to 500 ms, 100 ms to 1 s, or 100 ms to 500 ms. In some cases, one or more pulses in a dose are emitted for a period of about 100, 150, 200, 250, 300, 350, 400, 450, 500, 550, or 600 ms.

Certain methods provided herein comprise treating an individual affected with a disease-associated bacteria by exposing an affected site of the individual to electromagnetic radiation at a wavelength from about 300 nm to about 1400 nm as generally described, the affected site being exposed to a continuous dose of the electromagnetic radiation. In some embodiments, the continuous dose is applied for a duration of about 0.5-60, 0.5-45, 0.5-30, 0.5-25, 0.5-20, 0.5-15, 0.5-10, 1-60, 1-45, 1-30, 1-25, 1-20, 1-15, 1-10, 5-60, 5-45, 5-30, 5-25, 5-20, 5-15, or 5-10 minutes. In some cases, a continuous dose is applied for a duration of about 5, 6, 7, 8, 9, 10, 11, 12, 13, 14, 15, 16, 17, 18, 19, 20, 21, 22, 23, 24, or 25 minutes.

Devices

In one aspect of the disclosure, provided herein are devices for applying selective wavelengths light to an individual in need thereof. The devices are useful, for example, in performing the electromagnetic radiation therapy methods targeting disease-associated bacteria as described herein. In certain embodiments, a device comprises a radiation source configured to emit electromagnetic radiation at a wavelength that targets the disease-associated bacteria and/or a target biomolecule thereof. As a non-limiting example, the radiation source emits light at a wavelength associated with an excitation maxima of the target bacteria and/or biomolecule thereof. Although exemplified in use with the disease-associated bacteria-target methods described herein the devices provided are not limited to such use and should not be so construed. The devices include both devices configured for use by a practitioner as well as devices configured for use at home by an individual. In both such cases, the devices optionally comprise a fail-safe mechanism to prohibit exposure to the individual at radiation levels above those prescribed. Devices further include those having radiation sources that emit electromagnetic radiation as a continuous dose and/or a dose spread out over multiple pulses.

In some embodiments, a device is provided that is configured to emit electromagnetic radiation at a wavelength from about 300 nm to about 1400 nm, from about 390 nm to about 1400 nm, from about 390 nm to about 700 nm or from about 390 nm to about 420 nm. In some cases, the radiation source is configured to emit electromagnetic radiation at a wavelength of about 400 nm, 420 nm, 430 nm, 445 nm, 475 nm, 590 nm, 635 nm, 655 nm, 660 nm, 670 nm, 780 nm, 785 nm, 810 nm, 830 nm, 840 nm, 860 nm, 904 nm, 915 nm, 980 nm, 1015 nm, 1060 nm, 1260 nm, 1400 nm, or a combination thereof.

In some embodiments, the electromagnetic radiation is emitted in a narrowband of about or less than about 100 nm, 90 nm, 80 nm, 70 nm, 60 nm, 50 nm, 40 nm, 30 nm, 20 nm, 10 nm, 5 nm or 1 nm. In some embodiments, the electromagnetic radiation source has a power output from about 0.5 mW to about 20 mW, from about 1 mW to about 10 mW, or about 1, 2, 3, 4, 5, 6, 7, 8, 9, or 10 mW. The electromagnetic radiation is emitted from one or more regions of a device. For some such instances, electromagnetic radiation is emitted from multiple areas of the device, each are having a spot size of about 3 mm, 5 mm, 7 mm, 10 mm, or the like. In some cases, electromagnetic radiation is emitted from about an 1 mm²-5 cm², 1 mm²-2 cm², 1 mm²-1 cm², 1 mm²-500 mm², 1 mm²-100 mm, 1 mm²-50 mm², 1 mm²-40 cm², 1 mm²-30 cm², 1 mm²-20 cm², or 1 mm²-10 cm² region of the device. The emitting region may comprise one or more individual areas of the same or differing sizes. In some embodiments, a device has a tip corresponding to the area of the device emitting the electromagnetic radiation. The tip size can then optionally be adjustable and/or interchangeable according to the needs of the device. In some embodiments, the radiation source comprises one or more light emitting diodes. In some cases, the radiation source comprises a blue light emitting diode. In some cases, the radiation source comprises a red light emitting diode. In other cases, the radiation source comprises both a blue and a red light emitting diode. As used herein, a light emitting diode includes one or more a plurality of light emitting diodes.

### Compositions for Treatments of Skin Disorders

In some aspects, the disclosure provides compositions comprising a strain of bacteria for use with the light therapy methods described herein, wherein the composition is formulated for application to the skin of a subject. These compositions can be applied before, during, or after the use of the light therapy methods described herein. In some embodiments, the composition comprises at least one probiotic strain of bacteria. In some embodiments, the composition comprises at least one strain of health-associated bacteria. In some embodiments, the at least one health-associated strain of bacteria is a strain of Propionibacterium bacteria. In some embodiments, the at least one health-associated strain of bacteria is a strain of Staphylococcus bacteria. In some embodiments, the at least one health-associated strain of bacteria is a strain of Lactobacillus bacteria. In some embodiments, the composition is formulated for application to the skin of a subject.

In some embodiments, compositions disclosed herein comprise a fungus. The fungus is generally a fungus that promotes, restores or improves skin health. In some embodiments, compositions disclosed herein comprise at least one health-associated fungus. In some embodiments, the fungus is Malassezia.

In some embodiments, compositions disclosed herein comprise a bacteriophage, also referred to herein as a phage for simplicity. In some embodiments, the bacteriophage is a Propionibacterium phage. In some embodiments, the bacteriophage is a health-associated Propionibacterium phage. In some embodiments, the health-associated Propionibacterium phage is a phage found on skin of a subject that is free of a skin disorder. In some embodiments, the health-associated Propionibacterium phage is a phage found on skin of a subject that is free of acne. In some embodiments, the health-associated Propionibacterium phage is a phage found on skin of a subject that is free of eczema. In some embodiments, the health-associated Propionibacterium phage is a phage found on skin of a subject that is free of psoriasis. In some embodiments, the health-associated Propionibacterium phage is a phage found on skin of a subject that is free of seborrheic dermatitis. In some embodiments, the health-associated Propionibacterium phage is a phage found on skin of a subject that is free of rosacea. In some embodiments, the health-associated Propionibacterium phage is a phage that is more abundant or prevalent on skin free of a skin disorder than skin with a skin disorder. In some embodiments, the bacteriophage is a Propionibacterium acnes phage. In some embodiments, the Propionibacterium acnes phage targets a select strain of P. acnes. In some embodiments, the Propionibacterium acnes phage targets a pathogenic strain of P. acnes. Non-limiting examples of Propionibacterium acnes phages are described in Farrar et al. (2007) J. Bacteriol., vol. 189, pp. 4161-4167, and Liu et al. (2015) ISME J, vol. 9, pp. 2078-2093.

In some embodiments, the at least one health-associated strain of bacteria is a strain of Propionibacterium acnes (P. acnes). In some embodiments, the at least one health-associated strain of bacteria is not a strain of P. acnes subsp. acnes. In some embodiments, the at least one health-associated strain of bacteria is a strain of Propionibacterium avidum (P. avidum). In some embodiments, the at least one health-associated strain of bacteria is a strain of Propionibacterium granulosum (P. granulosum). In some embodiments, the at least one health-associated strain of bacteria is a strain of Propionibacterium defendens (referred to herein as P. defendens). In some embodiments, the at least one health-associated strain of bacteria is a strain of Staphylococcus aureus. In some embodiments, the at least one health-associated strain of bacteria is a strain of Staphylococcus epidermidis. In some embodiments, the at least one health-associated strain of bacteria is a strain of Staphylococcus hominis. In some embodiments, the at least one health-associated strain of bacteria is a strain of Lactobacillus reuteri. In some embodiments, the strain of bacteria is associated with healthy skin or normal skin, and is referred to as a probiotic (or live biologic therapeutic). In some embodiments, the probiotic is associated with skin that is free of acne. In some embodiments, the probiotic is associated with skin that is essentially free of acne.

In some embodiments, a probiotic disclosed herein is selected or assessed for a composition for treatment by assessing an amount of inflammation that is caused by the probiotic to the skin of a subject. In some embodiments, the probiotic produces or induces a low amount of pro-inflammatory mediators when applied to a subject's skin. In some embodiments, the probiotic produces or induces a low amount of pro-inflammatory mediators when applied to a subject's skin. In some embodiments, the probiotic does not produce or induce pro-inflammatory mediators when applied to a subject's skin. In some embodiments, the probiotic does not produce or induce pro-inflammatory mediators when applied to a subject's skin. In some embodiments, the probiotic does not produce or induce an amount of pro-inflammatory mediators sufficient to cause acne of a subject when applied to a subject's skin. In some embodiments, the probiotic does not produce or induce an amount of pro-inflammatory mediators sufficient to worsen acne of a subject when applied to a subject's skin. The probiotic may not induce human inflammatory associated mRNAs such as interleukins, chemokine or cytokines when applied to a subject's skin. The probiotic may induce only low amounts of human inflammatory associated mRNAs such as interleukins, chemokine or cytokines, when applied to a subject's skin.

In some embodiments, a probiotic disclosed herein is selected or assessed by incubating a sample of keratinocytes with the probiotic. In some embodiments, the probiotic produces or induces a low amount of pro-inflammatory mediators when co-incubated with a subject's keratinocytes. In some embodiments, the probiotic produces or induces a low amount of pro-inflammatory mediators when co-incubated with skin keratinocytes that are pooled from multiple subjects. In some embodiments, the probiotic does not produce or induce pro-inflammatory mediators when co-incubated with a subject's keratinocytes. In some embodiments, the probiotic does not produce or induce pro-inflammatory mediators when co-incubated with skin keratinocytes pooled from multiple subjects (e.g., “pooled skin keratinocytes”). In some embodiments, the probiotic comprises does not produce or induce an amount of pro-inflammatory mediators sufficient to cause acne of a subject when co-incubated with the subject's keratinocytes or pooled skin keratinocytes. In some embodiments, the probiotic does not produce or induce an amount of pro-inflammatory mediators sufficient to worsen acne of a subject when co-incubated with a subject's keratinocytes or pooled skin keratinocytes. The probiotic may not induce human inflammatory associated mRNAs such as interleukins, chemokine or cytokines when incubated with a subjects own keratinocytes or pooled keratinocytes from a plurality of healthy volunteers. The probiotic may induce only low amounts of human inflammatory associated mRNAs such as interleukins, chemokine or cytokines, when incubated with a subjects own keratinocytes or pooled keratinocytes from a plurality of healthy volunteers. The probiotic may induce only low amounts of human inflammatory associated mRNAs or proteins such as interleukins, chemokine or cytokines, when incubated with primary human monocytes. In a certain embodiment, the inflammatory interleukin, chemokine or cytokine comprises IL-2, IL-12, or MCP-1. An exemplar assay for induction of IL-12 in monocytes by a bacterium can be found in U.S. Pat. No. 9,549,905.

Probiotic strains disclosed herein may be defined by 16S, 18S, or 23S ribosomal DNA sequences, metagenomic DNA, the presence of specific health-associated markers, the absence of disease-associated markers, or a combination thereof. Probiotic strains disclosed herein may be isolated and purified from culture.

Probiotics disclosed herein may comprise at least one strain of bacteria that inhibits the activity of a pathogenic strain of P. acnes. In some embodiments, the probiotic is bacteriostatic or bactericidal for a pathogenic strain of P. acnes. In some embodiments, the probiotic reduces growth, re-growth or metabolism of the pathogenic strain of P. acnes. In certain embodiments, the probiotic reduces growth (or re-growth) of the pathogenic P. acnes by at least about 20% as compared to placebo/no probiotic. In certain embodiments, the probiotic reduces growth (or re-growth) of the pathogenic P. acnes by at least about 30%. In certain embodiments, the probiotic reduces growth (or re-growth) of the pathogenic P. acnes by at least about 40%. In certain embodiments, the probiotic reduces growth (or re-growth) of the pathogenic P. acnes by at least about 50%. In certain embodiments, the probiotic reduces growth (or re-growth) of the pathogenic P. acnes by at least about 60%. In certain embodiments, the probiotic reduces growth (or re-growth) of the pathogenic P. acnes by at least about 70%. In certain embodiments, the probiotic reduces growth (or re-growth) of the pathogenic P. acnes by at least about 2-fold. In certain embodiments, the probiotic reduces growth (or re-growth) of the pathogenic P. acnes by at least about 3-fold. In certain embodiments, the probiotic reduces growth (or re-growth) of the pathogenic P. acnes by at least about 5-fold. In certain embodiments, the probiotic reduces growth (or re-growth) of the pathogenic P. acnes by at least about 10-fold. Inhibition or reduction of growth (or re-growth) of the pathogenic P. acnes may be assessed in an in vitro assay, either in liquid culture or on solid media.

In some embodiments, the probiotic comprises at least one healthy P. acnes strain. In some embodiments, the probiotic comprises not more than one healthy P. acnes strain. In some embodiments, the probiotic comprises at least two healthy P. acnes strains. In some embodiments, the at least one healthy P. acnes strain is not a strain of P. acnes subsp. acnes. In some embodiments, the probiotic comprises at least one healthy P. avidum strain. In some embodiments, the probiotic comprises not more than one healthy P. avidum strain. In some embodiments, the probiotic comprises at least two healthy P. avidum strains. In some embodiments, the probiotic comprises at least one healthy P. granulosum strain. In some embodiments, the probiotic comprises not more than one healthy P. granulosum strain. In some embodiments, the probiotic comprises at least two healthy P. granulosum strains. In some embodiments, the probiotic comprises at least one healthy P. defendens strain. In some embodiments, the probiotic comprises not more than one healthy P. defendens strain. In some embodiments, the probiotic comprises at least two healthy P. defendens strains.

P. acnes strains may be characterized by a ribotype (RT). Ribotypes are defined, for example, as in Fitz-Gibbon et al., J. Investigative Dermatology 133:2152-60 (2013). A ribotype can be determined by the nucleic acid sequence of the strain's 16S ribosomal subunit. In certain embodiments, the probiotic comprises healthy P. acnes of a ribotype selected from at least one of the following ribotypes of RT1, RT2, RT3, RT4, RT5, RT7, RT8, RT9, or RT10. In certain embodiments, the probiotic comprises healthy P. acnes of a ribotype selected from at least one of the following ribotypes of RT1, RT2, RT3, RT7, RT8, RT9, or RT10. In certain embodiments, the probiotic comprises healthy P. acnes of a ribotype selected from RT1 and RT2. In some instances, the probiotic does not comprise a P. acnes strain of ribotype RT6. In some instances, the probiotic does not comprise a P. acnes strain of a ribotype selected from RT3, RT4, RT5 and RT6. In some instances, the probiotic does not comprise a P. acnes strain of ribotype RT6. In some instances, the probiotic does not comprise a P. acnes strain of a ribotype selected from RT3, RT4, and RT5. In some embodiments, compositions disclosed herein comprise a strain of P. acnes of ribotype RT1. In some embodiments, compositions disclosed herein comprise a strain of P. acnes of ribotype RT2. In some embodiments, compositions disclosed herein comprise a strain of P. acnes of ribotype RT3. In some embodiments, compositions disclosed herein comprise a strain of P. acnes of ribotype RT4. In some embodiments, compositions disclosed herein comprise a strain of P. acnes of ribotype RT5. In some embodiments, compositions disclosed herein comprise a strain of P. acnes of ribotype RT6. In some embodiments, compositions disclosed herein comprise a strain of P. acnes of ribotype RT7. In some embodiments, compositions disclosed herein comprise a strain of P. acnes of ribotype RT8. In some embodiments, compositions disclosed herein comprise a strain of P. acnes of ribotype RT9. In some embodiments, compositions disclosed herein comprise a strain of P. acnes of ribotype RT10. In certain embodiments, the probiotic comprises a strain of P. acnes of a ribotype selected from at least one of RT1, RT2, and RT3. In certain embodiments, the probiotic comprises a P. acnes strain of ribotype RT1. In certain embodiments, the probiotic comprises a P. acnes strain of ribotype RT2. In certain embodiments, the probiotic comprises a P. acnes strain of ribotype RT3. In certain embodiments, the probiotic comprises a P. acnes strain of ribotype RT3. In certain embodiments, the probiotic does not comprise an RT6 ribotype.

In certain embodiments, the probiotic does not comprise a strain with a sequence that is 95%, 97%, 98%, 99% or 100% homologous to a sequence selected from SEQ ID NO: 1, SEQ ID NO: 2, SEQ ID NO: 3, and SEQ ID NO: 4. In certain embodiments, the probiotic does not comprise a strain with a sequence that is more than 50% homologous to a sequence selected from SEQ ID NO: 51, SEQ ID O: 2, SEQ ID NO: 3, and SEQ ID NO: 4. In certain embodiments, the probiotic does not comprise a strain with a sequence selected from SEQ ID NO: 1, SEQ ID NO: 2, SEQ ID NO: 3.

In certain embodiments, probiotics disclosed herein comprise a plurality of microbes of different ribotypes. In certain embodiments, the plurality of microbes is a mixture of two or more P. acnes strains of different ribotypes. In certain embodiments, the probiotic comprises a first strain of P. acnes of ribotype RT1 and a second strain of P. acnes of ribotype RT2. In certain embodiments, the probiotic comprises a first strain of P. acnes of ribotype RT1 and a second strain of P. acnes of ribotype RT3. In certain embodiments, the probiotic comprises a first strain of P. acnes of ribotype RT1 and a second strain of P. acnes is not ribotype RT6. In certain embodiments, the probiotic comprises a first strain of P. acnes of ribotype RT2 and a second strain of P. acnes of ribotype RT3. In certain embodiments, the probiotic comprises a first strain of P. acnes of ribotype RT2 and a second strain of P. acnes is not ribotype RT6. In certain embodiments, the probiotic comprises a first strain of P. acnes of ribotype RT2 and a second strain of P. acnes of ribotype RT3. In certain embodiments, the probiotic consists essentially of a first strain of P. acnes of ribotype RT1 and a second strain of P. acnes of ribotype RT2. In certain embodiments, the probiotic consists essentially of a first strain of P. acnes of ribotype RT1 and a second strain of P. acnes of ribotype RT3. In certain embodiments, the probiotic consists essentially of a first strain of P. acnes of ribotype RT1 and a second strain of P. acnes is not ribotype RT6. In certain embodiments, the probiotic consists essentially of a first strain of P. acnes of ribotype RT2 and a second strain of P. acnes of ribotype RT3. In certain embodiments, the probiotic consists essentially of a first strain of P. acnes of ribotype RT2 and a second strain of P. acnes is not ribotype RT6. In certain embodiments, the probiotic consists essentially of a first strain of P. acnes of ribotype RT2 and a second strain of P. acnes of ribotype RT3. In some embodiments, the first strain of P. acnes and the second strain of P. acnes are provided separately. In some embodiments, the first strain of P. acnes and second strain of P. acnes are mixed.

In some embodiments, probiotics disclosed herein comprise a health-associated P. acnes strain that is ribotype RT1, deoR-positive, type II lipase-positive, does not comprise Cas5 and has less than 5% pIMPLE plasmid. In some embodiments, deoR-positive means that the strain comprises a nucleic acid encoding deoR. In some embodiments, deoR-positive means that the strain comprises a deoR protein. In some embodiments, type II lipase-positive means that the strain comprises a nucleic acid encoding type II lipase. In some embodiments, type II lipase—positive means that the strain comprises a type II lipase protein. In some embodiments, the strain does not comprise a nucleic acid encoding a Cas 5 protein. In some embodiments, probiotics disclosed herein comprise a health-associated P. acnes strain that is ribotype RT2, deoR-positive, type II lipase-positive, Cas5-positive and has less than 5% pIMPLE plasmid.

In certain embodiments, the probiotic is a plurality of microbes of different ribotypes. In certain embodiments, the plurality of microbes comprises a mixture of three or more P. acnes strains of different ribotypes. In certain embodiments, the different ribotypes are RT1, RT2, and RT3. In certain embodiments, the different ribotypes are RT2, RT3, and not RT6. In certain embodiments, the different ribotypes are RT1, RT2, and not RT6. In certain embodiments, the different ribotypes are RT1, RT3, and not RT6. In certain embodiments, the mixture consists essentially of three P. acnes strains, wherein the three P. acnes strains are of ribotypes RT1, RT2, and RT3. In certain embodiments, the mixture consists essentially of three P. acnes strains, wherein the three P. acnes strains are of ribotypes RT2, RT3, and not RT6. In certain embodiments, the mixture consists essentially of three P. acnes strains, wherein the three P. acnes strains are of ribotypes RT1, RT2, and not RT6. In certain embodiments, the mixture consists essentially of three P. acnes strains, wherein the three P. acnes strains are of ribotypes RT1, RT3, and not RT6.

In some embodiments, probiotics disclosed herein comprise at least one health-associated strain of P. acnes, wherein the health-associated strain of P. acnes has a ribotype of RT1 or RT2. In some embodiments, probiotics disclosed herein comprise at least two health-associated strains of P. acnes, wherein a first health-associated strain of P. acnes has a ribotype of RT1 and a second health-associated strain of P. acnes has a ribotype of RT2. In some embodiments, probiotics disclosed herein do not comprise a strain of P. acnes, other than a health-associated strain of P. acnes disclosed herein that has a ribotype selected from RT1 and RT2. In some embodiments, the probiotic does not comprise a strain of P. acnes that has a ribotype RT6. In some embodiments, the probiotic does not comprise a strain of P. acnes that comprises a nucleic acid encoding a DNA binding response regulator or a phosphoglycerate kinase, as described herein. In some embodiments, the probiotic comprises a strain of P. acnes comprising a nucleic acid encoding an ATP binding cassette transporter, as described herein. In some embodiments, the probiotic does not comprise a strain of P. acnes that expresses DNA binding response regulator or phosphoglycerate kinase, as described herein. In some embodiments, the probiotic comprise a strain of P. acnes that expresses an ATP binding cassette transporter, as described herein.

In some embodiments, probiotics disclosed herein comprise at least one strain of bacteria that can be identified by a genetic signature. The genetic signature can be described as one or more genes that are at least present, and optionally expressed, in a strain of bacteria. The one or more genes may comprise a gene encoding a deoxyribose operon repressor (deoR). The one or more genes may comprise type I lipase. The one or more genes may comprise type II lipase. The one or more genes may encode Cas 5. In some instances, the one or more genes do not comprise a gene encoding a deoxyribose operon repressor (gene abbreviated deoR, protein abbreviated herein as deoR). In some instances, the one or more genes do not comprise a gene encoding a type I lipase. In some instances, the one or more genes do not comprise a gene encoding a type II lipase. In some instances, the one or more genes do not comprise a gene encoding a Cas5 protein.

In some embodiments, probiotics disclosed herein comprise at least one strain of bacteria expressing a type II lipase. In some embodiments, probiotics disclosed herein comprise at least one strain of bacteria expressing a deoR and a type II lipase. In some embodiments, probiotics disclosed herein comprise at least one strain of bacteria expressing a Cas5 and a type II lipase. In some embodiments, probiotics disclosed herein comprise at least one strain of bacteria expressing a deoR and a type II lipase and a Cas5.

In some embodiments, compositions disclosed herein comprise at least one strain of bacteria, wherein less than about 25% of a number of reads align per number of reads tested when the at least one strain is sequenced for pIMPLE plasmid. In some embodiments, compositions disclosed herein comprise at least one strain of bacteria, wherein less than about 20% of a number of reads align per number of reads tested when the at least one strain is sequenced for pIMPLE plasmid. In some embodiments, compositions disclosed herein comprise at least one strain of bacteria, wherein less than about 15% of a number of reads align per number of reads tested when the at least one strain is sequenced for pIMPLE plasmid. In some embodiments, compositions disclosed herein comprise at least one strain of bacteria, wherein less than about 10% of a number of reads align per number of reads tested when the at least one strain is sequenced for pIMPLE plasmid. In some embodiments, compositions disclosed herein comprise at least one strain of bacteria, wherein less than about 9% of a number of reads align per number of reads tested when the at least one strain is sequenced for pIMPLE plasmid. In some embodiments, compositions disclosed herein comprise at least one strain of bacteria, wherein less than about 8% of a number of reads align per number of reads tested when the at least one strain is sequenced for pIMPLE plasmid. In some embodiments, compositions disclosed herein comprise at least one strain of bacteria, wherein less than about 7% of a number of reads align per number of reads tested when the at least one strain is sequenced for pIMPLE plasmid. In some embodiments, compositions disclosed herein comprise at least one strain of bacteria, wherein less than about 6% of a number of reads align per number of reads tested when the at least one strain is sequenced for pIMPLE plasmid. In some embodiments, compositions disclosed herein comprise at least one strain of bacteria, wherein less than about 5% of a number of reads align per number of reads tested when the at least one strain is sequenced for pIMPLE plasmid. In some embodiments, compositions disclosed herein comprise at least one strain of bacteria, wherein less than about 4% of a number of reads align per number of reads tested when the at least one strain is sequenced for pIMPLE plasmid. In some embodiments, compositions disclosed herein comprise at least one strain of bacteria, wherein less than about 3% of a number of reads align per number of reads tested when the at least one strain is sequenced for pIMPLE plasmid. In some embodiments, compositions disclosed herein comprise at least one strain of bacteria, wherein less than about 2% of a number of reads align per number of reads tested when the at least one strain is sequenced for pIMPLE plasmid. In some embodiments, compositions disclosed herein comprise at least one strain of bacteria, wherein less than about 1% of a number of reads align per number of reads tested when the at least one strain is sequenced for pIMPLE plasmid. In some embodiments, compositions disclosed herein comprise at least one strain of bacteria, wherein less than about 0.5% of a number of reads align per number of reads tested when the at least one strain is sequenced for pIMPLE plasmid.

In some embodiments, the composition comprises a strain of bacteria that is ribotype RT1, expresses type I lipase, and does not express deoR and Cas5, and wherein less than 5% of a number of reads align per number of reads tested when the strain is sequenced for pIMPLE plasmid. In some embodiments, the composition comprises a strain of bacteria that is ribotype RT1, expresses deoR and type I lipase, and does not express Cas5, and wherein less than 5% of a number of reads align per number of reads tested when the strain is sequenced for pIMPLE plasmid. In some embodiments, the composition comprises a strain of bacteria that is ribotype RT1, expresses deoR and type II lipase, and does not express Cas5, and wherein less than 5% of a number of reads align per number of reads tested when the strain is sequenced for pIMPLE plasmid. In some embodiments, the composition comprises a strain of bacteria that is ribotype RT2, expresses deoR, type II lipase, and Cas5, and wherein less than 5% of a number of reads align per number of reads tested when the strain is sequenced for pIMPLE plasmid. In some embodiments, the composition comprises a strain of bacteria that is ribotype RT4 and expresses type I lipase, but not deoR. In some embodiments, the composition comprises a strain of bacteria that is not ribotype RT6, expresses deoR, type II lipase, and Cas5, and wherein less than 5% of a number of reads align per number of reads tested when the strain is sequenced for pIMPLE plasmid.

In some embodiments, the composition comprises a strain of bacteria that is ribotype RT1, comprises a nucleic acid encoding type I lipase, and does not comprises a nucleic acid encoding deoR or Cas5, and wherein less than 5% of a number of reads align per number of reads tested when the strain is sequenced for pIMPLE plasmid. In some embodiments, the composition comprises a strain of bacteria that is ribotype RT1, comprises at least one nucleic acid encoding deoR and type I lipase, does not comprise a nucleic acid encoding Cas5, and wherein less than 5% of a number of reads align per number of reads tested when the strain is sequenced for pIMPLE plasmid. In some embodiments, the composition comprises a strain of bacteria that is ribotype RT1, a nucleic acid encoding deoR and type II lipase, does not comprise a nucleic acid encoding Cas5, and wherein less than 5% of a number of reads align per number of reads tested when the strain is sequenced for pIMPLE plasmid. In some embodiments, the composition comprises a strain of bacteria that is ribotype RT2, comprises at least one nucleic acid encoding deoR, type II lipase, and Cas5, and wherein less than 5% of a number of reads align per number of reads tested when the strain is sequenced for pIMPLE plasmid. In some embodiments, the composition comprises a strain of bacteria that is ribotype RT4 and comprises a nucleic acid encoding type I lipase, but does not comprise a nucleic acid encoding deoR. In some embodiments, the composition comprises a strain of bacteria that is not ribotype RT6, comprises at least one nucleic acid encoding deoR, type II lipase, and Cas5, and wherein less than 5% of a number of reads align per number of reads tested when the strain is sequenced for pIMPLE plasmid.

Table 1 below provides a summary of non-limiting examples of P. acnes bacteria, or populations of bacteria comprising P. acnes bacteria, that can be distinguished by analysis of different genetic markers. The +symbol indicates the presence of deoR or Cas5 (or nucleic acids encoding deoR or Cas5) in the columns labeled deoR and Cas5 (or nucleic acids encoding deoR or Cas5), respectively. The − symbol indicates absence of deoR or Cas5 in the columns labeled deoR and Cas5, respectively. % pIMPLE plasmid refers to the number of reads aligned/number of reads tested when bacteria is sequenced for pIMPLE plasmid.

| | | | | | % | | | | |
|---|---|---|---|---|---|---|---|---|---|
| | | | | | pIMPLE | ABC | | | Exemplary |
| Group | RT | deoR | Lipase | Cas5 | plasmid | XP | DBRR | PGK | strains |
| A | 1 | − | I | − | <5% | + | − | − | |
| B | 1 | + | I | − | <5% | + | − | − | |
| C | 1 | + | II | − | <5% | + | − | − | HP3A11 |
| D | 2 | + | II | + | <5% | + | − | − | HP4G1, |
| | | | | | | | | | HP5G4 |
| E | 4 | − | I | − | >1% | + | − | − | HL045PA1 |
| F | 5 | − | I | − | >1% | + | − | − | HL043PA1 |
| G | 6 | + | II | + | >5% | − | + | + | HL110PA3, |
| | | | | | | | | | HL110PA4 |

ABC XP = ATP binding cassette transporter
DBRR = DNA binding response regulator
PGK = phosphoglycerate kinase

In certain embodiments, the composition comprises at least one strain of a P. acnes microbe that corresponds to group A of Table 1. In certain embodiments, the composition comprises at least one strain of a P. acnes microbe that corresponds to group B of Table 1. In certain embodiments, the composition comprises at least one strain of a P. acnes microbe that corresponds to group C of Table 1. In certain embodiments, the composition comprises at least one strain of a P. acnes microbe that corresponds to group D of Table 1.

In certain embodiments, the composition comprises at least one strain of a P. acnes microbe that corresponds to groups A, B, C or D of Table 1. In certain embodiments, the composition comprises at least two strains of a P. acnes microbe that correspond to groups A, B, C or D of Table 1. In certain embodiments, the composition comprises at least three strains of a P. acnes microbe that correspond to groups A, B, C or D of Table 1. In certain embodiments, the composition comprises at least four strains of a P. acnes microbe that correspond to groups A, B, C or D of Table 1. In certain embodiments, the composition comprises at least five strains of a P. acnes microbe that correspond to groups A, B, C, or D of Table 1.

In some embodiments, the composition does not comprise a strain of P. acnes that corresponds to group E of Table 1. In some embodiments, the composition does not comprise a strain of P. acnes that corresponds to group F of Table 1. In some embodiments, the composition does not comprise a strain of P. acnes that corresponds to group G of Table 1.

In some embodiments, the composition comprises at least one strain of P. acnes that corresponds to group A of Table 1, but does not comprise a strain of P. acnes that corresponds to groups E, F or G of Table 1. In some embodiments, the composition comprises at least one strain of P. acnes that corresponds to group B of Table 1, but does not comprise a strain of P. acnes that corresponds to groups E, F or G of Table 1. In some embodiments, the composition comprises at least one strain of P. acnes that corresponds to group C of Table 1, but does not comprise a strain of P. acnes that corresponds to groups E, F or G of Table 1. In some embodiments, the composition comprises at least one strain of P. acnes that corresponds to group D of Table 1, but does not comprise a strain of P. acnes that corresponds to groups E, F or G of Table 1. In some embodiments, the composition comprises at least one strain of P. acnes that corresponds to group C of Table 1 and at least one strain of P. acnes that corresponds to group D of Table 1, but does not comprise a strain of P. acnes that corresponds to groups E, F or G of Table 1.

### Health-Associated Microbes

Provided herein are compositions for treating and preventing skin disorders, wherein the compositions comprise a health-associated microbe. These strains are useful in conjunction with the light therapy methods described herein. Generally, the term, “health-associated microbe,” as used herein, refers to a microbe that is more prevalent in healthy or individuals free of a skin disease than in individuals diagnosed with the skin disease. In some embodiments, health-associated microbes disclosed herein are associated with desirable or optimal oral health. In some embodiments, health-associated microbes disclosed herein are associated with desirable or optimal gastrointestinal health.

Health-associated microbes include probiotics described herein. Health-associated microbes include healthy P. acnes described herein. In some embodiments, health-associated microbes are microbes that can replace at least a portion of pathogenic microbes. In some embodiments, health-associated microbes are microbes that can alter a microbiome to increase a percentage of health-associated strains. In some embodiments, health-associated microbes inhibit the growth of pathogenic microbes or disease associated microbes. In some embodiments, health-associated microbes out-compete pathogenic or disease associated microbes. In certain embodiments, the skin disorder is selected from at least one of acne vulgaris (acne), eczema, seborrheic dermatitis, psoriasis, or rosacea. In some embodiments, the skin disorder is acne.

In certain embodiments, health-associated microbes disclosed herein are associated with desirable health, optimal health or improved health relative to the health of a subject with a disease, disorder or condition disclosed herein. Desirable health, optimal health or improved health may be characterized as free of a condition, disorder or disease. Desirable health, optimal health or improved health may be characterized as free of one or more symptoms of a condition, disorder or disease. Desirable health, optimal health or improved health may be characterized as free of all symptoms of a condition, disorder or disease. Desirable health, optimal health or improved health may be characterized as improved health relative to health with a disease, disorder or condition. In certain embodiments, the health-associated microbe is associated with optimal, desirable or improved skin health. In certain embodiments, the health-associated microbe is associated with optimal, desirable or improved oral health. In certain embodiments, the health-associated microbe is associated with optimal, desirable or improved digestive health. In certain embodiments, the health-associated microbe is a P. acnes strain associated with skin health, oral health, digestive health, or any combination thereof, that is optimal, desirable or improved relative to respective health-associated with a condition, disorder or disease.

In certain embodiments, there is a statistically significant difference in the presence of the health-associated microbe on the skin of an individual free of a disease when compared to an individual with the disease. In certain embodiments, there is at least about a 10% greater quantity of a health-associated microbe on the skin of an individual free of a disease when compared to an individual with the disease. In certain embodiments, there is at least about a 50% greater quantity of a health-associated microbe on the skin of an individual free of a disease when compared to an individual with the disease. In certain embodiments, there is at least about a 100% greater quantity of a health-associated microbe on the skin of an individual free of a disease when compared to an individual with the disease. In certain embodiments, there is at least about a 2-fold greater presence of the health-associated microbe on the skin of an individual free of a disease when compared to an individual with the disease. In certain embodiments, there is at least about a 3-fold greater presence of the health-associated microbe on the skin of an individual free of a disease when compared to an individual with the disease. In certain embodiments, there is at least about a 5-fold greater presence of the health-associated microbe on the skin of an individual free of a disease when compared to an individual with the disease. In certain embodiments, there is at least about a 10-fold greater presence of the health-associated microbe on the skin of an individual free of a disease when compared to an individual with the disease.

In certain embodiments, the health-associated microbe is an isolated species of bacteria. In certain embodiments, the health-associated microbe is a purified species of bacteria. In certain embodiments, the health-associated microbe is an isolated and purified species of bacteria. In certain embodiments, the health-associated microbe is an isolated strain of bacteria. In certain embodiments, the health-associated microbe is a purified strain of bacteria. In certain embodiments, the health-associated microbe is an isolated and purified strain of bacteria. In certain embodiments, the health-associated microbe is an isolated species of Propionibacterium. In certain embodiments, the health-associated microbe is a purified species of Propionibacterium. In certain embodiments, the health-associated microbe is an isolated and purified species of Propionibacterium. In certain embodiments, the health-associated microbe is an isolated strain of P. acnes. In certain embodiments, the health-associated microbe is a purified strain of P. acnes. In certain embodiments, the health-associated microbe is an isolated and purified strain of P. acnes.

As described herein, some strains of P. acnes are associated with acne and some strains of P. acnes are associated with skin free of acne or disease. These P. acnes strains can be differentiated at the genetic level by using nucleic acid sequence determination methods known in the art such as PCR, restriction mapping, Sanger sequencing, and next-generation sequencing. In some instances, a health-associated microbe disclosed herein is beneficial for the treatment of a specific skin disorder, but not all skin disorders. In some instances, a health-associated microbe disclosed herein is beneficial for the treatment of a plurality of skin disorder, but not all skin disorders. In some instances, a health-associated microbe disclosed herein is beneficial for the treatment any skin disorder. By way of non-limiting example, a health-associated microbe disclosed herein may be beneficial only for acne, but not for eczema, seborrheic dermatitis, or psoriasis. In another instance, a health-associated microbe disclosed herein is beneficial only for eczema, but not for acne, seborrheic dermatitis, or psoriasis. In another instance, a health-associated microbe disclosed herein is beneficial only for psoriasis, but not for acne, seborrheic dermatitis, or eczema. In another instance, a health-associated microbe disclosed herein is beneficial only for seborrheic dermatitis, but not for psoriasis, acne, or eczema. In some instances, a health-associated microbe disclosed herein is beneficial for eczema, acne and psoriasis. In some instances, a health-associated microbe disclosed herein is beneficial for acne and a condition selected from eczema, seborrheic dermatitis. In some instances, a health-associated microbe disclosed herein is beneficial for eczema, seborrheic dermatitis, acne and psoriasis.

In certain embodiments, compositions disclosed herein for use in conjunction with the light therapy methods disclosed herein comprise at least one health-associated microbe, wherein the health-associated microbe is a strain of P. acnes or bacteria that is associated with healthy or normal skin. In certain embodiments, compositions disclosed herein comprise at least one health-associated microbe, wherein the health-associated microbe is a strain of P. acnes or bacteria that produces low levels of inflammatory mediators when incubated with a subject's own keratinocytes or pooled keratinocytes from multiple subjects.

In certain embodiments, compositions comprise an isolated P. acnes strain. In some embodiments the isolated P. acnes strain is a purified strain. In certain embodiments, compositions comprise a mixture of about 2 to about 10 isolated P. acnes strains. In certain embodiments, compositions comprise a mixture of about 3 to about 8 isolated P. acnes strains. In certain embodiments, compositions comprise a mixture of about 2 to about 5 isolated P. acnes strains. In certain embodiments, compositions comprise a mixture of about 3 to about 6 isolated P. acnes strains. In certain embodiments, the isolated strain is isolated based on its phylotype or ribotype.

In certain embodiments, compositions disclosed herein comprise at least one P. acnes strain having a health-associated phylotype. In certain embodiments, the health-associated phylotype is selected from type I, type II, and type III. In some embodiments, compositions disclosed herein comprise at least two P. acnes strains having health-associated phylotypes, wherein the health-associated phylotypes are a combination of type I, type II, and type III. In certain embodiments, the type I phylotype is selected from type IA, type IB, and type IC. In certain embodiments, the type IA phylotype is selected from type IA₁ and type IA₂. Strains can be phylotyped as in McDowell et al. (PLoS ONE 8(9): e70897 (2013)).

In some embodiments, compositions disclosed herein comprise a combination of health-associated microbes, wherein the health-associated microbes comprise a combination of healthy strains of P. acnes. In some embodiments, combinations of healthy strains of P. acnes comprise a combination of strains of P. acnes of a plurality of ribotypes. In some embodiments, the plurality of ribotypes comprises at least two ribotypes selected from RT1, RT2, RT3, RT7, RT8, RT9, and RT10. In some embodiments, the plurality of ribotypes comprises at least two ribotypes selected from RT1, RT2 and RT3. In certain embodiments, the plurality of ribotypes comprises at least two ribotypes selected from RT1, RT2, RT3 and not RT6. In certain embodiments, the plurality of ribotypes comprises ribotypes selected from RT1 and RT2. In certain embodiments, the plurality of ribotypes comprises ribotypes selected from RT1 and RT3. In certain embodiments, the plurality of ribotypes comprises ribotypes selected from RT2 and RT3. In certain embodiments, the plurality of ribotypes comprises RT1, but not RT6. In certain embodiments, the plurality of ribotypes comprises RT2, but not RT6.

In some embodiments, compositions disclosed herein comprise a combination of health-associated microbes, wherein the health-associated microbes comprise a combination of healthy strains of P. acnes. In some embodiments, the combination comprises a first strain of P. acnes and a second strain of P. acnes. In some embodiments, the first strain of P. acnes is of a first ribotype and a second strain of P. acnes is of a second ribotype. In some embodiments, the first ribotype and the second ribotype are the same. In some embodiments, the first ribotype and the second ribotype are different. In some embodiments, the first ribotype is RT1 and the second ribotype is RT1. In some embodiments, the first ribotype is RT2 and the second ribotype is RT2. In some embodiments, the first ribotype is RT1 and the second ribotype is RT2. In some embodiments, the first ribotype is RT1 and the second ribotype is RT3. In some embodiments, the first ribotype is RT2 and the second ribotype is RT3. In some embodiments, the first ribotype is RT1 and the second ribotype is not RT6. In some embodiments, the first ribotype is RT2 and the second ribotype is not RT6. In some embodiments, the first ribotype is RT3 and the second ribotype is not RT6.

In some embodiments, compositions disclosed herein comprise healthy strains of P. acnes, and do not comprise any other type of microbe or bacteria. In some embodiments, health-associated microbes disclosed herein comprise at least one health-associated strain of P. acnes, wherein the health-associated strain of P. acnes has a ribotype of RT1 or RT2. In some embodiments, health-associated microbes disclosed herein do not comprise a strain of P. acnes, other than a health-associated strain of P. acnes disclosed herein that has a ribotype selected from RT1 and RT2. In some embodiments, the health-associated microbes do not comprise a strain of P. acnes that has a ribotype RT6. In some embodiments, the health-associated microbes do not comprise a strain of P. acnes that expresses DNA binding response regulator or phosphoglycerate kinase, as described herein. In some embodiments, the health-associated microbes comprise a strain of P. acnes that expresses an ATP binding cassette transporter, as described herein.

In certain embodiments, the health-associated microbe does not comprise a strain with a sequence that is 95%, 97%, 98%, 99% or 100% homologous to a sequence selected from SEQ ID NO: 1, SEQ ID NO: 2, SEQ ID NO: 3, and SEQ ID NO: 4. In certain embodiments, the health-associated microbe does not comprise a strain with a sequence that is more than 50% homologous to a sequence selected from SEQ ID NO: 1, SEQ ID NO: 2, SEQ ID NO: 3, and SEQ ID NO: 4. In certain embodiments, the health-associated microbe does not comprise a strain with a sequence selected from SEQ ID NO: 1, SEQ ID NO: 2, SEQ ID NO: 3 and SEQ ID NO: 4.

In certain embodiments, compositions disclosed herein comprise a health-associated microbe, wherein the health-associated microbe is Lactobacillus reuteri (L. reuteri), or a strain thereof. In certain embodiments, compositions disclosed herein comprise at least one L. reuteri strain selected from Korean Collection for Type Cultures (KCTC) deposited strains, such as KCTC 3679, KCTC 3594, KCTC 3678, and any combination thereof.

In certain embodiments, compositions disclosed herein comprise a health-associated microbe, wherein the health-associated microbe is Staphylococcus epidermidis (S. epidermidis). In certain embodiments, compositions disclosed herein comprise at least one S. epidermidis strain selected from 14.1.R1, AS1, AU 10, AU16, AU21, AU23, AU24, AU35, AU36, AU39, AU40, AU 44, AU48, AU53, AU60, AU73, AU81, FS1, G53, IS2, and a combination thereof.

### Selected, Transformed, or Engineered Bacteria

In certain embodiments, the compositions described herein comprise one or more strains of bacteria that is selected, transformed or engineered with a gene or gene mutation that is beneficial for a skin disorder. Thus, the bacteria have been transformed into a “non-pathogenic” form, or a health-associated form from a disease-associated form. In certain embodiments, a gene that contributes to pathogenesis of a skin disorder is deleted or mutated to inactivate or reduce the corresponding gene product. In certain embodiments, a gene that reduces the pathogenesis of a skin disorder is added, or mutated to activate or increase levels of the corresponding gene product. In certain embodiments, the bacteria are grown and selected from culture or selected from healthy disease free individuals.

In certain embodiments, selected, transformed, or engineered bacteria are to be delivered as a probiotic via compositions and methods disclosed herein. In certain embodiments, selected, transformed, or engineered bacteria disclosed herein comprise a gene encoding a deoxyribose operon repressor (deoR). In certain embodiments, selected, transformed, or engineered bacteria disclosed herein express a deoxyribose operon repressor. In certain embodiments, selected, transformed, or engineered bacteria disclosed herein comprise a gene encoding a Type II lipase. In certain embodiments, selected, transformed, or engineered bacteria disclosed herein express Type II lipase. By way of non-limiting example, the Type II lipase may be a glycerol-ester hydrolase B (GehB). In certain embodiments, selected, transformed, or engineered bacteria disclosed herein do not comprise a gene encoding Type I lipase. In certain embodiments, selected, transformed, or engineered bacteria disclosed herein do not express a Type I lipase. By way of non-limiting example, the type I lipase may be a glycerol-ester hydrolase A (GehA). In certain embodiments, selected, transformed, or engineered bacteria do not comprise a pIMPLE plasmid. In certain embodiments, selected, transformed, or engineered bacteria disclosed herein comprise a gene encoding an ABC transporter. In certain embodiments, selected, transformed, or engineered bacteria express an ABC transporter. In certain embodiments, selected, transformed, or engineered bacteria disclosed herein do not comprise a gene encoding a phosphoglycerate kinase. In certain embodiments, selected, transformed, or engineered bacteria do not express a phosphoglycerate kinase. In certain embodiments, selected, transformed, or engineered bacteria disclosed herein do not comprise a gene encoding a DNA binding response regulator. In certain embodiments, selected, transformed, or engineered bacteria do not comprise a DNA binding response regulator.

In certain embodiments, selected, transformed, or engineered bacteria disclosed herein do not express a dermatin-sulfate adhesin (e.g., DSA1, DSA2). In certain embodiments, selected, transformed, or engineered bacteria disclosed herein do comprise a nucleic acid encoding a dermatin-sulfate adhesin (e.g., DSA1, DSA2). The absence or deletion of dermatin-sulfate adhesins may disable adhesion of microbes to keratinocytes.

In certain embodiments, selected, transformed, or engineered bacteria disclosed herein express a hyaluronidase. In certain embodiments, selected, transformed, or engineered bacteria disclosed herein do not express a hyaluronidase. In certain embodiments, selected, transformed, or engineered bacteria disclosed herein comprise a nucleic acid encoding a hyaluronidase. In certain embodiments, selected, transformed, or engineered bacteria disclosed herein do not comprise a nucleic acid encoding a hyaluronidase. In certain embodiments, a strain of bacteria that is selected, transformed, or engineered bacteria is present or has increased expression of a hyaluronidase gene relative to the strain of bacteria when it is not selected, transformed, or engineered. In certain embodiments, a strain of bacteria that is selected, transformed, or engineered bacteria is present or has increased hyaluronidase activity relative to the strain of bacteria when it is not selected, transformed, or engineered. In certain embodiments, a strain of bacteria that is selected, transformed, or engineered bacteria is present or has reduced hyaluronidase activity relative to the strain of bacteria when it is not selected, transformed, or engineered. In certain embodiments, selected, transformed, or engineered bacteria disclosed herein lack a hyaluronidase gene.

In certain embodiments, selected, transformed, or engineered bacteria disclosed herein have reduced transposase 2 enzyme activity. In certain embodiments, selected, transformed, or engineered bacteria disclosed herein lack transposase 2 enzyme activity. In certain embodiments, a selected, transformed, or engineered strain of bacteria disclosed herein has reduced activity relative to the strain when it is not selected, transformed, or engineered, respectively.

In certain embodiments, selected, transformed, or engineered bacteria disclosed herein confer an antibiotic sensitivity to macrolide and tetracycline antibiotics. In certain embodiments, selected, transformed, or engineered bacteria disclosed herein have an absence or deletion of a thiopeptide encoding island, respectively. In certain embodiments, selected, transformed, or engineered bacteria disclosed herein have a presence or addition of a tyrosine decarboxylase island (which increases intracellular pH under stress to tolerate acidic environments), respectively. In certain embodiments, selected, transformed, or engineered bacteria disclosed herein have an ability to metabolize gelatin.

In certain embodiments, selected, transformed, or engineered bacteria comprise selected, transformed, or engineered S. epidermis, respectively. In certain embodiments, the selected, transformed, or engineered bacteria comprise selected, transformed, or engineered P. acnes.

In certain embodiments, the bacteria are transformed using recombinant DNA techniques known in the art. In certain embodiments, the bacteria are transformed by growing bacteria under selective pressure to acquire or lose a gene, gene product, or expression thereof. In a certain embodiment, the bacteria are transformed using CRISPR technology. In a certain embodiment, the bacteria are transformed using homologous recombination.

In certain embodiments, the selected, transformed, or engineered bacteria comprise a selected, transformed, or engineered P. acnes strain. In certain embodiments, the selected, transformed, or engineered bacteria comprise a selected, transformed, or engineered P. acnes strain, wherein the selected, transformed, or engineered P. acnes strain has a ribotype selected from RT1, RT2, RT3, RT4, RT5, RT7, RT8, RT9, and RT10. In certain embodiments, the selected, transformed, or engineered bacteria comprise a selected, transformed, or engineered P. acnes strain, wherein the selected, transformed, or engineered P. acnes strain has a ribotype selected from RT1, RT2, RT3, RT7, RT8, RT9, and RT10. In certain embodiments, the selected, transformed, or engineered bacteria comprise a selected, transformed, or engineered P. acnes strain, wherein the selected, transformed, or engineered P. acnes strain has a ribotype selected from RT1, RT2, RT3, RT7, RT8, RT9, and RT10. In certain embodiments, the selected, transformed, or engineered bacteria comprise a selected, transformed, or engineered P. acnes strain, wherein the selected, transformed, or engineered P. acnes strain has a ribotype selected from RT1, RT2, RT3, and not RT6. In certain embodiments, the selected, transformed, or engineered bacteria comprise a selected, transformed, or engineered P. acnes strain, wherein the selected, transformed, or engineered P. acnes strain has a ribotype selected from RT1 and RT2. In certain embodiments, the selected, transformed, or engineered bacteria comprise a selected, transformed, or engineered P. acnes strain, wherein the selected, transformed, or engineered P. acnes strain has a ribotype of RT1. In certain embodiments, the selected, transformed, or engineered bacteria comprise a selected, transformed, or engineered P. acnes strain, wherein the selected, transformed, or engineered P. acnes strain has a ribotype of RT2. In certain embodiments, the selected, transformed, or engineered bacteria are an RT1 strain of P. acnes. In certain embodiments, the selected, transformed, or engineered bacteria are an RT2 strain of P. acnes. In certain embodiments, the selected, transformed, or engineered bacteria are an RT3 strain of P. acnes. In certain embodiments, the selected, transformed, or engineered bacteria are not an RT6 strain of P. acnes.

### Markers

Disclosed herein are compositions that comprise probiotics and health-associated microbes. Probiotics and health-associated microbes disclosed herein generally comprise at least one strain of bacteria, wherein the at least one strain of bacteria exhibits a health-associated presence, health-associated absence or health-associated expression level of at least one marker. In some instances, health-associated expression of the at least one marker is a lack of expression. In some instances, health-associated expression of the at least one marker is expression that is low as compared to expression of the at least one marker in a reference strain. In some instances, the reference strain is a pathogenic strain. In some instances, the reference strain is not a health-associated strain. In some instances, health-associated expression of the at least one marker is expression that is high as compared to expression of the at least one marker in a reference strain. In some embodiments, the marker is a nucleic acid. In some embodiments, the nucleic acid comprises a gene encoding the marker or a portion thereof. In some embodiments, the nucleic acid is a gene encoding the marker or a portion thereof. In some embodiments, the marker is a protein. In some embodiments, the marker is a peptide (e.g., less than or equal to 100 amino acids). In some embodiments, the marker is not a nucleic acid or a protein. Non-limiting examples of a marker that neither comprises a nucleic acid or protein include glycans and lipids.

Exemplary markers of health-associated microbes disclosed herein include, but are not limited to, a deoxyribose operon repressor, a CRISPR associated protein (Cas), a lipase, an ATP binding cassette transporter, a DNA binding response regulator, a phosphoglycerate kinase, dermatin-sulfate adhesin, and hyaluronidase. In some instances, the at least one strain of bacteria comprises a plasmid. In some instances, presence or absence of the plasmid is a marker. By way of non-limiting example, the plasmid may be a pIMPLE plasmid disclosed herein. As further described herein, presence of a deoR, a type II lipase, an ABC transporter, or a Cas5, or a combination thereof, is generally associated with probiotics and health-associated microbes disclosed herein. In contrast, probiotics and health-associated microbes disclosed herein are generally associated with an absence or low expression of a pIMPLE plasmid, a type I lipase, a DNA binding response regulator, a phosphoglycerate kinase, or dermatin-sulfate adhesin, or a combination thereof. However, it would be understood to one of skill in the art that nature presents exceptions to such generalities. Therefore, expression patterns of these markers that are alternative or contrary to those described herein are contemplated as well. Compositions comprising one or more strains characterized by such markers are further characterized herein, including the description as follows.

deoR

In some embodiments, the marker is a deoxyribose operon repressor (deoR) protein. In some embodiments, the marker is a nucleic acid encoding a deoxyribose operon repressor (deoR). In some embodiments, the deoR is a deoR family transcriptional regulator expressed in Propionibacterium acnes subsp. defendens (ATCC 11828, GenBank: AER05724.1). In some embodiments, compositions disclosed herein comprise at least one strain of bacteria, wherein the at least one strain of bacteria comprises a nucleic acid encoding a deoxyribose operon repressor (deoR). In certain embodiments, the at least one strain of bacteria has been selected, transformed, or engineered to acquire the presence of the deoR. In certain embodiments, the deoR has a sequence that is at least 80% homologous to SEQ ID NO: 5. In certain embodiments, the deoR has a sequence that is at least 90% homologous to SEQ ID NO: 5. In certain embodiments, the deoR has a sequence that is at least 95% homologous to SEQ ID NO: 5. In certain embodiments, the deoR has a sequence that is at least 97% homologous to SEQ ID NO: 5. In certain embodiments, the deoR has a sequence that is at least 98% homologous to SEQ ID NO: 5. In certain embodiments, the deoR has a sequence that is at least 99% homologous to SEQ ID NO: 5. In certain embodiments, the deoR has a sequence that is 100% homologous to SEQ ID NO: 5. In certain embodiments, the at least one strain of bacteria has greater expression or activity of a deoxyribose operon repressor than a reference strain (e.g., pathogenic strain, not a health-associated strain). In certain embodiments, the at least one strain has at least about 1.5-fold, at least about 2-fold, at least about 3-fold, at least about 5-fold or at least about 10-fold greater expression or activity of the deoxyribose operon repressor as compared to the reference strain.

pIMPLE Plasmid

In some embodiments, the marker is a pIMPLE plasmid. In some embodiments, the marker is an absence of a pIMPLE plasmid. In some embodiments, the marker is a low percentage of a pIMPLE plasmid (e.g., <10%). In some embodiments, compositions disclosed herein comprise at least one strain of bacteria, wherein the at least one strain of bacteria does not comprise a pIMPLE plasmid. In certain embodiments, the pIMPLE plasmid has a sequence that is at least about 80% homologous to SEQ ID NO: 6. In certain embodiments, the pIMPLE plasmid has a sequence that is at least about 90% homologous to SEQ ID NO: 6. In certain embodiments, the pIMPLE plasmid has a sequence that is at least about 95% homologous to SEQ ID NO: 6. In certain embodiments, the pIMPLE plasmid has a sequence that is at least about 97% homologous to SEQ ID NO: 6. In certain embodiments, the pIMPLE plasmid has a sequence that is at least about 98% homologous to SEQ ID NO: 6. In certain embodiments, the pIMPLE plasmid has a sequence that is at least about 99% homologous to SEQ ID NO: 6. In certain embodiments, the pIMPLE plasmid has a sequence that is 100% homologous to SEQ ID NO: 6. In certain embodiments, a plasmid with at least 80%, 90%, 95%, 98%, 99%, or 100% homology to SEQ ID NO: 6 is partially or completely deleted from the at least one strain of bacteria. In certain embodiments, a plasmid with at least 80%, 90%, 95%, 98%, 99%, or 100% homology to SEQ ID NO: 6 is disrupted by an insertion of one or more nucleotides or a introduction of a frameshift mutation in the a selected, transformed, or engineered strain of bacteria. In certain embodiments, the at least one strain of bacteria contains portions of a complete pIMPLE plasmid (SEQ ID NO: 6). In certain embodiments, the at least one strain of bacteria may comprise less than about 25%, 20%, 15%, 14%, 13%, 12%, 11%, 10%, 9%, 8%, 7%, 6% of the complete pIMPLE sequence set forth in SEQ ID NO: 6. In certain embodiments, the at least one strain of bacteria does not comprise a specific portion of the pIMPLE plasmid that is present in an RT6 strain or any other disease associated strain. In certain embodiments, the at least one strain of bacteria comprises a low copy number of a pIMPLE plasmid (SEQ ID NO: 6). In certain embodiments, the at least one strain of bacteria comprises less than 5 copies of pIMPLE plasmid per bacterial genome. In certain embodiments, the at least one strain of bacteria comprises less than 4 copies of pIMPLE plasmid per bacterial genome. In certain embodiments, the at least one strain of bacteria comprises less than 3 copies of pIMPLE plasmid per bacterial genome. In certain embodiments, the at least one strain of bacteria comprises less than 2 copies of pIMPLE plasmid per bacterial genome. In certain embodiments, the at least one strain of bacteria comprises 1 copy of pIMPLE plasmid per bacterial genome. In certain embodiments, the at least one strain of bacteria comprises a low percentage of pIMPLE plasmid (SEQ ID NO: 6). In certain embodiments, the bacteria comprise less than about 15%, 14%, 13%, 12%, 11%, 10%, 9%, 8%, 7%, 6%, 5%, 4%, 3%, 2%, 1% pIMPLE plasmid. In certain embodiments, the at least one strain of bacteria comprises less than about 1.0%, 0.9%, 0.8%, 0.7%, 0.6%, 0.5%, 0.4%, 0.3%, 0.2%, 0.1% pIMPLE plasmid. pIMPLE plasmid percentage can be determined by next-generation sequencing of P. acnes bacteria, as % reads aligned. In certain embodiments, the pIMPLE percentage is percentage of total sequencing reads that align to pIMPLE from HL096PA1 (an RT5).

### Lipases

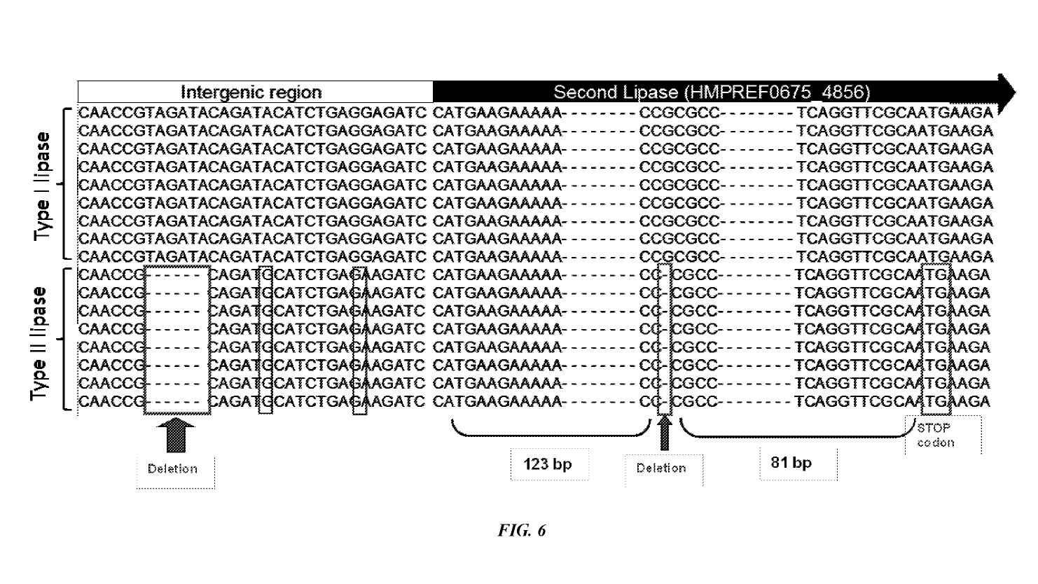

In some embodiments, the marker is a lipase. In some embodiments, the marker is a nucleic acid encoding a lipase. In some embodiments, the marker is an absence of a nucleic acid encoding a lipase. In some embodiments, compositions disclosed herein comprise a strain of bacteria that expresses a lipase. In some embodiments, the lipase is Type I lipase. In some embodiments, the lipase is Type II lipase. In some embodiments, the compositions disclosed herein comprise a strain of bacteria that does not express a lipase. In some embodiments, the compositions disclosed herein comprise a strain of bacteria that does not express a Type I lipase. In some embodiments, the compositions disclosed herein comprise a strain of bacteria that does not express a Type II lipase. In some embodiments, the strain of bacteria comprises a nucleic acid encoding a Type II lipase. Type I lipase and Type II lipase, as described herein, may be encoded by a similar nucleic acid. For example, a gene encoding Type I lipase will encode a Type II lipase upon a 6 bp deletion in the intergenic region and a single base deletion, the latter causing a frameshift that creates premature STOP codon, see, e.g., FIG. 6.

### Type I Lipase

In some embodiments, the marker is a Type I lipase. In some embodiments, compositions disclosed herein comprise at least one strain of bacteria, wherein the at least one strain of bacteria does not express a Type I lipase. In some embodiments, compositions disclosed herein comprise at least one strain of bacteria, wherein the at least one strain of bacteria does not comprise a nucleic acid encoding a Type I lipase. In certain embodiments, compositions disclosed herein comprise at least one strain of bacteria, wherein the at least one strain of bacteria has been selected, transformed, or engineered for absence of Type I lipase expression or activity. In certain embodiments, compositions disclosed herein comprise at least one strain of bacteria, wherein the at least one strain of bacteria has been selected, transformed, or engineered for the presence of Type I lipase expression or activity. In certain embodiments, compositions disclosed herein comprise at least one strain of bacteria, wherein the strain has been selected, transformed, or engineered for lower expression or activity of Type I lipase relative to a reference strain (e.g., pathogenic strain, not a health-associated strain). In certain embodiments, at least one strain of bacteria has at least about 1.5-fold lower expression or activity of Type I lipase compared to the reference strain. In certain embodiments, at least one strain of bacteria has at least about 2-fold lower expression or activity of Type I lipase compared to the reference strain. In certain embodiments, at least one strain of bacteria has at least about 3-fold lower expression or activity of Type I lipase compared to the reference strain. In certain embodiments, at least one strain of bacteria has at least about 5-fold lower expression or activity of Type I lipase compared to the reference strain. In certain embodiments, at least one strain of bacteria has at least about 10-fold lower expression or activity of Type I lipase compared to the reference strain.

In some embodiments, compositions disclosed herein comprise at least one strain of bacteria, wherein the at least one strain of bacteria expresses a Type I lipase. In certain embodiments, compositions disclosed herein comprise at least one strain of bacteria, wherein the at least one strain of bacteria has been selected, transformed, or engineered for the presence of Type I lipase expression or activity. In certain embodiments, compositions disclosed herein comprise at least one strain of bacteria, wherein the strain has been selected, transformed, or engineered for greater expression or activity of Type I lipase relative to the reference strain.

In some embodiments, at least a portion of the Type I lipase is encoded by a sequence of SEQ ID NO: 14. In some embodiments, at least a portion of the Type I lipase is encoded by a sequence that has at least 80% homology to SEQ ID NO: 14. In some embodiments, at least a portion of the Type I lipase is encoded by a sequence that has at least 90% homology to SEQ ID NO: 14. In some embodiments, at least a portion of the Type I lipase is encoded by a sequence that has at least 95% homology to SEQ ID NO: 14. In some embodiments, at least a portion of the Type I lipase is encoded by a sequence that has at least 97% homology to SEQ ID NO: 14. In some embodiments, at least a portion of the Type I lipase is encoded by a sequence that has at least 98% homology to SEQ ID NO: 14. In some embodiments, at least a portion of the Type I lipase is encoded by a sequence that has at least 99% homology to SEQ ID NO: 14. In some embodiments, at least a portion of the Type I lipase is encoded by a sequence that has at least 80% homology to SEQ ID NO: 14.

In some embodiments, at least a portion of the Type I lipase is encoded by a sequence of SEQ ID NO: 31. In some embodiments, at least a portion of the Type I lipase is encoded by a sequence that has at least 80% homology to SEQ ID NO: 31. In some embodiments, at least a portion of the Type I lipase is encoded by a sequence that has at least 90% homology to SEQ ID NO: 31. In some embodiments, at least a portion of the Type I lipase is encoded by a sequence that has at least 95% homology to SEQ ID NO: 31. In some embodiments, at least a portion of the Type I lipase is encoded by a sequence that has at least 97% homology to SEQ ID NO: 31. In some embodiments, at least a portion of the Type I lipase is encoded by a sequence that has at least 98% homology to SEQ ID NO: 31. In some embodiments, at least a portion of the Type I lipase is encoded by a sequence that has at least 99% homology to SEQ ID NO: 31. In some embodiments, at least a portion of the Type I lipase is encoded by a sequence that has at least 80% homology to SEQ ID NO: 31.

In certain embodiments, a nucleic acid with at least 80%, 90%, 95%, 98%, 99%, or 100% homology to SEQ ID NO: 14 is partially or completely deleted from the at least one strain. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 3 prime end of the nucleic acid. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 5 prime end of the nucleic acid.

In certain embodiments, a nucleic acid with at least 80%, 90%, 95%, 98%, 99%, or 100% homology to SEQ ID NO: 14 is disrupted by an insertion of one or more nucleotides or a introduction of a frameshift mutation in the at least one strain. In certain embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the nucleic acid with at least 80%, 90%, 95%, 98%, 99%, or 100% homology to SEQ ID NO: 14 is disrupted.

### Type II Lipase

In some embodiments, compositions disclosed herein comprise at least one strain of bacteria, wherein the at least one strain of bacteria expresses a Type II lipase. A non-limiting example of a Type II lipase is Lipase ADE00051, HMPREF0675_4856. In some embodiments, compositions disclosed herein comprise at least one strain of bacteria, wherein the at least one strain of bacteria comprises a nucleic acid encoding a Type II lipase. In certain embodiments, the at least one strain of bacteria has been selected, transformed, or engineered to express a Type II lipase. In certain embodiments, the at least one strain of bacteria has at least about 1.5-fold greater expression or activity of Type II lipase compared to a reference strain (e.g., pathogenic strain, not a health-associated strain). In certain embodiments, the at least one strain of bacteria has at least about 2-fold greater expression or activity of Type II lipase compared to the reference strain. In certain embodiments, the at least one strain of bacteria has at least about 3-fold greater expression or activity of Type II lipase compared to the reference strain. In certain embodiments, the at least one strain of bacteria has at least about 5-fold greater expression or activity of Type II lipase compared to the reference strain. In certain embodiments, the at least one strain of bacteria has at least about 10-fold greater expression or activity of Type II lipase compared to the reference strain.

In some embodiments, at least a portion of the type II lipase is expressed from a nucleic acid comprising SEQ ID NO: 32. SEQ ID NO: 32 is found in the complete circular genome of Propionibacterium acnes ATCC 11828 and starts at position 390,423 of ATCC 11828. The following subsequent positions are in reference to the first nucleotide of SEQ ID NO:32. The lipase coding sequence is bases 22-1032, referred to herein as ADE0051, HMPREF0675_4856, and SEQ ID NO: 7. Bases 1-21 is an intergenic region. Type II Lipase has a G in position 7 and an A in position 16. In some embodiments, at least a portion of the type I lipase is expressed from a nucleic acid comprising SEQ ID NO: 31. In contrast, relative to SEQ ID NO: 32, SEQ ID NO: 31 has a 6 bp sequence TAGATA inserted between base pairs 1 and 2, an A in position 7, a G in position 16, and a G between base pairs 145 and 146. SEQ ID NO: 31 and SEQ ID NO: 32 are shown in Table 9. FIG. 6 also illustrates the differences between sequences encoding Type I lipase and Type II lipase.

In certain embodiments, at least a portion of the Type II lipase is encoded by a nucleic acid with at least about 90% homology to SEQ ID NO: 7. In certain embodiments, at least a portion of the Type II lipase is encoded by a nucleic acid with at least about 95% homology to SEQ ID NO: 7. In certain embodiments, at least a portion of the Type II lipase is encoded by a nucleic acid with at least about 97% homology to SEQ ID NO: 7. In certain embodiments, at least a portion of the Type II lipase is encoded by a nucleic acid with at least about 97% homology to SEQ ID NO: 7. In certain embodiments, at least a portion of the Type II lipase is encoded by a nucleic acid with at least about 99% homology to SEQ ID NO: 7. In certain embodiments, at least a portion of the Type II lipase is encoded by a nucleic acid with 100% homology to SEQ ID NO: 7. In certain embodiments, at least a portion of the Type II lipase is encoded by a nucleic acid with at least about 90% homology to SEQ ID NO: 32. In certain embodiments, at least a portion of the Type II lipase is encoded by a nucleic acid with at least about 95% homology to SEQ ID NO: 32. In certain embodiments, at least a portion of the Type II lipase is encoded by a nucleic acid with at least about 97% homology to SEQ ID NO: 32. In certain embodiments, at least a portion of the Type II lipase is encoded by a nucleic acid with at least about 97% homology to SEQ ID NO: 32. In certain embodiments, at least a portion of the Type II lipase is encoded by a nucleic acid with at least about 99% homology to SEQ ID NO: 32. In certain embodiments, at least a portion of the Type II lipase is encoded by a nucleic acid with 100% homology to SEQ ID NO: 32.

### CRISPR/Cas5

In some embodiments, compositions disclosed herein comprise at least one strain of bacteria, wherein the at least one strain of bacteria comprises a CRISPR locus or a portion of a CRISPR locus. In some embodiments, compositions disclosed herein comprise at least one strain of bacteria, wherein the at least one strain of bacteria expresses a CRISPR-associated protein (Cas). By way of non-limiting example, the CRISPR-associated proteins include Cas5, Cas9, Cpf1, Cas3, Cas8a, Cas8b, Cas8c, Cas10d, Cse1, Cse2, Csy1, Csy2, Csy3, GSU0054, Cas10, Csm2, Cmr5, Cas10, Csx11, Csx10, Csf1, Csn2, Cas4, C2c1, C2c3, and C2c2.

In some embodiments, compositions disclosed herein comprise at least one strain of bacteria, wherein the at least one strain of bacteria expresses a Cas5. In some embodiments, compositions disclosed herein comprise at least one strain of bacteria, wherein the at least one strain of bacteria comprises a nucleic acid encoding a Cas5. In certain embodiments, the at least one strain of bacteria has been selected, transformed, or engineered to express a Cas5. In certain embodiments, the at least one strain of bacteria has at least about 1.5-fold greater expression or activity of Cas5 compared to a reference strain (e.g., pathogenic strain, not a health-associated strain). In certain embodiments, the at least one strain of bacteria has at least about 2-fold greater expression or activity of Cas5 compared to the reference strain. In certain embodiments, the at least one strain of bacteria has at least about 3-fold greater expression or activity of Cas5 compared to the reference strain. In certain embodiments, the at least one strain of bacteria has at least about 5-fold greater expression or activity of Cas5 compared to the reference strain. In certain embodiments, the at least one strain of bacteria has at least about 10-fold greater expression or activity of Cas5 compared to the reference strain.

In some instances, the at least one strain of bacteria expresses Cas5. In some embodiments, a strain of P. acnes is characterized as a health-associated P. acnes or a disease-associated P. acnes based on the presence of Cas5. In some embodiments, Cas5 is found in P. acnes strain ATCC 11828. In some embodiments, Cas5 is encoded by a sequence as set forth in SEQ ID NO: 12. In some embodiments, Cas5 is encoded by a sequence that is at least about 70%, 80%, 85%, 90%, 91%, 92%, 93%, 94%, 95%, 96%, 97%, 98%, 99%, or 100% homologous to SEQ ID NO: 12. n some embodiments, Cas5 is encoded by a sequence that is about 70%, 80%, 85%, 90%, 91%, 92%, 93%, 94%, 95%, 96%, 97%, 98%, 99%, or 100% sequence identity to SEQ ID NO: 12. In some embodiments, Cas5 is encoded by a sequence that is about 95% homologous to SEQ ID NO: 12. In some embodiments, Cas5 is encoded by a sequence that is about 97% homologous to SEQ ID NO: 12. In some embodiments, Cas5 is encoded by a sequence that is about 99% homologous to SEQ ID NO: 12. In some embodiments, Cas5 is encoded by a sequence that is about 100% homologous to SEQ ID NO: 12.

In some instances, the at least one strain of bacteria comprises a nucleic acid encoding Cas5, wherein the nucleic acid comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 550, at least about 650, at least about 700, or more than about 700 consecutive bases of SEQ ID NO: 12. In some instances, the at least one strain of bacteria comprises a nucleic acid encoding Cas5, wherein the nucleic acid comprises about 10, about 20, about 30, about 40, about 50, about 60, about 70, about 80, about 90, about 100, about 110, about 120, about 130, about 140, about 150, about 160 about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 550, about 650, about 700, or more than about 700 consecutive bases of SEQ ID NO: 12.

### ABC Transporter

In some embodiments, compositions disclosed herein comprise at least one strain of bacteria, wherein the at least one strain of bacteria comprises an ATP-binding cassette transporter. In certain embodiments, the at least one strain of bacteria comprises a nucleic acid encoding an ATP-binding cassette transporter (ABC transporter). In certain embodiments, the at least one strain of bacteria is selected for expression or overexpression of a nucleic acid encoding an ABC transporter. In certain embodiments, the at least one strain of bacteria is selected for increased activity of an ABC transporter. In certain embodiments, the at least one strain of bacteria is selected for the presence of a nucleic acid encoding an ABC transporter. In certain embodiments, the at least one strain of bacteria is transformed for overexpression of a nucleic acid encoding an ABC transporter. In certain embodiments, the at least one strain of bacteria is transformed for increased activity of an ABC transporter. In certain embodiments, the at least one strain of bacteria is transformed for the presence of a nucleic acid encoding an ABC transporter. In certain embodiments, the at least one strain of bacteria is engineered for overexpression of a nucleic acid encoding an ABC transporter. In certain embodiments, the at least one strain of bacteria is engineered for increased activity of an ABC transporter. In certain embodiments, the at least one strain of bacteria is engineered for the presence of a nucleic acid encoding an ABC transporter. In some embodiments, the ABC transporter is a portion of a known ABC transporter. In some embodiments, the ABC transporter is a portion of a known ABC transporter, wherein the portion of the known ABC transporter can perform an activity of the known ABC transporter. In some embodiments, the ABC transporter is a portion of a known ABC transporter, wherein the portion of the known ABC transporter can perform an enzymatic activity of the known ABC transporter. In some embodiments, the ABC transporter is a portion of a known ABC transporter, wherein the portion of the known ABC transporter can perform a transport activity of the known ABC transporter.

In certain embodiments, the at least one strain has at least about 1.5-fold greater expression or activity of ABC transporter compared to a reference strain (e.g., pathogenic strain, not a health-associated strain). In certain embodiments, the at least one strain has at least about 2-fold greater expression or activity of ABC transporter compared to the reference strain. In certain embodiments, the at least one strain has at least about 3-fold greater expression or activity of ABC transporter compared to the reference strain. In certain embodiments, the at least one strain has at least about 5-fold greater expression or activity of ABC transporter compared to the reference strain. In certain embodiments, the at least one strain has at least about 10-fold greater expression or activity of ABC transporter compared to the reference strain.

In some embodiments, ABC transporters disclosed herein are encoded by a sequence of SEQ ID NO: 10 or a sequence that is homologous to SEQ ID NO: 10. In some embodiments, the ABC transporter is encoded by a sequence that is at least about 80% homologous to SEQ ID NO: 10. In some embodiments, the ABC transporter is encoded by a sequence that is at least about 90% homologous to SEQ ID NO: 10. In some embodiments, the ABC transporter is encoded by a sequence that is at least about 95% homologous to SEQ ID NO: 10. In some embodiments, the ABC transporter is encoded by a sequence that is at least about 96% homologous to SEQ ID NO: 10. In some embodiments, the ABC transporter is encoded by a sequence that is at least about 97% homologous to SEQ ID NO: 10. In some embodiments, the ABC transporter is encoded by a sequence that is at least about 98% homologous to SEQ ID NO: 10. In some embodiments, the ABC transporter is encoded by a sequence that is at least about 99% homologous to SEQ ID NO: 10. In some embodiments, the ABC transporter is encoded by a sequence that is 100% homologous to SEQ ID NO: 10. In certain embodiments, the at least one strain is (completely or partially) selected, transformed, or engineered with a nucleic acid that is at least about 80%, 90%, 95%, 98%, 99%, or 100% homology to SEQ ID NO: 10 is partially or completely present in the at least one strain.

### DNA Binding Response Regulator

In some embodiments, compositions disclosed herein comprise at least one strain of bacteria, wherein the at least one strain of bacteria does not comprise a DNA binding response regulator. In certain embodiments, the at least one strain does not comprise a nucleic acid encoding a DNA binding response regulator. In certain embodiments, the at least one strain has been selected for reduced expression or activity of a DNA binding response regulator. In certain embodiments, the at least one strain has been selected for an absence of a DNA binding response regulator. In certain embodiments, bacteria disclosed herein have been transformed for reduced expression or activity of a DNA binding response regulator. In certain embodiments, bacteria disclosed herein have been transformed for an absence of a DNA binding response regulator. In certain embodiments, bacteria disclosed herein have been engineered for reduced expression or activity of a DNA binding response regulator. In certain embodiments, bacteria disclosed herein have been engineered for an absence of a DNA binding response regulator.

In certain embodiments, the at least one strain of bacteria has at least about 1.5-fold less expression or activity of a DNA binding response regulator relative to a reference strain (e.g., pathogenic strain, not a health-associated strain). In certain embodiments, the at least one strain has at least about 2-fold less expression or activity of a DNA binding response regulator relative to the reference strain. In certain embodiments, the at least one strain has at least about 3-fold less expression or activity of a DNA binding response regulator relative to the reference strain. In certain embodiments, the at least one strain has at least about 5-fold less expression or activity of a DNA binding response regulator relative to the reference strain. In certain embodiments, the at least one strain has at least about 10-fold less expression or activity of a DNA binding response regulator relative to the reference strain.

In certain embodiments, the at least one strain has been selected, transformed, or engineered to remove a nucleic acid with at least 90% homology to SEQ ID NO: 11. In certain embodiments, the at least one strain has been selected, transformed, or engineered to express a nucleic acid with at least 90% homology to SEQ ID NO: 11 at a lower level relative to the reference strain. In certain embodiments, the at least one strain has been selected, transformed, or engineered to remove a nucleic acid with at least 95% homology to SEQ ID NO: 11. In certain embodiments, the at least one strain has been selected, transformed, or engineered to express a nucleic acid with at least 95% homology to SEQ ID NO: 11 at a lower level relative to the reference strain. In certain embodiments, the at least one strain has been selected, transformed, or engineered to remove a nucleic acid with at least 97% homology to SEQ ID NO: 11. In certain embodiments, the at least one strain has been selected, transformed, or engineered to express a nucleic acid with at least 97% homology to SEQ ID NO: 11 at a lower level relative to the reference strain. In certain embodiments, the at least one strain has been selected, transformed, or engineered to remove a nucleic acid with at least 99% homology to SEQ ID NO: 11. In certain embodiments, the at least one strain has been selected, transformed, or engineered to express a nucleic acid with at least 99% homology to SEQ ID NO: 11 at a lower level relative to the reference strain. In certain embodiments, the at least one strain has been selected, transformed, or engineered to remove a nucleic acid with 100% homology to SEQ ID NO: 11. In certain embodiments, the at least one strain has been selected, transformed, or engineered to express a nucleic acid with 100% homology to SEQ ID NO: 11 at a lower level relative to the reference strain.

In certain embodiments, the at least one strain comprises a nucleic acid that is disrupted by an insertion of one or more nucleotides or a introduction of a frameshift mutation, wherein the nucleic acid has 80%, 90%, 95%, 98%, 99%, or 100% homology to SEQ ID NO: 11, before being disrupted. For example, in certain embodiments, a nucleic acid with at least 80%, 90%, 95%, 98%, 99%, or 100% homology to SEQ ID NO: 11 is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 3 prime end of the nucleic acid. In certain embodiments, the nucleic acid with at least 80%, 90%, 95%, 98%, 99%, or 100% homology to SEQ ID NO: 11 is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 5 prime end of the nucleic acid.

### Phosphoglycerate Kinase

In some embodiments, compositions disclosed herein comprise at least one strain of bacteria, wherein the at least one strain of bacteria does not comprise a phosphoglycerate kinase. In certain embodiments, the at least one strain does not comprise a gene encoding a phosphoglycerate kinase. In certain embodiments, the at least one strain has been selected for reduced expression or activity of a phosphoglycerate kinase. In certain embodiments, the at least one strain has been selected for absence of a phosphoglycerate kinase. In certain embodiments, bacteria disclosed herein have been transformed for reduced expression or activity of a phosphoglycerate kinase. In certain embodiments, bacteria disclosed herein have been transformed for absence of a phosphoglycerate kinase. In certain embodiments, bacteria disclosed herein have been engineered for reduced expression or activity of a phosphoglycerate kinase. In certain embodiments, bacteria disclosed herein have been engineered for absence of a phosphoglycerate kinase.

In certain embodiments, the at least one strain of bacteria has at least about 1.5-fold less expression or activity of a phosphoglycerate kinase relative to a reference strain (e.g., pathogenic strain, not a health-associated strain). In certain embodiments, the at least one strain has at least about 2-fold less expression or activity of a phosphoglycerate kinase relative to the reference strain. In certain embodiments, the at least one strain has at least about 3-fold less expression or activity of a phosphoglycerate kinase relative to the reference strain. In certain embodiments, the at least one strain has at least about 5-fold less expression or activity of a phosphoglycerate kinase relative to the reference strain. In certain embodiments, the at least one strain has at least about 10-fold less expression or activity of a phosphoglycerate kinase relative to the reference strain.

In certain embodiments, the at least one strain has been selected, transformed, or engineered to remove a nucleic acid with at least 90% homology to SEQ ID NO: 13. In certain embodiments, the at least one strain has been selected, transformed, or engineered to express a nucleic acid with at least 90% homology to SEQ ID NO: 13 at a lower level relative to the reference strain. In certain embodiments, the at least one strain has been selected, transformed, or engineered to remove a nucleic acid with at least 95% homology to SEQ ID NO: 13. In certain embodiments, the at least one strain has been selected, transformed, or engineered to express a nucleic acid with at least 95% homology to SEQ ID NO: 13 at a lower level relative to the reference strain. In certain embodiments, the at least one strain has been selected, transformed, or engineered to remove a nucleic acid with at least 97% homology to SEQ ID NO: 13. In certain embodiments, the at least one strain has been selected, transformed, or engineered to express a nucleic acid with at least 97% homology to SEQ ID NO: 13 at a lower level relative to the reference strain. In certain embodiments, the at least one strain has been selected, transformed, or engineered to remove a nucleic acid with at least 99% homology to SEQ ID NO: 13. In certain embodiments, the at least one strain has been selected, transformed, or engineered to express a nucleic acid with at least 99% homology to SEQ ID NO: 13 at a lower level relative to the reference strain. In certain embodiments, the at least one strain has been selected, transformed, or engineered to remove a nucleic acid with 100% homology to SEQ ID NO: 13. In certain embodiments, the at least one strain has been selected, transformed, or engineered to express a nucleic acid with 100% homology to SEQ ID NO: 13 at a lower level relative to the reference strain.

In certain embodiments, the at least one strain comprises a nucleic acid that is disrupted by an insertion of one or more nucleotides or a introduction of a frameshift mutation, wherein the nucleic acid has 80%, 90%, 95%, 98%, 99%, or 100% homology to SEQ ID NO: 13, before being disrupted. For example, in certain embodiments, a nucleic acid with at least 80%, 90%, 95%, 98%, 99%, or 100% homology to SEQ ID NO: 13 is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 3 prime end of the nucleic acid. In certain embodiments, the nucleic acid with at least 80%, 90%, 95%, 98%, 99%, or 100% homology to SEQ ID NO: 13 is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 5 prime end of the nucleic acid.

### Dermatin-Sulfate Adhesin

In some embodiments, compositions disclosed herein comprise at least one strain of bacteria, wherein the at least one strain of bacteria does not comprise a dermatin-sulfate adhesin (DSA1 and DSA2). In certain embodiments, the at least one strain does not comprise a nucleic acid encoding a dermatin-sulfate adhesin. In certain embodiments, the at least one strain has been selected for reduced expression or activity of a dermatin-sulfate adhesin. In certain embodiments, the at least one strain has been selected for an absence of a dermatin-sulfate adhesin. In certain embodiments, bacteria disclosed herein have been transformed for reduced expression or activity of a dermatin-sulfate adhesin. In certain embodiments, bacteria disclosed herein have been transformed for an absence of a dermatin-sulfate adhesin. In certain embodiments, bacteria disclosed herein have been engineered for reduced expression or activity of a dermatin-sulfate adhesin. In certain embodiments, bacteria disclosed herein have been engineered for an absence of a dermatin-sulfate adhesin.

In certain embodiments, the bacteria have been selected, transformed, or engineered for lower expression or activity or deletion of a dermatin-sulfate adhesin. In certain embodiments, the selected, transformed, or engineered bacteria has 1.5-fold, 2-fold, 3-fold, or 10-fold less expression or activity of the DSA1 or DSA2 compared to a reference strain. In certain embodiments, DSA1 or DSA2 is partially or completely deleted from the genome of the selected, transformed, or engineered, or engineered bacteria. In certain embodiments, DSA1 or DSA2 is disrupted by an insertion of one or more nucleotides or an introduction of a frameshift mutation in the genome of the selected, transformed, or engineered, or engineered bacteria.

### Hyaluronidase

In some embodiments, compositions disclosed herein comprise at least one strain of bacteria, wherein the at least one strain of bacteria does not comprise a hyaluronidase. Hyaluronidase is also known as hyaluronate lyase (locus tag PPA_RS01930). In some embodiments, compositions disclosed herein comprise at least one strain of bacteria, wherein the at least one strain of bacteria does not have hyaluronidase activity. In some embodiments, compositions disclosed herein comprise at least one strain of bacteria, wherein the at least one strain of bacteria does not comprise a nucleic acid encoding a hyaluronidase. In certain embodiments, the bacteria have been selected, transformed, or engineered for lower expression or activity of hyaluronidase relative to the bacteria before selecting, transforming or engineering, respectively.

In some embodiments, compositions disclosed herein comprise at least one strain of bacteria, wherein the at least one strain of bacteria comprises a hyaluronidase. In some embodiments, compositions disclosed herein comprise at least one strain of bacteria, wherein the at least one strain of bacteria has hyaluronidase activity. In some embodiments, compositions disclosed herein comprise at least one strain of bacteria, wherein the at least one strain of bacteria has a nucleic acid encoding a hyaluronidase. In certain embodiments, the bacteria have been selected, transformed, or engineered for greater expression or activity of hyaluronidase relative to the bacteria before selecting, transforming or engineering, respectively.

In certain embodiments, the selected, transformed, or engineered bacteria have 1.5-fold greater or lower expression or activity of hyaluronate lyase compared to a non-selected, transformed, or engineered strain. In certain embodiments, the selected, transformed, or engineered bacteria have 2-fold greater or lower expression or activity of hyaluronate lyase compared to a non-selected, transformed, or engineered strain. In certain embodiments, the selected, transformed, or engineered bacteria have 3-fold greater or lower expression or activity of hyaluronate lyase compared to a non-selected, transformed, or engineered strain. In certain embodiments, the selected, transformed, or engineered bacteria have 5-fold greater or lower expression or activity of hyaluronate lyase compared to a non-selected, transformed, or engineered strain. In certain embodiments, the selected, transformed, or engineered bacteria have 10-fold greater or lower expression or activity of hyaluronate lyase compared to a non-selected, transformed, or engineered strain. In certain embodiments, the bacteria have been selected, transformed, or engineered to acquire or express at a greater level, a nucleic acid with at least 90% homology to SEQ ID NO: 8. In certain embodiments, the bacteria have been selected, transformed, or engineered to acquire or express at a greater level, a nucleic acid with at least 95% homology to SEQ ID NO: 8. In certain embodiments, the bacteria have been selected, transformed, or engineered to acquire or express at a greater level, a nucleic acid with at least 97% homology to SEQ ID NO: 8. In certain embodiments, the bacteria have been selected, transformed, or engineered to acquire or express at higher level, a nucleic acid with at least 97% homology to SEQ ID NO: 8. In certain embodiments, the bacteria have been selected, transformed, or engineered to acquire or express at a higher level, a nucleic acid with at least 99% homology to SEQ ID NO: 8. In certain embodiments, the bacteria have been selected, transformed, or engineered to acquire or express at a higher level, a nucleic acid with 100% homology to SEQ ID NO: 8. In certain embodiments, the bacteria is a P. acnes bacteria. In certain embodiments, a gene with at least 80%, 90%, 95%, 98%, 99%, or 100% homology to SEQ ID NO: 8 is partially or completely deleted from the genome of the synthetic bacteria. In certain embodiments, a gene with at least 80%, 90%, 95%, 98%, 99%, or 100% homology to SEQ ID NO: 8 is disrupted by an insertion of one or more nucleotides or a introduction of a frameshift mutation in the genome of the selected, transformed, or engineered, or engineered bacteria. In certain embodiments, the selected, transformed, or engineered or selected bacteria are deoR+, Type II lipase positive, pIMPLE negative, or CRISPR Cas5 positive. In certain embodiments, the selected, transformed, or engineered or selected bacteria comprise P. acnes of ribotype RT1 and/or RT2.

In certain embodiments, the bacteria have been selected, transformed, or engineered for lesser expression or absence of hyaluronate lyase. In certain embodiments, the bacteria are selected, transformed, or engineered, or engineered to acquire the presence of a hyaluronidase gene. Hyaluronidase is also known as hyaluronate lyase (locus tag PPA_RS01930). In certain embodiments, the selected, transformed, or engineered, or engineered bacteria have 1.5-fold greater or lower expression or activity of hyaluronate lyase compared to a non-selected, transformed, or engineered strain. In certain embodiments, the selected, transformed, or engineered bacteria have 2-fold greater or lower expression or activity of hyaluronate lyase compared to a non-selected, transformed, or engineered strain. In certain embodiments, the selected, transformed, or engineered bacteria have 3-fold greater or lower expression or activity of hyaluronate lyase compared to a non-selected, transformed, or engineered strain. In certain embodiments, the selected, transformed, or engineered bacteria have 5-fold greater or lower expression or activity of hyaluronate lyase compared to a non-selected, transformed, or engineered strain. In certain embodiments, the selected, transformed, or engineered bacteria have 10-fold greater or lower expression or activity of hyaluronate lyase compared to a non-selected, transformed, or engineered strain. In certain embodiments, the bacteria have been selected, transformed, or engineered to acquire or express at a greater level, a nucleic acid with at least 90% homology to SEQ ID NO: 8. In certain embodiments, the bacteria have been selected, transformed, or engineered to acquire or express at a greater level, a nucleic acid with at least 95% homology to SEQ ID NO: 8. In certain embodiments, the bacteria have been selected, transformed, or engineered to acquire or express at a greater level, a nucleic acid with at least 97% homology to SEQ ID NO: 8. In certain embodiments, the bacteria have been selected, transformed, or engineered to acquire or express at higher level, a nucleic acid with at least 97% homology to SEQ ID NO: 8. In certain embodiments, the bacteria have been selected, transformed, or engineered to acquire or express at a higher level, a nucleic acid with at least 99% homology to SEQ ID NO: 8. In certain embodiments, the bacteria have been selected, transformed, or engineered to acquire or express at a higher level, a nucleic acid with 100% homology to SEQ ID NO: 8. In certain embodiments, the bacteria is a P. acnes bacteria. In certain embodiments, a nucleic acid with at least 80%, 90%, 95%, 98%, 99%, or 100% homology to SEQ ID NO: 8 is partially or completely deleted from the genome of the synthetic bacteria. In certain embodiments, a nucleic acid with at least 80%, 90%, 95%, 98%, 99%, or 100% homology to SEQ ID NO: 8 is disrupted by an insertion of one or more nucleotides or a introduction of a frameshift mutation in the genome of the selected, transformed, or engineered, or engineered bacteria. In certain embodiments, the selected, transformed, or engineered or selected bacteria are deoR+, Type II lipase positive, pIMPLE negative, or CRISPR Cas5 positive. In certain embodiments, the selected, transformed, or engineered or selected bacteria comprise P. acnes of ribotype RT1 and/or RT2.

### Alanine Dehydrogenase

In some embodiments, compositions disclosed herein comprise at least one strain of bacteria, wherein the at least one strain of bacteria does not comprise an alanine dehydrogenase. In some embodiments, compositions disclosed herein comprise at least one strain of bacteria, wherein the at least one strain of bacteria does not have alanine dehydrogenase activity. In some embodiments, compositions disclosed herein comprise at least one strain of bacteria, wherein the at least one strain of bacteria does not comprise a nucleic acid encoding an alanine dehydrogenase. In certain embodiments, the bacteria have been selected, transformed, or engineered for lower expression or activity of alanine dehydrogenase relative to the bacteria before selection, transformation or engineering, respectively.

In certain embodiments, bacteria are selected, transformed, or engineered for the absence or deletion of a nucleic acid encoding alanine dehydrogenase or a portion thereof. In some embodiments, the alanine dehydrogenase is encoded by a sequence of SEQ ID NO: 9. In some embodiments, the alanine dehydrogenase is encoded by a sequence that is at least 80% homology to SEQ ID NO: 9. In certain embodiments, the bacteria are selected, transformed, or engineered for the absence or deletion of an alanine dehydrogenase or a portion thereof with at least 90% homology to SEQ ID NO: 9. In certain embodiments, the bacteria are selected, transformed, or engineered for the absence or deletion of an alanine dehydrogenase or a portion thereof with at least 95% homology to SEQ ID NO: 9. In certain embodiments, the bacteria are selected, transformed, or engineered for the absence or deletion of an alanine dehydrogenase or a portion thereof with at least 97% homology to SEQ ID NO: 9. In certain embodiments, the bacteria are selected, transformed, or engineered for the absence or deletion of an alanine dehydrogenase or a portion thereof with at least 98% homology to SEQ ID NO: 9. In certain embodiments, the bacteria are selected, transformed, or engineered for the absence or deletion of an alanine dehydrogenase or a portion thereof with at least 99% homology to SEQ ID NO: 9. In certain embodiments, a nucleic acid with at least 80%, 90%, 95%, 98%, 99%, or 100% homology to SEQ ID NO: 9 is partially or completely deleted from the selected, transformed, or engineered, or engineered bacteria. In certain embodiments, a nucleic acid with at least 80%, 90%, 95%, 98%, 99%, or 100% homology to SEQ ID NO: 9 is disrupted by an insertion of one or more nucleotides or a introduction of a frameshift mutation in the selected, transformed, or engineered bacteria. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 3 prime end of the nucleic acid. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 5 prime end of the nucleic acid.

### Transposase 2

In some embodiments, compositions disclosed herein comprise at least one strain of bacteria, wherein the at least one strain of bacteria does not comprise a transposase 2. In some embodiments, compositions disclosed herein comprise at least one strain of bacteria, wherein the at least one strain of bacteria does not have transposase 2 activity. In some embodiments, compositions disclosed herein comprise at least one strain of bacteria, wherein the at least one strain of bacteria does not comprise a nucleic acid encoding a transposase 2. In certain embodiments, the bacteria have been selected, transformed, or engineered for lower expression or activity of transposase 2 relative to the bacteria before selection, transformation or engineering, respectively. In some embodiments, compositions disclosed herein comprise at least one strain of bacteria, wherein the at least one strain of bacteria does not have transposase 2 activity, expresses deoR and is of ribotype RT1.

In certain embodiments, bacteria are selected, transformed, or engineered for the absence or deletion of a nucleic acid encoding transposase 2 or a portion thereof. In some embodiments, the transposase 2 is encoded by a sequence of SEQ ID NO: 19. In some embodiments, the transposase 2 is encoded by a sequence that is at least 80% homology to SEQ ID NO: 19. In certain embodiments, the bacteria are selected, transformed, or engineered for the absence or deletion of a transposase 2 or a portion thereof with at least 90% homology to SEQ ID NO: 19. In certain embodiments, the bacteria are selected, transformed, or engineered for the absence or deletion of a transposase 2 or a portion thereof with at least 95% homology to SEQ ID NO: 19. In certain embodiments, the bacteria are selected, transformed, or engineered for the absence or deletion of a transposase 2 or a portion thereof with at least 97% homology to SEQ ID NO: 19. In certain embodiments, the bacteria are selected, transformed, or engineered for the absence or deletion of a transposase 2 or a portion thereof with at least 98% homology to SEQ ID NO: 19. In certain embodiments, the bacteria are selected, transformed, or engineered for the absence or deletion of a transposase 2 or a portion thereof with at least 99% homology to SEQ ID NO: 19. In certain embodiments, a nucleic acid with at least 80%, 90%, 95%, 98%, 99%, or 100% homology to SEQ ID NO: 19 is partially or completely deleted from the selected, transformed, or engineered, or engineered bacteria. In certain embodiments, a nucleic acid with at least 80%, 90%, 95%, 98%, 99%, or 100% homology to SEQ ID NO: 19 is disrupted by an insertion of one or more nucleotides or a introduction of a frameshift mutation in the selected, transformed, or engineered bacteria. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 3 prime end of the nucleic acid. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 5 prime end of the nucleic acid.

### Additional Markers

In some embodiments, compositions disclosed herein comprise at least one strain of bacteria, wherein the at least one strain of bacteria comprises at least one protein selected from a protein that mediates biosynthesis of a polysaccharide, a protein that mediates biosynthesis of cell wall, a protein that mediates biosynthesis of amino acids, a protein that mediates carbohydrate metabolism, and a protein that mediates glycerol transportation. In some embodiments, compositions disclosed herein comprise at least one strain of bacteria, wherein the at least one strain of bacteria comprises at least one nucleic acid that encodes a protein, wherein the protein mediates biosynthesis of a polysaccharide, a protein that mediates biosynthesis of cell wall, a protein that mediates biosynthesis of amino acids, a protein that mediates carbohydrate metabolism, and a protein that mediates glycerol transportation. In some embodiments, the protein that mediates biosynthesis of a polysaccharide is a glycosyl transferase. In some embodiments, the protein that mediates biosynthesis of cell-wall is a D-alanin-D-alanine ligase. In some embodiments, the protein that mediates amino acid biosynthesis is a cobalamin-independent methionine synthase. In some embodiments, the protein is a glycerol uptake facilitator protein. In some embodiments, the protein is a protoporphyrinogen oxidase. In some embodiments, the protoporphyrinogen oxidase is encoded by a hemY gene.

In some embodiments, compositions disclosed herein comprise at least one strain of bacteria, wherein the at least one strain of bacteria comprises at least one nucleic acid encoding a protein that is selected from a glycosyl transferase, a D-alanin-D-alanine ligase, and a cobalamin-independent methionine synthase.

In certain embodiments, the bacteria have been selected, transformed, or engineered for greater expression or activity of a protein, wherein the protein is selected from a protein that mediates biosynthesis of a polysaccharide, a protein that mediates biosynthesis of cell wall, and a protein that mediates biosynthesis of amino acids. In certain embodiments, the bacteria have been selected, transformed, or engineered for greater expression or activity of a protein selected from a glycosyl transferase, a D-alanin-D-alanine ligase, and a cobalamin-independent methionine synthase.

In some embodiments, compositions disclosed herein comprise at least one strain of bacteria, wherein the at least one strain of bacteria does not comprise a Christie-Atkins-Munch-Petersen (CAMP) protein. In some embodiments, compositions disclosed herein comprise at least one strain of bacteria, wherein the at least one strain of bacteria does not comprise a CAMP1 protein. In some embodiments, compositions disclosed herein comprise at least one strain of bacteria, wherein the at least one strain of bacteria does not comprise a CAMP2 protein. In some embodiments, compositions disclosed herein comprise at least one strain of bacteria, wherein the at least one strain of bacteria does not comprise a CAMP3 protein.

In some embodiments, compositions disclosed herein comprise at least one strain of bacteria, wherein the at least one strain of bacteria does not comprise a nucleic acid encoding a CAMP protein. In certain embodiments, the bacteria have been selected, transformed, or engineered for less expression or activity of a CAMP protein relative to the bacteria that is not selected, transformed or engineered. In certain embodiments, the bacteria have been selected, transformed, or engineered for no expression or activity of a CAMP protein. In certain embodiments, the bacteria have been mutated to remove at least a portion of a nucleic acid encoding a CAMP protein. In some embodiments, the CAMP protein is selected from CAMP1, CAMP2, and CAMP3.

In some embodiments, compositions disclosed herein comprise at least one strain of bacteria, wherein the at least one strain of bacteria does not comprise a sialidase. In some embodiments, compositions disclosed herein comprise at least one strain of bacteria, wherein the at least one strain of bacteria does not comprise a nucleic acid encoding a sialidase. In certain embodiments, the bacteria have been selected, transformed, or engineered for less expression or activity of a sialidase relative to the bacteria that is not selected, transformed or engineered. In certain embodiments, the bacteria have been selected, transformed, or engineered for no expression or activity of a sialidase. In certain embodiments, the bacteria have been mutated to remove at least a portion of a nucleic acid encoding a sialidase.

In some embodiments, compositions disclosed herein comprise at least one strain of bacteria, wherein the at least one strain of bacteria does not comprise a neuramidase. In some embodiments, compositions disclosed herein comprise at least one strain of bacteria, wherein the at least one strain of bacteria does not comprise a nucleic acid encoding a neuramidase. In certain embodiments, the bacteria have been selected, transformed, or engineered for less expression or activity of a neuramidase relative to the bacteria that is not selected, transformed or engineered. In certain embodiments, the bacteria have been selected, transformed, or engineered for no expression or activity of a neuramidase. In certain embodiments, the bacteria have been mutated to remove at least a portion of a nucleic acid encoding a neuramidase.

In certain embodiments, the bacteria has been selected, transformed, or engineered for higher activity or expression of any of the following proteins: Adhesion (NCBI Accession No. 50842581); CAMP factor (NCBI Accession No. 50842175, 50842711, 50842820, 50843546); Endoglycoceramidase (NCBI Accession No. 50842131); Iron transport lipoprotein (NCBI Accession No. 50841911); Lysozyme M1 (NCBI Accession No. 50843125); Protein PAGK_237 (NCBI Accession No. 482891444); Protein PPA0532 (NCBI Accession No. 50842016); Protein PPA0533 (NCBI Accession No. 50842017); or Protein PPA1498 (NCBI Accession No. 50842976). In certain embodiments, the bacteria has been selected, transformed, or engineered with a nucleic acid encoding any of the following protein any of the following proteins: Adhesion (NCBI Accession No. 50842581); CAMP factor (NCBI Accession No. 50842175, 50842711, 50842820, 50843546); Endoglycoceramidase (NCBI Accession No. 50842131); Iron transport lipoprotein (NCBI Accession No. 50841911); Lysozyme M1 (NCBI Accession No. 50843125); Protein PAGK_237 (NCBI Accession No. 482891444); Protein PPA0532 (NCBI Accession No. 50842016); Protein PPA0533 (NCBI Accession No. 50842017); or Protein PPA1498 (NCBI Accession No. 50842976).

In certain embodiments, the bacteria has been selected, transformed, or engineered for lower activity or expression of any of the following proteins: Adhesion (NCBI Accession No. 50843565 or 50843645); Cell wall hydrolase (NCBI Accession No. 50843410); Lipase/acylhydrolase (NCBI Accession No. 50843480); NPL/P60 protein (NCBI Accession No. 50842209); Peptide ABC transporter (NCBI Accession No. 50843590); Protein PPA1197 (NCBI Accession No. 50842677); Protein PPA1281(NCBI Accession No. 50842762); Protein PPA1715 (NCBI Accession No. 50843175); Protein PPA1939 (NCBI Accession No. 50843388); Protein PPA2239 (NCBI Accession No. 50843674); Rare lipoprotein A rlpa (NCBI Accession No. 50843612); or Triacylglycerol lipase (NCBI Accession No. 50843543). In certain embodiments, the bacteria has been selected, transformed, or engineered with a nucleotide to delete or disrupt a gene encoding any of the following proteins: Adhesion (NCBI Accession No. 50843565 or 50843645); Cell wall hydrolase (NCBI Accession No. 50843410); Lipase/acylhydrolase (NCBI Accession No. 50843480); NPL/P60 protein (NCBI Accession No. 50842209); Peptide ABC transporter (NCBI Accession No. 50843590); Protein PPA1197 (NCBI Accession No. 50842677); Protein PPA1281(NCBI Accession No. 50842762); Protein PPA1715 (NCBI Accession No. 50843175); Protein PPA1939 (NCBI Accession No. 50843388); Protein PPA2239 (NCBI Accession No. 50843674); Rare lipoprotein A rlpa (NCBI Accession No. 50843612); or Triacylglycerol lipas (NCBI Accession No. 50843543).

In certain embodiments, bacteria disclosed herein have been selected, transformed, or engineered for lower activity or expression of any of the following proteins: HMPREF0675_4855; HMPREF0675_4856; HMPREF0675_4479; HMPREF0675_4480; HMPREF0675_4481; HMPREF0675_3655/3657; HMPREF0675_4816; HMPREF0675_4817; HMPREF0675_5205; HMPREF0675_5206; HMPREF0675_5014; HMPREF0675_5101; HMPREF0675_5159; HMPREF0675_4093/4094; HMPREF0675_4163; HMPREF0675_5031; HMPREF0675_5390; HMPREF0675_3037. In certain embodiments, the bacteria have been selected, transformed, or engineered with a nucleotide to delete or disrupt a gene encoding any of the following proteins: HMPREF0675_4855; HMPREF0675_4856; HMPREF0675_4479; HMPREF0675_4480; HMPREF0675_4481; HMPREF0675_3655/3657; HMPREF0675_4816; HMPREF0675_4817; HMPREF0675_5205; HMPREF0675_5206; HMPREF0675_5014; HMPREF0675_5101; HMPREF0675_5159; HMPREF0675_4093/4094; HMPREF0675_4163; HMPREF0675_5031; HMPREF0675_5390; HMPREF0675_3037.

In certain embodiments, the bacteria have been selected, transformed, or engineered for higher activity or expression of any of the following proteins HMPREF0675_4855; HMPREF0675_4856; HMPREF0675_4479; HMPREF0675_4480; HMPREF0675_4481; HMPREF0675_3655/3657; HMPREF0675_4816; HMPREF0675_4817; HMPREF0675_5205; HMPREF0675_5206; HMPREF0675_5014; HMPREF0675_5101; HMPREF0675_5159; HMPREF0675_4093/4094; HMPREF0675_4163; HMPREF0675_5031; HMPREF0675_5390; HMPREF0675_3037. In certain embodiments, the bacteria has been selected, transformed, or engineered with a nucleic acid encoding any of the following proteins: HMPREF0675_4855; HMPREF0675_4856; HMPREF0675_4479; HMPREF0675_4480; HMPREF0675_4481; HMPREF0675_3655/3657; HMPREF0675_4816; HMPREF0675_4817; HMPREF0675_5205; HMPREF0675_5206; HMPREF0675_5014; HMPREF0675_5101; HMPREF0675_5159; HMPREF0675_4093/4094; HMPREF0675_4163; HMPREF0675_5031; HMPREF0675_5390; HMPREF0675_3037.

In certain embodiments, the selected, transformed, or engineered bacteria do not comprise an antibiotic resistance gene. In certain embodiments, the selected, transformed, or engineered bacteria lack an antibiotic resistance gene to any one or more of aminoglycoside, beta-lactam, colistin, fluoroquinolone, fosfomycin, fusidic acid, macrolide, lincosamide, streptogramin B, nitroimidazole, oxazolidinone, phenicol, rifampicin, sulphonamide, tetracycline, trimethoprim, or glycopeptide. In certain embodiments, an antibiotic can be applied to halt treatment with selected, transformed, or engineered bacteria disclosed herein. In certain embodiments, the antibiotic is aminoglycoside, beta-lactam, colistin, fluoroquinolone, fosfomycin, fusidic acid, macrolide, lincosamide, streptogramin B, nitroimidazole, oxazolidinone, phenicol, rifampicin, sulphonamide, tetracycline, trimethoprim, or glycopeptide.

In certain embodiments, the bacteria are selected, transformed, or engineered in order to reduce expression or release of pro-inflammatory mediators by human cells of which the bacteria contact. Bacteria may either directly or indirectly contact human cells (e.g., human skin cells). For instance, bacteria may indirectly contact human cells via factors secreted or released from the bacteria. Non-limiting example of pro-inflammatory mediators from human cells are IL-8, IL-1, IL-6, TNF-alpha, INF-alpha, and human beta defensin.

### Combination Treatments

It is envisioned that the any of the compositions, health-associated microbes or probiotics disclosed herein can be used in combination with other standard treatments. For example, in some embodiments, methods comprise administering compositions disclosed herein before, after, or simultaneously with administration of an additional therapeutic agent. In some embodiments, the methods may comprise topically administering an antibiotic. Non-limiting examples of topical antibiotics include clindamycin, doxycycline, erythromycin, and tetracycline. In some embodiments, the methods may comprise orally administering an antibiotic. Non-limiting examples of oral antibiotics include erythromycin; or a tetracycline, such as doxycycline or minocycline. Other standard treatments may comprise administering an anti-inflammatory agent, an antioxidant, an acid, light therapy, or a combination thereof.

In some embodiments, methods comprise treating the subject with a laser. In some embodiments, methods comprise treating the subject with blue light. In some embodiments, methods comprise treating the subject with red light. In some embodiments, methods comprise treating the subject with a targeted laser device and/or active ingredient directed to at least one pro-inflammatory strain of P. acnes. In some embodiments, methods comprise treating with a laser that targets porphyrins.

In exemplary embodiments, a probiotic is administered to the individual after a radiation treatment as described elsewhere herein. For example, the probiotic is administered within about 1 min and about 24 hours after treatment. Treatment includes a session where a dose is administered, either continuously or as multiple pulses. In some embodiments, the health-associated bacteria in an affected site of the individual is increased after treatment with the probiotic. In some cases, the health-associated bacteria replenishes, at least in part, health-associated bacteria killed or otherwise attenuated during radiation therapy. In some embodiments, the colony count of the health-associated bacteria is not decreased or is decreased by less than about 10%, 20%, 30%, 40% or 50% after treatment of the affected site with radiation therapy and administration of the probiotic.

Probiotics described herein include health-associated bacteria and optionally one or more engineered bacteria. In some cases, about 10⁶-10⁹ of the health-associated and/or synthetic bacteria is administered to the individual. In some methods, such as those targeting acne in an individual, the health-associated bacteria belong to P. acnes Type II clade, including, for example, P. acnes ribotype 2 and P. acnes ribotype 6. In some embodiments, a probiotic comprises an organism having at least about 90% sequence identity to any one of SEQ ID NOS: 1-4. In some embodiments, a probiotic comprises an organism having at least about 95%, 98%, 99% or about 100% homology to any one of SEQ ID NOS. 1-4. In some cases, the probiotic comprises an organism having at least about 95% homology to SEQ ID NO: 1. In some cases, the probiotic comprises an organism having at least about 95% homology to SEQ ID NO: 2. In some cases, the probiotic comprises an organism having at least about 95% homology to SEQ ID NO: 3. In some cases, the probiotic comprises an organism having at least about 95% homology to SEQ ID NO: 4. In some embodiments, a probiotic comprises an organism having at least about 95%, 98%, 99% or about 100% homology to any one of SEQ ID NOS. 1-4. In some embodiments, a probiotic comprises one or more synthetic bacteria. In some embodiments, a probiotic comprises one or more synthetic bacteria engineered from a source organism having at least about 90%, 95%, 98%, 99% or about 100% homology to any one of SEQ ID NOS. 1-4. In some embodiments, a probiotic comprises a bacteria in a composition having a bacterial concentration from about 10⁵ to about 10¹⁰ colony forming units/mL of the composition. The bacteria in the composition is inclusive of one or more types and/or strains of bacteria.

Probiotic compositions comprising a bacteria often further comprise one or more additional agents. Additional agents include, without limitation, antioxidants, benzoyl peroxide, and salicylic acid. Antioxidants include ascorbic acid, alpha-tocopherol, lipoic acid, glutathione, uric acid, ubiquinol, green tea extract, coffee berry extract, polyphenols such as resveratrol, and derivatives thereof, including 3,4,5 trihydroxystilbene (trans-resveratrol) and pterostilbene. In some embodiments, an additional agent is present in a probiotic composition at a concentration of between about 0.00005% and about 75%, between about 0.0005% and about 75%, between about 0.005% and about 75%, between about 0.05% and about 75%, between about 0.5% and about 75%, between about 0.00005% and about 25%, between about 0.0005% and about 25%, between about 0.005% and about 25%, between about 0.05% and about 25%, or between about 2.5% and about 25%. For example, resveratrol, or a derivative thereof, is present in a probiotic composition at a concentration of between about 0.05% and about 5%, or about 0.05%, 0.1%, 0.2%, 0.5%, 0.7%, 1%, 1.2%, 1.5%, 2%, 2.5%, 3%, 3.5%, 4%, 4.5%, or 5%.

Probiotics and compositions thereof are administered to a subject during treatments described herein in any manner suitable to provide a therapeutic effect. In certain methods, a probiotic is administered topically to affected tissue or skin. As non-limiting example, a probiotic is administered in a gel, ointment, cream, tincture, emulsion, foam or the like. In some methods, a probiotic is administered to affected skin by injection into or pricking of the skin. In some methods, a probiotic is administered orally. The probiotic composition can be administered prior to, or following treatment.

In some embodiments, a probiotic composition comprising a therapeutically effective amount of P. acnes Type II clade bacteria sufficient to provide P. acnes Type II clade bacteria to a target site of an individual at a colony count of at least about 10⁶-10⁹, after the composition is administered to the individual for a therapeutically effective period of time. In some cases, a therapeutically effective period of time is a one-time application of a topical composition comprising the bacteria. In some cases, a therapeutically effective period of time is a plurality of applications of a the topical composition. As a non-limiting example, the target site receives about 1 to 2 daily doses of 10⁶-10⁹ bacteria. The probiotic composition can be administered in combination with the radiation therapy of this disclosure; either following or prior to radiation treatment. The probiotic can also be administered in combination with an antibiotic. In many instances, the target site comprises skin affected with, or previously affected with, acne. The probiotic composition can comprise P. acnes of an RT1, RT2, RT3, or RT6 strain, or any combination thereof.

Additionally the probiotic composition can comprise an engineered or transformed bacteria. In certain embodiments, a gene that contributes to pathogenesis of a skin disorder is deleted or mutated to inactivate or reduce the corresponding gene product. In certain embodiments, a gene that reduces the pathogenesis of a skin disorder is added, or mutated to activate or increase levels of the corresponding gene product. In certain embodiments, transformed bacteria to be delivered as a probiotic comprise: presence or addition of the deoR repressor operon; presence or addition of Type II lipase, such as: a glycerol-ester hydrolase B (GehB); absence or deletion of type I lipase, such as: a glycerol-ester hydrolase A (GehA); absence or deletion of a pIMPLE plasmid; antibiotic sensitivity to macrolide and tetracycline antibiotics; absence or deletion of a thiopeptide encoding island; presence or addition of a tyrosine decarboxylase island (which increases intracellular pH under stress to tolerate acidic environments); ability to metabolize gelatin; absence or deletion of dermatin-sulfate adhesins (DSA1 and DSA2) which enable adhesion of microbes to keratinocytes; presence or increased expression of a hyaluronidase gene; increased hyaluronidase enzyme activity; absence of a hyaluronidase gene; reduced hyaluronidase activity; or any combination thereof. In certain embodiments, the transformed bacteria are S. epidermis. In certain embodiments, the transformed bacteria are P. acnes. In certain embodiments, the bacteria are transformed using recombinant DNA techniques known in the art. In certain embodiments, the bacteria are transformed by growing bacteria under selective pressure to acquire or lose a gene, gene product, or expression thereof. In a certain embodiment, the bacteria are transformed using CRISPR technology. In a certain embodiment, the bacteria are transformed using homologous recombination.

### Mixtures of Different Microbes

Provided herein are compositions of a plurality of health-associated microbes. The composition of health-associated microbes may be a mixture of a plurality of different health-associated microbes. In a certain embodiment, the mixture comprises at least one selected, transformed, or engineered bacteria. In a certain embodiment, the mixture comprises at least one selected, transformed, or engineered P. acnes. In certain embodiments, the mixture comprises 2, 3, 4, 5, 6, 7, 8, 9, 10 or more isolated and purified species, strains, ribotypes, or phylotypes of bacteria. In a certain embodiment, the mixture comprises at least one strain of bacteria that normally colonizes a tissue or body area other than the skin. In a certain embodiment, the mixture comprises at least one strain of bacteria that normally colonizes the oral cavity. In certain embodiments, the at least one bacteria that normally colonizes the oral cavity is S. salivarius. In a certain embodiment, the mixture comprises at least one strain of bacteria that normally colonizes the lumen of the gastrointestinal system. In a certain embodiment, the mixture comprises at least one bacteria that normally colonizes the lumen of the gastrointestinal system is a Lactobacillus or a Bifidobacterium. In certain embodiments, the Bifidobacterium is Bidifobacterium lactis Bb-12, Bifidobacterium animalis, Bifidobacterium breve, Bifidobacterium bifidum, or any combination thereof. In certain embodiments, the Lactobacillus is Lactobacillus acidophilus, Lactobacillus bulgaricus, Lactobacillus rhamnosus GG, Lactobacillus fermentumi, Lactobacillus Sakei, Lactobacillus casei, Lactobacillus salivarius, L rhamnosus LC705, Lactobacillus F19 L, Lactobacillus acidophilus La-5, or any combination thereof. In a certain embodiment, the mixture comprises at least 2, 3, 4, 5, 6, 7, 8, 9, 10, or more different bacterial ribotypes. In a certain embodiment, the mixture comprises at least 2, 3, 4, 5, 6, 7, 8, 9, 10, or more different bacterial phylotypes. In a certain embodiment, the mixture comprises at least 2, 3, 4, 5, 6, 7, 8, 9, 10, or more different bacterial species. In a certain embodiment, the mixture comprises at least 2, 3, 4, 5, 6, 7, 8, 9, 10, or more different bacterial species. In a certain embodiment, the mixture comprises a mixture of at least 2, 3, 4, 5, 6, 7, 8, 9, 10, or more different bacterial strains. In a certain embodiment, the mixture contains at least one non-bacterial microbe such as a fungus, virus, or bacteriophage. Any defined mixture of a plurality of probiotic strains may be recited to “consist essentially of.” This means that the mixture includes only the specified strains plus any non-active ingredient necessary for proper administration as a topical or oral formulation, such as an excipient or diluent.

In some embodiments, compositions disclosed herein comprise health-associated-microbes and probiotics especially useful for treating eczema. In some embodiments, compositions for treating eczema disclosed herein comprise Staphylococcus Aureus. In some embodiments, compositions for treating eczema disclosed herein comprise dead Staphylococcus Aureus bacteria. In some embodiments, compositions for treating eczema disclosed herein comprise Staphylococcus hominis. In other embodiments, the probiotic comprises of one more of a Dermacoccus, Methlyobacterium or Propionibacterium as they have a negative correlation with S. aureus. In other embodiments, a topical probiotic composition of S. mitis, S. sanguinis or S. cristatus are included in the probiotic.

### Formulations

Provided herein, in some aspects, are compositions that comprise at least one probiotic or health-associated microbe disclosed herein, wherein the compositions are formulated for administration to a subject in need thereof. Generally, the subject is a human afflicted with acne, eczema, psoriasis, seborrheic dermatitis, rosacea, or any combination thereof. In some embodiments, a composition is formulated for topical administration to a subject in need thereof. In some embodiments, the compositions are formulated for topical administration to the skin of the subject. In some embodiments, the compositions are formulated for topical administration to the scalp of the subject. In some embodiments, a composition is formulated for oral administration. By way of non-limiting example, compositions disclosed herein comprising strains of Lactobacillus may be preferentially administered by oral administration. In some embodiments, a composition is formulated for transdermal administration. In some embodiments, a composition is formulated for injectable administration. In certain embodiments, the composition is a formulation selected from a gel, ointment, lotion, emulsion, paste, cream, foam, mousse, liquid, spray, suspension, dispersion and aerosol. In certain embodiments, the formulation comprises one or more excipients to provide a desired form and a desired viscosity, flow or other physical or chemical characteristic for effective application, coverage and adhesion to skin.

Compositions disclosed herein may be presented in a formulation that includes one or more excipients to improve any one or more of shelf-life, application, skin penetration, and therapeutic effect. In some embodiments, the excipient is necessary to improve any one or more of shelf-life, application, skin penetration, and therapeutic effect.

In certain embodiments, the health-associated microbe or probiotic compositions described herein are formulated for oral ingestion. The oral ingestion form may be a pill, tablet, capsule, paste, liquid suspension, colloid, or mixed with various foods such as candies, chews, yogurt, milk, cottage cheese or non-dairy based or lactose reduced substitutes. The formulation may contain additional non-active ingredients that improve flavor, smell, or texture of the edible composition. The formulation may also include binding agents, encapsulating films, or excipients that preserve shelf-life and bioavailability.

In certain embodiments, health-associated-microbes and probiotic compositions disclosed herein that are administered orally comprise a species of bacteria selected from L. acidophilus La-5, Bifidobacterium animalis, Lactobacillus rhamnosus, Lactobacillus F19, Lactobacillus fermentum, Lactobacillus Sakei, Lactobacillus reuteri, Bifidobacterium breve, Propionibacterium freudenreichii ssp. Shermanii JS, Bifidobacterium bifidum, Lactobacillus acidophilus, Lactobacillus casei, and Lactobacillus salivarius, and combinations thereof. In some embodiments, the composition comprises Lactobacillus salivarius and a fructo-oligosaccharide. In some embodiments, the composition comprises Lactobacillus rhamnosus GG. In some embodiments, the composition comprises Lactobacillus rhamnosus LC705. In some embodiments, the composition comprises Lactobacillus salivarius and a prebiotic. In some embodiments, the composition comprises hydrolyzed whey formula with Lactobacillus rhamnosus GG. In some embodiments, the composition comprises hydrolyzed whey formula with Bifidobacterium lactis Bb-12. In certain embodiments, health-associated-microbes and probiotic compositions disclosed herein that are administered orally comprise bacteria selected from Lactobacillus rhamnosus GG and bifidobacteria Bb-12, and a combination thereof.

An emulsion may be described as s a preparation of one liquid distributed in small globules throughout the body of a second liquid. In some embodiments, the dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A lotion may be described as a low- to medium-viscosity liquid formulation. In some embodiments, the lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A cream may be described as a viscous liquid or semi-solid emulsion of either the “oil-in-water” or “water-in-oil type”. In some embodiments, the cream may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An ointment may be described as a semisolid preparation containing an ointment base and optionally one or more active agents of this disclosure. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A gel may be described as a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams may be described as an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Emollients may be described as externally applied agents that soften or soothe the skin and are generally known in the art and listed in compendia, such as the “Handbook of Pharmaceutical Excipients”, 4.sup.th Ed., Pharmaceutical Press, 2003. In certain embodiments, the emollients are almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

Surfactants are surface-active agents that lower surface tension and thereby increase the emulsifying, foaming, dispersing, spreading and wetting properties of a product. In certain embodiments, suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

Emulsifiers are surface active substances which promote the suspension of one liquid in another and promote the formation of a stable mixture, or emulsion, of oil and water. In certain embodiments, the emulsifiers are metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate. In one embodiment, the emulsifier is glycerol. In one embodiment, the emulsifier is glycerin.

In some embodiments, compositions disclosed herein are formulated to be applied to a subject's scalp. In some embodiments, the composition is formulated to be used as a product selected from a shampoo, a conditioner, a mousse, a gel, and a spray. Such compositions would be useful for the treatment of seborrheic dermatitis. Treatment of seborrheic dermatitis with such compositions may result in the reduction of a symptom selected from dandruff and cradle cap. However, compositions disclosed herein may be used to treat seborrheic dermatitis at other areas of the body besides the scalp. Non-limiting examples of other areas include the chest, stomach, skin folds, arms, legs, groin area and under breasts.

In some embodiments, compositions disclosed herein comprise a buffer, wherein the buffer controls a pH of the composition. Buffers are used to control pH of a composition. Preferably, the buffers buffer the composition from a pH of about 4 to a pH of about 7.5, from a pH of about 4 to a pH of about 7, and from a pH of about 5 to a pH of about 7.

In some embodiments, compositions disclosed herein are formulated to provide or maintain a desirable skin pH. In some embodiments, the desirable skin pH is between about 4.5 and about 6.5. In some embodiments, the desirable skin pH is between about 5 and about 6. In some embodiments, the desirable skin pH is about 5.5. In some embodiments, compositions disclosed herein are formulated with a skin pH modulating agent. Non-limiting examples of pH modulating agents include salicylic acid, glycolic acid, trichloroacetic acid, azeilic acid, lactic acid, aspartic acid, hydrochloride, stearic acid, glyceryl stearate, cetyl palmitate, urea phosphate, and tocopheryl acetate.

In some embodiments, compositions disclosed herein are formulated to provide more oxygen to the skin. In some embodiments, compositions disclosed herein are formulated to provide more oxygen exposure to the skin. In some embodiments, compositions disclosed herein are formulated to provide more oxygen diffusion into the skin. In some embodiments, compositions disclosed herein are formulated to provide more oxygen diffusion through the skin. In some embodiments, compositions disclosed herein are formulated with an agent that provides more oxygen to the skin. In some embodiments, compositions disclosed herein are used with an agent that provides more oxygen to the skin. In some embodiments, compositions disclosed herein are used before use of an agent that provides more oxygen to the skin. In some embodiments, compositions disclosed herein are used after use of an agent that provides more oxygen to the skin. A non-limiting example of an agent that provides oxygen to the skin is chlorophyll.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal. In one embodiment, a concentration of a preservative that is effective to prevent fungal growth is selected, without affecting the effectiveness of the composition for its intended purposed upon topical application.

Excipients in the formulation are selected based on the type of formulation intended. In certain embodiments, the excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars, and starches.

In some embodiments, compositions disclosed herein are formulated with glycerol. In some instances, a strain of bacteria in the composition ferments the glycerol, thereby producing short chain fatty acids. Non-limiting examples of short-chain fatty acids include acetic acid, lactic acid, and propionic acid. In some embodiments, the strain of bacteria is a Propionibacterium strain. In some embodiments, the strain of bacteria is a P. acnes strain.

Penetration enhancers are frequently used to promote transdermal delivery of drugs across the skin, in particular across the stratum corneum. Some penetration enhancers cause dermal irritation, dermal toxicity and dermal allergies. However, the more commonly used ones include urea, (carbonyldiamide), imidurea, N,N-diethylformamide, N-methyl-2-pyrrolidine, 1-dodecal-azacyclopheptane-2-one, calcium thioglycate, 2-pyyrolidine, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.).

The composition can be formulated to comprise the health-associated microbe or probiotic at a particular concentration. For example, the composition can comprise an amount of probiotic such that the microorganisms may be delivered in effective amounts. In certain embodiments, the amount of probiotic delivered is at least 1×10³, 1×10⁴, 1×10⁵, 1×10⁶, 1×10⁷, 1×10⁸, 1×10⁹, 1×10¹⁰ colony forming units per unit dose. The composition may be formulated with the health-associated microbe or probiotic in a proportion of at least about 0.0001% (expressed by dry weight), from about 0.0001% to about 99%, from about 0.001% to about 90% by weight, from about 0.01% to about 80% by weight, and from about 0.1% to about 70% by weight, relative to the total weight of the composition. In general, a composition intended to be administered topically comprises at least 1×10³, 1×10⁴, 1×10⁵, 1×10⁶, 1×10⁷, 1×10⁸, 1×10⁹, 1×10¹⁰ microorganisms per gram of carrier, or at equivalent doses calculated for inactive or dead microorganisms or for bacterial fractions or for metabolites produced.

Microbes disclosed herein may be delivered in effective amounts per unit dose, of at least about 1×10² colony forming units (cfu) to about 1×10²⁰ cfu. In the particular case of the compositions that have to be administered topically, the concentration of each bacterial strain and/or corresponding fraction and/or metabolite can be adjusted so as to correspond to doses (expressed as bacterial equivalent) ranging from about 1×10⁵ to about 1×10¹² cfu/dose.

Compositions disclosed herein for topical application generally comprise from about 1×10² to about 1×10¹⁵ cfu/g, from about 1×10⁵ to about 1×10¹² cfu/g, or from about 1×10⁶ to about 10×10¹² cfu/g of bacteria.

In certain embodiments, compositions disclosed herein are formulated in order to deliver at least 10⁴ microbes per cm² of skin. In certain embodiments, compositions disclosed herein are formulated in order to deliver at least 10⁵ microbes per cm² of skin. In certain embodiments, compositions disclosed herein are formulated in order to deliver at least 10⁶ microbes per cm² of skin. In certain embodiments, the composition is formulated in order to deliver at least 10⁷ microbes per cm² of skin. In certain embodiments, the composition is formulated in order to deliver at least 10⁸ microbes per cm² of skin. In certain embodiments, the composition is formulated in order to deliver at least 10⁹ microbes per cm² of skin. In certain embodiments, the composition is formulated in order to deliver less than 10¹⁰ microbes per cm² of skin. In certain embodiments, the composition is formulated in order to deliver less than 10⁹ microbes per cm² of skin. In certain embodiments, the composition is formulated in order to deliver less than 10⁸ microbes per cm² of skin. In certain embodiments, the composition is formulated in order to deliver less than 10⁷ microbes per cm² of skin. In certain embodiments, the composition is formulated in order to deliver between about 10⁴ and 10¹⁰ microbes per cm² of skin. In certain embodiments, the composition is formulated in order to deliver between about 10⁴ and 10⁹ microbes per cm² of skin. In certain embodiments, the composition is formulated in order to deliver between about 10⁵ and 10¹⁰ microbes per cm² of skin. In certain embodiments, the composition is formulated in order to deliver between about 10⁶ and 10¹⁰ microbes per cm² of skin. In certain embodiments, the composition is formulated in order to deliver between about 10⁷ and 10¹⁰ microbes per cm² of skin. In certain embodiments, the composition is formulated in order to deliver between about 10⁷ and 10⁸ microbes per cm² of skin. In certain embodiments, the composition is formulated in order to deliver between about 10⁶ microbes per cm² of skin and about 10¹⁰ microbes per cm² of skin. In certain embodiments, the composition is formulated in order to deliver between about 10⁶ microbes per cm² of skin and about 10⁹ microbes per cm² of skin. In certain embodiments, the composition is formulated in order to deliver between about 10′ microbes per cm² of skin and about 10¹⁰ microbes per cm² of skin. In certain embodiments, the composition is formulated in order to deliver between about 10⁷ microbes per cm² of skin and about 10⁹ microbes per cm² of skin.

In certain embodiments, compositions disclosed herein are formulated at a concentration of about 10⁵ microbes per milliliter to about 10¹² microbes per milliliter. In certain embodiments, compositions disclosed herein are formulated at a concentration of about 10⁶ microbes per milliliter. In certain embodiments, compositions disclosed herein are formulated at a concentration of about 10⁷ microbes per milliliter. In certain embodiments, compositions disclosed herein are formulated at a concentration of about 10⁸ microbes per milliliter. In certain embodiments, compositions disclosed herein are formulated at a concentration of about 10⁹ microbes per milliliter. In certain embodiments, compositions disclosed herein are formulated at a concentration of about 10¹⁰ microbes per milliliter.

In certain embodiments, compositions disclosed herein for topical or oral use contain biologic stability compounds including but not limited to carbohydrates such as trehalose, mannose, fructose, glucose, sucrose, lactose, raffinose, stachyose, melezitose, dextran, and sugar alcohols; and/or cryopreservatives such as glycerol, bovine-free media, (e.g., tryptic soy broth), whey protein, NaCl, phosphate buffer, MgCl, lyophilized bacteria, or other inactive/killed bacteria.

In some embodiments, compositions disclosed herein can be added to an applicator before packaging. Non-limiting examples of applicators include a cotton pad, a polyester pad, a q-tip, a sponge, and a brush. In some embodiments, the applicator is placed in a package. Non-limiting examples of a package includes bags and foil or wax lined paper packets. The interior of the package may be sterile. In some embodiments, air in the package is removed with a vacuum before sealing. In some embodiments, the package is heat-sealed. In some embodiments, the package is sealed with adhesive.

After formulation, composition disclosed herein may be packaged in a manner suitable for delivery and use by an end user. In one embodiment, the composition is placed into an appropriate dispenser and shipped to the end user. Examples of a final container may include a pump bottle, squeeze bottle, jar, tube, capsule or vial.

In another embodiment, compositions disclosed herein are lyophilized or freeze dried, for reconstitution before application to the skin. In one embodiment, lyophilization or freeze drying is conducted with one or more excipients, such as glycerol or other sugar alcohols, to improve the shelf life of the selected, transformed, or engineered bacteria. In one embodiment, the lyophilized composition does not include trehalose (.alpha.-D-glucopyranosyl-1,1-.alpha.-D-glucopyranosyde). In some embodiments, the composition does not have to be frozen.

Compositions disclosed herein may be packaged in one or more containers. For example, a single bottle, tube, container, or capsule may be divided to two equal or unequal parts wherein one part contains the bacteria, in their packing form (freeze dried/inactive, etc.), and the other part contains an activation material, which can be a liquid or a gel. The single bottle or container can be designed so that an end user can dispense with a single force applied to the container all or a portion of the contents in the two container parts, to dispense onto the skin or other surface the selected, transformed, or engineered bacteria and the activation material. The kit may also be of the form that comprises two or more containers, one container with the population(s) of selected, transformed, or engineered bacteria and the other with a formulation for admixture with the populations of selected, transformed, or engineered bacteria. In another example, two or more containers, one container with the population of selected, transformed, or engineered bacteria, the other container with natural non pathogenic skin bacteria that are not selected, transformed, or engineered, and a third container with a formulation for admixture with the populations of selected, transformed, or engineered bacteria. In another example, the two or more containers composing the single bottle had one pump connected to two separate tubes, each draining from a different chamber. The kit may also include one or more complementary products, such as soaps, body washes or moisturizing lotions with certain pH, lotions or creams containing active compounds, bacteria and limiting factors etc. In another embodiment, the complementary product is a limiting factor that will enhance the growth, activity and/or expression of the compound of interest to provide a lasting or continuous expression of the compound. The complementary product may include any compound beneficial to the activity of the original product, and enhance its activity for lasting efficacy. Another contemplated packaging is one wherein the population of selected, transformed, or engineered bacteria is maintained as a layer on a bandage or film that is combined with a second layer of bandage/film that will allow activation of the bacteria, and that optionally may also limit reproduction/growth factors. In another embodiment, the final product is stored refrigerated, with the bacteria being in their active state. In another embodiment, the bacteria is stored in a small bead of water soluble cellulose. The beads can be mixed in any solution such as sunscreen/moisturizing/body wash or soap.

### Additional Active Ingredients

Compositions disclosed herein may comprise additional active ingredients. In certain embodiments, compositions disclosed herein comprise at least one non-living, non-microbial constituent. By way of non-limiting example, the non-living, non-microbial constituent may be selected from a small molecule, a fatty acid, an antibiotic, a metabolite, an antioxidant, and a retinoid. Non-limiting examples of antioxidants are vitamin C and vitamin E. Non-limiting examples of a retinoid are tretinoin, tazarotene, adapalene, and retinol. In some embodiments, the non-living, non-microbial constituent is vitamin D, which may be in the form of calciptotriene. In some embodiments, the additional active ingredient is an agent that has anti-inflammatory activity.

In certain embodiments, the composition contains an antibiotic. Non-limiting examples of antibiotics include macrolide, tetracycline, β-lactam, aminoglycoside, cephalosporin, carbapenems, quinolone/fluoroquinolone, sulfonamides, salicylic acid, glycolic acid, azaleic acid, live phage therapy, synthetic phage contractile nanotubes, laser, dapsone, benzoyl peroxide, benzoyl peroxide/resveratrol combinations, and any combination thereof. In some embodiments, the antibiotic is selected from clindamycin, doxycycline, erythromycin, and tetracycline, wherein the antibiotic is formulated for topical administration. In some embodiments, the antibiotic is selected from erythromycin, tetracycline, doxycycline and minocycline, wherein the antibiotic is formulated for oral administration.

In certain embodiments, a composition disclosed herein comprises a topical anti-acne medication such as benzoyl peroxide or salicylic acid. The concentration of benzoyl peroxide or salicylic acid included in a composition may be lower than that commonly included in a single formulation (without a health-associated microbe or probiotic). In some embodiments, the concentration of the anti-acne medication is between about 0.1% and about 3%. In some embodiments, the concentration of the anti-acne medication is between about 0.1% and about 2.5%. In some embodiments, the concentration of the anti-acne medication is between about 0.5% and about 2.5%. In some embodiments, the concentration of the anti-acne medication is between about 1% and about 2.5%. In some embodiments, the concentration of the anti-acne medication is less than about 2.5%. In some embodiments, the concentration of the anti-acne medication is less than about 2%. In some embodiments, the concentration of the anti-acne medication is less than about 1.5%. In some embodiments, the concentration of the anti-acne medication is less than about 1%. In some embodiments, the concentration of the anti-acne medication is less than about 0.5%. In some embodiments, the concentration of the anti-acne medication is less than about 0.1%.

In certain embodiments, compositions disclosed herein comprise a topical anti-acne medication such as a retinoid. Non-limiting examples of topical retinoid compounds include retinoic acid, tretinoin, adapalene, and tazarotene. In certain embodiments, compositions disclosed herein comprise resveratrol or trans-resveratrol. In some embodiments, the concentration of the retinoid or resveratrol in the composition is less than about 10%. In some embodiments, the concentration of the retinoid or resveratrol in the composition is less than about 5%. In some embodiments, the concentration of the retinoid or resveratrol in the composition is less than about 2.5%. In some embodiments, the concentration of the retinoid or resveratrol in the composition is less than about 1%. In some embodiments, the concentration of the retinoid or resveratrol in the composition is less than about 0.5%. In some embodiments, the concentration of the retinoid or resveratrol in the composition is between about 0.5% and about 10%. In some embodiments, the concentration of the retinoid or resveratrol in the composition is between about 1% and about 10%. In some embodiments, the concentration of the retinoid or resveratrol in the composition is between about 0.5% and about 2.5%.

In some embodiments, compositions disclosed herein comprise at least one omega-3 fatty acid. Non-limiting examples of omega-3 fatty acids include hexadecatrienoic acid (HTA), α-Linolenic acid (ALA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA), clupanodonic acid, docosahexaenoic acid (DHA), tetracosapentaenoic acid, tetracosahexaenoic acid (nisinic acid), and phytosphingosine.

In some embodiments, compositions disclosed herein comprise an acid selected from glycolic acid, azaelic acid, and trichloroacetic acid. In some embodiments, compositions disclosed herein comprise a natural extract, such as tea tree oil or green tea extract.

In some embodiments, the additional active ingredient comprises a drug targeting at least one strain of P. acnes. In some embodiments, the drug targeting at least one strain of P. acnes is a small molecule drug. In some embodiments, the drug targeting at least one strain of P. acnes is a small molecule inhibitor of an enzyme expressed by P. acnes. In some embodiments, the enzyme expressed by P. acnes is required for P. acnes growth or P. acnes energy metabolism. In some embodiments, the drug targeting at least one strain of P. acnes is a biologic. In some embodiments, the biologic comprises a peptide. In some embodiments, the biologic comprises an antibody or antigen binding fragment. In some embodiments, the biologic comprises and antibody-small molecule conjugate. In some embodiments, the biologic comprises and antibody-drug conjugate. In some embodiments, the biologic comprises a nucleic acid. In some embodiments, then nucleic acid comprises an antisense nucleic acid molecule, wherein the antisense nucleic acid molecule inhibits an enzyme expressed by P. acnes. In some embodiments, the enzyme expressed by P. acnes is required for P. acnes growth or P. acnes energy metabolism. In some embodiments, the antisense nucleic acid molecule comprises siRNA. In some embodiments, the antisense nucleic acid molecule comprises a shRNA. In some embodiments, the antisense nucleic acid molecule comprises a guide RNA to be used with a CRISPR-associated protease. In some embodiments, the additional active ingredient (e.g., guide RNA and CRISPR-associated protease) and targets a genomic element specific for strains of P. acnes associated with acne.

### Methods of Treating Skin Disorders

Provided herein, in some aspects, are methods for treating or preventing skin disorders disclosed herein. In some aspects, the disclosure provides methods for treating or preventing acne. Methods for treating or preventing acne generally comprise administering an effective amount of a strain of bacteria disclosed herein to the skin of a subject in need thereof. In some embodiments, methods comprise administering at least one composition disclosed herein. In some embodiments, methods comprise administering at least one species of bacteria disclosed herein. In some embodiments, methods comprise administering at least one strain of bacteria disclosed herein. In some embodiments, methods comprise administering at least one ribotype of bacteria disclosed herein. In some embodiments, methods comprise administering at least one probiotic disclosed herein. In some embodiments, methods comprise administering at least one health-associated microbe disclosed herein. In some embodiments, methods comprise administering at least one selected species of selected bacteria disclosed herein. In some embodiments, methods comprise administering at least one selected strain of selected bacteria disclosed herein. In some embodiments, methods comprise administering at least one selected, transformed, or engineered bacteria disclosed herein. In some embodiments, methods comprise administering at least one genetically modified bacteria disclosed herein. In some embodiments, methods comprise administering at least one genetically engineered bacteria disclosed herein. In some embodiments, methods comprise administering at least one health-associated strain of bacteria comprises a bacteria described herein.

In some embodiments, methods comprise administering at least one health-associated strain of bacteria selected from a health-associated strain of Propionibacterium, Staphylococcus and Lactobacillus bacteria. In some embodiments, methods comprise administering at least one health-associated strain of Propionibacterium bacteria. In some embodiments, methods comprise administering at least one health-associated strain selected from a health-associated strain of P. acnes, a health-associated strain of P. granulosum, a health-associated strain of P. avidum, and a health-associated Staphylococcus, as described herein. In some embodiments, the health-associated strain of P. acnes is P. acnes subsp. acnes. In some embodiments, methods comprise administering at least one health-associated strain of P. acnes. In some embodiments, methods comprise administering at least one health-associated fungus. The health-associated fungus may be Malassezia.

In some embodiments, the methods comprise administering a microbe disclosed herein. In some embodiments, the methods comprise administering a mixture of at least two microbes disclosed herein. In some embodiments, the methods comprise administering at least one health-associated microbe disclosed herein. In some embodiments, the microbe is a fungus. In some embodiments, the microbe is a protist. In some embodiments, the microbe is bacteria. In some embodiments, the bacteria comprises P. acnes.

In some embodiments, methods comprise administering a composition disclosed herein. In some embodiments, methods comprise administering a composition having a formulation disclosed herein.

In certain embodiments, methods disclosed herein comprise applying an effective amount of a composition containing a plurality of microbes of different ribotypes. In certain embodiments, the strain is a P. acnes strain of a ribotype selected from RT1, RT2, RT3, RT4, RT5, RT7, RT8, RT9, and RT10. In certain embodiments, the strain is a P. acnes strain of a ribotype selected from RT1, RT2, RT3, RT7, RT8, RT9, and RT10. In certain embodiments, the plurality of microbes is a mixture of two or more P. acnes strains of different ribotypes. In certain embodiments, the different ribotypes comprise RT1 and RT2. In certain embodiments, the different ribotypes comprises RT1 and RT3. In certain embodiments, the different ribotypes comprise RT1 and not RT6. In certain embodiments, the different ribotypes comprise RT2 and RT3. In certain embodiments, the v comprises RT2 and not RT6. In certain embodiments, the different ribotypes comprise RT2 and RT3. In certain embodiments, the different ribotypes comprise RT3 and not RT6. In certain embodiments, the different ribotypes consist essentially of RT1 and RT2. In certain embodiments, the different ribotypes consist essentially of RT1 and RT3. In certain embodiments, the different ribotypes consist essentially of RT1 and not RT6. In certain embodiments, the different ribotypes consist essentially of RT2 and RT3. In certain embodiments, the different ribotypes consist essentially of RT2 and not RT6. In certain embodiments, the different ribotypes consist essentially of RT2 and RT3. In certain embodiments, the different ribotypes consist essentially of RT3 and not RT6. In certain embodiments, the mixture does not comprise ribotype RT6.

In certain embodiments, methods described herein comprise applying an effective amount of a composition containing a plurality of microbes of different ribotypes. In certain embodiments, the plurality of microbes is a mixture of three or more P. acnes strains of different ribotypes. In certain embodiments, the mixture comprises RT1, RT2, and RT3. In certain embodiments, the mixture comprises RT2, RT3, and not RT6. In certain embodiments, the mixture comprises RT1, RT2, and RT6. In certain embodiments, the mixture comprises RT1, RT3, and not RT6. In certain embodiments, the mixture consists essentially of RT1, RT2, and RT3. In certain embodiments, the mixture consists essentially of RT2, RT3, and not RT6. In certain embodiments, the mixture consists essentially of RT1, RT2, and not RT6. In certain embodiments, the mixture consists essentially of RT1, RT3, and not RT6. In certain embodiments, the mixture does not comprise RT6.

In some embodiments, described herein, methods for treating or preventing acne comprising: administering an effective amount of bacteria associated with healthy or normal skin. Healthy or normal skin may be skin essentially free of acne. In some embodiments, described herein, methods for treating or preventing acne comprising: administering an effective amount of bacteria that produces low to absent levels of pro-inflammatory mediators when co-incubated with a subjects keratinocytes.

Methods disclosed herein may comprise administering a drug to the subject. In some embodiments, the drug is administered orally. In some embodiments, the drug is administered topically. In some embodiments, the drug is administered topically to the skin of the subject that is affected by acne. In certain embodiments, described herein, are methods for preventing or treating acne in a subject comprising: administering an effective amount of a drug specifically targeting P. acnes. In certain embodiments, described herein, are methods for preventing or treating acne in a subject comprising: administering an effective amount of a drug targeting P. acnes of a ribotype selected from RT1, RT2, RT3, RT4, RT5, RT7, RT8, RT9, and RT10, and combinations thereof. In certain embodiments, described herein, are methods for preventing or treating acne in a subject comprising: administering an effective amount of a drug targeting P. acnes of a ribotype selected from RT1, RT2, RT3, RT7, RT8, RT9, and RT10, and combinations thereof. In certain embodiments, described herein, are methods for preventing or treating acne in a subject comprising: administering an effective amount of a drug targeting a strain of bacteria or fungus that induces or promotes production of inflammatory mediators from a subject's keratinocytes when co-incubated with a subject's keratinocytes. In certain embodiments, described herein, are methods for preventing or treating acne in a subject comprising: administering an effective amount of a drug targeting a strain of bacteria or fungus that induces or promotes production of inflammatory mediators from a subject's keratinocytes when a factor produced by the bacteria or fungus is co-incubated with a subject's keratinocytes. Such drugs are disclosed herein, e.g., small molecules, antisense molecules, siRNA, biologics, antibodies, and combinations thereof.

Methods disclosed herein may comprise performing laser therapy on the skin of the subject. In some embodiments, laser therapy is performed prior to administration of a drug or bacteria disclosed herein to a subject. In some embodiments, laser therapy is performed after administration of a drug or bacteria disclosed herein to a subject. In some embodiments, laser therapy is performed simultaneously with administration of a drug or bacteria disclosed herein to a subject. The laser therapy may be a blue or red-light laser targeting porphyrins.

Compositions disclosed herein may be administered, daily, weekly, or monthly. In certain embodiments, the composition is administered twice daily. In certain embodiments, the composition is administered at least twice weekly. In certain embodiments, the composition is administered at least twice monthly.

### Diagnosis of Acne and Other Diseases

Health-associated P. acnes can be differentiated from disease-associated P. acnes based on genetic differences. For example, disease-associated P. acnes have been shown to harbor genomic elements that encode for multiple virulence genes. Many health-associated P. acnes, on the other hand, carry clustered regularly interspaced short palindromic repeat (CRISPR) elements that prevent health-associated P. acnes from acquiring virulence genes. In addition to genetic differences, an increase in P. acnes derived molecules such as porphyrins, proteases, lipases, and hemolysins, may be associated with disease. Therefore, genes encoding for biosynthesis of P. acnes derived molecules (e.g., porphyrins, proteases, lipase, and hemolysins) may be useful to distinguish between health-associated P. acnes and disease-associated P. acnes.

In one aspect, provided herein are methods of differentiating health-associated P. acnes bacteria from disease-associated P. acnes bacteria based on genetic markers. Exemplary methods comprise measuring expression of one or more genetic markers including, but not limited to, deoR, Cas5, pIMPLE, type I lipase, type II lipase, and alanine dehydrogenase. In some instances, methods for differentiating between health-associated P. acnes and disease-associated P. acnes comprise differentiation based on ribotype. Also provided herein are methods of differentiating between health-associated P. acnes and disease-associated P. acnes comprising quantitative PCR or sequencing of at least one genetic marker, such as, deoR, Cas5, pIMPLE, type I lipase, type II lipase, alanine dehydrogenase, or a combination thereof. In some cases, methods comprise differentiating between strains and/or ribotypes of P. acnes bacteria. As a non-limiting example, P. acnes RT6 comprises a DNA-binding response regulator and/or phosphoglycerate kinase, which is absent in RT1, RT2, RT3, RT4, and RT5. As another example, an ABC transporter gene is absent from RT6 and present in RT1, RT2, RT3, RT4, and RT5. In some cases, such methods are used to select for bacteria for use in a probiotic described herein. For example, RT6 is not present in a probiotic. In some cases, such methods are used to monitor treatment of an individual with the probiotic. Moreover, in some cases, the methods are used to distinguish between a health-associated and disease-associated bacteria of the same ribotype. For example, P. acnes RT1 has both a disease-associated and health-associated P. acnes that are distinguishable based on the presence and/or absence of genetic markers.

Methods of diagnosing and/or monitoring treatment of a disease in an individual comprising quantifying health-associated and/or disease-associated P. acnes in a sample from the individual are also provided. Certain methods for quantification as provided herein comprise measuring at least one of deoR, a CRISPR-associated protein, pIMPLE, type I lipase, type II lipase, alanine dehydrogenase, DNA-binding response regulator, phosphoglycerate kinase, and ABC transporter. 23S ribosomal RNA may be measured to determine quantity of total bacteria in a sample. In some embodiments, the treatment comprises administration of a health-associated microbe or probiotic provided herein. As a non-limiting example, the probiotic comprises a P. acnes of RT1 and RT2. In some cases, the probiotic does not comprise a P. acnes of RT6.

Further provided herein are methods for determining a microbiome profile in an individual by detecting and/or quantifying at least one of deoR, a CRISPR-associated protein, pIMPLE, type I lipase, type II lipase, alanine dehydrogenase, DNA-binding response regulator, phosphoglycerate kinase, and ABC transporter in a sample from the individual. For determining relative quantities of bacteria in a sample, 23S ribosomal RNA sequences may be used to estimate total bacteria in a sample from the individual. Determination of a microbiome profile may be useful before and/or during treatment, for example, treatment with a probiotic or health-associated bacteria, such as P. acnes. Methods for monitoring a treatment regimen following determination of a microbiome profile are also provided.

In certain embodiments, methods comprise detecting or characterizing a microbe in a sample of a subject. In some embodiments, the methods comprise detecting the microbe in an ex vivo sample. In some embodiments, the methods comprise detecting the microbe in an in vivo sample. An exemplary method comprises: obtaining a stool sample from the subject; isolating bacterial DNA from the sample; amplifying DNA selected from 16S, ribosomal DNA, 18S ribosomal DNA, and/or 23S ribosomal DNA, sequencing the amplified DNA products; and typing the individual's gut bacteria. In certain embodiments, are methods for determining whether a subject has acne comprising: obtaining a stool sample from the subject; isolating bacterial DNA from the sample and whole genome shotgun sequencing DNA in the sample. Another exemplary method comprises obtaining a swab of skin sample from the subject, such as from the face, back or other affected area.

### Biological Samples for Diagnosis

In certain embodiments, diagnosing an individual comprises obtaining a biological sample. In certain embodiments, the biological sample is a skin sample or biopsy. In certain embodiments, the biological sample is a stool or fecal sample. In certain embodiments, the biological sample is an oral or oral mucosal sample. In certain embodiments, the biological sample is a swab, obtained using a cotton swab or other compatible swab. In certain embodiments, the sample contains bacteria. In certain embodiments, the sample contains a fungus. In certain embodiments, the sample contains a virus. In certain embodiments, the virus is a bacteriophage. In certain embodiments, the sample is stored after it has been collected, but before additional steps are performed. In certain embodiments, storage occurs at less than 8° C. In certain embodiments, storage occurs at less than 4° C. In certain embodiments, storage occurs at less than 0° C. In certain embodiments, storage occurs at less than −20° C. In certain embodiments, storage occurs at less than −70° C. In certain embodiments, this storage is in glycerol, glycol, dimethyl sulfoxide, growth media, nutrient broth or any combination thereof. In certain embodiments, the sample is stored for at least about 1, 2, 3, 4, 5, 6, or 7 days. In some embodiments, the sample is stored for at least about 1, 2, 3, or 4 weeks. In some embodiments, the sample is stored for at least about 1, 2, 3, 4, 5, 6, 7, 8, 9, 10, 11 or 12 months. In some embodiments, the sample is stored for up to about 1, 2, 3, or 4 weeks. In some embodiments, the sample is stored for up to about 1, 2, 3, 4, 5, 6, 7, 8, 9, 10, 11 or 12 months. In some embodiments, the sample is cultured at a physiological temperature suitable for bacterial growth such as 30° C., 31° C., 32° C., 33° C., 34° C., 35° C., 36° C., 37° C., 38° C., 39° C. or 40° C. In some embodiments, the sample is cultured at about 35° C. to about 39° C. In some embodiments, the sample is cultured at about 36° C. to about 38° C. In some embodiments, the sample is cultured in a liquid growth media. In some embodiments, the sample is cultured on a solid growth media such as an agar plate. In some embodiments, the plates are blood agar.

In certain embodiments, samples disclosed herein are processed further before analysis. In some embodiments, no extraction or processing procedures are performed on the sample. In some embodiments, nucleic acid is extracted from the sample. In some embodiments, the nucleic acid is DNA. In some embodiments, the DNA is bacterial DNA. In some embodiments, the Bacterial DNA is 16S ribosomal DNA. In some embodiments, the Bacterial DNA is 18S ribosomal DNA. In some embodiments, the Bacterial DNA is 23S ribosomal DNA. In some embodiments, the nucleic acid is ribosomal DNA. In some embodiments, the nucleic acid is RNA. In some embodiments, the nucleic acid is ribosomal RNA. In certain embodiments, the nucleic acid is extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In certain embodiments, this storage is less than 8° C. In certain embodiments, this storage is less than 4° C. In certain embodiments, this storage is less than 0° C. In certain embodiments, this storage is less than −20° C. In certain embodiments, this storage is less than −70° C. In certain embodiments, the nucleic acid is stored for 1, 2, 3, 4, 5, 6, or 7 days. In some embodiments, the nucleic acid is stored for 1, 2, 3, or 4 weeks. In some embodiments, the nucleic acid is stored for 1, 2, 3, 4, 5, 6, 7, 8, 9, 10, 11 or 12 months.

In some embodiments, a composition disclosed herein is stable at room temperature (−25° C.) for at least about one day. In some embodiments, a composition disclosed herein is stable for at least about three days. In some embodiments, a composition disclosed herein is stable for at least about six days. In some embodiments, a composition disclosed herein is stable for at least about nine days. In some embodiments, a composition disclosed herein is stable for at least about twelve days.

In some embodiments, compositions disclosed herein possess therapeutic activity when applied to skin with a skin disorder. In some embodiments, compositions disclosed herein possess an initial therapeutic activity at the time the composition is produced or packaged. In some embodiments, a composition disclosed herein maintains its initial therapeutic activity at room temperature (−25° C.) for at least about one day. In some embodiments, a composition disclosed herein maintains its initial therapeutic activity for at least about three days. In some embodiments, a composition disclosed herein maintains its initial therapeutic activity for at least about six days. In some embodiments, a composition disclosed herein maintains its initial therapeutic activity for at least about nine days. In some embodiments, a composition disclosed herein maintains its initial therapeutic activity for at least about twelve days.

In some embodiments, a composition disclosed herein maintains at least about 90% of its initial therapeutic activity at room temperature (−25° C.) for at least about one day. In some embodiments, a composition disclosed herein maintains at least about 90% of its initial therapeutic activity for at least about three days. In some embodiments, a composition disclosed herein maintains at least about 90% of its initial therapeutic activity for at least about six days. In some embodiments, a composition disclosed herein maintains at least about 90% of its initial therapeutic activity for at least about nine days. In some embodiments, a composition disclosed herein maintains at least about 90% of its initial therapeutic activity for at least about twelve days.

In some embodiments, a composition disclosed herein maintains at least about 80% of its initial therapeutic activity at room temperature (−25° C.) for at least about one day. In some embodiments, a composition disclosed herein maintains at least about 80% of its initial therapeutic activity for at least about three days. In some embodiments, a composition disclosed herein maintains at least about 80% of its initial therapeutic activity for at least about six days. In some embodiments, a composition disclosed herein maintains at least about 80% of its initial therapeutic activity for at least about nine days. In some embodiments, a composition disclosed herein maintains at least about 80% of its initial therapeutic activity for at least about twelve days.

In some embodiments, a composition disclosed herein maintains at least about 70% of its initial therapeutic activity at room temperature (−25° C.) for at least about one day. In some embodiments, a composition disclosed herein maintains at least about 70% of its initial therapeutic activity for at least about three days. In some embodiments, a composition disclosed herein maintains at least about 70% of its initial therapeutic activity for at least about six days. In some embodiments, a composition disclosed herein maintains at least about 70% of its initial therapeutic activity for at least about nine days. In some embodiments, a composition disclosed herein maintains at least about 70% of its initial therapeutic activity for at least about twelve days.

In some embodiments, a composition disclosed herein maintains at least about 50% of its initial therapeutic activity at room temperature (−25° C.) for at least about one day. In some embodiments, a composition disclosed herein maintains at least about 50% of its initial therapeutic activity for at least about three days. In some embodiments, a composition disclosed herein maintains at least about 50% of its initial therapeutic activity for at least about six days. In some embodiments, a composition disclosed herein maintains at least about 50% of its initial therapeutic activity for at least about nine days. In some embodiments, a composition disclosed herein maintains at least about 50% of its initial therapeutic activity for at least about twelve days.

After the nucleic acids are sequenced, in certain embodiments, described herein diagnosis is made. In certain, embodiments, diagnosis requires the use of a computer, statistical analysis, statistical analysis software, sequence analysis software, or any combination thereof. In certain embodiments, different organisms are identified. In certain embodiments, Malassezia fungi are identified. In certain embodiments, different strains of bacteria are identified during compiling of the microbiome. In certain embodiments, Staphylococcus bacteria are identified. In certain embodiments, different strains of P. acnes are identified during compiling of the microbiome. In certain embodiments, different strains of P. acnes are identified based upon their 16S ribosomal sequence or ribotype. In certain embodiments, P. acnes are identified as belonging to RT1-RT10. In certain embodiments, the P. acnes are identified as belonging to RT1, RT2, RT3, RT6, or any combination thereof. In further embodiments, a diagnostic report may be sent through or accessed by the internet. A diagnostic report may be sent through the mail to a health care provider, physician, or patient.

In certain embodiments, this disclosure provides a method for determining the presence of antibiotic resistant bacteria in an individual with acne comprising, obtaining a skin sample from an individual, isolating bacterial DNA from the sample, amplifying the relevant regions of the 16S, 18S or 23S ribosomal RNA from the sample, sequencing the amplified DNA products and determining antibiotic resistance to tetracycline and erythromycin/clindamycin classes of antibiotics respectively. In certain embodiments, bacteria present on or isolated from an individual's skin is tested or determined to be resistant to antibiotics. In certain embodiments, the antibiotic is erythromycin. In certain embodiments, the antibiotic is clindamycin. In certain embodiments, antibiotic resistance is determined from the sequence of the 16S, 18S or 23S ribosomal subtype.

### Methods of Preserving Preparations of Microbes

Provided herein, in some aspects, are methods of producing desired preparations of P. acnes bacteria. In some embodiments, methods comprise adding a sample of the P. acnes bacteria to a glycerol solution to produce a P. acnes glycerol stock, and storing the P. acnes glycerol stock at a temperature of about 4° C. or less. Producing a desired preparation of P. acnes may comprise at least one of cooling, freezing, and storing a P. acnes sample, a composition thereof or a stock thereof.

By way of non-limiting example, methods are provided herein for producing a desired preparation of a P. acnes bacteria comprising adding a sample of the P. acnes bacteria to a glycerol solution to produce a P. acnes glycerol stock, and storing the P. acnes glycerol stock at a temperature of about 4° C. or less, wherein more than about 50% of the P. acnes bacteria is viable when the P. acnes bacteria in the glycerol solution is brought to ambient temperature.

Also by way of non-limited examples, methods are provided herein for producing a desired preparation of preserved P. acnes, wherein about 90% of said P. acnes is viable after sixty days of storage, said method comprising: adding a sample of P. acnes bacteria to a solution of about 50% glycerol to produce a P. acnes glycerol stock, freezing the P. acnes glycerol stock at −20° C., thereby forming said desired preparation wherein greater than about 90% of the sample of P. acnes bacteria are viable after a thawing of the P. acnes glycerol stock.

In some embodiments, methods comprise storing the P. acnes bacteria, wherein at least about 50% of the P. acnes bacteria is viable when the P. acnes bacterium in the glycerol solution is brought to ambient temperature. In some embodiments, at least about 60% of the P. acnes bacteria is viable when the P. acnes bacteria in the glycerol solution is brought to ambient temperature. In some embodiments, at least about 70% of the P. acnes bacteria is viable when the P. acnes bacteria in the glycerol solution is brought to ambient temperature. In some embodiments, at least about 80% of the P. acnes bacteria is viable when the P. acnes bacteria in the glycerol solution is brought to ambient temperature. In some embodiments, more than about 90% of the P. acnes bacteria are viable when the P. acnes bacteria in the glycerol solution is brought to ambient temperature.

In some embodiments, methods comprise adding the P. acnes bacteria to a glycerol solution, wherein the glycerol solution is between about 25% and about 75% glycerol. In some embodiments, the glycerol solution is between about 30% and about 70% glycerol. In some embodiments, the glycerol solution is between about 35% and about 65% glycerol. In some embodiments, the glycerol solution is between about 40% and about 60% glycerol. In some embodiments, the glycerol solution is between about 45% and about 50% glycerol. In some embodiments, the glycerol solution is about 25% glycerol. In some embodiments, the glycerol solution is about 30% glycerol. In some embodiments, the glycerol solution is about 35% glycerol. In some embodiments, the glycerol solution is about 40% glycerol. In some embodiments, the glycerol solution is about 45% glycerol. In some embodiments, the glycerol solution is about 50% glycerol. In some embodiments, the glycerol solution is about 55% glycerol. In some embodiments, the glycerol solution is about 60% glycerol.

In some embodiments, methods comprise storing P. acnes bacteria, or a composition thereof, disclosed herein, at a selected temperature. In some embodiments, methods comprise storing the P. acnes glycerol stock at a selected temperature. In some embodiments, the temperature is between about 10° C. and about −80° C. In some embodiments, the temperature is between about 10° C. and about −40° C. In some embodiments, the temperature is between about 10° C. and about −30° C. In some embodiments, the temperature is between about 10° C. and about −20° C. In some embodiments, the temperature is between about 4° C. and about −80° C. In some embodiments, the temperature is between about 4° C. and about −25° C. In some embodiments, the temperature is between about 4° C. and about −20° C. In some embodiments, the temperature is about 4° C. In some embodiments, the temperature is about −20° C. In some embodiments, the temperature is between about −80° C. In some embodiments, methods comprise thawing a composition of P. acnes bacteria disclosed herein. In some embodiments, methods comprise warming a composition of P. acnes bacteria disclosed herein. In some embodiments, methods comprise thawing a P. acnes glycerol stock at room temperature. In some embodiments, methods comprise rapid thawing the P. acnes glycerol stock in a bath. The bath temperature may be between about 25° C. and about 40° C. In some embodiments, methods comprise rapidly thawing a composition of P. acnes bacteria disclosed herein. By way of non-limiting example, a subject may apply a composition disclosed herein, wherein the composition is frozen, directly to skin. In some embodiments, methods comprise slowly thawing a composition of P. acnes bacteria disclosed herein. By way of non-limiting example, a subject may transfer a composition disclosed herein that is frozen to a refrigerator to reach a refrigerated temperature before being brought to room temperature, before being applied to skin, or before being combined with another composition (e.g., emollient, lotion, gel). The term “frozen” includes compositions at temperatures at which the composition is in a solid form or semi-solid form. Frozen may include compositions at temperatures of less than 0° C., and less than −15° C. The term “refrigerated temperature” refers to a temperature of about 0° C. to about 10° C., e.g., 4° C. A refrigerated temperature does not necessarily need to be achieved with a refrigerator. By non-limiting example, an ice bucket could similarly cool a sample.

In some embodiments, methods comprise storing a P. acnes glycerol stock, wherein at least about 60% to at least about 90% of the P. acnes sample is viable after the P. acnes glycerol stock is brought to ambient temperature. In some embodiments, the at least about 70% to at least about 90% of the P. acnes sample is viable after the P. acnes glycerol stock is brought to ambient temperature. In some embodiments, the at least about 80% to at least about 90% of the viable after the P. acnes glycerol stock is brought to ambient temperature. In some embodiments, at least about 60% of the P. acnes sample is viable after the P. acnes glycerol stock is brought to ambient temperature. In some embodiments, at least about 70% of the P. acnes sample is viable after the P. acnes glycerol stock is brought to ambient temperature. In some embodiments, at least about 80% of the P. acnes sample is viable after the P. acnes glycerol stock is brought to ambient temperature. In some embodiments, at least about 90% of the P. acnes sample is viable after the P. acnes glycerol stock is brought to ambient temperature. Ambient temperature is considered an acceptable room temperature. In some embodiments, the ambient temperature is between about 25° C. and about 35° C. In some embodiments, the ambient temperature is between about 20° C. and about 30° C. In some embodiments, the ambient temperature is between about 22° C. and about 28° C. In some embodiments, the ambient temperature is about 25° C.

In some embodiments, methods comprise storing the Propionibacterium, wherein at least about 1% of the Propionibacterium is viable when the Propionibacterium in the glycerol solution is brought to ambient temperature. In some embodiments, methods comprise storing the Propionibacterium, wherein at least about 5% of the Propionibacterium is viable when the Propionibacterium in the glycerol solution is brought to ambient temperature. In some embodiments, methods comprise storing the Propionibacterium, wherein at least about 10% of the Propionibacterium is viable when the Propionibacterium in the glycerol solution is brought to ambient temperature. In some embodiments, methods comprise storing the Propionibacterium, wherein at least about 15% of the Propionibacterium is viable when the Propionibacterium in the glycerol solution is brought to ambient temperature. In some embodiments, methods comprise storing the Propionibacterium, wherein at least about 20% of the Propionibacterium is viable when the Propionibacterium in the glycerol solution is brought to ambient temperature. In some embodiments, methods comprise storing the Propionibacterium, wherein at least about 30% of the Propionibacterium is viable when the Propionibacterium in the glycerol solution is brought to ambient temperature. In some embodiments, methods comprise storing the Propionibacterium, wherein at least about 40% of the Propionibacterium is viable when the Propionibacterium in the glycerol solution is brought to ambient temperature. In some embodiments, methods comprise storing a P. acnes glycerol stock, wherein at least about 50% of the P. acnes sample is viable after at least about 30 days of storing. In some embodiments, at least about 60% of the P. acnes sample is viable after at least about 30 days of storing. In some embodiments, at least about 70% of the P. acnes sample is viable after at least about 20 days of storing. In some embodiments, at least about 80% of the P. acnes sample is viable after at least about 30 days of storing. In some embodiments, at least about 90% of the P. acnes sample is viable after at least about 30 days of storing. In some embodiments, at least about 95% of the P. acnes sample is viable after at least about 30 days of storing. In some embodiments, at least about 50% of the P. acnes sample is viable after at least about 60 days of storing. In some embodiments, at least about 60% of the P. acnes sample is viable after at least about 60 days of storing. In some embodiments, at least about 70% of the P. acnes sample is viable after at least about 60 days of storing. In some embodiments, at least about 80% of the P. acnes sample is viable after at least about 60 days of storing. In some embodiments, at least about 90% of the P. acnes sample is viable after at least about 60 days of storing. In some embodiments, at least about 95% of the P. acnes sample is viable after at least about 60 days of storing. In some embodiments, at least about 50% of the P. acnes sample is viable after at least about 90 days of storing. In some embodiments, at least about 60% of the P. acnes sample is viable after at least about 90 days of storing. In some embodiments, at least about 70% of the P. acnes sample is viable after at least about 90 days of storing. In some embodiments, at least about 80% of the P. acnes sample is viable after at least about 90 days of storing. In some embodiments, at least about 90% of the P. acnes sample is viable after at least about 90 days of storing. In some embodiments, at least about 95% of the P. acnes sample is viable after at least about 90 days of storing. In some embodiments, at least about 50% of the P. acnes sample is viable after at least about 120 days of storing. In some embodiments, at least about 60% of the P. acnes sample is viable after at least about 120 days of storing. In some embodiments, at least about 70% of the P. acnes sample is viable after at least about 120 days of storing. In some embodiments, at least about 80% of the P. acnes sample is viable after at least about 120 days of storing. In some embodiments, at least about 90% of the P. acnes sample is viable after at least about 120 days of storing. In some embodiments, at least about 95% of the P. acnes sample is viable after at least about 120 days of storing. In some embodiments, at least about 50% of the P. acnes sample is viable after at least about 180 days of storing. In some embodiments, at least about 60% of the P. acnes sample is viable after at least about 180 days of storing. In some embodiments, at least about 70% of the sample is viable after at least about 180 days of storing. In some embodiments, at least about 80% of the P. acnes sample is viable after at least about 180 days of storing. In some embodiments, at least about 90% of the P. acnes sample is viable after at least about 180 days of storing. In some embodiments, at least about 95% of the P. acnes sample is viable after at least about 180 days of storing. In some embodiments, at least about 50% of the P. acnes sample is viable after at least about a year of storing. In some embodiments, at least about 60% of the P. acnes sample is viable after at least about a year of storing. In some embodiments, at least about 70% of the P. acnes sample is viable after at least about a year of storing. In some embodiments, at least about 80% of the P. acnes sample is viable after at least about a year of storing. In some embodiments, at least about 90% of the P. acnes sample is viable after at least about a year of storing. In some embodiments, at least about 95% of the P. acnes sample is viable after at least about a year of storing.

In some embodiments, methods comprise storing P. acnes bacteria in a solution, wherein the solution is between about 10% glycerol v/v and about 90% glycerol v/v in solution. In some embodiments, the solution is between about 20% glycerol v/v and about 80% glycerol v/v in solution. In some embodiments, the solution is between about 25% glycerol v/v and about 75% glycerol v/v in solution. In some embodiments, the solution is between about 30% glycerol v/v and about 70% glycerol v/v in solution. In some embodiments, the solution is between about 35% glycerol v/v and about 65% glycerol v/v in solution. In some embodiments, the solution is between about 40% glycerol v/v and about 60% glycerol v/v in solution. In some embodiments, the solution is between about 45% glycerol v/v and about 55% glycerol v/v in solution. In some embodiments, the solution is between about 15% glycerol v/v and about 35% glycerol v/v in solution. In some embodiments, the solution is between about 20% glycerol v/v and about 30% glycerol v/v in solution. In some embodiments, the solution is about 20% glycerol v/v in solution. In some embodiments, the solution is about 25% glycerol v/v in solution. In some embodiments, the solution is about 30% glycerol v/v in solution. In some embodiments, the solution is about 35% glycerol v/v in solution. In some embodiments, the solution is about 40% glycerol v/v in solution. In some embodiments, the solution is about 45% glycerol v/v in solution. In some embodiments, the solution is about 50% glycerol v/v in solution. In some embodiments, the solution is about 50% glycerol v/v in solution. In some embodiments, the solution is about 55% glycerol v/v in solution. In some embodiments, the solution is about 60% glycerol v/v in solution. In some embodiments, the solution is about 65% glycerol v/v in solution. In some embodiments, the solution is about 75% glycerol v/v in solution.

In some embodiments, methods comprise storing P. acnes bacteria in a solution, wherein the solution comprises glycerol and water. In some embodiments, the solution consists essentially of glycerol and water. In some embodiments, methods comprise storing P. acnes bacteria in a solution, wherein the solution comprises glycerol and a saline solution. In some embodiments, the solution consists essentially of glycerol and a saline solution. In some embodiments, the solution comprises glycerol and a buffered saline solution. In some embodiments, the solution consists essentially of glycerol and a buffered saline solution. n some embodiments, the solution comprises glycerol and a buffered solution. In some embodiments, the buffered solution comprises sodium bicarbonate, citric acid or triethanolamine. In some embodiments, the solution comprises glycerol and a phosphate buffered saline solution. In some embodiments, the solution consists essentially of glycerol and a phosphate buffered saline solution.

In some embodiments, methods comprise storing P. acnes bacteria in a solution, wherein the solution has a pH of between about 3.5 and about 7. In some embodiments, the solution has a pH of between about 4 and about 6.5. In some embodiments, the solution has a pH of between about 4 and about 6. In some embodiments, the solution has a pH of between about 4 and about 5.5. In some embodiments, the solution has a pH of between about 4.5 and about 5.5. In some embodiments, the solution has a pH of between about 4.8 and about 5. In some embodiments, the solution has a pH of about 4. In some embodiments, the solution has a pH of about 4.2. In some embodiments, the solution has a pH of about 4.4. In some embodiments, the solution has a pH of about 4.6. In some embodiments, the solution has a pH of about 4.8. In some embodiments, the solution has a pH of about 5. In some embodiments, the solution has a pH of about 5.2. In some embodiments, the solution has a pH of about 5.4. In some embodiments, the solution has a pH of about 5.6. In some embodiments, the solution has a pH of about 5.8. In some embodiments, the solution has a pH of about 6.

In some embodiments, methods comprise storing P. acnes bacteria in a solution, wherein the solution comprises a salt or ion thereof. In some embodiments, the solution comprises an ion selected from potassium, calcium, magnesium, sodium, and boron. In some embodiments, the solution comprises potassium. In some embodiments, the solution comprises potassium. In some embodiments, the concentration of the salt or ion thereof is between about 0.001 mM and about 1 mM. In some embodiments, the concentration of the salt or ion thereof is between about 0.001 mM and about 0.1 mM. In some embodiments, the concentration of the salt or ion thereof is between about 0.01 mM and about 0.1 mM. In some embodiments, the concentration of the salt or ion thereof is between about 0.05 mM and about 0.1 mM. In some embodiments, the concentration of the salt or ion thereof is between about 0.01 mM and about 1 mM. In some embodiments, the concentration of the salt or ion thereof is between about 0.1 mM and about 1 mM. In some embodiments, the concentration of the salt or ion thereof is between about 100 mM and about 250 mM. In some embodiments, the concentration of the salt or ion thereof is between about 125 mM and about 225 mM. In some embodiments, the concentration of the salt or ion thereof is between about 150 mM and about 200 mM. In some embodiments, the concentration of potassium is between about 100 mM and about 250 mM. In some embodiments, the concentration of potassium is between about 125 mM and about 225 mM. In some embodiments, the concentration of potassium is between about 150 mM and about 200 mM. In some embodiments, the solution comprises calcium at a concentration of about 0.001 mM to about 1 mM. In some embodiments, the solution comprises calcium at a concentration of about 0.01 mM to about 0.5 mM. In some embodiments, the solution comprises calcium at a concentration of about 0.05 mM to about 0.1 mM.

In some embodiments, methods comprise storing P. acnes bacteria in a solution, wherein the solution comprises a prebiotic stabilizing agent. In some embodiments, the prebiotic stabilizing agent is selected from a polysaccharide or oligosaccharide. In some embodiments, the prebiotic stabilizing agent is inulin. In some embodiments, the stabilizing agent is present in the solution at a concentration of about 0.01% v/v to about 1% v/v. In some embodiments, the stabilizing agent is present in the solution at a concentration of about 0.01% v/v to about 0.5% v/v. In some embodiments, the stabilizing agent is present in the solution at a concentration of about 0.05% v/v to about 0.2% v/v. In some embodiments, the solution comprises inulin at a concentration of about 0.01% v/v to about 1% v/v. In some embodiments, the solution comprises inulin at a concentration of about 0.01% v/v to about 0.5% v/v. In some embodiments, the solution comprises inulin at a concentration of about 0.05% v/v to about 0.2% v/v. For clarity, the term % v/v, as used herein, represent the percentage of a total volume of a solution that is represented by a volume of a component of the solution.

In some embodiments, methods comprise storing P. acnes bacteria in a solution, wherein the solution comprises an anti-acne agent, wherein the anti-acne agent is an agent that prevents, reduces or abolishes acne. In some embodiments, the anti-acne agent is selected from a retinoid, a vitamin, an antioxidant, a peroxide, an acid, an oil, an alcohol, an extract, and analogs thereof. For clarity, the term, “analog,” as used herein, refers to a compound having a structure similar to that of another one, but differing from it by less than about 10% of the total structure. In some embodiments, the retinoid is selected from tretinoin, tazarotene, adapalene, and retinol. In some embodiments, the vitamin or analog thereof is selected from a Vitamin A, Vitamin C, Vitamin D, Vitamin E, and calciptotriene. In some embodiments, the antioxidant is selected from Vitamin C and Vitamin E. peroxide is benzoyl peroxide. In some embodiments, the acid is selected from salicylic acid, azaelic acid, trichloracetic acid, and glycolic acid. In some embodiments, the alcohol is selected from cetyl alcohol, stearyl alcohol, and cetearyl alcohol. In some embodiments, the alcohol is selected from retinol (also known as Vitamin A₁) and resveratrol. In some embodiments, the oil is selected from lavender oil, clary sage oil, juniper berry oil, bergamot oil, jojoba oil, rosemary oil, coconut oil, avocado oil, peppermint oil, and tea tree oil. In some embodiments, the oil is tea tree oil. In some embodiments, the extract is selected from an extract of aloe, garlic, amaranth, neem, coriander, lemon, basil, grapefruit, cucumber, grape, beet, green tea or a combination thereof. In some embodiments, the extract is a green tea extract.

In some embodiments, methods comprise storing P. acnes bacteria in a solution, wherein the solution is incorporated in a biologic stability platform. In some embodiments, the biologic stability platform eliminates a need for temperature control, e.g., cold chain storage. In some embodiments, the biologic storage platform comprises foam drying or foam formation of the solution or glycerol stock solution. In some embodiments, the biologic stability platform comprises at least one of a glyconanoparticle, a liposome, a nanoparticle, trehalose, sucrose, stachyose, hydroxyethyl starch, and a combination of glycine and mannitol.

In some embodiments, methods comprise storing or preserving a sample of P. acnes of at least one selected ribotype. the sample of P. acnes bacteria comprises P. acnes bacteria of ribotype RT1. In some embodiments, the sample of P. acnes bacteria comprises P. acnes bacteria of ribotype RT2. In some embodiments, the sample of P. acnes bacteria comprises P. acnes bacteria of ribotypes RT1 and RT2. In some embodiments, the sample of P. acnes bacteria consists essentially of P. acnes bacteria of ribotype RT1. In some embodiments, the sample of P. acnes bacteria consists essentially of P. acnes bacteria of ribotype RT2. In some embodiments, the sample of P. acnes bacteria consists essentially of P. acnes bacteria of ribotypes RT1 and RT2.

In some embodiments, methods may comprise culturing an initial culture of the sample of P. acnes bacteria. The initial culture may be a smaller aliquot of the P. acnes bacteria and the method may comprise proliferating the P. acnes bacteria to obtain a desired amount. In some embodiments, methods may comprise culturing the initial culture in a culture medium. In some embodiments, the cell culture medium comprises reinforced clostridial medium. In some embodiments, the cell culture medium consists essentially of reinforced clostridial medium. In some embodiments, the cell culture medium comprises Luria broth. In some embodiments, the cell culture medium comprises tryptone broth. In some embodiments, methods comprise at least one step of splitting, diluting or passaging the initial culture or product thereof in the culture medium. In some embodiments, the methods comprise at least one step of washing the sample of the initial culture or product thereof. In some embodiments, the methods comprise at least one step of centrifuging or pelleting the initial culture or product thereof. In some embodiments, the culture is centrifuged at about 3500 rcf to about 4500 rcf. In some embodiments, the culture is centrifuged at about 3800 rcf to about 4200 rcf. In some embodiments, the culture is centrifuged at about 4000 rcf. In some embodiments, the methods comprise at least one step of vortexing the initial culture or product thereof. In some embodiments, the methods comprise at least one step of pipetting the initial culture of product thereof. Any one of the steps described herein may be performed at least one time. Any one of the steps described herein may be performed two times. Any one of the steps described herein may be performed three times. In some embodiments, methods comprise adding a sachet to a culture comprising the sample of P. acnes bacteria or an initial culture thereof. In some embodiments, the sachet reduces oxygen exposure to the P. acnes bacteria.

### Compositions of Preserved Propionibacterium

Provided herein, in some aspects, are compositions that comprise a preserved sample of bacteria, wherein the bacteria comprises P. acnes bacteria. In some embodiments, compositions disclosed herein comprise P. acnes bacteria of ribotype RT1. In some embodiments, the compositions comprise P. acnes bacteria of ribotype RT2. In some embodiments, the compositions comprise P. acnes bacteria of ribotype RT1 and RT2. In some embodiments, the bacteria of the compositions consist essentially of P. acnes bacteria of ribotype RT1. In some embodiments, the bacteria of the compositions consist essentially of P. acnes bacteria of ribotype RT2. In some embodiments, the compositions comprise P. acnes bacteria of ribotype RT1 and RT2.

In some embodiments, compositions disclosed herein comprise a sample of bacteria preserved in at least one cryopreservative agent. In some embodiments, the cryopreservative agent is glycerol. In some embodiments, the cryopreservative agent is glycol. In some embodiments, the cryopreservative agent is glycerol. In some embodiments, the cryopreservative agent is polyethylene glycol. In some embodiments, the cryopreservative agent is polyol.

Non-limiting examples of polyols include DMSO, ethylene glycol, glycerol, propylene (PEG) glycol, sucrose, trehalose, and 2-Methyl-2,4-pentanediol (MPD). In various embodiments, the PEG may have a molecular weight between about 10 g/mol and about 10,000 g/mol. In various embodiments, the PEG may have a molecular weight between about 10 g/mol and about 5,000 g/mol. In various embodiments, the PEG may have a molecular weight between about 10 g/mol and about 1,000 g/mol. In various embodiments, the PEG may have a molecular weight between about 10 g/mol and about 500 g/mol.

In some embodiments, compositions disclose herein comprise polyethylene glycol. In some embodiments, a composition comprising polyethylene glycol allows for a reduced amount of glycerol, whilst maintaining viability of bacteria in the composition that is similar to viability of bacteria in compositions without polyethylene glycol and a greater amount of glycerol. In some embodiments, reducing or minimizing the amount of glycerol in a composition disclosed herein results in a formulation that has a texture that is more preferable to a subject. In some embodiments, reducing or minimizing the amount of glycerol in a composition disclosed herein results in a formulation that less comedogenic relative to a composition with a greater amount of glycerol.

In some embodiments, compositions disclosed herein comprise a sample of bacteria preserved in a mixture of a first polyol and a second polyol. In some embodiments, the first polyol or the second polyol is glycerol. In some embodiments, the first polyol or the second polyol is a polyethylene glycol. In some embodiments, compositions disclosed herein comprise a sample of bacteria preserved in a mixture of glycerol and polyethylene glycol. In some embodiments, the mixture is between about 1% glycerol v/v and about 50% glycerol v/v, and between about 1% polyethylene glycol w/v and about 50% w/v polyethylene glycol. In some embodiments, the mixture is between about 5% glycerol v/v and about 50% glycerol v/v, and between about 5% polyethylene glycol w/v and about 50% w/v polyethylene glycol. In some embodiments, the mixture is between about 5% glycerol v/v and about 30% glycerol v/v, and between about 5% polyethylene glycol w/v and about 40% w/v polyethylene glycol. In some embodiments, the mixture is between about 10% glycerol v/v and about 35% glycerol v/v, and between about 10% polyethylene glycol w/v and about 35% w/v polyethylene glycol.

In some embodiments, compositions disclose herein comprise a solution, wherein the solution is between about 1% glycerol v/v and about 75% glycerol v/v in solution. In some embodiments, the solution is between about 1% glycerol v/v and about 50% glycerol v/v in solution. In some embodiments, the solution is between about 1% glycerol v/v and about 40% glycerol v/v in solution. In some embodiments, the solution is between about 1% glycerol v/v and about 35% glycerol v/v in solution. In some embodiments, the solution is between about 1% glycerol v/v and about 30% glycerol v/v in solution. In some embodiments, the solution is between about 1% glycerol v/v and about 25% glycerol v/v in solution. In some embodiments, the solution is between about 1% glycerol v/v and about 20% glycerol v/v in solution. In some embodiments, the solution is between about 1% glycerol v/v and about 15% glycerol v/v in solution. In some embodiments, the solution is between about 5% glycerol v/v and about 50% glycerol v/v in solution. In some embodiments, the solution is between about 5% glycerol v/v and about 40% glycerol v/v in solution. In some embodiments, the solution is between about 5% glycerol v/v and about 35% glycerol v/v in solution. In some embodiments, the solution is between about 5% glycerol v/v and about 30% glycerol v/v in solution. In some embodiments, the solution is between about 5% glycerol v/v and about 25% glycerol v/v in solution. In some embodiments, the solution is between about 10% glycerol v/v and about 40% glycerol v/v in solution. In some embodiments, the solution is between about 10% glycerol v/v and about 35% glycerol v/v in solution. In some embodiments, the solution is between about 10% glycerol v/v and about 30% glycerol v/v in solution. In some embodiments, the solution is between about 10% glycerol v/v and about 25% glycerol v/v in solution. In some embodiments, the solution is between about 15% glycerol v/v and about 40% glycerol v/v in solution. In some embodiments, the solution is between about 15% glycerol v/v and about 35% glycerol v/v in solution. In some embodiments, the solution is between about 15% glycerol v/v and about 30% glycerol v/v in solution. In some embodiments, the solution is between about 15% glycerol v/v and about 25% glycerol v/v in solution. In some embodiments, the solution is between about 20% glycerol v/v and about 50% glycerol v/v in solution. In some embodiments, the solution is between about 20% glycerol v/v and about 40% glycerol v/v in solution. In some embodiments, the solution is between about 20% glycerol v/v and about 30% glycerol v/v in solution.

In some embodiments, compositions disclose herein comprise a solution, wherein the solution is between about 1% polyethylene glycol w/v and about 75% polyethylene glycol w/v in solution. In some embodiments, the solution is between about 1% polyethylene glycol w/v and about 50% polyethylene glycol w/v in solution. In some embodiments, the solution is between about 1% polyethylene glycol w/v and about 40% polyethylene glycol w/v in solution. In some embodiments, the solution is between about 1% polyethylene glycol w/v and about 35% polyethylene glycol w/v in solution. In some embodiments, the solution is between about 1% polyethylene glycol w/v and about 30% polyethylene glycol w/v in solution. In some embodiments, the solution is between about 1% polyethylene glycol w/v and about 25% polyethylene glycol w/v in solution. In some embodiments, the solution is between about 1% polyethylene glycol w/v and about 20% polyethylene glycol w/v in solution. In some embodiments, the solution is between about 1% polyethylene glycol w/v and about 15% polyethylene glycol w/v in solution. In some embodiments, the solution is between about 5% polyethylene glycol w/v and about 50% polyethylene glycol w/v in solution. In some embodiments, the solution is between about 5% polyethylene glycol w/v and about 40% polyethylene glycol w/v in solution. In some embodiments, the solution is between about 5% polyethylene glycol w/v and about 35% polyethylene glycol w/v in solution. In some embodiments, the solution is between about 5% polyethylene glycol w/v and about 30% polyethylene glycol w/v in solution. In some embodiments, the solution is between about 5% polyethylene glycol w/v and about 25% polyethylene glycol w/v in solution. In some embodiments, the solution is between about 10% polyethylene glycol w/v and about 40% polyethylene glycol w/v in solution. In some embodiments, the solution is between about 10% polyethylene glycol w/v and about 35% polyethylene glycol w/v in solution. In some embodiments, the solution is between about 10% polyethylene glycol w/v and about 30% polyethylene glycol w/v in solution. In some embodiments, the solution is between about 10% polyethylene glycol w/v and about 25% polyethylene glycol w/v in solution. In some embodiments, the solution is between about 15% polyethylene glycol w/v and about 40% polyethylene glycol w/v in solution. In some embodiments, the solution is between about 15% polyethylene glycol w/v and about 35% polyethylene glycol w/v in solution. In some embodiments, the solution is between about 15% polyethylene glycol w/v and about 30% polyethylene glycol w/v in solution. In some embodiments, the solution is between about 15% polyethylene glycol w/v and about 25% polyethylene glycol w/v in solution. In some embodiments, the solution is between about 20% polyethylene glycol w/v and about 50% polyethylene glycol w/v in solution. In some embodiments, the solution is between about 20% polyethylene glycol w/v and about 40% polyethylene glycol w/v in solution. In some embodiments, the solution is between about 20% polyethylene glycol w/v and about 30% polyethylene glycol w/v in solution.

In some embodiments, the solution is about 5% glycerol v/v in solution. In some embodiments, the solution is about 10% glycerol v/v in solution. In some embodiments, the solution is about 15% glycerol v/v in solution. In some embodiments, the solution is about 20% glycerol v/v in solution. In some embodiments, the solution is about 25% glycerol v/v in solution. In some embodiments, the solution is about 30% glycerol v/v in solution. In some embodiments, the solution is about 35% glycerol v/v in solution. In some embodiments, the solution is about 40% glycerol v/v in solution. In some embodiments, the solution is about 45% glycerol v/v in solution. In some embodiments, the solution is about 50% glycerol v/v in solution. In some embodiments, the solution is about 55% glycerol v/v in solution. In some embodiments, the solution is about 60% glycerol v/v in solution. In some embodiments, the solution is about 70% glycerol v/v in solution.

In some embodiments, compositions disclosed herein comprise a solution, wherein the solution comprises glycerol and water. In some embodiments, the solution consists essentially of glycerol and water. In some embodiments, methods comprise storing P. acnes bacteria in a solution, wherein the solution comprises glycerol and a saline solution. In some embodiments, the solution consists essentially of glycerol and a saline solution. In some embodiments, the solution comprises glycerol and a buffered saline solution. In some embodiments, the solution consists essentially of glycerol and a buffered saline solution. In some embodiments, the solution comprises glycerol and a buffered solution. In some embodiments, the buffered solution comprises sodium bicarbonate, citric acid or triethanolamine. In some embodiments, the solution comprises glycerol and a phosphate buffered saline solution. In some embodiments, the solution consists essentially of glycerol and a phosphate buffered saline solution.

In some embodiments, compositions disclosed herein comprise a solution, wherein the solution has a pH of between about 3.5 and about 7. In some embodiments, the solution has a pH of between about 4 and about 6.5. In some embodiments, the solution has a pH of between about 4 and about 6. In some embodiments, the solution has a pH of between about 4 and about 5.5. In some embodiments, the solution has a pH of between about 4.5 and about 5.5. In some embodiments, the solution has a pH of between about 4.8 and about 5. In some embodiments, the solution has a pH of about 4. In some embodiments, the solution has a pH of about 4.2. In some embodiments, the solution has a pH of about 4.4. In some embodiments, the solution has a pH of about 4.6. In some embodiments, the solution has a pH of about 4.8. In some embodiments, the solution has a pH of about 5. In some embodiments, the solution has a pH of about 5.2. In some embodiments, the solution has a pH of about 5.4. In some embodiments, the solution has a pH of about 5.6. In some embodiments, the solution has a pH of about 5.8. In some embodiments, the solution has a pH of about 6.

In some embodiments, compositions disclosed herein comprise a solution, wherein the solution comprises a salt or ion thereof. In some embodiments, the solution comprises an ion selected from potassium, calcium, magnesium, sodium, and boron. In some embodiments, the solution comprises potassium. In some embodiments, the solution comprises potassium. In some embodiments, the concentration of the salt or ion thereof is between about 0.001 mM and about 1 mM. In some embodiments, the concentration of the salt or ion thereof is between about 0.001 mM and about 0.1 mM. In some embodiments, the concentration of the salt or ion thereof is between about 0.01 mM and about 0.1 mM. In some embodiments, the concentration of the salt or ion thereof is between about 0.05 mM and about 0.1 mM. In some embodiments, the concentration of the salt or ion thereof is between about 0.01 mM and about 1 mM. In some embodiments, the concentration of the salt or ion thereof is between about 0.1 mM and about 1 mM. In some embodiments, the concentration of the salt or ion thereof is between about 100 mM and about 250 mM. In some embodiments, the concentration of the salt or ion thereof is between about 125 mM and about 225 mM. In some embodiments, the concentration of the salt or ion thereof is between about 150 mM and about 200 mM. In some embodiments, the concentration of potassium is between about 100 mM and about 250 mM. In some embodiments, the concentration of potassium is between about 125 mM and about 225 mM. In some embodiments, the concentration of potassium is between about 150 mM and about 200 mM. In some embodiments, the solution comprises calcium at a concentration of about 0.001 mM to about 1 mM. In some embodiments, the solution comprises calcium at a concentration of about 0.01 mM to about 0.5 mM. In some embodiments, the solution comprises calcium at a concentration of about 0.05 mM to about 0.1 mM.

In some embodiments, compositions disclosed herein comprise a solution, wherein the solution comprises at least one stabilizing agent. In some embodiments, the stabilizing agent is selected from inulin, sucrose, trehalose, cornstarch, maltodextrin, guar guy, locust bean gum, and xanathan gum. In some embodiments, trehalose or sucrose stabilizes bacteria for cold-chain free stability. In some embodiments, the stabilizing agent is inulin. In some embodiments, the stabilizing agent is present in the solution at a concentration of about 0.01% v/v to about 1% v/v. In some embodiments, the stabilizing agent is present in the solution at a concentration of about 0.01% v/v to about 0.5% v/v. In some embodiments, the stabilizing agent is present in the solution at a concentration of about 0.05% v/v to about 0.2% v/v. In some embodiments, the solution comprises inulin at a concentration of about 0.01% v/v to about 1% v/v. In some embodiments, the solution comprises inulin at a concentration of about 0.01% v/v to about 0.5% v/v. In some embodiments, the solution comprises inulin at a concentration of about 0.05% v/v to about 0.2% v/v.

In some embodiments, compositions disclosed herein comprise a solution, wherein the solution comprises an anti-acne agent, wherein the anti-acne agent is an agent that prevents, reduces or abolishes acne. In some embodiments, the anti-acne agent is selected from a retinoid, a vitamin, an antioxidant, a peroxide, an acid, an oil, an alcohol, an extract, and analogs thereof. In some embodiments, the retinoid is selected from tretinoin, tazarotene, adapalene, and retinol. In some embodiments, the vitamin or analog thereof is selected from Vitamin D, Vitamin C, Vitamin E, and calciptotriene. In some embodiments, the antioxidant is selected from Vitamin C and Vitamin E. peroxide is benzoyl peroxide. In some embodiments, the acid is selected from salicylic acid, azaelic acid, trichloracetic acid, and glycolic acid. In some embodiments, the alcohol is selected from retinol and resveratrol. In some embodiments, the oil is tea tree oil. In some embodiments, the extract is a green tea extract.

In some embodiments, compositions disclosed herein comprise a solution, wherein the solution is incorporated in a biologic stability platform. In some embodiments, the biologic stability platform eliminates a need for temperature control, e.g., cold chain storage. In some embodiments, the biologic storage platform comprises foam drying or foam formation of the solution or glycerol stock solution. In some embodiments, the biologic stability platform comprises at least one of a glyconanoparticle, a liposome, a nanoparticle, trehalose, sucrose, stachyose, hydroxyethyl starch, and a combination of glycine and mannitol.

In some embodiments, compositions disclosed herein have a temperature of about −80° C. to about 10° C. In some embodiments, the composition is at a temperature of about −80° C. to about 4° C. In some embodiments, the composition is at a temperature of about −40° C. to about 10° C. In some embodiments, the composition is at a temperature of about −25° C. to about 10° C. In some embodiments, the composition is at a temperature of about −20° C. to about 4° C. In some embodiments, the composition is at a temperature of about −90° C. to about −70° C. In some embodiments, the composition is at a temperature of about −30° C. to about −10° C. In some embodiments, the composition is at a temperature of about −80° C. In some embodiments, the composition is at a temperature of about −20° C. In some embodiments, the composition is at a temperature of about 4° C.

In some embodiments, compositions disclosed herein comprise a P. acnes glycerol stock, wherein at least about 60% to at least about 90% of the P. acnes sample is viable after the P. acnes glycerol stock is brought to ambient temperature. In some embodiments, the at least about 70% to at least about 90% of the P. acnes sample is viable after the P. acnes glycerol stock is brought to ambient temperature. In some embodiments, the at least about 80% to at least about 90% of the viable after the P. acnes glycerol stock is brought to ambient temperature. In some embodiments, at least about 60% of the P. acnes sample is viable after the P. acnes glycerol stock is brought to ambient temperature. In some embodiments, at least about 70% of the P. acnes sample is viable after the P. acnes glycerol stock is brought to ambient temperature. In some embodiments, at least about 80% of the P. acnes sample is viable after the P. acnes glycerol stock is brought to ambient temperature. In some embodiments, at least about 90% of the P. acnes sample is viable after the P. acnes glycerol stock is brought to ambient temperature. Ambient temperature is considered an acceptable room temperature. In some embodiments, the ambient temperature is between about 25° C. and about 35° C. In some embodiments, the ambient temperature is between about 20° C. and about 30° C. In some embodiments, the ambient temperature is between about 22° C. and about 28° C. In some embodiments, the ambient temperature is about 25° C.

In some embodiments, compositions disclosed herein comprise a P. acnes glycerol stock, wherein at least about 50% of the P. acnes sample is viable after at least about 30 days of storing. In some embodiments, at least about 60% of the P. acnes sample is viable after at least about 30 days of storing. In some embodiments, at least about 70% of the P. acnes sample is viable after at least about 20 days of storing. In some embodiments, at least about 80% of the P. acnes sample is viable after at least about 30 days of storing. In some embodiments, at least about 90% of the P. acnes sample is viable after at least about 30 days of storing. In some embodiments, at least about 95% of the P. acnes sample is viable after at least about 30 days of storing. In some embodiments, at least about 50% of the P. acnes sample is viable after at least about 60 days of storing. In some embodiments, at least about 60% of the P. acnes sample is viable after at least about 60 days of storing. In some embodiments, at least about 70% of the P. acnes sample is viable after at least about 60 days of storing. In some embodiments, at least about 80% of the P. acnes sample is viable after at least about 60 days of storing. In some embodiments, at least about 90% of the P. acnes sample is viable after at least about 60 days of storing. In some embodiments, at least about 95% of the P. acnes sample is viable after at least about 60 days of storing. In some embodiments, at least about 50% of the P. acnes sample is viable after at least about 90 days of storing. In some embodiments, at least about 60% of the P. acnes sample is viable after at least about 90 days of storing. In some embodiments, at least about 70% of the P. acnes sample is viable after at least about 90 days of storing. In some embodiments, at least about 80% of the P. acnes sample is viable after at least about 90 days of storing. In some embodiments, at least about 90% of the P. acnes sample is viable after at least about 90 days of storing. In some embodiments, at least about 95% of the P. acnes sample is viable after at least about 90 days of storing. In some embodiments, at least about 50% of the P. acnes sample is viable after at least about 120 days of storing. In some embodiments, at least about 60% of the P. acnes sample is viable after at least about 120 days of storing. In some embodiments, at least about 70% of the P. acnes sample is viable after at least about 120 days of storing. In some embodiments, at least about 80% of the P. acnes sample is viable after at least about 120 days of storing. In some embodiments, at least about 90% of the P. acnes sample is viable after at least about 120 days of storing. In some embodiments, at least about 95% of the P. acnes sample is viable after at least about 120 days of storing. In some embodiments, at least about 50% of the P. acnes sample is viable after at least about 180 days of storing. In some embodiments, at least about 60% of the P. acnes sample is viable after at least about 180 days of storing. In some embodiments, at least about 70% of the sample is viable after at least about 180 days of storing. In some embodiments, at least about 80% of the P. acnes sample is viable after at least about 180 days of storing. In some embodiments, at least about 90% of the P. acnes sample is viable after at least about 180 days of storing. In some embodiments, at least about 95% of the P. acnes sample is viable after at least about 180 days of storing. In some embodiments, at least about 50% of the P. acnes sample is viable after at least about a year of storing. In some embodiments, at least about 60% of the P. acnes sample is viable after at least about a year of storing. In some embodiments, at least about 70% of the P. acnes sample is viable after at least about a year of storing. In some embodiments, at least about 80% of the P. acnes sample is viable after at least about a year of storing. In some embodiments, at least about 90% of the P. acnes sample is viable after at least about a year of storing. In some embodiments, at least about 95% of the P. acnes sample is viable after at least about a year of storing.

In some embodiments, compositions disclosed herein have a storage life of at least about thirty days to at least about ninety days. In some embodiments, the compositions disclosed herein have a storage life of at least about 30 days to at least about 120 days. In some embodiments, the compositions disclosed herein have a storage life of at least about 30 days to at least about 180 days. In some embodiments, the compositions disclosed herein have a storage life of at least about thirty days to about ninety days. In some embodiments, the compositions disclosed herein have a storage life of at least about 30 days to about 120 days. In some embodiments, the compositions disclosed herein have a storage life of at least about 30 days to about 180 days. In some embodiments, the compositions disclosed herein have a storage life of at least about thirty days. In some embodiments, the compositions disclosed herein have a storage life of at least about sixty days. In some embodiments, the compositions disclosed herein have a storage life of at least about ninety days. In some embodiments, the compositions disclosed herein have a storage life of at least about 120 days. In some embodiments, the compositions disclosed herein have a storage life of at least about 180 days. In some embodiments, the compositions disclosed herein have a storage life of at least about 240 days. In some embodiments, the compositions disclosed herein have a storage life of at least about one year. In some embodiments, the compositions disclosed herein have a storage life of up to about one year.

In some embodiments, the compositions disclosed herein are capable of being thawed and subsequently applied to a subject in need thereof. In some embodiments, the compositions disclosed herein are capable of being warmed and subsequently applied to a subject in need thereof. In some embodiments, the compositions disclosed herein are capable of being refrigerated and subsequently applied to a subject in need thereof. In some embodiments, subsequently applied to the subject comprises applying the composition directly to the skin of the subject. In some embodiments, subsequently applied to the subject comprises applying the composition to an application composition before being applied to the skin. The application composition may be selected from a liquid, gel, lotion, emollient, paste, mask, and virtually any solution that can be applied to the skin of a subject. In some embodiments, the application composition is free of any anti-acne agent. In some embodiments, the application composition comprises an anti-acne agent. In some embodiments, the compositions disclosed herein are capable of being applied directly from a frozen stock to skin of a subject without thawing or warming.

### EXAMPLES

The following examples are set forth to illustrate more clearly the principle and practice of embodiments disclosed herein to those skilled in the art and are not to be construed as limiting the scope of any claimed embodiments.

### Example 1: Inactivation of P. acnes In Vitro

Cultures of P. acnes strains are exposed to electromagnetic radiation at different wavelengths between 400 nm and 700 nm. The bacteria are exposed to one or more doses of energy, with each dose being continuous or a plurality of pulses. The viability of the bacteria is measured to determine effectiveness of inactivation at the tested wavelengths.

### Example 2: Treatment of Acne in an In Vivo Animal Study

Inactivation of P. acnes in an animal model of acne is tested. Sprague-Dawley rats are intradermally injected with P. acnes. The rats are exposed to electromagnetic radiation at a wavelength of light between 400 nm and 700 nm for a treatment period. During the treatment period, one or more doses of the electromagnetic radiation are applied to the rats. The doses are continuous or intermittent pulses. During and after the treatment period, markers of inflammation associated with acne are assessed to evaluate effectiveness of the treatments.

For some rats, a probiotic is administered to the skin after one or more doses. The combined effect of electromagnetic radiation therapy and probiotic administration is evaluated.

### Example 3: Selective Targeting of Acne-Associated P. acnes

The experiment of Example 1 is performed using both acne-associated strains and health-associated strains of P. acnes. The viability of the acne-associated strains is compared to the viability of the health-associated strains after exposure to the electromagnetic radiation to evaluate selective targeting of the acne-associated P. acnes.

### Example 4: Clinical Trial

Purpose: The purpose of this study is to assess effectiveness of light therapy on acne lesions in subjects having disease-associated strains of P. acnes.

Intervention: Acne affected areas of subjects are exposed to electromagnetic radiation over a treatment period of 8-12 weeks. Doses of the electromagnetic radiation are provided every 2-4 weeks during the treatment period.

Detailed Description:

Subjects are assessed prior to administration for the presence of acne lesions in an affected area. The affected area is exposed to 2-6 doses of electromagnetic radiation at a wavelength between 400 and 700 nm. Each dose is administered as a continuous pulse, or as a plurality of intermittent pulses. After the treatment period, an assessment is performed to evaluate the number of acne lesions in the affected area.

Eligibility and Inclusion Criteria:

Male and female subjects that are 18 to 40 years old that are diagnosed as having acne lesions and a disease-associated strain of P. acnes.

Exclusion Criteria:

Patients with a psychiatric disorder that might cause difficulty in obtaining informed consent or in conducting the trial. Patients having a medical condition that could be affected by exposure to the prescribed wavelengths of electromagnetic radiation.

Primary Outcome Measures:

Determine percentage change is number of acne lesions before, during and after treatment. Monitor long-term effects of acne lesions after treatment, such as evaluating the number of acne lesions in the affected area days, weeks, and/or months after treatment.

Secondary Outcome Measures:

Determine adverse effects.

### Example 5: Benzoyl Peroxide and Light Therapy Combination: Clinical Trial

Purpose:

The purpose of this study is to assess effectiveness of light therapy alone or in combination with a benzoyl peroxide on acne lesions in subjects having disease-associated strains of P. acnes.

Intervention:

Acne affected areas of subjects are exposed to electromagnetic radiation. A first cohort has active acne lesions prior to electromagnetic radiation. A second cohort is treated for acne lesions with benzoyl peroxide, followed by electromagnetic radiation. Each cohort has 40 subjects.

Detailed Description:

Subjects are assessed prior to administration of benzoyl peroxide and/or radiation for the presence of acne lesions in an affected area. The affected area is exposed to one or more doses of electromagnetic radiation at a wavelength between 400 and 700 nm. Each dose is administered as a continuous pulse, or as a plurality of intermittent pulses. Subjects are assessed again at 4, 8 and 12 weeks. Each assessment comprises grading of acne lesions using a global acne grading scale (GAGS), determining adverse effects, and profiling of bacteria from each subject.

Eligibility and Inclusion Criteria:

Male and female subjects that are diagnosed as having acne lesions and a disease-associated strain of P. acnes.

Exclusion Criteria:

Subjects with a psychiatric disorder that might cause difficulty in obtaining informed consent or in conducting the trial. Subjects having a medical condition that could be affected by exposure to the prescribed wavelengths of electromagnetic radiation. Subjects having contraindications with benzoyl peroxide.

Primary Outcome Measures:

Determine adverse effects.

Secondary Outcome Measures:

Profile bacteria population after treatment to determine ability of exogenously applied bacteria, such as probiotics, to populate the skin. Evaluate viability of applied bacteria.

For the active disease cohorts, determine efficacy in treatment. Determine percentage change is number of acne lesions before, during and after treatment. Monitor long-term effects of acne lesions after treatment, such as evaluating the number of acne lesions in the affected area days, weeks, and/or months after treatment.

For the post benzoyl peroxide treated cohort, determine efficacy in prevention and/or maintenance of decrease in quantity and/or frequency of occurrence of acne lesions with light therapy.

### Example 6: Probiotic and Light Therapy Combination: Clinical Trial

Purpose:

The purpose of this study is to assess effectiveness of light therapy alone or in combination with a probiotic on acne lesions in subjects having disease-associated strains of P. acnes.

Intervention:

Acne affected areas of subjects are exposed to electromagnetic radiation. A first cohort is treated with electromagnetic radiation. A second cohort is treated with electromagnetic radiation followed by probiotic administration. The probiotic optionally comprises a P. acnes bacteria having reduced expression of porphyrin as compared to disease-associated P. acnes bacteria. The probiotic is synthetic or naturally derived.

Detailed Description:

Subjects are assessed prior to administration of radiation and/or probiotic for the presence of acne lesions in an affected area. The affected area is exposed to one or more doses of electromagnetic radiation at a wavelength between 400 and 700 nm. Each dose is administered as a continuous pulse, or as a plurality of intermittent pulses. Subjects are assessed again at 4, 8 and 12 weeks. Each assessment comprises grading of acne lesions using a global acne grading scale (GAGS), determining adverse effects, and profiling of bacteria from each subject.

Eligibility and Inclusion Criteria:

Male and female subjects that are diagnosed as having acne lesions and a disease-associated strain of P. acnes.

Exclusion Criteria:

Subjects with a psychiatric disorder that might cause difficulty in obtaining informed consent or in conducting the trial. Subjects having a medical condition that could be affected by exposure to the prescribed wavelengths of electromagnetic radiation.

Primary Outcome Measures:

Determine adverse effects.

Secondary Outcome Measures:

Profile bacteria population after treatment to determine ability of exogenously applied probiotic to populate the skin. Evaluate viability of applied probiotic. Determine efficacy in treatment. Determine percentage change is number of acne lesions before, during and after treatment. Monitor long-term effects of acne lesions after treatment, such as evaluating the number of acne lesions in the affected area days, weeks, and/or months after treatment.

### Example 7: Laser Devices

The electromagnetic radiation performed in each of Examples 1-6 is performed using a laser from, or derived from, Table 2. Exemplary dose regimen includes initial dosage and maintenance dose regimens may vary as necessary depending on the device and condition of acne. Each dosage is applied to an area of the skin having acne lesions. Multiple dosages may be administered to multiple areas of the skin.

TABLE 2
Handheld Laser Devices

| Device Name | Light | Exemplary Dose Regimen |
|---|---|---|
| Tria | Blue | about 5 mins per day |
| Sirius Nova | Blue/Red/Green | about 3-4 mins, 3-7 times per |
| | | week |
| Tanda | Blue | 3 mins per day |
| Project E-Light | 7 colors | about 3-20 mins, 3-7 times per |
| | | week |
| Silk'n Blue | Blue | about 3-4 mins per day |
| Bright Pad | Blue/Red/Green | 15-30 mins per day |

For the clinical trials of Examples 4-6, subjects treated with electromagnetic therapy from one or more of the devices may show improvement in acne lesions within days of therapy. The satisfaction of the subjects with the treatment may be at least 50%.

### Example 8: Identification of Health-Associated Strains

Characteristics that may predispose a particular microbe to be a health-associated microbe can be determined using samples from healthy and disease afflicted individuals, culturing the microbes from each, and performing a comparative genomic analysis. In the present example, samples were collected from individuals afflicted with acne vulgaris in order to determine health-associated P. acnes strains.

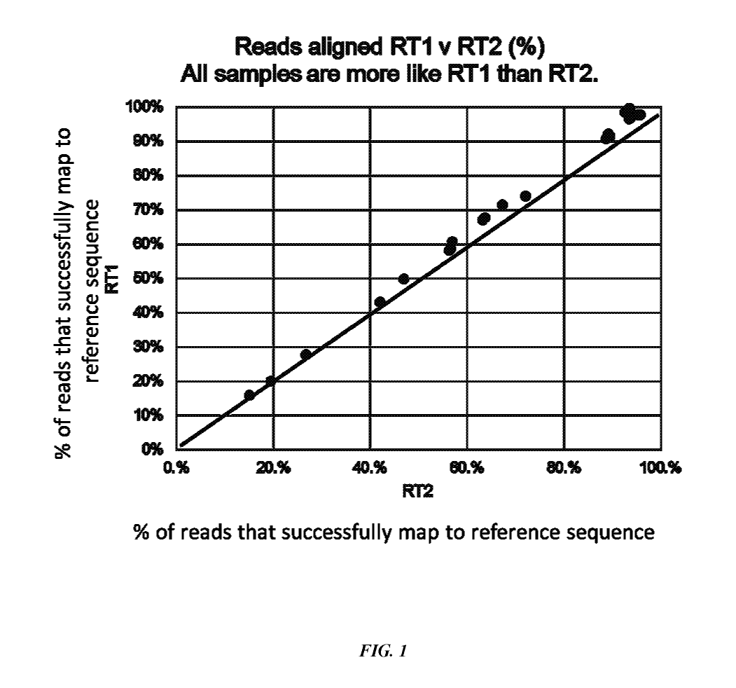

Microcomedone or swab samples were collected from consented adult subjects. Clonal samples were isolated by limiting dilution on plates, and then grown in 200 μL of liquid culture. Microbial DNA was isolated from 96 individual cultures. DNA was isolated using QIAgen's DNeasy Blood & Tissue kit, following the manufacturer's instructions. QIAgen's DNeasy Blood & Tissue kit, following the manufacturer's instructions. Paired-end DNA sequencing (2×300 bp) was done on an Illumina MiSeq using reagent kit v3, following the manufacturer's instructions, yielding 200,000 to 600,000 reads for each of the 96 samples. Initial analysis was performed in Illumina's Basespace Sequence Hub, all reads from each sample are aligned with a BWA Aligner to:
a. deoR;
b. Propionibacterium acnes ATCC 11828 (accession CP003084); or c. pIMPLE and other reference genomes.
   Alignments were interrogated with the Broad Institute's Integrative Genomics Viewer and confirmed using Biomatter's Geneious version 9.1. All 96 clones were analyzed for the presence or absence of the deoR sequence, type I lipase or type II lipase sequence, and presence or absence of pIMPLE plasmid. Sequence alignments were performed between sequences of P. acnes from healthy volunteers and the deoR gene. Analysis revealed that approximately half of all healthy clones were positive for deoR (greater than 0.4% of reads mapping to deoR locus). Sequence alignments were also performed between P. acnes of healthy volunteers and the lipase gene locus P. acnes were positive for type I Lipase and for type II Lipase. With regard to the pIMPLE plasmid sequence alignments of reads from healthy volunteers performed against pIMPLE-HL096PA1 (GenBank: CP003294.1), revealed P. acnes from healthy volunteers are free of pIMPLE plasmid. Reads from healthy volunteers map P. acnes to ribotype RT1. FIG. 1 corroborates this by showing that more RT1 strains are deoR positive and type II lipase positive when compare to RT2. Some results are summarized in Table 3.

TABLE 3
summary of sequencing data for the P. acnes isolated from
healthy volunteers

| | | RT1; | RT1; | | | | |
|---|---|---|---|---|---|---|---|
| | RT1; | deoR+; | deoR+; | | | | |
| | deoR− | LP1 | LP2 | RT2 | Staph. | Other | sum |
| reads | 112 | 160 | 42 | 1 | 7 | 48 | 370 |
| % of total | 30.3% | 43.2% | 11.4% | 0.3% | 1.9% | 13.0% | |

RT1 = ribotype 1;
RT2 = ribotype 2;
deoR− = no deoR;
deoR+ = deoR;
LP1 = type I;
Lipase; PL2 = type II lipase;
Staph = Staphylococcus;
other P. avidum, P. acidipropionici, or Staphylococcus

### Example 9: Identification of Health-Associated Strains with Hyaluronidase Genes

Health-associated P. acnes clones that were RT1 or RT2 positive were further examined for presence of a gene encoding hyaluronidase. Unexpectedly most health-associated strains that were positive for Type II lipase also possessed a hyaluronidase gene. See Table 4.

TABLE 4
Hyaluronidase presence in health-associated P. acnes
strains also positive for type II lipase

| Clone | genotype | Hyaluronidase |
|---|---|---|
| 1 | RT1; deoR+; L2 | Yes |
| 2 | RT1; deoR+; L2 | Yes |
| 3 | RT1; deoR+; L2 | Yes |
| 4 | RT1; deoR+; L2 | Yes |
| 5 | RT1; deoR+; L2 | Yes |
| 6 | RT1; deoR+; L1 | No |
| 7 | RT1; deoR+; L2 | Yes |
| 8 | RT1; deoR+; L2 | Yes |
| 9 | RT1; deoR+; L2 | Yes |
| 10 | RT1; deoR+; L2 | Yes |
| 11 | RT1; deoR+; L2 | Yes |
| 12 | RT2 | Yes |
| 13 | RT2 | Yes |
| 14 | RT2 | Yes |
| 15 | RT2 | Yes |

RT1 = Ribotype 1;
RT2 = Ribotype 2;
L1—Lipase type I;
L2 = Lipase type 2;
DeoR+ = DeoRepressor positive

### Example 10: P. acnes Viability Assay

Viability of P. acnes was assessed each week over two months of storage as shown in Table 5. At least three samples were tested at each time point.

TABLE 5
Assessed P. acnes storage conditions

| Solution | Temperature |
|---|---|
| 25% glycerol in water | 4° C. |
| 50% glycerol in water | 4° C. |
| 25% glycerol in 75% PBS | 4° C. |
| 25% glycerol in water | −20° C. |
| 50% glycerol in water | −20° C. |
| 25% glycerol in water | −80° C. |

Samples were prepared according to the following:

1. P. acnes of ribotypes RT1(HP3A11) and RT2 (HP5G4) were started at 0.066 OD600 and grown to ˜1.0 OD600 in exponential phase in reinforced clostridial medium (RCM).
2. A day later, cultures displayed a dense turbidity, and they were split 1:2 with RCM to produce four liquid culture (LC) samples of each ribotype: 4 RT2 LC and 4 RT1 LC.
3. Two days later, resulting pellets and media were separated. The media of the LC was split between two tubes (˜3 ml), and tubes were filled with 9 ml fresh media and vortexed. Pellets remained in original test tubes and were resuspended by pipetting with 8 ml fresh RCM. All LC (the 8 pellet LCs (4 RT1 and 4RT2), and 16 media-derived LCs) were placed into ajar with two sachets given a large quantity of oxygen filled the jar.
4. LCs were vortexed, split and fed fresh media as they became very turbid and large pellets formed.
5. A day before the experiment, cultures were vortexed, split, spun down at 4,300 g for 5 minutes, and media replaced.
6. On the day of initiating storage: LCs were split into sterile 50 ml conical tube (e.g., 50 ml aliquots of RT1 or RT2), avoiding the pelleted cells. Conical tubes were vortexed lightly and OD600 measured. Optionally, LCs may be diluted if OD600 is greater than 1.0.
7. LCs were split into aliquots and spun down at 4,000 rcf for 5 minutes. Media was discarded and pellets washed with 5 ml 25% v/v glycerol/water to wash the cells. Cells were centrifuged once more, and wash solution discarded.
8. Cells were added to 8.75 ml 25% glycerol in water, 6 ml 50% glycerol in water or 3.25 ml 25% glycerol in PBS to produce live bacteria solutions.
9. 250 microliters of live bacteria solutions were added to 1.5 ml eppendorf tubes, and placed at 4° C., −20° C. or −80° C.

Cell viability was assessed according to the following:

1. At each time point, Eppendorf tubes were selected from each treatment, and allowed to come to room temperature. Tubes were inverted six times.
2. 20 microliters of the thawed stocks were serially diluted in 96 well plates with RCM.
3. Thawed stocks were also spotted on Brucella plates at various dilutions.
4. Plates were imaged with a digital camera, and cells counted with 95% Confidence Interval.

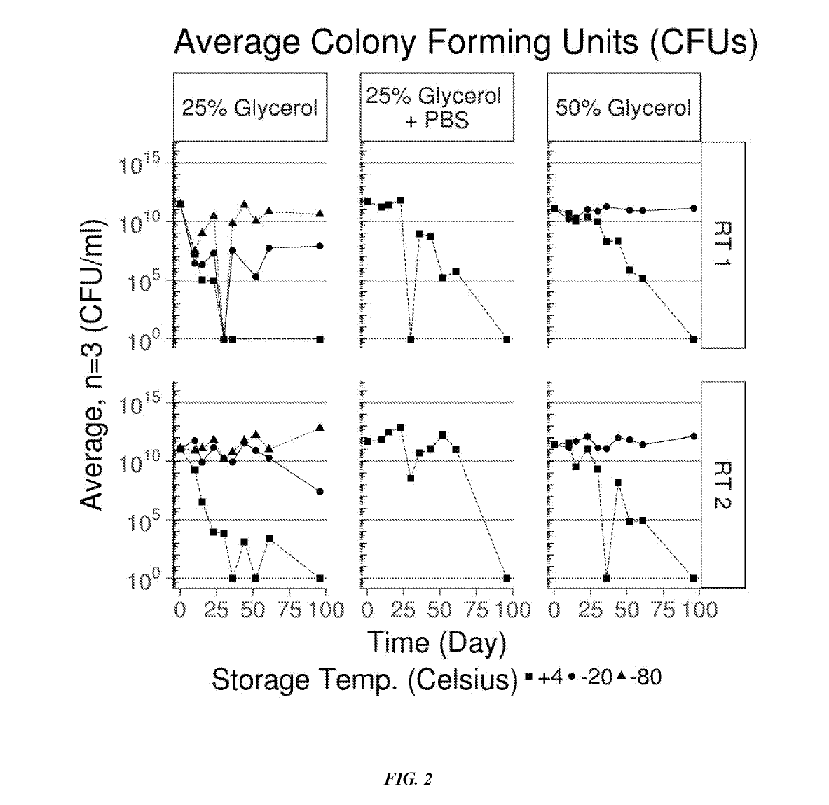

FIG. 2 shows the viability of a variety of Ribotype 1 and Ribotype 2 P. acnes preparations after 30 days, 60 days and 90 days of preservation. Heat shock of a sample, simulating direct application to skin, demonstrated that these samples would retain reported viability if used for acne treatment.

### Example 11. Identification of P. acnes RT6

In an effort to isolate and purify health-associated strains of P. acnes, (e.g., strains associated with acne) it may be useful to identify undesirable strains of P. acnes in a sample (e.g., strains found on skin of subjects with acne). For instance, in some cases, P. acnes of ribotype RT6 is undesirable. To this end, genes can be identified that are specific to strains of interest. The following example demonstrates how this can be performed.

Identities of genes that distinguish P. acnes of ribotype RT6 from healthy strains were confirmed. Genes encoding DNA binding response regulator and phosphoglycerate kinase were identified in P. acnes of ribotype RT6, but not RT1, RT2, RT3, RT4 and RT5. In addition a gene encoding ABC transporter is absent in RT6, but present in RT1, RT2, RT3, RT4 and RT5. Sequences for these genes are provided as SEQ ID NOS: 10 (ABC transporter), 11 (DNA binding response regulator), and 13 (phosphoglycerate kinase)

The presence or absence of these genes was confirmed by sequence alignment using BLAST, Megablast, (a registered trademark of the National Library of Medicine) either the whole complete genome or all of the scaffolds of a completed genome against each of these three gene sequences; the results are shown in Table 6. “Y” is a perfect match for the entire sequence OR >60 bp continuous perfect sequence alignment. “N” means there is <60 bp perfect alignment. The best match of a “N” was 26 bp.

TABLE 6
Genotypes of P. acnes strains

| | | recA | ABC | DNA binding | Phosphoglycerate |
|---|---|---|---|---|---|
| Strain Name | Ribotype | type | transporter | response regulator | kinase |
| HL002PA2 | 1 | IA | Y | N | N |
| HL025PA1 | 1 | IB | Y | N | N |
| HL030PA1 | 1 | IB | Y | N | N |
| HL050PA2 | 1 | II | Y | N | N |
| HL096PA3 | 1 | IA | Y | N | N |
| HP3A11 | 1 | IB | Y | N | N |
| HP3B4 | 1 | | Y | N | N |
| KPA171202 | 1 | IB | Y | N | N |
| ATCC 11828 | 2 | II | Y | N | N |
| HL001PA1 | 2 | II | Y | N | N |
| HL103PA1 | 2 | II | Y | N | N |
| HP4G1 | 2 | II | Y | N | N |
| HP5G4 | 2 | II | Y | N | N |
| HL002PA1 | 3 | IB | Y | N | N |
| HL005PA1 | 4 | IA | Y | N | N |
| HL007PA1 | 4 | IA | Y | N | N |
| HL038PA1 | 4 | IA | Y | N | N |
| HL045PA1 | 4 | IA | Y | N | N |
| HL053PA1 | 4 | IA | Y | N | N |
| HL056PA1 | 4 | IA | Y | N | N |
| HL074PA1 | 4 | IA | Y | N | N |
| HL099PA1 | 4 | IA | Y | N | N |
| HL043PA1 | 5 | IA | Y | N | N |
| HL043PA2 | 5 | IA | Y | N | N |
| HL072PA1 | 5 | IA | Y | N | N |
| HL072PA2 | 5 | IA | Y | N | N |
| HL096PA1 | 5 | IA | Y | N | N |
| HL096PA2 | 5 | IA | Y | N | N |
| HL097PA1 | 5 | IC | Y | N | N |
| PRP-38 | 5 | IC | Y | N | N |
| HL110PA3 | 6 | II | N | Y | Y |
| HL110PA4 | 6 | II | N | Y | Y |

### Example 12. Pan Bacterial Assay to Characterize Skin Microbiome

Robust pan-sampling of the skin microbiome is demonstrated in the following example. This can be performed with or without the use of preservatives. This method is compatible with qPCR analysis and does not require DNA purification. TaqMan qPCR assays were used to quantitate most bacteria collected from the face. Performance was confirmed with two different bacterial phyla, all Propionibacterium and Staphylococcus. This method required the assessment of only a single locus to recognize most bacteria commonly found on the face (P. acnes strains and Staphylococcus), whereas current methods in the field use multiple primer pairs to achieve similar coverage. The majority of the bacteria on the skin of a subject's face is described in the following Table 7.

TABLE 7
Bacteria on Human Facial Skin

| | P. acnes | P. avidum | S. epidermidis | S. aureus |
|---|---|---|---|---|
| Kingdom | Bacteria | Bacteria | Bacteria | Bacteria |
| Phylum | Actinobacteria | Actinobacteria | Firmicutes | Firmicutes |
| | | | Bacilli | Bacilli |
| Order | Actinomycetales | Actinomycetales | Bacillales | Bacillales |
| Family | Propionibacteriaceae | Propionibacteriaceae | Staphylococcaceae | Staphylococcaceae |
| Genus | Propionibacterium | Propionibacterium | Staphylococcus | Staphylococcus |
| Species | P. acnes | P. avidum | S. epidermidis | S. aureus |

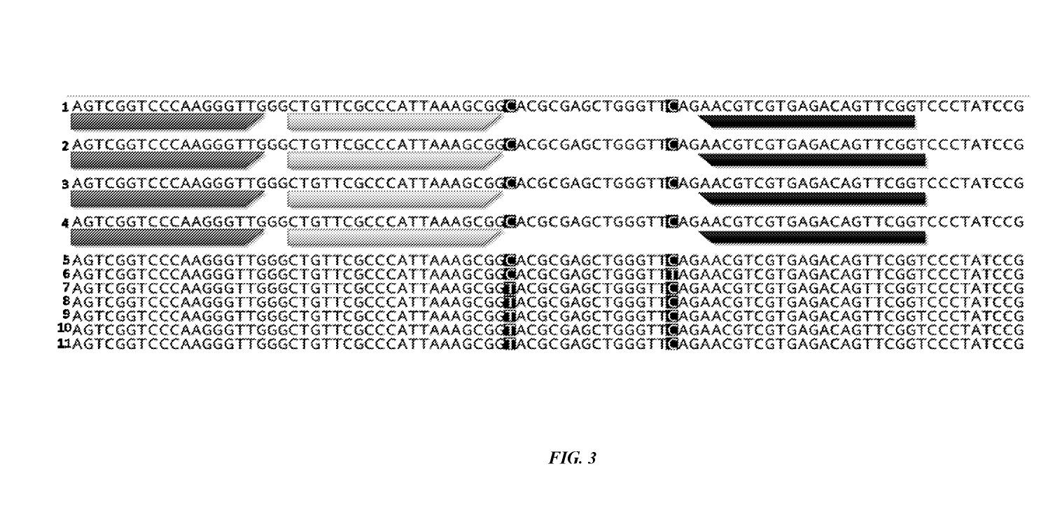

A portion of a 23S sequence from bacteria commonly found on the human face was aligned with known sequences, see FIG. 3. Despite two Single Nucleotide Polymorphisms at this loci (denoted by bold and underlined letters), careful placement of primers (gray and black) and TaqMan reporter (white) enable quantification of widely diverse bacteria from both Actinobacteria and Firmicutes.

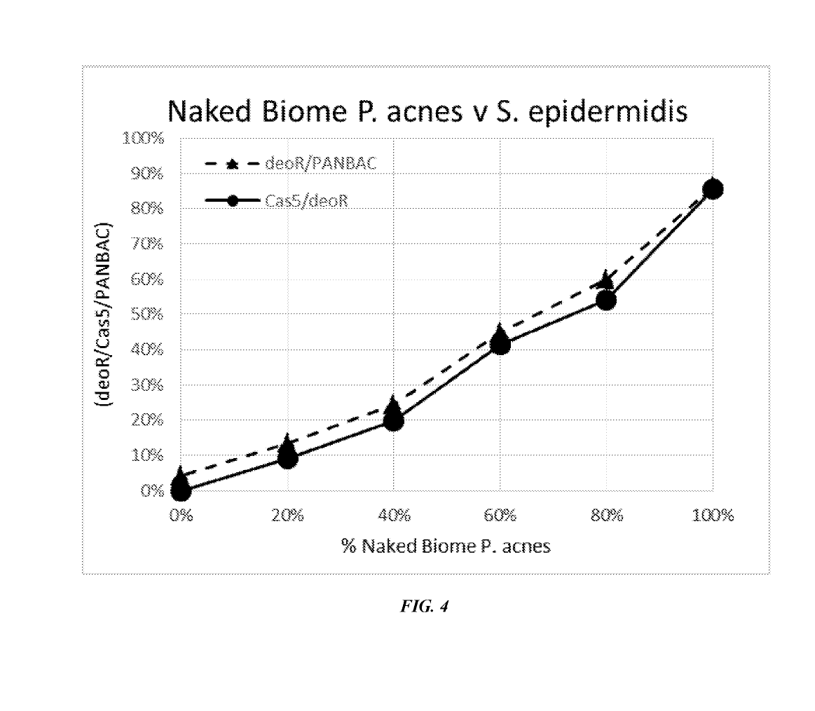

A standard curve for all assays was generated with P. acnes. Percentages of health-associated P. acnes were computed using a dilution series with S. epidermidis or pathogenic P. acnes which were used to quantitate a percentage of health-associated P. acnes in a collected sample. These percentages were determined by measuring deoR+ or Cas5+ bacteria in the overall sample of bacteria (PANBAC), see, e.g., FIG. 4.

### Example 13. Determination of Percentage of pIMPLE Plasmid

The percentage of pIMPLE plasmid was determined from biological samples. Biological samples were collected and grown in 200 μL of liquid culture. DNA was isolated using QIAgen's DNeasy Blood & Tissue kit, following the manufacturer's instructions. Paired-end DNA sequencing (2×300 bp) was done on an Illumina MiSeq using reagent kit v3, following the manufacturer's instructions, yielding 200,000 to 600,000 reads for each sample. Initial analysis was performed in Illumina's Basespace Sequence Hub, all reads from each sample are aligned with a BWA Aligner to pIMPLE. Alignments were interrogated with the Broad Institute's Integrative Genomics Viewer and confirmed using Biomatter's Geneious version 9.1.

The percentage of pIMPLE was determined by the percentage of total sequencing reads that aligned to pIMPLE plasmid from HL096PA1. The percentage of pIMPLE was also calculated as the coverage * copy number. Using these methods, the percentage of pIMPLE in the different ribotypes was determined as seen in Table 8.

TABLE 8
Presence of pIMPLE plasmid in different P. acnes strains.

| Ribotype | Strain | % pIMPLE |
|---|---|---|
| 1 | HP3A11 | 0.23% |
| 1 | HP3A11 | 0.24% |
| 2 | HP5G4 | 0.26% |
| 2 | HP5G4 | 0.24% |
| 2 | HP4G1 | 0.26% |
| 2 | HP4G1 | 0.25% |
| 4 | HL045PA1 | 3.62% |
| 4 | HL045PA1 | 3.22% |
| 5 | HL043PA1 | 4.32% |
| 5 | HL043PA1 | 3.75% |
| 6 | HL110PA3 | 12.94% |
| 6 | HL110PA3 | 12.59% |
| 6 | HL110PA4 | 13.19% |
| 6 | HL110PA4 | 14.06% |

### Example 14. Genetic Modification of P. acnes

In order to improve healthy P. acnes clones, the expression of a gene in the porphyrin synthetic pathway was knocked out. This was accomplished by inserting stop codons in the middle of the open reading frame of the gene HemY (protoporphyrinogen oxidase, EC:1.3.3.4 1.3.3.15) in the P. acnes genome. Briefly, the RNA-guided DNA endonuclease Cas9 (CRISPR associated protein 9) was targeted to HemY with specific CRISPR RNA (crRNA), and trans-activating RNA (tracrRNA) cleaving a double stranded break at the desired location in the HemY gene. A specific sequence was inserted at the site of the cleavage with a Homology Directed Repair cassette (HDR).

The Cas9, crRNA, tracrRNA, and HDR donor template were introduced into P. acnes using electroporation to transform the cells. Cells must be electrocompetent before undergoing electroporation. Electrocompetent P. acnes were prepared by growing them to stationary phase and washing them in a buffer of sucrose, magnesium chloride, and monosodium phosphate.

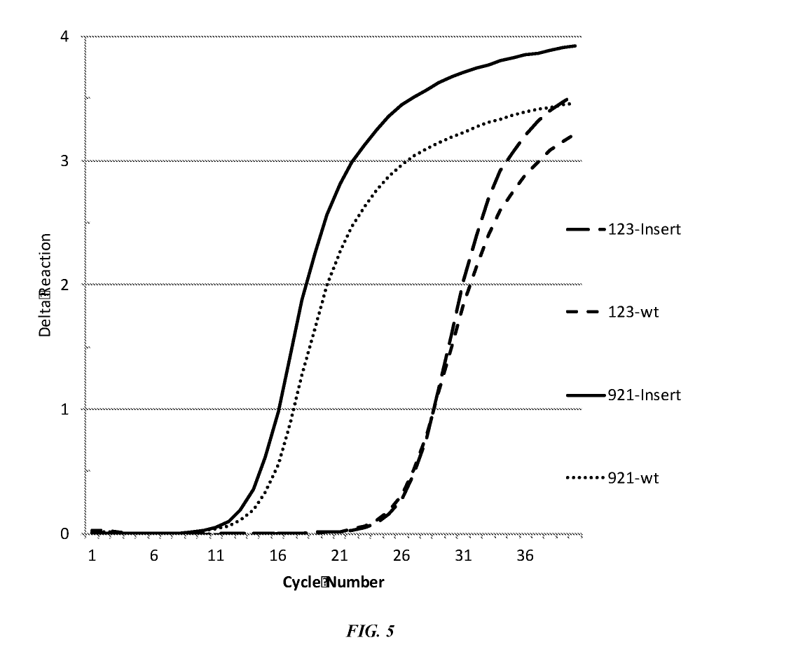

The tracrRNA and crRNA were duplexed using IDT's duplex-forming buffer. Then the tracrRNA:crRNA duplex was incubated in a solution of Cas9 and phosphate-buffered saline, forming the ribonucleoprotein (RNP) complex. The RNPs, HDR, and electrocompetent P. acnes were combined, incubated on ice (transformation culture) and transferred to a pre-chilled BioRAD electroporation cuvette. The transformation culture was electroporated using a BioRAD Micropulser. Rich clostridium medium was immediately added to the transformation culture and transferred to separate container for a 24 hour, room temperature incubation. The transformation culture was evaluated with qPCR (see FIG. 5) and spread out over multiple Brucella plates for a final 72 hour anaerobic incubation at 37° C.

FIG. 5 compares a qPCR result from cells transformed with a 921 bp (921) or a 123 bp HDR. Each sample was evaluated with primers that recognized either the inserted sequence (Insert) or the untransformed or wild-type (wt) genomic sequence. Note, using the longer, 921 bp, HDR transformed a greater percentage of the cells. The ‘921’ sample had more cells resulting the leftward shift of both Insert and wt lines.

TABLE 9
Exemplary Sequences (Additional SEQ IDs provided in sequence listing filed
herewith). Bold characters highlight differences between Type I lipase and
Type II lipase.

| SEQ | | |
|---|---|---|
| ID | Descrip- | |
| NO: | tion | Sequence |
| 15 | | caaccgtaga tacagataca tctgaggaga tc |
| 16 | | catgaagaaa aa |
| 17 | | ccgcgcc |
| 18 | | tcaggttcgc aatgaaga |
| 19 | | atgacagaca ggtcctatcc ggcgatgatc cggcttcggc gcaacgcctg gaccgagttc |
| | | gtcccgttcc tggattacga cgtcgagatc cgcaagatcc tctgctcgac gaacgcgatc |
| | | aagtcgttga acacccgctt ccgcacggtc atgcgggcgc agggtcattt cccgacgcgc |
| | | tga |
| 20 | | agtcggtccc aagggttggg ctgttcgccc attaaagcgg cacgcgagct gggttcagaa |
| | | cgtcgtgaga cagttcggtc cctatccg |
| 21 | | agtcggtccc aagggttggg ctgttcgccc attaaagcgg cacgcgagct gggttcagaa |
| | | cgtcgtgaga cagttcggtc cctatccg |
| 22 | | agtcggtccc aagggttggg ctgttcgccc attaaagcgg cacgcgagct gggttcagaa |
| | | cgtcgtgaga cagttcggtc cctatccg |
| 23 | | agtcggtccc aagggttggg ctgttcgccc attaaagcgg cacgcgagct gggttcagaa |
| | | cgtcgtgaga cagttcggtc cctatccg |
| 24 | | agtcggtccc aagggttggg ctgttcgccc attaaagcgg cacgcgagct gggttcagaa |
| | | cgtcgtgaga cagttcggtc cctatccg |
| 25 | | agtcggtccc aagggttggg ctgttcgccc attaaagcgg cacgcgagct gggtttagaa |
| | | cgtcgtgaga cagttcggtc cctatccg |
| 26 | | agtcggtccc aagggttggg ctgttcgccc attaaagcgg tacgcgagct gggttcagaa |
| | | cgtcgtgaga cagttcggtc cctatccg |
| 27 | | agtcggtcccaagggttggg ctgttcgccc attaaagcgg tacgcgagct gggttcagaa |
| | | cgtcgtgaga cagttcggtc cctatccg |
| 28 | | agtcggtccc aagggttggg ctgttcgccc attaaagcgg tacgcgagct gggttcagaa |
| | | cgtcgtgaga cagttcggtc cctatccg |
| 29 | | agtcggtccc aagggttggg ctgttcgccc attaaagcgg tacgcgagct gggttcagaa |
| | | cgtcgtgaga cagttcggtc cctatccg |
| 30 | | agtcggtccc aagggttggg ctgttcgccc attaaagcgg tacgcgagct gggttcagaa |
| | | cgtcgtgagacagttcggtc cctatccg |
| 31 | Type I | gtagatacagatacatctgaggagatccatgaagaaaaactggttactcacaaccctccttgccaca |
| | lipase | atgatgatcgccatgggcacgacgaccaccgccttcgccagcccgcctaccgacatcactcccgaa |
| | | catccaggcggggttaccgcgcctcacagccccgacggaatcccctcgaatattgaggggccaagt |
| | | atgcccagctggacctctgcaatcaggttcgcaatgaagaaccccggcacgaaagtcccgggcacc |
| | | aacgacttcacctgcaaaccgaggaaaggcacccatcccgtcgtgctcatcccgggcacatccgag |
| | | gacgccttcatcacgtggtcgtactacggtccccgccaggattctgcgcctacacgttcaactacaac |
| | | ccggaaacacatccgcttgtggaagccgctgagaccagcggcaacatctactccacggcagctttc |
| | | atggcccacttcgttgacagagtgctcaaggcaaccggtgctcagaaggtcaacctcgtcggccatt |
| | | ctcagggcggcggccccctgccgcgcgcgtacatcaaatattacggggcgccaagaaagtcctcat |
| | | ctcgtcggtttggttccttccaacaggggaacacgcatgctcggcctggagaagttcctcaatgccag |
| | | cggaaacccgctcagcactatcttcaatgctgcagcacagtttcgaaagctggaatccctgccccaac |
| | | agttgcaagactccacatttctcagggaactcaacgcggatggaatgaccgtccccggcatcacata |
| | | caccgtcatcgccacccagttcgacaaccgagtatttccgtggactaataccttcatcaatgagcccg |
| | | gggtcaagaacatcgtcatccaagacgtctgtcccttggaccacagcgcccacacggatatccctag |
| | | gacccgatgacccttcagattgtcatcaacgccttggaccccgagcgggccgccccggtcacctgc |
| | | accattcgcccattcaggcccagttag |
| 32 | Type II | gcagatgcatctgagaagatccatgaagaaaaactggttactcacaaccctccttgccacaatgatga |
| | lipase | tcgccatgggcacgacgaccaccgccttcgccagcccgcctaccgacatcactcccgaacatccag |
| | | gcggggttacccgcctcacagccccgacggaatcccctcgaatattgaggggccaagtatgcccag |
| | | ctggacctctgcaatcaggttcgcaatgaagaaccccggcacgaaagtcccgggcaccaacgactt |
| | | cacctgcaaaccgaggaaaggcacccatcccgtcgtgctcatcccgggcacatccgaggacgcctt |
| | | catcacgtggtcgtactacggtccccgccaggattctgcgcctacacgttcaactacaacccggaaa |
| | | cacatccgcttgtggaagccgctgagaccagcggcaacatctactccacggcagctttcatggccca |
| | | cttcgttgacagagtgctcaaggcaaccggtgctcagaaggtcaacctcgtcggccattctcagggc |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. (canceled)

2. A method for treating an individual affected with disease-associated bacteria, the method comprising exposing an affected site of the individual to electromagnetic radiation at a wavelength from about 300 nm to about 1400 nm; provided that the wavelength corresponds to a wavelength between about 90 nm below and about 90 nm above an absorption maxima of a metabolite from the disease-associated bacteria; and provided that the electromagnetic radiation is emitted from a source having a power output from about 0.5 mW to about 20 mW; and provided that the disease-associated bacteria comprises a P. acnes ribotype 3, P. acnes ribotype 4, P. acnes ribotype 5, P. acnes ribotype 7, P. acnes ribotype 8, P. acnes ribotype 9, P. acnes ribotype 10, or a combination thereof.

3. (canceled)

4. (canceled)

5. The method of claim 2, provided that: the metabolite has an absorption maxima between about 390 nm and about 700 nm, the metabolite comprises a porphyrin, or the metabolite comprises a porphyrin and has an absorption maxima between about 390 nm and about 700 nm.

6. (canceled)

7. The method of claim 2, provided that the electromagnetic radiation source comprises a light emitting diode that emits blue light, red light, both blue light and red light; and/or has a dominant wavelength selected from the group consisting of 390 nm, 400 nm, 420 nm, 430 nm, 445 nm, 475 nm, 590 nm, 635 nm, 655 nm, 660 nm, 670 nm, 780 nm, 785 nm, 810 nm, 830 nm, 840 nm, 860 nm, 904 nm, 915 nm, 980 nm, 1015 nm, 1060 nm, 1260 nm, and 1400 nm.

8. (canceled)

9. (canceled)

10. (canceled)

11. (canceled)

12. (canceled)

13. The method of claim 2, further comprising administering a health-associated bacteria to the individual, wherein the health-associated bacteria is optionally formulated with an excipient or biological stabilizer.

14. The method of claim 13, provided that the health-associated bacteria comprises a P. acnes ribotype 1, a P. acnes ribotype 2, or a combination thereof.

15. The method of claim 13, provided that the health-associated bacteria comprises at least one gene encoding at least one of a deoxyribose operon repressor and a type II lipase, and less than about 10% pIMPLE plasmid.

16. The method of claim 13, provided that the health-associated bacteria comprises less than about 1% pIMPLE plasmid.

17. (canceled)

18. (canceled)

19. The method of claim 13, provided that the health-associated bacteria comprises two separate strains of health-associated bacteria.

20. (canceled)

21. (canceled)

22. The method of claim 2, comprising administering to the individual: (i) a photomodulation enhancing agent prior to the electromagnetic radiation exposure, (ii) a penetration enhancing procedure prior to the electromagnetic radiation exposure, or (iii) i and ii.

23. (canceled)

24. (canceled)

25. (canceled)

26. (canceled)

27. (canceled)

28. A method of affecting the microbiome of an individual, the method comprising administering a composition comprising a health-associated bacteria and an excipient or biological stabilizer to the individual, provided that the individual has undergone a treatment targeting a disease-associated bacteria on an affected site of the individual.

29. The method of claim 28, provided that about 10⁴-10¹⁰ colony forming units of the health-associated bacteria is administered to the individual.

30. The method of claim 28, provided that the treatment comprises exposing the affected site of the individual to electromagnetic radiation at a wavelength from about 300 nm to about 1400 nm.

31. (canceled)

32. The method of claim 28, comprising administering to the individual a photomodulation enhancing agent and/or performing a penetration enhancing procedure prior to the electromagnetic radiation treatment.

33. The method of claim 28, provided that the health-associated bacteria comprises a P. acnes ribotype 1, a P. acnes ribotype 2, or a combination thereof.

34. The method of claim 28, provided that the health-associated bacteria comprises at least one gene encoding at least one of a deoxyribose operon repressor and a type II lipase, and less than about 10% pIMPLE plasmid.

35. The method of claim 28, provided that: (i) the health-associated bacteria comprises less than about 1% pIMPLE plasmid, (ii) the health-associated bacteria produces porphyrin at a level about equal to or less than P. acnes strain HL103PA1, or (iii) i and ii.

36. (canceled)

37. (canceled)

38. The method of claim 28, provided that the health-associated bacteria comprises two separate strains of health-associated bacteria.

39. (canceled)

40. (canceled)

41. (canceled)

42. (canceled)

43. A composition comprising:
(a) a bacteria comprising: (i) S. salivarius, (ii) Lactobacillus, (iii) Bifidobacterium, (iv) Staphylococcus hominis, (v) Propionibacterium freudenreichii ssp., (vi) Shermanii JS, (v) an organism comprising at least one gene encoding at least one of a deoxyribose operon repressor, a CRISPR associated Cas endonuclease, and a type II lipase, or (vi) any combination of (i) to (v); and
(b) an agent comprising at least one of: an antibiotic, an anti-inflammatory agent, an antioxidant, an acid, benzoyl peroxide, a penetration enhancer, a carbohydrate, a cryopreservative, a small molecule, a fatty acid, an antibiotic, a metabolite, a retinoid, and a molecule or phage targeting at least one strain of P. acnes;
wherein the composition is optionally formulated as a gel, ointment, cream, lotion, tincture, emulsion, emollient, or foam; or is optionally formulated for oral administration.

44. The composition of claim 43, provided that the composition comprises:
(i) the antibiotic, wherein the antibiotic comprises at least one of: clindamycin, doxycycline, erythromycin, doxycycline, minocycline and tetracycline;
(ii) the antioxidant, wherein the antioxidant comprises at least one of: ascorbic acid, alpha-tocopherol, vitamin D, lipoic acid, glutathione, uric acid, ubiquinol, green tea extract, coffee berry extract, polyphenol, resveratrol, 3,4,5 trihydroxystilbene (trans-resveratrol), and pterostilbene;
(iii) the carbohydrate, wherein the carbohydrate comprises at least one of: trehalose, mannose, fructose, glucose, sucrose, lactose, raffinose, stachyose, melezitose, dextran, and a sugar alcohol;
(iv) the cryopreservative, wherein the cryopreservative comprises at least one of: glycerol, bovine-free media, whey protein, NaCl, phosphate buffer, MgCl, and a second bacteria that is lyophilized, inactive, or killed;
(v) the retinoid, wherein the retinoid comprises at least one of: retinoic acid, tretinoin, tazarotene, adapalene, and retinol;
(vi) the acid, wherein the acid comprises salicylic acid, glycolic acid, azaelic acid, and trichloroacetic acid;
(vii) the fatty acid, wherein the fatty acid comprises an omega-3 fatty acid; or
(viii) any combination of (i) to (vii).

45. The composition of claim 43, provided that the composition:
(i) comprises the Lactobacillus, wherein the Lactobacillus comprises at least one of: L. acidophilus La-5, L. rhamnosus, L. F19, L. fermentum, L. Sakei, L. reuteri, L. acidophilus, L. casei, and L. salivarius;
(ii) comprises the Bifidobacterium, wherein the Bifidobacterium comprises at least one of: B. animalis, B. breve, and B. bifidum; or
(iii) (i) and (ii).


SEQUENCE LISTING (14 sequences)

SEQ ID NO: 1 - DNA, 2299 bp, Propionibacterium acnes
ttggtcagcgcccaccgatgccttacaccgtgacacgctcgaagtggcgatggaacacctacgcttcgaacctgattcgatcgcttcggtggcctcctgctgacgctttcatggatcgtgtcagcctggaggatggtaatgcgcccaaaactgtatgtcttcggtagatctgcacaagcggatcatctcgcgcaagaaggaaccgtgaggtactcgatccggtacactctccacatcaaaaccaacaatacaagtcacagtcatagatagttgtgtcattaacaagaaatcccaacagcaccccaatgcaaaattgcacagaacgacctagccagatgattttcagcaagtctaccatgacagcaaaggccgcagatagaaactgtgacatatacattgcacgctgaacttactaccccaccactaaatttacatagattccataccgacccgaacgccaccagtcaaccatcagtaatcatcatcgagaagtcaaaaataaccaacaccattgataagatccatgccatggacgggcagacgtctgtgatcagcagagtcgagaaacgaggtaaagcaaactatcacgagccaccagataatacccctagaagcttgtccaatatgttagctctggttaacatgccatcgccagcacaagattaacacgcttgttcatgtcttcagaaagcaacgaagctcgcctagatacttggacatttatatccttgcaaacccccccctaaagcctccaaaaatccttgatttacctcactaatataccattaatttgccccacctcaagccaacaaaagaacggagcatcagagccataggaagcctccgccagacataggggactaaacattatctccttctacgtcaccaactagaattcataaagcatcaacctacttatttaattcgatctaaaatccaatatccttagtatcagggatcaataaaaccaaaattaggaaacatacttaaaatcatgacatttcacttagtagacatactttgggattgtatcgacgcgtgacattgacacccatatttctgcggcagaatgcaccccggcaatccattgccaacatgcatgacacaccagcccatcagacccgaggtgaggtgatacactgttggagttagttcctggtacgcaaaccgcacagcccggtaggaagtccgggcggcgcaatagacgtccgtcgctcccctcatggagaatccagtgtcgccaggtccgcttcggcgtactgggagatgatatgcctacgatgttcactgtctacttcgatggccagttctgggtcggagtcctagagaggcgcgacgagggtttggtgcgtgccgtaaaagtcacgtttggcgccgaaccgtctgacacggaattgtacgagtgggttagccgtcatggcaacgcacttatagagcgattggagtctaccgctgctgtccctaccacccgcagtccccgagccaagcgactgaaccccaagagggcgttacgagatgcagcgcgagctgcccaagcaccccgtgccagcacggccgcacaggccgcgcttaaggccgatcaggaagctcggggtcgcgccgccacagtgaagcgcaagcaggcccggatcgacaaagctgcggagcagtgggctaagaaacgagagcgcgcgaaggcaaagcaccgtggccactaatgcgtcgaccgtgcccttcccaactgtgattcttggttcgggacctatggcttcacggtcaaggcggtcaaatcgctcacacatgagtactattcacccgacatgccatcgtctgggcaatcacccaactgcaagctgatgacgttttcctcctaggcttgatgcactagtttagtgtggacaggaagactcatggcgaagggaacggcctgttctcaagatcgccaccgcgaacgagattcacttcccacggtgtacgcatactaaggggcggagcttgtctgacatcatctgggccagcacgcacatggcccagatgatgcccaccggatggtgagcctgacccgtaacaagaacgcatcagcgagcacctcgccgacgcggctgcgcacgcggtcgtaacctcgccactgcctgcgtgaacaacgcgcttcggcgcattcaacacgccgtgaccgcggcgcccacggcttgcgcaaccgctacaactaccacctactgtcgcctcctggtcgttggcggcataacccataaccatcttcaaatccgaagcgccgagtcctgccataattcgaaccttcagcctgcatgactggaacaag

SEQ ID NO: 2 - DNA, 2299 bp, Propionibacterium acnes; identical to SEQ ID NO: 1

SEQ ID NO: 3 - DNA, 2299 bp, Propionibacterium acnes; identical to SEQ ID NO: 1

SEQ ID NO: 4 - DNA, 2298 bp, Propionibacterium acnes
ttggtcagcgcccaccgatgccttacaccgtgacacgctcgaagtggcgatggaacacctacgcttcgaacctgattcgatcgcttcggtggcctcctgctgacgctttcatggatcgtgtcagcctggaggatggtaatgcgcccaaaactgtatgtcttcggtagatctgcacaagcggatcatctcgcgcaagaaggaaccgtgaggtactcgatccggtacactctccacatcaaaaccaacaatacaagtcacagtcatagatagttgtgtcattaacaagaaatcccaacagcaccccaatgcaaaattgcacagaacgacctagccagatgattttcagcaagtctaccatgacagcaaaggccgcagatagaaactgtgacatatacattgcacgctgaacttactaccccaccactaaatttacatagattccataccgacccgaacgccaccagtcaaccatcagtaatcatcatcgagaagtcaaaaataaccaacaccattgataagatccatgccatggacgggcagacgtctgtgatcagcagagtcgagaaacgaggtaaagcaaactatcacgagccaccagataatacccctagaagcttgtccaatatgttagctctggttaacatgccatcgccagcacaagattaacacgcttgttcatgtcttcagaaagcaacgaagctcgcctagatacttggacatttatatccttgcaaaccccccctaaagcctccaaaaatccttgatttacctcactaatataccattaatttgccccacctcaagccaacaaaagaacggagcatcagagccataggaagcctccgccagacataggggactaaacattatctccttctacgtcaccaactagaattcataaagcatcaacctacttatttaattcgatctaaaatccaatatccttagtatcagggatcaataaaaccaaaattaggaaacatacttaaaatcatgacatttcacttagtagacatactttgggattgtatcgacgcgtgacattgacacccatatttctgcggcagaatgcaccccggcaatccattgccaacatgcatgacacaccagcccatcagacccgaggtgaggtgatacactgttggagttagttcctggtacgcaaaccgcacagcccggtaggaagtccgggcggcgcaatagacgtccgtcgctcccctcatggagaatccagtgtcgccaggtccgcttcggcgtactgggagatgatatgcctacgatgttcactgtctacttcgatggccagttctgggtcggagtcctagagaggcgcgacgagggtttggtgcgtgccgtaaaagtcacgtttggcgccgaaccgtctgacacggaattgtacgagtgggttagccgtcatggcaacgcacttatagagcgattggagtctaccgctgctgtccctaccacccgcagtccccgagccaagcgactgaaccccaagagggcgttacgagatgcagcgcgagctgcccaagcaccccgtgccagcacggccgcacaggccgcgcttaaggccgatcaggaagctcggggtcgcgccgccacagtgaagcgcaagcaggcccggatcgacaaagctgcggagcagtgggctaagaaacgagagcgcgcgaaggcaaagcaccgtggccactaatgcgtcgaccgtgcccttcccaactgtgattcttggttcgggacctatggcttcacggtcaaggcggtcaaatcgctcacacatgagtactattcacccgacatgccatcgtctgggcaatcacccaactgcaagctgatgacgttttcctcctaggcttgatgcactagtttagtgtggacaggaagactcatggcgaagggaacggcctgttctcaagatcgccaccgcgaacgagattcacttcccacggtgtacgcatactaaggggcggagcttgtctgacatcatctgggccagcacgcacatggcccagatgatgcccaccggatggtgagcctgacccgtaacaagaacgcatcagcgagcacctcgccgacgcggctgcgcacgcggtcgtaacctcgccactgcctgcgtgaacaacgcgcttcggcgcattcaacacgccgtgaccgcggcgcccacggcttgcgcaaccgctacaactaccacctactgtcgcctcctggtcgttggcggcataacccataaccatcttcaaatccgaagcgccgagtcctgccataattcgaaccttcagcctgcatgactggaacaag

SEQ ID NO: 5 - DNA, 1019 bp, Propionibacterium acnes
gggacagcgaccggtgaagggtccgctgggagcaaggggatcgtcagtcggtgtctgcaggctgaaggggcgcttggatgacgtggtgtccgctccggcggaaggcaccgagcttctcgctcagctcactgttcgacgactggacggtgacgatccccgtccggggcggtatcgatgcccaatatggttcgctaccggtggtgaggatcttggtgggatcagcgaggatccacgacgtgcgagcccgcgtcatcgcttcgtgtttgaactcgacggactccaggagtgggcagttgagcccgtcggtcgcagagactgcgtcgcagccgatgaacgcgacatcaggggtgatgctgcgcaggtttgatgacgcccacggtcccagaaggcttccgctgtgctgccttaacgccccgccgagcaccatgacgttggcggtgccttcggccacggcctggagcacggacaggccgcctacgaggatggtgatgtcgttacgagtcgacagaaggcggcccagtgctgcggtggacgttccggaatcgagcagaactaggtcactctcgttcacgaggtgtttggcggcatatgtggccattgctgccttggagacgtgctcgcattgttccttctcgtgccaggtaggctcgtgtcgtcggatcatgacgtctcccccggaaatcttccagatcttggattcatccgagagggcatcgacatcgcggcgaatcgtcgatgtagagacaccgaacagttgggaaagatctccgatggactccgagtgccccaaagaacgcagatggtcgacaatgtgtcggcgtcgggcagctgtcgccgtgcgcggctgtttgggtgttgtgctcatggtgtcttgatcggtgacgtggtttggctgatccaatttgaggaccgcctctaaggataacccagcggcatcactgctggactcggtctgtttgggtggtcggtccaagaatttctcaggtcacaggctaataaggttacctcgtcggtgcact

SEQ ID NO: 6 - DNA, 55585 bp, Propionibacterium acnes
tgccgacgccttcggcgtcgggtgagcgcgtggataggcgccccttcggggcacctgtaagcctgtatcgctgcggtttacagccttggccacccaaaggggcagcaaggggcccggagggggcattctgcgccgctgatagcatctcacatgtgagttattctctcaactatccttcaagtcaagcgtattagattatttatctagtactcttgacagtccgtctatcgggctatatatttataagcagaacgagagatcgggaaaggaatcaacgatgaagtacaccgcacacgtcaacatggtgaccaagccctacatcggacttcccactacccgaaccccctacaccctcgtcgacgccgactgggccacaacatggatgatcgacgaaacctacgaagcccactaccacggcaccagcgtggagatcagcatctacggcgaaggccccaactgctccgtactcaccaaccgcatctacaccgaccagcccaccgatgtgaccgctgcactcgccaactacggcttcaccatcgacaccaccaacacatccaaaaccatcctcacgctcacccgcacagccaccatctcccacctactagccatcctcgatggcctaccccaagccgaggggactcactggctcaacactcaacccgacctccaccacatcaccctcgacgactccaccaccggctgggccaccaaccgcaccgacgacaccgacaccggcgtctgctacgacaccagcacccacaactggcagatctgctgacagcataacgggcctgcccttgaagggttggactctgcacccatttcagccgggtacgttgccggctgggcgaaggaagcagacggcgccgcgattgaagctgccgccgagcacgtcgcgaagacctcccacgcgattgccgaggcgttgcagctcaccgcacagtgagccccacggggcgaccggctcaccccggtcgcccctcaccccccgacaggggaaccgttcccgccgcctgaatctccatacccctgatacgtactacgcggggtcgaggagtggactcccccaccctcgtgtagtacactatactacacagtcattgaggatagaggtgacgtcatgagtttttcgatccgtttaaccgaccatgagcgccgcttagctgattcctacgcccgcctgcactcgcagtccctgagtgaggctttcaagcaagccctcttcgaccgcatcgccgacgagttcgatctgcaagtagcccgtgaggcactcaccgactacgcccgcgacaactaccagtctcgccccatcagcgagctatgggacgagtgtgacctatgagctggcagatcgaaaccacgagccggttcgataaagaattcaaaaagctagaccgctacacccaaaaactcattcacggctggatcacgaaaaacatcgacaacaccgacgatccccgccgccacggcaagcccctcaccggtgatctttccggactgtggcgctaccgcatcggcgactaccgcctcatctgcacactcaacgataaccgcctcaccatcctcgccctgtccatcggtcaccgccgcgacatctaccgataagccgttgcaagactgcggcgtcgacaccgccgggaccctggcccaggccttgggcctggaggaggacgagagcgaggagcacgccgcctgagcggtcgccggcacttcccataacagcaccggtcccggcggcccggccccgcgtctggtgctcggaccgatgccggcgcctggcctccgacgcccgccggggccagcggctgggctacctcgacgtcgacgtacaacgggccgaacgaaccgtgcgctgatttaccaccgcttccgcacagagaacatgcctctgactaggagaaatgacgttttccacagggcctctattactaatatccctattatctctatccacgcgcgcgagcctcagcgttcaagcttcaggtcttgctcgtctccctcgcgcctcgctgcgctcgttgctactgccgccccacccggcaagccgggtgggttgccatcggaataccgtggaccatcccggcgcatacacccagacggtccaagatccaatccacgctcacccgcgacgccgctaacgatcctcatacaccgtagatgccgttctgagagccttccagacccgcacgtgggtttattgcaccgagacccaaatcccctcagaagcgatcctgaaaccgtctacgccccatttccgacggacgctggacaggctgggcggcttgtctcccgccgcagacatggccctggcatatgcggttgccgactggtgggatcagggattggatcacacggcagacggctggtccaaggcgggtgtgcaggtgcggtgagcggcagacgtgaccggtggggtgcactgtcatggccgggcatctgccgagccaccatgcgggccctggaccaggaggcagcccgctgcgaggactgggggaagacggcggcacggatgggccgtcgggatgcggcggccgaattcaccgagactgcggtgcagcatcgtggacagatgcaccacctcatgtcggcacaaaaagtgtgggtgtcgaccgacatggtgtcggctacactggacgccgcaggagacgtgccgagtttcgaggccgtcgacgtccctgccgccgggctcatgggtttggccagccccctgccgccggttaccctgcagcatccgctgtatctgcgcagcgacgagggtgtgacaaccttcactgatccggttctcgtcgacgcgctgggatggtggatggattctgggcgtgtgcacgtggtgatgtgcacacgcacaccccggctgcccaacccggtttatgcggtggcctccccactgacggtggtggagaaaatcacggtgcccaccggcatcgagttcgagaccatggagtccaggatcctgacccccggcggcgtgacaggcgtctcacggcagggcacacgcatgatgatccggttggcgtcatggctgggtgccgcgtgggtgctcatggcgactccgacggtgtcggagccgcaccctatggatggccggtggggaggcccagcaacggggcagacccggccgcgtgatcgggtgaccgtggtcgacatgcggcccatgaggcaggtgcacaccaccaccgaccccaccggcaggcggttgacgacccggcatgtcgtgcgaggacactggacacaccaaccctacggcccggcccgcagtctccgacgcctgcagtgggtcgccccattcatccgcggccccgaaggggctccgttcgtcggcaccgacaccgtcaccgtatggcgccgctagatacgcgggcgcggggggttgacgaacgatgcccccctctttgtgttgtgatcagtcgtgatcggcgggttttaggtcggggtaggtgtcgagaatggccttgaggacgaggtcttggattgtggtgtcattctgggctgccttgaccttcagtccaacgccgatgtcccgggggattctggtctgcaacttgaacaggtctgcctccttggttttgaacagtttggctgcagggttggcaacgggctgttcttggcgctggcgtccaaggttgcttttctttcgggcggtcatgacatcatctccaacagttcgtcggcaacatcggcgaagaaggcgatagtttccggctcaggggtacagccgtaggcgcgcttgatgacttgacgcttggggatgacgtgatcgaccaactgcgtctgggagtcgttggctagaaggtcgcgaccttccttgaacagggaggtattcggttcagctttggaaagcagcaccacgcgcggggttcctgcggcggcgtcgtgcgtctcccaggtgcgcgccatgtcgagcccggacgtgtcggtggggatgataaccagatcggcggcgcgaatggcggcggtgatgatgtcaccgttgcccggcggggtgtcaatgatgacaacttcttcgggtccggcgacgaggtgctggagcgtcgccttgttggccggttccacggggaaccccaaggggtcggcgttactcagtgtgcactcggcccaggatgatgctgacccttgagggtcggcatctttgacgcggaccatacggcccgtctcatggagagctgcagccagcaggatcgctgccgtggtcttaccgacgccacccttggcgttgataatgctccagatcatggtgacacctttcagtgaggatgcatatcagtttacctgcaaggagtaaagacggcaagcagttatgccgtttctgcggcaagccgtcttgcatccttggatgtatagatagttgtctacttatggttgtggatcgtggtgtctcgtaccaggatttagtgtgctcgctgatatgcacgactttcgggcggcctgatctggcggtggtgccactaacccaggagtctgatccgcaaggtgtcctcgactttgaggaaggggtctctgctttcgtgttgacctaaagtcaacggccctgggcgatcactgaatcctaataactttgtatccttccctgattgcatcttagcatccttgaattcaaacagggatggagccatgctgttttgacgacatggatgcactggggtttggttgttgccgatgcggagcgtcaggcggcccgggacgccgccaccgccgacacgctgcgggagatcgtgggactgctgggccggggcggcgaggtcccgcccgccaagacggggcgtaagacgaccggcaggccggccccggcccgcaagtcgtcggccacgcagtaaaggcagggatccgtccctacgcagagagcggaggcagatcctgggggatccgactcttcggttccgtgatcttcccggagatcttcccggatcccccagggctcatcctgatacggggtccgccgacggaccccgtatcagtgggggcgctgccaccagtgatggcgggcgggtcggtcggcgtctaggccccgcccgatggtcgcctggtctacctcgcaggcccgtgctgttgccgaggccgagaacgtctgcccacttgttgccgattcagtcccctctccgtctgccatgtcgccaacctttcctatctggcgtgctcatggacatgcccgggcacgccctcgctgctcagaggcctgctcgttccacctctggccgcgaaccgtatttctgctgaccactcctcgtgcgtgaatatttgccagccttggccattcatatttggactactctgcaccagatatcggactgggaaacctcttgcacatctctaattgtcatctattcgttgggcgcgcgctgttagatcgtgatcataccaatcagaagacaaagcgacgcccttttgagcggcttttcgaacgattctagaccgcgctatatcagcatttctaccgcgcggccagagtgctacactaaattcctctctgcaatccttaagtccattctttacttcctcatccaatttctgcgacagcatttttatggaagcatcgcactctgactcatcatcaaatgcctcaattgcgtgcgttcgccattcacggcgcgtattatcgtagccgtaatgaagtattacccaccgatccacagaatcatcgttaggatctacaactgtcatgaatggcccaactctcgctctggtgtattctcaattgcagttcagtgactttacttgaccagtcccggatttagtagacgtagacggagagtaccccgccgggaacttgactgacatcactgctgtaccgtactgctttcccgggtggcaaccatcgtaagcagcatttgccgtgacgtacctagtgctctttctctgagtgccggtaatcgccttatatggtacaggtgggtgaatcgtagccgttacggctaccgatgacacgtttgcatcacatgaagctgtggcgacataattagttgtcccattgacatgatgagagtcgtggacatactgtcgaggatcgagagtgcaggttattgttgacgccgttgttctggtaccagctgctttcgagctctttgttgcagttacagtctgcggccctgaaggttttactgacgcgactgatgtgttcgcgtgttttgcggcagtgctttgatttggttgtgcttgtgtagcagttgctgatgtcgccagtgatgcccccattaccatagctgtgattatggcgtagtctttacgcttcattgcgcccccaaataagcatgttagtttgctattttgaataggaaacgagacatcgccatgacttgtcaagggcagaagtagaaagaatcggggggtatcaagaaagatgtgctttcatcaggacgaaagcggatgggtccgttcgacttggtgacgaatgatccggccttgcgtcgcggagaccaggccggcgagtttgtcgagcagggccgccgtggcctcatcgtcgggcacatcactaaagggacgaacaaccatgacggctagggccagaaacgaccggaactgaatcgcatgtggacccccatctggcaccaggcacacacctaccgaccggaaaccctgcaccctgtcgctcccagttcacctcctaatatgcgcctatagtctgtgctagtagacattattttgtgtgttgtttcccatctggcatgtctggccccggacccgccgcgacacgcggtggtggataacagaccaggacccccggaccatagagaccagggggacgagacagtcccccggtgaggatgtctcgatgtgcagatcaggaacagaatcgccgggcacgacccggcgccggtgccggtgcagtgaacccgtggcccatcgactccacgaacgaacgtctcgccttcctcaactggtgttggcggcaaggctactcagcagccatcgtggaaacgatggacgccgcgggagcctacgaccacctcgacgacgaccagaccagcaaccagcacgactaacgccgctcccagcacgggggaaggcggcgcaccaggtgcgccgccttcccccgtgctggtgtctcgtcggcttactggtgggggtcgtaggggaacccgtaggtgtgttccagggccttgcggagaaggtcgttcatagttgtgtgttcggcgacggccttcatcttcagcgccgtgtgggtggcggtgggtagcttgacgttgaacgcggtgatcggggtcttggccgactggaaggtttcagagacactggctccctggctggccgccatttggtcggcgtaacggctggctttgtcggcggcggtgggtttgcggttcttactcacttcagcatctccttgattcggtcggcaacctgggcgaactctcctagatctgttggccgggttccgatggcttgccgataggcgacccgtcgatgaacctccgcatcgagaacgtctaggccctcaacggcggcggccccctcgtgggcatcacggtattgcctggtccaatcggctttgttgaagagcagggcccggcgtgtgccgtgggccacctggtaggtcattagcgctcggtccatgtccaggtcccccgactcggtcgggatgatgaccacatcggcgatgtcgagggctgcggtgatcgtctgagagtcaccggggggagtgtcgatgatgacaaggtcggcgtcgatgtcacggcagcgcctgacgataggggcattagccgggcggacctcgaagcccagcgggtcaccagtctcgaaagcgtcggtagcccatgacgtcgccgatccttgcgggtcggcgtcaagcaagatgactcggtactgagctgctaaggccgttgccaggtagaccgccgtggtcgttttcccgaccccgcccttggtgttgacaatagaaatactacgcataaatacatcattacacatgtgcatcgatgtatctgtatatatgtgtatcggcgtgttgtgagggaaaacccgggccacatacaacgatcccccagccaccacagccgggggatctctcagtaaagccagcaactcccacagaagttgcacccccacgatacccaccacatccgtgtcaagcccccacaacacccaccagcgccctaggcttcccccatgaacattgacatggacacaccaaccgaaaccgaagccgacaccttcgatgccgtcctcgccgacattaaccacatcaaccgcctcaccgccgacgaaatcgagtgctactccatgggagcctgtctcctcctcctcaaccaccaccccttctccgaactccgccaacactgggccgacaaccccaccgacgaaaccacgtggaccctctacggggccatcatcgccgcctccaccctggacccccacctcagcatcgacgccctagcccgcctctaccagctccccctcgacctcgtgacccaagccctcaccgtctaagcgtcgatgagcgggggagaggtagcgcactatcggctaaggaaccccacaaacaccaaacccttgtcactgggaagggtttggtgttatgctggtctcacaactcaacagaggaggtgagacatgggaaatgagtaccagaagtcgctcaaagtcctgttcaagaaactggaatctgagcaaggagcccgcatcgagacccggcgcaaaggatggatgatctacccgccggatacgtcacgatcagcggtcatgatccacaagactccatcagatcgcagagcatgggccaacatgctttccgaactgagacgctcaggattcaccgtctaactcagtgaagggggctggagctccagccccctttcaaccacctcccccggacaccacacaagaaaaggagaacatgatgtggcacgttcggttgtcgcttcgcgcaatcagcgaggacatgctgttcgacgccctggacaccctcgaccccttatccccggtggccacactgggccccgatgagcacaccggatcattggcggtgttcgtcgaggctgactcactgacagacgccatcgagactgcccgcacagccatcaccaacgcatgcaacaccgccaccataaccggcgtagaagcccgccccgaagacgaatgcttcgcagacgttgaccggcccctgttcccgccagtggtcggatacaccgaaatcgctgaaaccgcaggactctcacgtcagcgcatccgccagctcgcaggcaccgccgggttccccgcacctgtcatcaagaccgcggccggcccgctgtttcccaaagccgccgccgagcagtgggcgcggacacgccagcctaaggcaggccgcccaaaactccagaccaccacctcataacacatcccgtgtgctgaacctgttggccggcccagcacacacccccaggggccgggcccagcccggccccgccccacacccaaccgccccctgttgccgccgtggcgaaacgatcaggcgagccacgaccatcgccaagctgaatggagagcgagcatgagcaccacccctgccgggttcgacttcgacgctctggccgagtgggccgagagcgatgaggcgacccacaccccgcaaaccagccccgtgttccggggcaaggacgccgcgcgcgccagccgcgcattcctcggccgaggacggcccaccctcggctcagaccacgccaccggcgagggccggtcgccccgccgtcaggtacggctcgacgcacgcaccaacgcgcgactcgacgcctacgcagccgcaaccggcaccagcgcctcccagatcatccgcgacgccctcgctgactacctgcccgcctgatcccaccccaatccactcgggcgcctgtcagtgccccctcttctgggtggtggtccggggcgtcaccgccccggaccagttgggtagtcatctcagttgaaataagcgattagtgatacgcccctcatgctttccggtgctgtcatgagcgtctgacaacagcgacacctcggggcagccgacggacagtcctctggctgccgctctgatcccatgcccacggcacgaaggtgtcggacagctcggtctcgtcatgtgcccaaatagcgtcgtgggcctgtcgccaagcttctcgggagccccacacgggcacgaccgggccacaccggtgtcctggtctccagtgtcgcgacactgtcgatttcggtacgtgcaatgcggcggcggtttgacggatcgacatcccagactcccgcgcgtcccagatcgccatgtggagagtatttgcctccagtgcggccgccacacttgcacgctctcgcgcacgctcgaatgcccgcctagtgctctcaaatgtcgtcacgacgcctccccctgaaaatactcagggtgtcccacggtgggacaccctgagtatttcataccccaactcgcctcaccacacgtcgacctcgacaccgatcaagcgacccgtatcagcgacattgccgtaccgctgccccgcctggtcctccagccacgacaacgcatcccgaaacagcccgacgtcttccaaagctaaaccacagtcgtagccgcccttgtcccccaaccgcacaatctggcgctcccggtggcctccctcgtcgacctcgatccacagtttcgcgtgcatccgctggcctcggccctctgacaggatctcggtgagcaggtcggacgcctgccctcgcgcatcctcgttactgcgggggaattggagtatgtgggaggaacggtggtggggcaggtttgtgacctcgtgacgcagcaggttgcgcttgtgcatctgccgttcgagacgctcgggctgacctgttgacggggttgagccgtggcgccgcttcctcggcggggtctcgatccgttgctcttccggcggttcggcgtcaggttgtggtgcgcgatctcccctaggacgggggggctcgccacctatatccgagggacgatgagacggccttccacgcttgctgcgcactttcgcagcccgccccttgcggtcacatcgacgcggcggcggagtgacgtcctcaccgcgcgctaactgcgtcaacgccgccaccaggttggtgcccggtttcagccctttcagcacgaaccgcaccaggcgggggtctcggccgattcgtcgggcaatctcggcttggctccacccttcggctaacagggtgcgcaacaagtcttgggcccggttatcacgctcagtcactggtcgcctcctcggggagtcctgcctgctcggcctgctctgatagagcagcccgttccgatccggtctggggcgctttcaggggaccgggtaggacacctttgacacgcagtcgcccgtccttgccgtcgtcgccgcccccctgggccacagggagcgcccaggggggcacgttgctggtgtagatggcgtcgccgtggatagccagcagggtgcgggagttcatgcgtagtgctcctcccttggttgtggcgttttttccggtggtggggtcggcgatgggtgtgttgagggtgcgtacgcgggacagggcccagatacgggcggcgatttcggggtgccaggtgtcggggtcggggcggcggcgggcggtttggtattggtaggcgatggtgtcaccggttttgttgcggatgacgtcgaagccttcgatgtcggaggggatgtcgtcggcgtcgtagacgacggtggtggtgacggggttggaggccgacatttggccgatggtcacgcgcagcatgtttttcaacgcggaggtgaggattgtcgtggcggtggggttcttgtctgcccagcgtttctcggcgatgtcgatcatgtcttggatggttttgacccatcctcggatgggatcgactttggtcagtttgatccctccaaggaactcgatgggcttagtaacgggcccgtcaggaccgaattcggtgtcccagccttcgtcgatggctagtttcagttcggcggcgtcaacccatgtttcgtgggtggtgccgggcacgttgggccaaaaccatcctgctcgtcctttggtgcgtttgaccggtagtaggccgatgtcgtcccaccaggatggcacggtgaagcggatgtggtagcgggccgggtgccaggggttgttggtgaacaggtcgtgagcttcggtggcgctgagcagagtggcgggtgcggcaccgatctctccggtcactgatccggcatacatgaatcggccgtcgatgtacacgaagttagggatagggccggcaggcacggtggggcgccaaccttgttcgcggtcctcggggttgccgtcgtcgacgcatagttcgtagcggtgctgaggggaggtgtgttggatgagtgcgcccagttcggggtccatcggctccatgcgataggactcgggggtccgtagcttccagaggttgattccttctgctccggggcggtccatgagggcccagtcgcggtcgattctggtggcgatgatgtgggttaattcgcgccatgcccagcgggcctggattggggtgagggtctggccagggaaccattcatcagcgaccagaatgtcgaggttgcggtcggtttctttgtgggtgaaccggcctcgggcgggccgcccggacagcatgaacaggccccgcacgtgccagtcctggttgaactcgcgggtgtgagcgggggtgtcaggcagcagccaggattgaggctggtcggggatgttaccgcagacgatcaggcgtcgggcgttgtggtgtagcgcggttgttagcaggtctgacagggtcgcgtgcttggtgcgccgcccccctagcactggcaggatctcgtttccggccgcgtcgactccccgggcggtgtcgatgtctagccacactgtttcggggtcggggggccgatgtggggcgggacggcgctgctcctggtcgtctctcatggtttcctcctttgggccagggagcggccggcgtcgagggcgttcatgcgctcgcgttgttcttcgcgtagttggtagacccggcctcgtgacactcctgctgcctcggcgatggtggtggccggcaggtggccgatggcggcagcgtcggcaaggacgtcacgccaggccaggtcaatggttcccagctgggctagagcttcgtcgcggcggcgggtggcatcagccactacggaggcccaccctgtggggtggatggtcttgacgacgattcggttgtggcggatggggccccatgcatcagcggggacttgccagccgtgattggcggcggactcggtgatcttgaggcgtcgctcaggtgcgcgggtgcgggcagtgacgtcggtgttccaggtctggactggtacccacgtcccctctcggatgcgttcgatgacgcacgtggtgacctcgtcggtgactgtgatgaccagacgataatctttcccgtctggggtggttcggtagctgggcatgtcaccctcccctatctgtcaaatgagctagacacagtgtctatttaatttgacgcaaagtcaagtgcattagacgcctttgtgtggatccgccccaccagacaaacactcgcgcctcatgacccgccgcgaagccttaacgaaaccaaaccgcttatagaaccgctccgcagcgctccccgagggctttagctccaaggtaagaccttgctcgtcggcccattggcagacttgtgtgaggatctgtgagccgtagccctgatttcggggggcggcatgcacgcacatcagcatgtgaccatgacggcgggtcggcgggctgacataagccccaccacgccagtccggtgtgatctgccaacttcgccatgccgcagcgcgccagcggcgggcctcggtagccaccatgatcaccatgatgatggacagggttacgacgccgactatggtcagctcactccagccaggccagttcccggagatgatccagcctcccagcgcccagatgatcatggcttccacgcccgccgcaatcgggacgagtggggcagtacggatgcggcctgccatgatctcggtgatctcgctgcgatgccatgacaaggagaattcccacgactcttgaatcatgctgatgtgtgaacggcttcctcggcttcgccctgccatagcttctctagacggcgctcggctagctcgtcggggcacggcgacagcctgccgcactcggagcagaccccgaagtggcgcgagagagtgatgaccggctcatcgggagcatgaaccgattcaacgaccgggtgctgctcatcggtgagtcctgaggcgtgaccaatctcggctagacggaggtgaaccataccgtcgtagtagtcgaggccttcaacgtggaacgggtaggagccgtccgcggtgtggcctgcaaccaggtccccgacttccagctgactggcggtcatggttcgcaccggcggcagcggggtccgggtcgcgttgatcctcatggtctctccttgcttctgcgccttcatagatgaaggtgccacagccggggttgatgtgcggaaccacagccactttttcaataaatccatcaccacggtctcaccccttcccaaatgtgtaaaccgtggtttacgctttaaggcatgatcgaacacacaccgacacccgccgcgcctcgcgtggccatcatttcccctagcgggccaccacggttcttggccgagcctcccagcgccgcgctgcgacgccatggtcggacccgcatgaggtcccttcccctatcccctcacattcgcgcctgggtctccaccagcggccgtaaacccaatcccatagccaccgccctgatctggatgcataccggagccctgctgccggtgtgggacaccgccgttttgactgggcctgccctcggcagacgtgtgcgccctctcagcagtgacgccgaactgacctctcggcggtggctcgaccaggttgccgaccatcccggctttctcgacacccttatcgaggccgcccgcatcaccgatggatggcatcacgcgacgcctccgcacattcaggccccttaccggacccttcatcagtgaacggaggtacaacgacatgaccagcgacattgacattgaccggacccacaaacccgacgaccgcgccgaggctcctctttaccggtcctgggccaggagactcgtgcacgtctccgggatgccctggcgtatcgtcgccgccttggccggggtctcccccacctccatgcaccggcttctattcggccgcaacggtcgacccgtggaatggatcggcatcaatgatgctcgcgccctgatggatatcggcatcgatgatctcgcctcagcgtccaccgaccgtatcccggcccgcgaatcccgcgagctgttggtagccctgcacaccctcggctggaccgatgagcatctaagccgctggctgaccagttccgacctcgacctgaccaccgcccccaaagccctctacgtcactcgactatccgccgcccgcatccaggccacctacgacatgctcatcagccagcccgtccgccgctgcggccacccccgcaccccgcccgtctcgtccccgacccctgctgtcctaccccagcccagacccaacaacaccgaaccgttccaacccgccctgttcgagctggccgactgcgcatgaggaccctgcacagacgtgattaaatcgaagggctcaaccagtcatcaggagtcccccgtgcgtttgtaccgcgctgttctcgccgacaccgacatccatatcaccatccgtatctggaacactaccgatcgtgactggacctgggccccccttgacacttgggcccccgacccggccccgaccacaccagcccagttagccgatgaacttcaccgtcacggatggatcacccctgaggtccctactaccctcaccgaggtggctgtcatccccgagaactggcaagctttcgtcgagcatgccctcgctgtccggaaccagcaggctgaccaattgcgcgtcgccgagaacatcctcaccgacatcctcggcgacgccgccgacgccggcctgtctgtcaccgctctcgcccgcacgaccggactatcccgagtcgccgtctacaaacgcagcgccaaaaccatcgactccatgagacacgccacccaggccggcggaatcctcaccccctcctgtctcacccacgctgagagaacagcgctcggcctgcccgacgagtaacccatcgtttacatcctcgactggtccctgcgaccagtgaaccgtgcccccactgcgcccacaatgatcagcacacccacggcgatcatcaggtgggcagcactaactgatcccaacgaaccaagatcaaacaagccctgcccgggagcgtcgagcagtccgtgggcgcgaagccatgctccgagattgccaatgatcttggggatcatcttgttcagtagggctatccctccgatgccgacaacgaggaagacgaggatttcttcgcctgcgttgtttcttcctgtgtttgcggtcatgctgccttctcggggtggatggccagccagctgatgaggttgtcggtgatctgccctgagatttggatcagttcgacttgcccggcctgccatgcccgcgatgtgccggccagcaatgatgctgggacccctactgcgcggccacgggatgcaacctcagccaaggcggcgcgtaactcctcgacggtgctttcgatgtaccaactgccgtcagtactagtctcagcggcatcggtggtagcgataagagcatcggtgaggtcgtcgacggcgcggtccagatcagcgatgtccggtagggctacaggagtcatggtgtgtcttccttctagggtgccttcactggtgaagggtccctagccccggccgatgtgcggtgctgtgggaaaaatgcttcccattggtccgtcttagttaggaaacttggcctaagtgctgtcgagggtcgcgtgttcgtgaccggttcgaagacactgccgccttgtcttcttgcccgtggcgcttccgagatggtcagtgggcgatggctaggccgatgagggcgccgctgcacaggctgggcgcccagggaaggaatcgggtggtgtggtgtcggaaccggtcgattagggcatatagggcgatcagggtgaggccggctatgacagtgacctggatcgtgtcgatgccggttgctgcggccggggctgcccacagtggtgtgatgcgcacatcagagaaaccgaatccgcctcgggagatgagccaggcgatcatccacgctgcgaagatgatggcggtagcgatgacagcacgtatcgccgcagtatggtccgagagggcggtaacgactacagccccgatccatccggcccatccggtccacgctaggactgccggtatccatgtggtgatggcgtcaatgacggcaacccatgccccgatggtggctagcagagcccaggccggccaggtgggtgctggggcccacgctacggcggcgatcatggttgtcgcagatgcagcacagacagcgatcaagcttcggaggctgtaggtgtcgcggtagacgatcttgtcgggattggcggtagctggttctggcaggacgcgggtgagggcaggccatcctgcgaccgcaatagccgcgacggcgaccacccacacaatctgttgcatgggtctcatcatggcccatcatcgatgagtatgggtggccgacatggggtcactcgtagaggggatcgaggggtgtgcgggcgagcatggcttcctgtgtgccgagcatgagggtgccagaatcctcgccagccgattcccagtcgacgatgacctggtcgccgtcaacgctggattcggcgatggtcacccattctccgtcgatattgatgagcaccccagcggccagctccgtggcagcaaccgggtgaggtgggttgtagaagcggtcgatgtcgcaggcagggtcgtcgggttcctcgactaccggaggtggggtcgatctggctgtctcggtgtcgtcggccaggtcggctcccaggatgcgctcccattcggggctggttttcttgcccgctgaggccatcgtctcgtcgatctgttcgtcagtgggccacacaaatttctttttggtccaggtcgtggtgtcgggtcgcagctgatccagcagttccaggtcttgcggggagtgggccttgcgggggtcgggggtgtagaagaattggacttccttgggggctgattcgccggataggtaggtgccgcggccgcgtttgccgaacgggatgcccacgccgacgtgttcggagccgaacatcatgcgggctccgtcagcggacaggggtccggtggcggcacgcccggagaagttgtctcggatttcgccgcccaggaactcggcgtcggggcgttgggtccccagatcgacgtggatacggcaggcggctgccatgcgcagcagggagccgatccagccgaaaacggggcattctccgggcatcccagagaccttgatcgttgaccaccagtttttcgcgttgccgtagaactggcggtactcgtcgatgaggaccagcacgcgggtgaagtcggtctcgcgggccccttcttcttcgatgcggcggtagcggtcctccatgagagaccacagccagtggatgagggcgacctggtcggggacggtagtggcgaccatctcgatgttgggccactcccgcaggcccaggtactcgatgcgtttagggtcgatgacgaagacgcgccagttgcggcgggcaccctcgacggcgtcgccgatcatggacaccgtttttccggtacgggtgcggccggccttgagctggtgggccatgacaccggagatgtcccagaaacaggtgttgccgtcctcgtcgacggcctgggggattttctcccagtctggggagtctgggcccggaagggccacgggtcggggcaccatcgagggcagggggtggcggcgggtgacggtgatggtgtcgtgttccaggtcgaagtcgctgcgccactggccgagcatcatcgccgaggtcaccgcggccaggcgcaattggatgttcgggctggacaaatgggcgccacgttggtggtgaatggtgaagccgtccaggttcccccgatcggttttccaggaatcgacggtgaccgccgacccatagatctgagtggctacttctttagcccgttccttgtctcggtccagatcggttttgtcagggtcaggggtcggggcgcggtattgcaggacgaaccggccggccgggatcgttgcggatgtcaccgtgaactctgccggcgaccaggcagccgcggccgcctgggtgagggccattggcatggtctcgacggcagcctcctcgggggggatggtgagggtcaggcgtcgcggatgcgtcttcttgcgattccatgtgactttctgcctcaccggcaccttcgccgccgactgaatggctaggcgagctgattcggcagtttcggtacgccggccagcgcgtagcgcccgtcgtgcgtgaaggctgcccaccacagcacagcccatacacggcagccacgagcgcggatcagcggctgctgcatagcccccggtagctagtaccgcacctgaccagccggccgcgatacttttccacgccggtttcgcgccaccaggaccagcgttatggacggtctgcttcgaggctgcggagggacgagacagcggagtgctggtcatgccgcggcctccttgtggcaggtcatttcacaggcatgccggccagcgatgatgccgtggcgcaccgccccggtagccgcatagaccgcgcacaactcaaacacggggcacccatcgcaggccgccgggttcatgctttctttgtcccacgatggtgggtcatcggtgcacggcacggccaggccatcctgttgagcggcgctgatagcggtgaataggcgcagctgggcgcgaacttggtctggggtcaggttgacggtgcgggctacacgcatggggttccttcctcttcaccagtgaaggttccctagacccggccgatgtgcggagggatttcagtgttttatcgaggtgaccagccagccggtctgcgctgcgggttcaggtaccagggtcagatgcagcatggtgtggtcggctaggggcacggcgaccaggggcccgtcggcgctcgccccggtgagggtgggagttccggccacggtggtggcgggaatctggttgggtttcgcggccatcagggagcccatgagggtcggggcggcgatctggacgagggcgactttgcgggcggcgggatcgcgggtcaaccagccgcgggtgaaggtgagcgcagccgacgtgatatcggggctcagcggggctggtgtggcggcggggtgggccgttgaggagggccatacgcgcggtagcgctggcgacatcgagcgggtcggagtggggtcaggtgtggggcttagggtccgatgtggcgatggggctaccgccccggtggtgggagacgagactggttgggggtggtgttgagggcgtgggatcagggtgaaggcagccaaactggccgggatgaggatgacggccgcgccgacacttcggagcgaggtcatgtgacgatccttcggtagctcgacgggttgccgtagatcttttccacgttgatgccactatgtgggttgtcggcgctaaccatttggcctccaccgatgtagacggccacgtggtgggcagggcttccccagaggatgaggtcgccgactttgagctggtcacgggccaccgcggtgccgactttctgctggtcggcggcggtgcggggaaggctaatgccgatacgccggtacagcgccgaggtcaggcctgagcagtcgtagccgccatcggctttgccgttgccaccccacacgtaggcgtaggagttgttgtcggcgatcgatttggcccatgccacggcttcggccaggttcttcgacacaccggtctcgttagcggccccggtggcacagttgtttgacaggtgccctgtagtagtggatcccactatttgtttggccatggtttcccatttggcgtaggcatcgggcagggctgagccttgcactgcctgagctgcctgcgtcagcggcatcttctgccagtcagcgatgtcgactaaaccgggaatgtggaatccagggccgccggccgatccggggccagcgtaggtgatggtgtggcctttcaagaacgcctcggtggcgtaggcatcattgttgacctgatccagagtcccgtaccagcccggcaggatgcgctgctgaaaggggccggcgtcaccgtcaccgttagccttcgctgaggtagggtcggccccaagatctgattcttgtttggcggtcataatagcgatgaccgctgcctgggtaggcagtttgtcacgctggaccacagtgacgatggtggtcacgagctgcttctgcctggccgtgaggcctccggcacccacagtgcctccggtgacggtacccacgcaagcatccccgccgacactgccaccgccgacgccagagttgtcgtcgagggccccagagcctactgacagcagcggcacaatgatcacggtcgcgggcaagaccgccaggcccgccagcatcagcactgctgcactcaccacggcgatcacgtgagatgctttcaacgctgcccacctttcgctggtttgagggggcgacgcctgctggtacgcacaggcttggttggtccagtccgccgttggttggcaggcctgggtttgatcggcttgctgggacccggtttgggctccggagtcttaggctccggcttcacatgcctaggcacttcggctgcggtgggaggagtcagcacgtggcgtaccggcttggtgaccttctgggcacccgaaacaaccggcttagacaccaccttggcacctttgaccaccggtgcggctgccttcgcgccggccttcttgactgcacgatgcgctttgatagccccggcctcggcccggccaaccctgctagcagtagcatcgagggctctgtggcccgtcgttgccctcaccggtgcatccttgccctggcgggtctgcttagcgatcgccgacatgccgcctcgcaatgccgaaacatgattgtgggcgtgggcgatcgccgacatgccggccgtcgcgatttttgactggccctgcggggcccctgccccagcacgaatctgctggtaagccgccgacgtgccacgagcacccgccgccattcgcgccaccttcgcagccctagtcactcccgtcgacacgccacccgtggctaccgcagcagcacccgagaccacagtcgaggccatccgccttcgacccaatccggtgagcttgcgaccaccgctgcgcaccatccggcgagcaccccgcccagcccactggccgatcaccgagcccctttcggaacgcttgctgcgcatccaatcgttgatcttgtccctcagccccttctcgcccttgtgacgacgccacatggacatcaagaacccgataatccccacgagctcgagcatgttgacaaccatgaccttgatggcgacatcaccgtcagaggcgaaaaccgacttgatgatgagcatgaccacacccaacatcaccatcgacccgacaatgccgatcatcgagtacaagatcgaacacagatcccggatgagcggatctgtgcggccagggatagcccgcaacacgttgagcatcgacatgaacgccgaccacaccaacttgcacacgtagaaccagcacatgcccgcgaacaccagcaggaccacagccaaacccgccaccgtcgacgagtaaaagaagaaccccaccaaccacgaaccatccgcaatatcgccggcgtaatccgacaacccctcatcacagtcaccaaggtcgtcgcgagcatccgagacgtcatcgtaagggccacccttgagcacctcgatgtacttgtcgtggcacttcgcatcaataatgcggccatagttgagctgctgatgaaccggcgccacgaacgagtccacaatgatcgccgacagcttcggatccttcgagtctcccgtcgaggtcatgtcatcaggaatctcgacctcaccagcggtcgccgtcgcaggatcaaccgaactggtaatcatatgagacagcgccacaccgaactgctgggtgcgagccagcggagtagccaacgtcgtcgtatcgcctgcgaacttcgccaccggggccgccaagaaacccaacgccagcgccgcagcaatcgccgcacgcgcacccgtagcccacgcccctgccgtacgtcccctcatcatcttgacgaccacgcccacaccggcgcacgtcgtcagcaacgtcaacaccgttccaccccagctgtccgctcccagcggggccaaaaccttgtcgtggatgtcgttctgtaaccccaccagcggtccggtaatccacccgacccagctcatcgccacggtccagtccaacaaccggcacaacagggcaatgaccatcaggtacgtgttccaacacatgccaataatggaggcccacatagccttcagcggggccgtaacgccaccatcgtccatcgacatctggtactgaaacaagtgaataccgttggagtcagtcacattgaaaccgtcacccatccagcccagccccgggcccgatgccatggcaggcacactgctcagcgccaccaccagaaccagcgtggtgacgatgatacggaaccgccgctccccggccccggggcggcgtggatgacggctcacgctgtctcctggtcgtgctcgggcgggttggagctggcggcttcgcggtggggagcgtaggcaggtctcagtagtcgaatttcaccgacgttgccgtgttggtcggcgtacaggccacaaccggggcctccggggttgagggcaatcttgaccaggtcagcgaactcttcaggggacgttttagggtccacgcccaggaactcgacacctttctgggcgaggttgatgttgtcgcacagcaacacgatgcgatgctgaatgaggttgcggacggtctcgttggggtaatcggcctcggggtcgtgggagccggtgataagccaggcattggctcggcgggattcgcgcacgtactcgtcaatggcgtgggaggcttccggcgaggacgacatgtgatgaaactcgtcaacaatgaaagcggcgtcgcgggcccggtcgctgaagcacaccaggcgagccagacgggcaatgagcgcgtagagggctcggccaaacaccttgtgcgggggcagctgccgaaacagatgttctgacagcatctcctcggcgttaggaagagcgatgccacaagtgccaatgatgaggatgtcggcactcaggtcggcgggaggaaggtccgggtcaaagatggccgaagctagggaggccgagttctcgatgtcagcgaacacggcaatcctgtcagcgatcgcggtggcctcgggaaggtcgcactcctcggccaggtggcgggccaaacgcccacacgaggtgatctggtggtcacgcatgtaggccggtttgaggaccttggccagcgtggtcccggccacaccgatggtctccaagttaagcaaggtcagcaagaacgactgggcgacctgcccggccatctccgggggaaggatgcgcagcgggtccagcgacagcttggggtgggccgtgtcgacgatctggcgtctaaccccgtcgatagagttgacaaaggttacccactcccgctcatcggagttgtcggtggcaataatgcgcccgccccgggccgcgatggctgcgcacagccgcttctcggccatcgtcttgcccgcgcccatcttgccgatgaacgccaccgtgttgcctttctgatcctgagtgtcggcatgcaaatcgaggtaggccagcgactgcaacggggagcctgtgaccacagccaacgggatgccggtggtacggcccaaccggtactcaaccaccgggatagatgcggcgaacgcggaggcgtgagtcagctgacggtaggcgttaagcgaggaggagaaccggccaccgggccgggcagcccacagcaagtcctcctgacggcccacgggacgcacccacgtgaagtcaccgaacaccggatcattgacgaacgctcctgccagctcctcggtggtctcaaagtcaggagacgagacagtgacggtgataagagcctggatctcgacctcacgctcatcggcggccagctcggcgttgtagtcgacgagcacctgagcggaatgctcgatagacatgagggtcgaagcctgggtcatctcaccgccctcgacgttgtcgagctgatcgaccgtttgacggatagcccgctgatttttctgcatcgcagcgtgacgggtccgggccactccccggatcgccacgtcgaccggcacgccagtgtcgtcgatacggcccaaaaactcgtaggcaggccacgtcatcgtgcggggcaccttacccagcacgatatgggactggtagacggtctcgccggtgtcggcaccgattttgaggacgcgacgattccacgcctgagccgtccgggacatgtgggagactcgcttgtcatcctcgctggccagcaggtcggaggtcgcggcctcatcgatgaccaactcccccagcgcggcacgtccaacctcatccatgagggcgattccctcgccaggggcatcatcacgcaaccgggtcgggtggcctgacaggtgctgccgaagccagatctgttcatcaacggtgaccgggcggggttggaacgctgccggcagtttcgcaccgaattccttagcctgacggagggcccactcccgtgcgacacggccaggagcggtggtcatgagtccggcggcttcggtgattttgttgagggcggcggtccaggcggtgtgggtgcgtattttctggtcttggttgagtttgatcgataggaaccacacgcgccggcctagggattcggtttcaggctggtcgagggtggcgtcgatttcgtcggcccacacggggtgttcgctcaggtcaaggccggcggccatggcgtcgacgatttctgtggggtcggtccaggtgagggctccgttgaggcagaactcgtggcgggcgaggtggcggaataggtcgcggtgagctgcggcgacggtgagggaggcgttctcggtgacgggtcgctggaggggagtgagtcgccaggtggcccagatgtcgccggtggaggtccagacgaggttgtcgaagaggaaccgttcggggatggacatcatgactccttttcggcggcggcgaggaaggcttccaggccgctgcggcgtacagcgttcccggtagggtgcaggggttttctgggggtgggttcagatttcgaatgtgggaggggtttgtggatggtatcggtgtcctcggcgagggcttggtcgagcaggcaggtggccggggtgtcggtagtctcgtccagctcgtcgatcatctcgtcgaggaggttgggggtggtggatgcggcccacacaggtgggatagccggtgcgggtcgaggcggtgtggggaggcggagtttggtttggccccagaggggtgtgtgggtcatgccggcgggagtggtggcggcgtgagtggtcacacgccaccagcccgccagccatgccagaggtgaccaggtccagtcgatgcgaccaacaacgaactgggcgagccatccggcagccagcgcgaaaatgaaaccgcggatggtcatgcccccgtcgataattttgaagcccatcatcgcggcaggaactacgccgccgacagcgagcacctgatagatcgtgtagggcccgccaggaatccttcgacctgaggaatccttaccgataatgcggggctgctggcgggcctcggtgtgccactgaatatccacgatcagctcttgcccaatccttgcatcatctgtccgaagaattggatcgcgttaccttcgataagccagtgcgccataccacacacaaacagcatggagacgatcttggccaggctcataccgtgtttgaagatgttgaacgcggcaatgcccacgactacgaccatggcgacggctcgcaccgcggactgaccttcacctgccttgatcgtcgtccattcgaagaggttctgcgattccagtggcagcaggaggaattgatcgagcatgagaggttttcctttcaggaagccgaccgggatggtcccggtactgacgggctggacggcacgggtgtgccggccaggggcagaaggcgtgacggctggtcgagggaggcaacctcccagcggcctgcacggctgacaagagtcaggccgtactggctggtccgggtagtggattcgtcgccggtgggctggcatgtcacggtcaccgtgacggtaatcttctgccggtctgagggggtggggacctcgtcaggtcccaccacatgagtggttttcactgatacgcatggggctgggtcaaccgcggtgaaagcggtccctgggctgctccatctcgacaggtcggtattcccggtcagcatggcggttaggaagcctgaagaggcttgggcggccgggtcggcggtgcgaagctccgaggtgtctagggtgggggctgaggccggtgcaggcatggggatctgtccaggtagacgcaaggcgatggccttgccgttggaatcgacgtggaccgggactgaccaggcctgtccgctcccggattggccgcccatggtgaccacggtgacctgccacaggctcggatcagacggggagacggtagcgtcataggcggtgacggtggtgggcgctggaccaggtttgcggggcatgcgcgcatcggtgatcttggccgccaccaggtgttccagattgtccaagtgcccggagtcagaggtcagcagaagagtagtcacctcgaccgcggtggctgaggcgatctgagctgtgcggcggtcgtgatgggtggtgtcagtgctgacagctgtggtgggagcggatgccggggccgccgaacggaccagggcaacggggccgcatgcgactccgcacagcacgagggcaaggaagcctttggtggccatcgatccggtgtggggccagggacccacgttttcctgtgcctgttttttcttggccgaccaacggcgggacttagggttagcggatgcaccttctggggtgcatgctcgtctcaatcgcatcggtcacctccttcactggatgaagggaccaaggaggtgaccgatgtgcggtcgaacccgaaaaagttcaaacttttttctgggtctgcctcaagtacgacagaaggcctgcatcatcacggcgttgtggtagcagatcttgcgtcagcgcaaatgtatgggcctcgtaggagtagctcttaggtgggtcccggtggttgcggccagggcgagggtgtcgggtatgtgtagcgctagtcccgggttcgtggtgtgagggcttttgggtgctgacatgcactacccattgctagacgctgtgctgtggcatggctggacgtctagcagacgggttgacatcacctttaggctggcagcgcccgaggccgcaggccagaggaagcggacagcctaacgattgacgtccccctctcctttccccaacctcctggagtcgatgacccggagatagatcgatgtctatgcatctgtgcatcgatatacctatgtatctgtctgtggtttttagaggccactcccgtcgccccgggccgctcagcgataccgcaaggctgtgtgggatcagctgtgccgctctcccgacatggcagacagcaccctgcggactgactgacactgtggcgccgcggaggtctcccgcggcgtccgctggggcgagtgcatgcctgtcagctcatcttttatcgtcatgtttccccttgtcagagcggtctttgcaaagtgacaacgtcaggggtcttgacgacaacatcctcgacaacatcgtggcggtggtctgtgtctcgtcagttcaacgcaccggggaaagaggcaccgctgatgttcaccaccacaaacaccctcaacgatgagtgggaccagattcgcgccactgagatcgattggcatgactgccctgaactgatgcactgcatcgacgttgatgatgtgttggccgtcattcctgccgcccccgacgccatcttgggctacctcattgcccgggctcagggcggtgacgagttggccacccgcactatcatccaagcgttcacaggcaagcttatcttgatggccacggccaccaaggtgcgccgtactaatgacggcttcaatgatctgttggcgggtctgtgggagacgatcatcacgtacccgttggatcgtcgccctgacaagatcgctgcgaacctcatcctcgacacgttgcaccgggttacccggttctggcgtgctgattcccccgatgaggaagaggctcacggtctggtgcccttccctgacactctcatcgccccagagcccgatgaggacgtcacagctagccaggccatcgccttggcggtggaccgcaactggattaccgaggacctagcccgcctcatgagccatatttactgtgacggtatgactggagctgatgcgggtcgtctgcatggctgcgctccagctacggtgcgatctcgatgccgtgatggtcgggccgtgttgcagcgcaacgccgagacgatcctggccgtctgctgatccctctcttgctcatgcctgccgccaccctacccctacctgctagaaacatagccatgtctccagcaacgcatctagcgtatcttctagcaattgatctagctatgtatatagctgatgatctagctaccatatagctgtgtctccagcccaaaggacagcccaatggcatgcttgccacaggaaggtgcgtcagcctcccgaaatactggaaggagctttaccgtgcaggacatgacctcgaccagaccgattgaccgatccccgttcgaccgcactattgcggtgtggaaccacaaaggcggcacgttcaagacgtccgtcgtggcgaacctgggatacctgttcgccgccggtggcaacaaggtgctgctggtcgacatggacccccaggccaatctcgacattgacttcggtatacccgcgggagaacgtgaacggggcatgggattagccgaggcgctacgtgaggggacggcccttcccccaccgcagcatctcagcgaaaaccttcacctggtcagtggcggcgctgctctcaacgagttcaccgaccccgcatccttagcggccatcctcgaccgagtcaccaccgcacgctacgacctgctagctcaggcccttgccccgctggcctgggactatgacctcatcttcatcgactccgggccggcacaaactctgctgtcccagaccatccttggagtagcccgctggctggtagtgcccacccgcaccgataacgcctcaatcactggcctcgtcgacgttcaagacgccatcgacggggttgcctcctgcaaccctgaccttcaactactcggcgttgtcctagccggcgtgggggcccgggccacccggatcgccgcagataaacgccacgccatcgacacagtgctgggggcaggaaccgttttcgatgcggtcatccactactccgagaaggtgtccgtgctcgcacgccagcagggcaagaccgtcgccgagctagccaacgagtaccacaacacccagcccgcctacacctacctagctaaaggccagaacatccccaacgtcgccaaggcagccgtcagcatcgaaactgattatctgaggctggccaccgagatcagcgaccgcatgttcaccagcgacgagcaggagcctgatcatgactgacgatccgactccatccgagcagaacaaccccaccagcgacgacatgcgcttggccgaagaatgggccaacagcggccgcacagtgggagccctgccgcgccgtcaacggcctcagcgacccacaccccgcaccaccagccaaccccctgtctcgtcccctgacacaccggctccgaaaaaaccccagaagacccccaccagccagcctggccgtcaacggatcaacgtcaccgtctacgttgtgcccgaactccatgagcggctgcgctcccgatcggcggcgacgggggtgacggtctcggatctggtggtgcatgcgttagcgttcgtcgccgatcatgcgggggaggcgatcgccgatgatctgcgcgtcgagacgggccccggcatgggggccggactgttcgatgtgaccccatcgcggccagtaggggtcgccaaaacccagctgggggtgcgaatgacccgccacaacaaggatggtttggaccagctgacccggacgagtggagcccgtgaccgcagccatcttgtgtctgtcgccgtgcgcgactatctcgactccaaccctgtaaagaaaggccgacatgtccgctgatgaaactcctgccgcccggggtgggcgacgctttgatacgaccgtcaagcccaggcgccgtcccatgcttatcgccgccggtatcgccatcgtgatagtcggcgcgctcctcatcactcagttgattcgctccgcccaaaccgagcatcgcgtgctggaagtccacgccgatgtggctcgcggcgaggtcatccgtgacaccgatctggtatcggtgactgtcggccaggtcgacaacgtatcaacggttcccgcagaccagctcgactcactggtcggcaaacgcgccacggcagatcttcgagccggatcactcttgcctgctggggctatcggcccggccgatgtcgttccagctccgggcaagtcgctggtcgggctgaagctagccgccggacagatccctatcggagaccttgctgtcgggacgaagctgcgcctgatccagacgtcggcacccagcggctcatccaccaccactgatagcagcaccaataccgatggccagtcgtgggaggcaacaatggcgaccggcaccaaaaagaccgagcaggtcaccttgatcaacgccgaagtgaactcgcgtgatgccgcccggatcgcccaactgacgtcacagggccgtatcgccgtagtgaaggatccgatccgatgagcaccattgttttgaccagcgtctcgggagccccaggggtcaccacgacagcgatcgggctgggcagggtctggccacaatcgagcctggtcgtcgaggatgacacccaccacgccatgctggcgggctacctgcgagcctcccagcacgccgaaccgaacctggccgcagtggcgaacctgacgtcgactccaaccaacgcccaaacggtgtgggaatctatcgccagacccctacccaccgatgacccagtcggcgggctgcgacgcaaaggcatcctcggcccgcccacgccgtggtctcgcgcaggcatcgacccccgatggggattcatgctggccctgtggcggcaactggaggaggctggcatcgacaccatcatcgatttgggtcgcctagccacaccactgacctcaacgccacacctgatcgccgcacccatcgtcgaggacgccgacatgatcttggtgatgatcgaggcgacactacgcgacatcgccggcgcccgcacgatggtggagggactcgctgagcagatgaacttggccggcgcctaccgacgtctcggactgcttgtccaccgtggcggccaggcccgcggcgtcgccgagttcaatgacaaggagatcacctcggcgttacgactcccggtcatcggggccattacccacgacccggccggagccgcccaactctccgacggggtcgggcagcggttcgacaagtcacgtctggcccactcactatcaaaggtggcggccagcctcgtcgatgccataaacaagcgccacgctgatgacgaggaggatctgtgatggccagttttgctgataatccgttcgacaagctgcgctcccaggatgcggcaagagcttcggtagagcaggagcctgatgccggattagcgtcggagttgttttcgacgtcgtcggggtgggccagctctcagcaggtgtcccaagctcaaccggtgatgacccgttcggagaacgtggattggccggttgtcgcggagctagccagtacggccactgatgaggttgaggctgagatcagcaggtggagttccactcatgatggggtggcgacgctagacattcgccaggccatcgcggagcctgctattgcctcggcggtgtctacgtatgctgaccgacgccagatcgatgtaggggagacctggcctgatctggtacgccagcgataccgcaaggctgtgtgggatcagctgttcgggatggggcggttgcagccgctgttcgaaatcagcgacgccgaaaacatcatcgtggtgggtaaccatgaggtggttgtcgaccacaacgatggttcccggtcaacgctgccgccggtggccgactcggatgccgagctggaatcccagatcgcccggatggcgaggaatgcgaccccgcggcgagccttcgacgccgatcacaccgatgtgacgatcatgcaccaacagaagttccgtatccacgcgatctcttcggaggtgtcgttgcaaccgtcggtggtcatccgccaacatttgctcacccaaatcagtctgggggacctgtcccagcggggaatgatgccggtggaagtggcccggttcctcgatgaggcggtacaagctggcaagtccatcgtggtcgccggcgagcagggggcaggcaagacgacgtttttacgagcgttgatccacgccattccgatgcgggagcggttcgccacgttggagaccgatcaggagttgttcgctcatctgatgcctggccgggaaaacacgctggtgctgttcgcccgtgatggtaacggcgaggtggatccggcgactggcacccagcacggcgctatcgagatcgcccagctgattcctcctagcctgcgccaggcgttaactagggtcattgtcggtgaggtgcgcggcaatgaggcttcggccatgttccaggctatgcagtcggggactggcactatgagcagtatccactccccgcgagcctctgaggtgccgtcccggctagcccaaatgatttcgatgggcccggtctacgatctggatcaggccatgctgcagatcggtcattcgatcgactacatcgtctttgttcgtaagcgtgacctgcctgatgggagccggctgcgatttgttgagcagatccgttcagtctcgcctggtgattcgacaactcccagtcttggcgaggtgtacacggctgactcgtggacaggccagccgcttacgccactcatgcctggttcggccgccgacgagctgtctcacttcgcccgagacctggattactgcgaggcccactgatgaacaccccagcgctgatagcagccctcatcgtgaccggcctgggactgatcatggccatcaacggcctgatcccggccacacccaaacccggcaccacgctgacgctgtcacagcggtgggcccgggccacccaccgcccagccggggcagccgggcgccgacgcgacctgcgctgggcgctggccggtctcgtcggcatcgtgctgttcgtcctcacaggttgggtcgctgccctggccctgggccccatcaccgtgctgctagcacccaccctgctgggggcggccccaccgaccgatattccgctgatggaggcgttggatcggtgggtgcgtcaggtcgctgcggtgctgccacaggggcgagacatcatcacagcggtgcggatctcacggccgcgggccccgaagctgatcgccgggtcggtggatcaactggtgtcccggttggatggccgtatggagccgagggaggcgttccagcggatggccgacgagctggactcggctgaggctgacgcggtgctggcctcactagcgttggcggccactcatccgcggggagcctcgacaacgttgaaggctattgctggcaacatccaacagcggttgaaagtgctgcggcaggtggaggctgagcggtctaagccgcgtaactcggtgcgccagatcacactggtcacgatcgccatgttcggggggctgctcgtgatcggacggtcgtttctggccccgctggcgacccctcccggtcaggtgctggtggccgtggcggtggtcatgtatgtcatcgcactagtgcagatgtatcggatggcgcggccccgtcgacgcacccggatcatggtcaggaggacccgatgaccattactgctttgtccctgctggccggggccatcgtcactgtcggactgctggtcatcgtggcagcgttccgcccagccccaggccctgacctggttgccgctctggaggtggtgtcgggtcgcacctcaagcgtcgccgagaacgtcgaccagacccggatcgggcggataggtcgcagcgtgacccgcacattccatgtgtcggtgtccccggccatgcgggccgcactgcgactccaaggcacgacccccgaagcgttctacggtcgacgcctgatctgcgcactgatcggggcgatcctgccgtggctgctcaacgcggtagccatcgcggtaggggcgaattccccgaacctactcatcccttcggcgctgtgtgtggcgttagctggcgctggatggatgctgccggcagcacggttgaaggctgcggccgggcccaccaacgatgactcgttcgaagcgctgctggtattcatcgatttggtcgtgctggaacggctcgccaacgaaacctccgtcgacgcgctcaccaatgcggcaaacatgtcggactcgccgctattcgtccagatccgtcaagtcctcaaccgtgcctccctggaaaacgtcgacccgtggaacgggctagaccggctagccgaggacatcaaactgcccgaactcaccgatgtggtctccatcgcccgcctccaaaacgaaggcgcctccctcgtcgacagtttccgggcccgagtcgccgagttgcgcgacgcctacctgctgcgcctgcaacaagaatccaccgccatcacccagcggcttggtttgtggacgatcctgcaagccggctcggtcatgctcatcttgctgggggcggccgccctcacactcatcacagccggataaaccgacatcgtgtcgagcccccatatgggccgcgaacctttcataatgatgcctgtaagcaccacaatattgatcacaacctaacaatgaaggagaaatcatgatgaccgatctcgacctggctaaaaaacagatcacctggctggcggcgcacctgcgttcccggatcgttgacgagcgtggcggtggtagctctaccgtggagacactgatttggattggtgtcatcgtggcgctggtgattggtgtgggcgtcggggtgacggcctatatcaggtcgaagatgcctcactgacgacgatgcgtagcaggttcacaactgatcagcgtggcggtggtagcgcgtcggtggggatgctcctgctgatgcctgcgatcatgctgctggcattcggtgggatcgaggtcgggatgtggtgtcacgcccatcagtcgaccatcgcggcagcccaaagcgccgcggaggctcagcgtgtcgtccatccggtcccggggtcggcacaggaggcagcctcgcagatcactagtcatggcggggtgcgtgatacccggatatcggtcagtgatgacggggccactgtcacggtgaccgtctcaggtcgggccccatcgatgctggggctgcatcttccggctgtgtcgtcgacagcgtcgatgcccaaggagcgcctgtcgtgaggcgaagtgagcgcggtggagcaagtctaagcgtcgaggtgctcatgtgggcgccgatcgcgctggtgatcatcgggtttgttgtgggtatcggccggatgtcgatggcccaggacgcggtgaatggcgcggcgggtgctgcggcccgtgcggcgtcgttggagcgtgatggacagtcggctcagtctgcggcgcagcaggcggcgtcggcgaacctgtcgtccggtgggctggcatgtgcgccgaatgtgagcgtggacacgtcagtgtttgcccgtcctgccgggcaggcgggcacggttcacgccacggtcacgtgtgtcacctcgctgggtcttgggttcgggtcccggacggtgcacgcaacggggtctgccccggtagacacctatcgggaaaggagaggctgagatggcccgtgatgagcgtggtggcggctcggtgtcggtgtggatgctgctcatggtgccggtgattctggtcatggccggcctggtttttgacgggtcccgtcagatatcggcaacccaggcggcccaggacgcggcggttgcggcatcccgcgccggaactgatgcggcagcgacaccgcagcttgctggccacgacggggcggccgtagcagtccaagcggcccgccaggcactatctgctgccggggtggacgggtcggtgcaggaggacgggtcgaccatcacggtgaccacgtctcaaagtcggccgacagtgttcctgtcagcgatcggaatcagccaggtaagggggcatgggcaggcccatgctcagcttgtgggaccgggagaacgcccatgacagtgatacgtcgtatcggtgccctggtggcactgctggtggtgatagccggcttaccggccgggctcgtggccgcaggcgccccgctagtaccggcgggcctgtcatgggcccatgtgcggcatctgttggttaccccagacatgaccgggtcggtgctggtgtggctggtcagcctcatcggctggatcgggtgggcatggttcgccctggccgtcgcagccgaagcggtgacgatgctctcagggcagcggctgcactggcacatgcccggccctcgcctagtgcgccgagtggcagccggtcttctcattgccgcgttcgcggccgcaccggcagccacaagcacggctcacgccgccgaggccacccatgtggcggtcgccgcccaggctggaccggcacacgcggcccccgcccaggacagccccgccaccacctctcaggccccggacgcctccaccactccgaaaatctggaagacctacacggtgcgggccaacgactacctgtggaagatcgccgagcactactacggtgacggcgcccaattccgtcgtatcgccgaagcctccggtatcgacccgcactcagagctgaaagtcggccagaaactgatcctgccggttcctaagaacacggccgcagttcattcggttaaagcgggggagtatctgtgggagatcgccgagcactactacggtgacggcgcccagtaccataaaatcgctgaagcttccggtatcgatgcccattccgatctagccgtgggccaaaaactcgtcatccccgggcccctccgccacgacgccacgccgccacaccactcggcgtcgacccacgcgaaggccgccaccccagcccacacgcccacgcgacggcctgcgcctgcccacaaagccacgcccgccccgacgccggccaccccaactactccgacccacacggtgaccccagctccggcctctgccggccactcgaccagcgacgagcacccactccatacggataccgccgcagcggacaaggccagcaacgaggacgccctgtccccaatccaggtgggtttgaccgccagcgtcggactggtgctggtcgccggactgatcaccaccctcaaccgccgctaccggacccggttcacccgccagccccgcggcaaggccatgaccctgcccagcccggacgcccaaactgccgagatagcgctgcgcagcaccggggcgactgacactctgacgatcacctgcctcgaccaggcgctccgcgcgatcggcgcatggtgccaccactgcgggcacccgctgccacccctgctggccgcccgcgtcgatgacgaccggatcgacctgctgctatcccaggcggcccccgaccatcccgaggcggtggagctggccgccgacggatcggtgtggaccctcaccgccgaccggatcgacgacctgctagcccacaccgatgacaaccaagcggctccgtggccgtctctggtcacactgggacgcgacgacgacggcgcccacatcctcatcgacttggaagccgcaggaaccttgcacctgatcgccgatgacgaccaggtcgacgcggccctcgccgcgatcgccgtggaactggccacctgcgactggtccgacgaagtcaacgtcaccctcgtcggccaggtgtgcccgggcctagaagacgcactagagtctccgaccctgacccgcgccaccgacgtggacaccctcctcaccaccctagaagctcgcgccgacgaccagcgacacatcctcaccgaaggaaaccccctcgccgctcaccgagccgaccccgccatctccgacgggttcgacgccgaagtgatcctcctcgacaccgaactcaccgaagaccaccgcaaccgcctcgccagcctcgtcgaggctcttccccgcgtgtcagtcgccgcggtaacaaccagccccaccagctcagacgaatggtccctcaccctgacaggtgacccactagccgccgacctggctcccctcggctggcacatccacccccaaaccctctcccccgacctgtacaaccgcatggttgaactactcgccaactccgccgccgcagactacgaacccgccagctggtggaaccacgacgccgacgacgagcccaccaccgaccccaccaacgaggaagaatctaccccgagcaggcgagcccgcccgaatatccgcctgaccaccctgaccggcggcatcgacctatccagcgtcgacatcgacgacatcatcgacgaagccaaccaggccctcgacaacaccgaccagaccaccaaccaggttcgcgtcgacaccaaccaacccggcaccgacacccccttccccatcgacgacatcgacccgctgagctccgaccatccggtcctatccatcatcggccaccccgacatcacaggtgccactggaaccgttgggcgctccccgtggcgctgccaacagttggctctctacatcgctgagcatccgggagcatcgggggcgactatcgccgatgatttggggctatcagcgtcgacggtgcgctcgattgctacacacctgcgacactggttgggagccgacgatgccggggtggcttacatgccggcggcgacgcgcggctaccggctcgatgagcggatcgtcacagatgtggacctgatcgacgctgctgtggccggggcaggcattaacaccgctgagacggccacactggtggccattttgaagctgggccgcggacgggtcttcgctggggttcctgactcggaacttcgccagttcagggcgtcgatgtaccacgtggaggcccgcatcgtcgatgctgccctccaagtgacagaccgggccctggaagccggggacctggggctggcccggtgggcgttgacccaagggctactggtgtctcccgatcatgaggacctggtcaccggttgtctgcgcaccgagtaccaggccggcaacatggacaaggtttccgagctggtcgatcacctgtcggctaccgcacgacgcctgggcgtcgatttgagcgccgacaccacccggatcatcgactccgttattacccatacacgcaggagagcatcatgatctcccgttgcactcatctccgtcaggaaggttcctatgcaccattatcgtcttggtatcggcactgccctcatcgcggtagcagccgtggccggctgtcgctcaacagactcgacacccacaccacctccatcccgtgtctccgccacgacatcagcgagctctaccctcagcccgaccccgtcagccggagcgatcagtgccgccgacgccgaaaacatctaccgcaccgtccaagctaacaagatcgcactgtacaaaaagggtggcctggcacccggggaacccgcacccgcgacactgtcgaactacgccaccggccaggctctcaccgactacatgacctacatgcaccaaatctcggggcagggcatcaaaatcacatcggggaactcctcggttaccgcggtgcgggagaaaaagggtgataccacctaccccgaagccgctatcgccttggaatcgtgcgaagacgacagttcgattcgtaccgtggaccgccacggaaaggctgatcatggacgcattttccatgtcgacagttggtatgcccgcgataagaagggcaccgtcaagatgatcgcattcaactcaacggaggtccccacatgcgacgtcaagtaatcaccgcggccgtactgggcctggccctggccaccacctccctgcccgccctcgcaggcggcggcgacttgaacggcagtgtggacacggatggccctggttacgatgtgcgtactgaggtggattaccatcacaccaccggtggtgcgggtgggggtgccacgacagggggccatcatgggggcaaaacccacggtgatggtgaaggtgaggctattgatgggcgttctcgcgccgatgccgaagccgaactagacgaagccgaccgtaaccaggacttgacttgtgatgtcttgctcccaccggcgccgaataccccagccggccaggaatgggcgaaagaatgcaatcccccagcgaaaaaggggcagaaggccaccccgaagcccgatccggtgttagtgggccgtcacgcggccttgcagctcacccttcccggcgcggcccctcgtatcgatccgtcccccgaccttaaccggtggcactcggcagcagtgggccaggctttgtggttgtcggttgatgaccccaccgccactacgcagaaatcgatcacgtttatgggccaggttgtctcgttgagcgccaaacgtaacggtttgtcgtttgatatgggtgatggtcatgtcgtgcactgtgatgccacgacgacatggacggagtcggtggaaccggggaccccctcacccacgtgcggttacacctaccaacaggcagccccggccgggggctataaggtcaccgcaatcacgtcgtgggatgtgacctggtcggtactaggacacacgggcacagtccacatccagaaggccggtgggcagatgcttccggtcggcgagttggagtctgttgttacacggcgatgactcctgatagtccgacgctgtatgaggtgttggaggtctgccctgatgccactgatgaccagatcaagacggcgtggcgtcgtgccgcgaaagtgactcaccctgatgccggtggcactagtgaggcgttcgcggccgcccggcacgcctgggaggtgttgtctgatcctgcccggcgcaccgcctacgatgccgacctggcgggggaggatgatcccgacgacgaagccgccgacgccttcgacgttgatccgttcacggcatggccctggtgcgccccgtacatcgacgccacccccaccctgcgccacacctgccccggcacgatcaaggccatcgtggtcgccatcgccgggatcatggccggaatcggctggcaggaagcgctggaggccgtggtgtcgccgccggggtttctgggggcgtgctggatagtctcagtgccggttgtcgtcgcagtgacatggctggcagcggtgacgggtctggcatttttcaccatggcggccatctggtgtggatggtcgttgataggtggtgcgttgctgtggggccagacgtcggtggtgtggctgattgctgcggggtggctggcatggctggtgtgtgcgtgggtgtggtggaaccgtcatctggcgtggagtagcgagctgatgcaggaaggcaatttgtacggccttcccgaggaagacccgttgtggggcgaggccgtggagctggtagcgggacttatcccctctgtgcgggcgatgtggtcgcacgatgggcagaccgtgactgtgtcggctggccggcgggtggcgacactggggatgccgatgcggggatggcgtggtatcgagatgcgcggctggaatccagtgggcgccgatacgtggtggatcgttgaccaggtgggtagttggctggtcgatcaggatgagccgatggtcgttgacgggagggtgctccatgaggcgtgggaacggtcgcagatggtccgttcaggccgctgagaaaaaatttaggtccgccctgcatatcggggcctcttcataccttcaaaagtgtgaagggaatacagactcttcgactggaattaaaatggtatatggcgtgtccggtccgcacccagctccacgatgcgccataggggagccataacttcaccaatcgtatacgatatgagtgtaggtgagcatctaccggtggccgacagtcagatacaagcatgggcggatgaagcagaagctgattatgacctcagcatgcttccgccgtctcgacgtggtcgcccaccggtggggaggggcccgggcactgtggtccctgtgcgcttcgacgcggacactctcaaggcgttgtcacaacgagcccctgacgaaggattgaccacgcgttctgacgcgatccgtgctgcggttaaccagtggctcggcctcggctcgtaaacattatcggcgcgacaaccttactgatgacgctgttggttacgccgtagagcacatcttgtactcgtgggcgctcgacgatgctgatgatcctcgttgctggatgatgattcggtgttgacccagctggcctcctcatggagctagttatcctcatctacgacgatggctatgaactcctgatccacgccatgaaagcccgccctcagtatctgagctacttcgctatctagtgttgttgggcgagttgaactcaacttttctccgacgcaagcgccaataaaaggctgatccatccgaataccaccaaacacggctgacgtgtggaacagattcagcaagacagggtgcggtgctcgggtgcgttggggagatgaggatattcaggcttctggcgggatgtggcttgatgacgtccaggatcaccggttcggttttgaaggtgccttgcattaatgtcgacagggtcgtacgactcgaggactggatcgaccagcccgttgcgtctgaagaacttcatccaccccggtggagctccggccgcgtactggtgcaacggatcattagcatgggtagcgtgagtatgccttcgatcgtggtcagtgctgtcatcatccaaggttctgacgggcggcttctcactgtccgtaaacgaggcaccgaggcattcatgctgccaggcggcaagccggagcccggcgaggactcgcgtcaggcagtagtccgggaagtgcacgaagagctcggcgtcgcattgtcttccgacgacctgcgtcgggtgggagtgttcaccacgcgagcagcgaatgaggccggccatcaggtggtggcgacgattttcacccacaccccggttgcggtgagtgagccagctgctgagatcgagcagattcgttggctcgattggagtgtcgacgccctgcctgatgacctggccccgctgctggtcgaggcagtcatcccgtggctgcggcgccgtatccggtcggtcgctgtattcacgggtgcgaaggatggaaccgaccctcattatcgtgtcgaagcaaccgccttgggccggggtctcgcacacgccgggatcaccctggtttatggcggcgggaaggtcgggatgatgggtgctgttgctgatgcggccctcgctgctggcggcgctgtgatcggagtgatgccgcagcacttggtggacggggagatcgcccatcctagtctgactcacctcgaggtagtgcggactatgcatgagcgcaagcagcggatgagtgacctagctgacgcgttcgtcgcgctacccggcggcggcggaacccttgacgagctattcgaagcatggacatggcagcagctcggtgtacacagcaaacccgtggccttgtacgactcgaccttctgggcaccgctgaccgcgctactcaaccacatgaccatcgaaggcttcatccgccctgaggaccgcgcctcgctcgtgatcgccgataccatacatcagctgatggccgatcttgagggatggaccccaccaccaccgaagtggcgctcgtgacatagaacaaatgattctgactatggctcattgacatctgcgcagcggctactagctccattgacttcaaatcgggccttggccgaggctcggttcaggtggcccggaatggatccccacaaattgaatgctcatgatcgaggtgatgaacgcccactctattgtcctacgcccggttgtatccagcgagcaattgaaggtgctgcgcctgttggtcgaccaggccgagccactcatcatcgacgggcgcatcttgtacgaggcatgggacaggctgcaaacagcccgtccggcccgctagaaaaaatttttagggcccgctccgcacatcccggtctgtacccacaccttcaagggtgtgaagggaataaaaacacacctaacaacaactttgtttcatgatttggcgcgtcggaccctttctgaccgatgtcgtgcgcaacaatggacccatgagttcgcctaccaagatgcgccggaaggggacacgcggcccgaaacctcgcagcgacgaggagctaacggctgtgatgttccgcgtcccagagtcagtggccgaacgtgtcagcgatatggcttgggagctgaggctctcccgatccgatcttgtgggctggtcggccctctatgcactcaactcgatgctcagacagcgcggagaggacacgattccggttccggagtatctggacaaggccgtgctcgctgcgttgtatcccgatgggttgcccttcgacgaggacaccgaggagccgggcgagaatgccgggcaggaggagcttgcgatgactggatgagcccgaacatggttgaaggccgccccgccaggcgaccttcaatcaatggtttcggttgtcagctgcagccacccttgccaggtgctgtggttcttctccagaggaggagggaccgccatgaagacccagaagacctagaagatccaaaagaaccggtttcggctgttgcttcggtcttcatggtagctgatagcccgcgtcgttggaagtgcgacgacgccgatcctcatcggtatcgcaccctgttccagggtcgagaatgtgaaggattaacaaccatgacaagtcatgagacgccctcccggagggcgtcatgaccgctttaattcaagctctgccgacagctgaactgtctgctggacctggtgctagcagtccagctagaccctctgctgagggcttcatcaagattcaccacgacctcattagtgcgggcgtgtcgggtaacgcgatggcgttgttcgtcgcgttacgtaaccagcccggctgtgatcagtggacccgtcactcgtacctgcggctagcccaatggtgcggctgggatgggctgtcggaggcggcgggctgtaagcgggtgcagcgggccgctgctgagctcgctagcggtggttggctggaatcgcgggtcggccatgatcgtcgcaccgctaagacgctggtgtggcatcggttgaccagccccgacaccgaccgctgggagcaattgccgcggattgtgtgggcgaggatctgccagatcgccggggagacgtcgggagagtgggtgaggcattggctggtgtggcggatgctggccggtcgtactggtgtggctcaggcccctacgtccatcgtggcccggttcctgggctgttcttcgcgccaggtctctcatatccgtggggctctcgttgatgccggtctgctcggctgtgacgaggtctcgggggcggcgagccgggtgtggttcccctcgatggccgaggagtgtgcagcggatcgtgaggtggtcgatggggccgtcgtcgatgctgaggtgggctctggtgaaggtgtcgaggagggcggggaacccctgtcgattttgtccacccacccctgtcgattttgtccacccacccctgtcgattttgtccgccaagtattagacaaaccattagacagtgaattagaccctgggtcgcgtcagcgtgtgaactcaccaacgcgcgcgcgcgcggcggccgagccgccgaagccaaagaaacccataccaccaacgcctgaaccagcagcccccgcttcagacagtcctgtgtcagcgcacgctgatgaggctgccgagttggcgtggcagttcgtggggtggtgtcctcagctggtaggggcaccgaagaaggtgcgtggccagctccgccagatggtcgctgccgagatccgccggaatccgggctggctggatgcgccagcgttggaggttgttgcccagcgggtacgtgctgaaggtgcgctgggggtccagcactgtgagattgtgcgagccgagctgcacggggtgatcgccgaccagaaggccgcccccgcccgccccgacgccctgcacgcgcgggagcccaagtggtgggacgacgtgtactacgaaggaaacacaaccgtgtggtacgactctgacgaaaccagtgacgacgacgaggcgtggaaccggtcgcaggtatgtcatgttgctgagcgtgacgacatggctggcgggattgctaccaacctgctggcctcccacgctgatgatccgatggcctggctgactcgccgtcaaaccatgctgatggctcgtttcccccacgcccagaacgaggtcatcgcgatgtgccacatcgtgcgcaccgccctggaggccgacatggccgccggaacccggtgggaggactgatgagggtctcccggcgcgccctcatggccgtggcccgtgccgacgctgccgcagcagaccatgccaccggccgatgggtggccacagccaacgccaccgtcgcccggctgaccggactgtgcgaacgcaccgtccaatacgcccgcgccaccctggtccacctgggcctgtgccgcgtcgtggccaccggccgctacctcaccgccgacgagcggcgcacggctgctcgcacccatggcggccgccagatgcgcgccgcctccacccgcgtcctcaccatgtcctgcgcaatcgtttgcgccctaccccgaaggggtcaccactacaggaaagttaagttcctagatggtcaccaacgcacgcgcaggcgcgtgcgaaggccagcaacaccagagaagaaacccctgtggctgcaaaaactcgcagccctcctcgaccagcacctgccctggctggtgagaaaccatcacatcggccgactgtgccacgccctcaccgccctgggcatcgacgaacactggcaacccgcagacctcgacgccctcctcgacgccatgaaccgcaggaaccgtcgcctcggcctcgacgtgccatgcgccgacgaccagcacaaccccttgggactgttcatccatcaggcccgagacaccctcacccacgacaacccccatgaacgccgcctagcccgtgacgctgaacgcgcccggctcatcgacgaccaactccgccgccgcgcagaaaccgccacctgcgcagcccgcctcgcagccgagcacgccgaccccgactaccagacccgccgtcgtgaacgcagggaaaccctgcgagccacactcagggccgcccagcagcatgtcaggacacactgatacccgctcatacgcacaccatgaaacacgacaagccacctgacagtatcagctgcaagacgcatgacaacatagaaggacatgatgaaatacacatagacacaaccccgcacagcagtttcgcaacaaatcatgatagtctcatggcagtatcgcttgatacacacatagcatcactcccacataggcagttatgctgatatactcctactaggttatccgtccaacatgctgagtgggttcctgtacagcatgaaaggagcatggcatgatatgggccgttctcaactgcaagggtggtgtcggcaagacgaccagtgccctcctgctggcggcagccgccgccaagcagggccacaccaccctggtggccgacgctgacccccagggcaccgcatcccagtggtcagccctggcaaccaaaaacgacgaacccctgcccttcccggtgcaagcggtcaacatcgccaccatggaggcactgcccaccaccaccgacgcccatgatctcatcctcgttgacacacccccctcggcaggcgatctgatgttccaggcatgccgcatcgccgacctcatcatcatccccaccgccacctccggacccgacatctcccgaacatgggtcaccatggacgccacacaaggcacaccacgcgcgatcctcctcacccaaaccgaacacaaccgtgtcgtctaccgccaagctcgcactgccctcgccgccgacgacaccgtcgttctcctggaccacgacatcccccgccgtgaatccatccgcacagcatggggcaccaaccccccagacgacgtcatcacctactacctgcccgtcctcaacgaactcatggaggccctgtcatgaccaccaagaaaaccgacccattcgcccgccagaaagcagcagaagaccgcgcccaccaactccaagacgaagcccaacgccacgcccaccccaaacgcgtcatcggccccaccgcacgattccagttcgtcctgccagccgacgtgctcaccggcctcaaacacctcgccctcacccgcggcaccaccgcccgccaactcaccctcgacgccctcgacaccgcctacggcctcgacaccctcgccaacagcgggaagaaaacccacccatgacccgtgggtccccctgccaccaccccctcgacggcaactgtgtgcgagccacaggcccgcacacaggcggcagcccagtgccgtccccgccaggaaaggaactgaacttttctggagtgctggctccctatcaccccttatggtgctatcagatccagtagcgccagcccgagaggaaacccatgagctacatcgtccgcaccggaaatctagccggcacccccgagctgcgcgaggggtagccgacacggcggaccgtgcccctgacagtaagcggcactactggagacatcaccatgaccgctcagatcctcgccccgtgcaaccagaaggctgccgtcatcgaaggagccctgccatgaaccaacctaccccccacaaggccgacgagtcacagacgagcaacaccacgaggaagtggcggcacaaggtgtcgttctaccaagacccggccgacaccgaccgagtccgcggagcgatcctccacaccatgacaaccgaaggcaaccggaacctgagccagttcgtcaacgatgccgtcatggccaaggtgaccgaactggaagcaaaatacaaccacggcgagccattccccgccgtgggagcccgaggactcccccagggcggcgcagccgccaacagataagagggcggggaccaccaatagcagtcgatttcggatccagatcgggtagacaggacccatgatgaatttcatatcctccctgctctcggccatcctgaccctctttgcgatggtagccgtggcgataggagtggtgaacattgtgccgggaatatgcgccatcatcggtgcacggaaatacggcgaccaggaccgtgtgcgggcaggacaacgaaagatccttcttggtgctatcgccttggtggtagcagtaggcattcacccgtgacccacggttaccggaagccccgccgcaatcgataccactgtcagtaccatcaggggcgataggggtcaagctctccttgtcacaatcccttccccggcagcccagtaaccgtcgcttacagccatacatggatatgcgcccattcgtcggcaccgacgccgtgcgggttggcgacgctgacccgtctgctcacaaacgacccttccagcccagcaccacccactacaccgacacgatccacgaagctgaaaccgaacctcccatccccgtccgaaacatcaacgaactcatcaaccccaccaaccatgcatgaacgcacgatcgcctaccggccagccttcctgcgcgacgtgaagcggctcaaaaacaagcactacaacatgaacacacactcaaatgggcggtaaggtcacgaagtcgacgtggctgcctcacggatggcttcggcgagctcttggggtcgactccacatgggccagtggccggttggcagatccatcgcggtgaagttggtcaggtgcgcgacggcgctgaacatcggattgcctgctgcggccagctcgaggacctggacgctgggaatggagcagcagatcaacgtggttagtacatcatggcgagcgctattggacagcgcgacccgtgcccgggccacttgcgcgggttcaggaaccgcccgtgcccggaaccggtcaaggtgctcgttgctgaggccctctaaactggcctgcttgccgagagtatcgaagtcaggaagtggcagctcagctgtttcctcaggtagatctggtgcgaaggcacctccatcgctcatgggaccggagtccacccagacgatccgatggaccaactctgggtgcctgtctacaactaggctcaccggcccgttcgcgccactgtgtcccaccaggaccgcatcgccgcccatctcgtccaggatggctgagatggcgtcagcttggtcgtcgagcgtgcgcgtagtgcgctgggtgtcctccggatcgaggccaggcagcgtcagccccacggctcgactgccagcagtgttcaataactcgactacctcgtcccaagcccaggctccgagccagtaaccaggaacgaggacgataggccgatgctggtcaggtcgcgatgttgaagtcatggagacatagttgcacagtgcgcggacaagggagtggcactatttctggcatgaatttagatctgggtgtccggtgaggcgatcagaacgcctccatgtactgtccgagtgtctacgatgcagcggacacagtcgagcagctggctcacaagtttccaggtgtccttctgcccctatgagggaagtcggctgctctcgcaacctttgagggctgctctggggtcggaaccagttacagatcgttcaagttccgggaactaccgccggtcacgctgattgattcccgggcgatctcgcttctgacgacagtggtcggtaaccgagaggcgtcctactctgaccccgctgctgcggtgagtaaatcacgggcgtcccggatccggccacccgtccaagtgccagccagcttgatcggctaggcatcgtcaaggagcgacgatgaggtgatgatgcgatgtgagcgcaccgcgaagtgaggtataaagaagccttcccgggcgcatgcgtcgcccaggaaggccaggttggcccctggacggctgaatggttgggcggatcaggctgtagcggagacgtgcagtacctcgtaggcgatggcgttggttgggctctggtctgcatcgtggctactgagttcgacgaggatgacggtccaggtggtgatgtcgacggcggtgacgcgggcgatggtggaatcagtggatgctgcaaggaagtcggcggttatgtcttcgatgcagtgggacggagcagtgctggtgggcaggggcacggtcttgacggtcaggcttcgggcagcagagtaatcggcggttccgttaacaagctggtggacggtccagtcctggatcgggtgccggccgaagtcgcgtacgatagccgagtaaccaagttcgccccagcagaactggcgcatcccctctatgtcgcgccagacgtagaagggcgcgtaggagttctggggtgcgcctttgaccttttcttggatgaggtaggccttgaactccagtccatggtagccgtccatgaggtgtccagtctggatgacgcggtcgcggatgatctgcatgtcatagtcggtgggcagggtgatctgatattgcatggcgtacatcagcggttctccttggtttcgtgggagtaggtcgtggggatgacgggttcctgccaatcgatggttgacaagtcgtcggcggcttgttggtaggtaaggtggatcattggctgtttgagtgcagcgccatgccagtgtctctggccggcgtcgatccagatggattcgcccttggtcaggtgctggggtgcttgtccttcaatatggaccagggcaacgccgcccgtgacgtagaggccctgtcccttggggtgtgtgcctcagcggcaaacgtgacggcgtgggcggcacgcccggcgtcttccgtggcacgatcggccggatccatggcggcgcgcagctgctgttctgcagccaggcgtccaggtcacggcgggcagcatcgacacccagggcagcctcgacggcttcagggctacgacgctggccgggctggttgaccgcagaccactggtgccgggcgtcgtccagatcatggccggcggcgtcgatgagggcggtggtggcttcggcgcagcatcgcctgatccactccgaacgtgacaggccagctgtctcggcggcactgtcaatgcgcgccaggagagatttcatgggcgctcatctgtagcctccagataggcgttgatgtagcgcagagcctgatccgtcatgtcgatggcggctgcctcggcagacgacacaacgggctgtggatgataagaccacccaacatagacggcgaacatcaatttctcgacagctttcatgaggtccccggcggcaaccgcatccgcctcatcatccgaggagaaccacttcaggtctgaacggctgtacatagcccctaggtcctgatgtgggtcgaggtccttggaatgatggaagttcctacacacccatcgccaaagacctcaacatggacaacgctaccttcaccactccctcatctgtgaagagccagtttatgcacggtgttgaaagaggcgtcggtctacgacgaatgcgggttaatggtcttgcggtgtggggcgctgggcgatggcatcgagaacgcgtgagtgggtggcgatccattccgcgtcggggttgatgttgcgggcgacggtgtcgatcgctgttacgtagtgggcaagcctgcgaatctcagcttgttggtcagggtgatcgctgatgagaggctcttgatgggaggcgcggttacggacgtggcggatgagctccacgttttcctcgaagtcgcgtcggctacgccccgggtagtgaggcatacctccgttacgtaagtcagtgagagctttccatacggttgcttccaggcgcggggtgagcagaaagcgccagttgtccagtgacagactcgccacgatctggatcaggggtcgcatcgagtcctcgcgcgcccgcagtcgcttctcggccctggcgacgttacgcgggaaaccaccgagccgatagctcggggattcccaccaacgccgtatgccagtatcggcgctcaggcgcacgtcaatgaagtttctcaggagtacctcgacatggctgatgtcttccaagaacgctttcgagatgcgggtgttccacacgtaccacgcatcgactggttcgatccggtagcggctgagcctggcgggtgagaaccactcctgcaacgtctcacgactcaccgtgggagcgtggtaggattgatgacgataggcctcgggaaggtcaatgccgcgaaatatcggtatacccccgaggtcattgttttgcccaggcccctcctcgcatacttgcggggcgtcattggctgccgtcatctcgtctcttcccggtacgtggctgtccatgttcacgggcaggtacggcaatgaggttcccgcacacaaacctctaccaagcatacgctggcaagccccgacgagacgacgcctccgcccgccacaccaacggtcaccgccacggccacggtgaccgtgaccgtggcaccgaaacccaagccaaagcccaaggccaagccaaaggtaaaacccaaaccgaagcccaaggcccattcatgatcgcagtgcccgatacaaggtggagcggcccacaccaagctgggccgccaccttcgttttcgggacaccctcggcaacgaggcggcgagcgcgggcaagctggccctcgctcaatgccggtttccggcctcgatagcggccctgcttcttcgccaccgcgatcccctcggcctgtcgttcatggatcaacgcccgctcgaactcggccaaggcacccagcagatgcaacatgagctggctcaccggatcagcggaccccggcccatagacctgcccttcaagggcaggcccgttatgctgtcagcagatctgccagttgtgggtgctggtgtcgtagcagacgccggtgtcggtgtcgtcggtgcggttggtggcccagccggtggtggagtcgtcgagggtgatgtggtggaggtcgggttgagtgttgagccagtgagtcccctcggcttggggtaggccatcgaggatggctagtaggtgggagatggtgg

SEQ ID NO: 7 - DNA, 1010 bp, Propionibacterium acnes
atgaagaaaaactggttactcacaaccctccttgccacaatgatgatcgccatgggcacgacgaccaccgccttcgccagcccgcctaccgacatcactcccgaacatccaggcggggttacccgcctcacagccccgacggaatcccctcgaatattgaggggccaagtatgcccagctggacctctgcaatcaggttcgcaatgaagaaccccggcacgaaagtcccgggcaccaacgacttcacctgcaaaccgaggaaaggcacccatcccgtcgtgctcatcccgggcacatccgaggacgccttcatcacgtggtcgtactacggtccccgcctcaaggcagcaggattctgcgcctacacgttcaactacaacccggaaacacatccgcttgtggaagccgctgagaccagcggcaacatctactccacggcagctttcatggcccacttcgttgacagagtgctcaaggcaaccggtgctcagaaggtcaacctcgtcggccattctcagggcggcggccccctgccgcgcgcgtacatcaaatattacggggcgccaagaaagtcctcatctcgtcggtttggttccttccaacaggggaacacgcatgctcggcctggagaagttcctcaatgccagcggaaacccgctcagcactatcttcaatgctgcagcacagtttcgaaagctggaatccctgccccaacagttgcaagactccacatttctcagggaactcaacgcggatggaatgaccgtccccggcatcacatacaccgtcatcgccacccagttcgacaaccgagtatttccgtggactaataccttcatcaatgagcccggggtcaagaacatcgtcatccaagacgtctgtcccttggaccacagcgcccacacggatatccctaggacccgatgacccttcagattgtcatcaacgccttggaccccgagcgggccgccccggtcacctgcaccattcgcccattcaggcccagttag

SEQ ID NO: 8 - DNA, 2387 bp, Propionibacterium acnes
atggcgctcgccgcttctcccacagtgacggacgccattgccgcccccgggcccgacagctggtcggcgctgtgcgagcgatggatcgacatcatcaccggacgcagagccgcccggacctctgacccacgtgcccgagcgatcatcgccaagaccgaccggaaggtcgccgagatcctcaccgacctcgtatccggctcgagccgtcgaccgttctgatctcggcagacctccgcaaggagcagtcgcccttcatcaccaagacagcccgagccatcgagtcgatggcctgcgcctgggccacacccgggtccagctaccacaaggatcccgaaatcctctccgcgtgcatcgaggggctcagggacttctgccgacttcggtacaacccctcccaggacgagtatgggaactggtgggactgggaggacggcgcgtcaagagctgtcgccgatgtcatgtgcatcctgcacgacgtcctgccgcccgaggtcatgtccgcagcggcagccggcatcgaccacttcatccccgacccctggttccagcagccggcgtcggtcaagcccactgccaaccccgttcagcccgtggtctcgacaggcgcgaatcgcatggacctgacccgtgccgtcatgtgccgttccatcgcgaccggcgacgagaagaggctgcgtcatgccgttgacggattgcctgacgcctggcgcgtcaccaccgaaggtgacggtttccgtgccgacggcggattcatccagcactcccacatcccctacaccggcggctacggcgacgtcctgttcagcggactggcaatgctcttcccgctggtctccgggatgaggttcgacatcgtcgaatcggctcgtaaggctttccacgaccaggtcgaacgcggcttcatccccgtcatgtacaacggccagatcctcgacgacgtgcgcggccgatccatctcgcgcatcaacgagtctgccgccatgcacggcatctcgatcgcccgtgccatgctcatgatggctgatgccctgccgacacaccgcgccgaacagtggcgagggatcgtgcacggttggatggctcgaaacaccttcgatcacctgtccgagccgtccacccttgtcgacatctccctgttcgacgccgccgccaaggcgcgccccgtcccggagtcgtcgacgccgagctacttcgcgtccatggaccgtctcgtccaccgcaccgcggactggctaatcaccgtctccaactgttcggatcgcattgcctggtacgagtacggcaacggggagaacgaatgggcgtccaggaccagccagggaatgcgttacctcctgctgcccggagacatgggacagtacgaggacgggtactgggccaccgtcgactactcagcaccgacggggacgacggtggactccactccgctcaaacgcgccgtcggagcctcgtgggcggccaagaccccgaccaacgaatggtccgggggcctcgcatcggggtcgtggtctgccgccgcgtcccacatcacctcccaggactccgccctcaaggcacgccgcctatgggtgggtctgaaggacgccatggtagagctgacgaccgacgtgaccaccgacgcatcgcgggccataaccgtcgtcgagcaccgcaaggtggccagctcgtcgacgaaactcctcgtcgacggcaaccgggtctcatccgcgacctccttccagaacccccggtgggcccatctggacggagtcggcggttacgtcttcgccactgacaccgatctctccgcagatgtggcgacgagaaagggaacgtggatcgacgtcaatccctcccgcaaggtcaagggggctgacgaggtcatcgagcgcgcctacgcatccctgcacgtcacccaccacgatcgtccagtcgcgtgggcgctgcttcccactgccagccgttcccacacgatggccctggccacgcgcccaggagtcgagccgttcaccgtgctccggaatgacgcaaccgtccaggccgtccgctctgcgggtgccctcttgacgaaggaccccactgtcgtcaccaccttggctttttggaagccagctacctgcggcggcgtggcagttaaccgtcctgcgctggtgcagactcgggagagcgcaaaccaaatggaggtcgtcatcgtcgaacccacccagaagaggggatcacttaccgtaactattgagggaagctggaaggtcaaaaccgcagatagccacgttgatgtcagctgcgaaaacgcggccgggactctgcatgtcgacacggcggggctaggcggccagtccgtgcgagtaacgctggcacgccaggtaactcaaactccctccggcggcggccgccacgaccgagcctga

SEQ ID NO: 9 - DNA, 1113 bp, Propionibacterium acnes
atgcgtgttggtgttcctactgaggttaagaatagtgagtttcgtgtggctgtgacgccggcgggtgttcatgcgttggttggtcgtggtcatgaggtgttggttcaggctggtgctggtgtgggttcgggtattccggattcggattttgtgggtgctggtgcgcgggttgtgggtgatgtggagtcggtgtggggtgatgctgatttggtgttgaaggtgaaggagcctgttgcggaggagtatgggcggttgcatgagggtttggttctttttacgtatcttcatttggctgctgatgaggcgttgactcgtgagcttttggggcgtggggtgacgtcgattgcgtatgagacggtggagttggctgatcattcgttgccgttgttgtctccgatgtcggagattgcgggtcggttggctgctcaggtgggtgcgaattgtttgttgcagtctgctgggggtcgtggtgtgttgtttggtggtggttcgggtgtgcgtcgtggtcgggtgagtgtgcttggtggtggtgtggctgggttgtgtgcggctcgtgtggctgcgggtatgggtgctgatgtgacggtgtttgatgtggatgtggcgcggatgcgttatatcgatgaggtgtggggtgggcgtattggtacgcggttttcgagtccgttggcggttcgggaggcgtgtggtgagtctgatgtggtgattgggtcggtgttggtgcctggtgctcggactccgcatttggtggatcatgagatggtgttggggatggtgccggggtcggtgttggtggatgttgcggtggatcagggtgggtgttttgaggattctcatccgacgacgcatgcggatccgacgtttgtggtggggggttcggtgttttattgtgtggcgaatatgccgggtgcggtgccgcatacgtcgacgtatgcgttgacgaatgcgacgatgcggtatgtgttgttgttggctgatgagggttggaggggtgcgtgtgggtcgcgtgatgatttgcggcgtggtttggcgacttgtgatgggaagttggtgagtgcgccggtgggtgaggcgttgggtattgagtgtgtgcctgtgtctgaggtgttgtga

SEQ ID NO: 10 - DNA, 952 bp, Propionibacterium acnes
atggacaaaccagcgatagagatacgcgatctcgtcaagtcgttcccccaggccggcagccgcgaacgcctcattgccgtcgaccacttgtcaatgacgatcaaccgcggagaggtcgtggccttcctcgggcccaacggcgccggtaaatccacgaccgtcgacatgctccttggcatgaccagacccgacagtgggaaggtcactgtcctgggctccgatccgagaacagccgcccgtactggttgcatcagcgccgtctttcaaactggcggactgctacccgacttcaccgtcgcagagaccatgaaggccatcgctgccgcacacgggcagcgctcgagggtcaagcccctcaccgagaggtgggagttggccccattcgccggaaccaaggtcggcaaatgctcggggggggggtcagcggcagcgacttcgtttcgccctcgcgatgctgcctaaccccgatgtgcttattctcgacgagccgacaaccggtctggacgttgaagctcgtcgacgcttctggcaggtcatgggtgaggaggccgacgccggacgtacggtcattttcgccacccactacatcgaggaggccgattccttcgcccgtcgcgtcgtcctcgtcagtggtggacagctcgtcgcggacggtcccatcaatgaggttcgcgcctcggtgtccggatccactgtcagagcgactctcactgatccctccgtactggccgagggtctgcgcaccttccctgggattaacgacatcaccgtccagggccagcaactcatcgtgcacactagtcagcctgacgacctggcacgtcaccttctgacctacaccgacgctcacgggctgctcatctcgacgatgacccttgaggacgccttcgtccggctcacaggctccaatgattcccgtgacgatgttgacgactgggaggcagcagcatga

SEQ ID NO: 11 - DNA, 621 bp, Propionibacterium acnes
atgatcgataagaccatgatcaagctcgtgctggccgacgaccagaccctcgtacgcggcgctctggcagccctgcttagcatggagaacgacctggagatcgttggtacgtgtggtcggggagacgaggtgttcgcccttgtgcaggccacccatgccgacgtgtgtctgctcgacatcgagatgccaggtatcgacgggatcaccgtcgccgctgagctgagagaccaggcatcgtggtgtcgagttctcatcgtgacgaccttcgggcggccgggttatctgcggcgggccatggatgccggtgtggctgggttcctcgtcaaggacaccccagctgaggacctggcccgagtggtgcgcgaggtccatgccggaggtcgagttatcgacccagctttggccgccgaatccctcatcgagggacacaatcccttgtcggagcgggaacgggagattctgcggctggccgagtcgggggcctccatctccctcatcgcctcccagctctatctgtcggtgggcacggtgcgcaaccacgtgtcctcggccatcggcaagacgggtgctgcgaatcgcaccgaggctgccgtcacggctcgacagcgggggtggttgtga

SEQ ID NO: 12 - DNA, 707 bp, Propionibacterium acnes
gtgaccgtcctgctcttacgcctcgcggggcccttgcaatcctggggagattccagccgatttacgacccgggccacacgacgggaaccgacgaagtccggcgtcatcgggttgttggctgcggcgcagggacgacgtcgcaccgacagtctcgaagacctgctcaccctgcgattcggtgtgcgaaccgaccagccggggtcgatcgtgcgggacttccagacagccatggactgggcacatcccaaaaaggacggccgcgtcaaagccatgccgttgtcaaaccgctactacttggccgacgcagtcttcgtggcggcggtcgaaggagacccgtcctcctccaggcgttggacgaggcaatcagagacccggagttcccgctctacctcggccgaagatcctgtcccacggaaggacaggtgtccttaggcgtgcgagagagcgagctggtgaagaccttggaaaatgagccttggcacgccaagctgtggcaccaacgccgactgggacgatccgtccgacttcccatcgcctatgacgccgggccgggacagatcggcgacaccgtccgcgacataccgctgagcttcaaccctgagcggcgcgagtacggctggcgcgacgtcacgacgacaacgattgtcgtcgacaaccccaacggaagcgacgagcccgactggttcgcaggcctagaaggagcttga

SEQ ID NO: 13 - DNA, 588 bp, Propionibacterium acnes
gtgagtcgcatcgtcatcgtcaggcacgggcagtcgacgtggaatcgtcaagggcgcatccaagggcagacaatgggtattcccctgacgatgctcgggagacgccaggctcgccaggccgctcacacggtggcaggtctggtaccccacgacaccccgatcatcgcctccgaccagaagcgggcgcgtcagacagctcgtcctatcgcgcgggtgctgggcgtaccagcgacgaccgatccacggctgcgcgagcaagggttgggagccatggagggtcacaccgcggatgatctcgagccccttccccagccaacgggtgtacatccggccgacgtgcgatgggctggtggggagtcgctcgcggatgtggcggagaggtgccgcagcctgttggatgacgtggcagctcgcgacctaccggcgatcgtcctcgtcacccacggtgacaccatgcgggtcctgctggggattctcgacggtcgcagtcaccgcgacctcgactgggaccttccactgacgaacggaagtgtcatagcgcgagatgtgaacctcagcgagtcgcatcggcggctttcgctgtcgtaa

SEQ ID NO: 14 - DNA, 1010 bp, Propionibacterium acnes
atgaagaaaaactggttactcacaaccctccttgccacaatgatgatcgccatgggcacgacgaccaccgccttcgccagcccgcctaccgacatcactcccgaacatccaggcggggttacccgcctcacagccccgacggaatcccctcgaatattgaggggccaagtatgcccagctggacctctgcaatcaggttcgcaatgaagaaccccggcacgaaagtcccgggcaccaacgacttcacctgcaaaccgaggaaaggcacccatcccgtcgtgctcatcccgggcacatccgaggacgccttcatcacgtggtcgtactacggtccccgcctcaaggcagcaggattctgcgcctacacgttcaactacaacccggaaacacatccgcttgtggaagccgctgagaccagcggcaacatctactccacggcagctttcatggcccacttcgttgacagagtgctcaaggcaaccggtgctcagaaggtcaacctcgtcggccattctcagggcggcggccccctgccgcgcgcgtacatcaaatattacggggcgccaagaaagtcctcatctcgtcggtttggttccttccaacaggggaacacgcatgctcggcctggagaagttcctcaatgccagcggaaacccgctcagcactatcttcaatgctgcagcacagtttcgaaagctggaatccctgccccaacagttgcaagactccacatttctcagggaactcaacgcggatggaatgaccgtccccggcatcacatacaccgtcatcgccacccggttcgacaaccgagtatttccgtggactaataccttcatcaatgagcccggggtcaagaacatcgtcatccaagacgtctgtcccttggaccacagcgcccacacggatatccctaggacccgatgacccttcagattgtcatcaacgccttggaccccgagcgggccgccccggtcacctgcaccattcgcccattcaggcccagttag